(19) United States
(12) Patent Application Publication
(10) Pub. No.: US 2023/0002374 A1
(43) Pub. Date: Jan. 5, 2023

(54) GPR52 MODULATOR COMPOUNDS

(71) Applicant: Heptares Therapeutics Limited, Cambridge, Cambridgeshire (GB)

(72) Inventors: Sarah Joanne Bucknell, Cambridge, Cambridgeshire (GB); Stephen Paul Watson, Cambridge, Cambridgeshire (GB); Michael Alistair O'Brien, Cambridge, Cambridgeshire (GB); Chris De Graaf, Cambridge, Cambridgeshire (GB); Nigel Alan Swain, Cambridge, Cambridgeshire (GB)

(21) Appl. No.: 17/774,081

(22) PCT Filed: Nov. 9, 2020
(86) PCT No.: PCT/GB2020/052832

§ 371 Date: May 3, 2022

(30) Foreign Application Priority Data
Nov. 8, 2019 (GB) 1916298.1
Dec. 20, 2019 (GB) 1918974.5
Dec. 20, 2019 (GB) 1918992.7

(51) Int. Cl. C07D 471/04; C07D 487/04; C07D 498/04

(52) U.S. Cl. CPC C07D 471/04; C07D 487/04; C07D 498/04

(57) ABSTRACT

The disclosures herein relate to novel compounds of Formula (1): (1) and salts thereof, wherein R¹, Q, X, Y and Z are defined herein, and their use in treating, preventing, ameliorating, controlling or reducing the risk of disorders associated with GPR52 receptors.

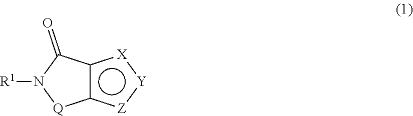

This application relates to novel compounds and their use as G-protein coupled receptor 52 (GPR52) modulators. Compounds described herein may be useful in the treatment or prevention of diseases in which GPR52 receptors are involved or in which modulation of GPR52 receptors may be beneficial. The application is also directed to pharmaceutical compositions comprising these compounds and the manufacture and use of these compounds and compositions in the prevention or treatment of diseases in which GPR52 receptors are involved or in which modulation of GPR52 receptors may be beneficial.

### BACKGROUND OF THE INVENTION

G-protein coupled receptor 52 (GPR52) is a constitutively active Gs coupled orphan receptor which is highly expressed in the striatum and cortex. In the striatum GPR52 is expressed exclusively on dopamine D2 medium spiny neurons and in the cortex it is found on cortical pyramidal neurons expressing dopamine D1 receptors (Komatsu et al, 2014, PLoS One 9:e90134). Based on its localization and functional coupling, GPR52 is proposed to play a role in the modulation of fronto-striatal and limbic dopamine and may therefore have utility in the treatment of neuropsychiatric disorders. GPR52 agonists are thought to be particularly relevant to the treatment of schizophrenia, where they are hypothesized to improve cognition and negative symptoms indirectly by potentiating D1 signalling but alleviate positive symptoms through inhibition of D2-mediated signalling in the striatum.

GPR52 agonists could be used to treat psychiatric disorders related to dysfunction of the mesolimbic and mesocortical pathways. Examples include treatment of the positive, negative and cognitive symptoms of schizophrenia, depression, attention-deficit hyperactivity disorder, anxiety disorders (generalised anxiety disorder, obsessive compulsive disorder, panic disorder), bipolar disorder, addiction/impulse-control disorders and autism spectrum disorders. Neuropsychiatric symptoms (e.g. psychosis, anhedonia, agitation, etc) of neurodegenerative diseases (e.g. Alzheimer's disease, Parkinson's disease, Huntington's disease, etc) could also be treated by GPR52 agonists. GPR52 expression in the pituitary gland and hypothalamus suggests utility for GPR52 modulators in pituitary and hypothalamic disorders, and there is preclinical evidence (Xiong et al, 2016, WO2016/176571) to suggest that GPR52 agonists could be useful in the treatment of hyperprolactinemia.

### THE INVENTION

The present invention provides compounds having activity as G protein-coupled receptor 52 (GPR52) modulators.

Provided is a compound of Formula (1):

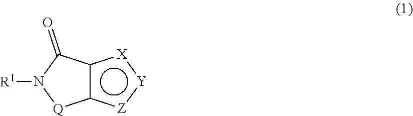

or a salt thereof, wherein;

X is N or CR²;

Y is N, NR³ or CR²;

Z is N or NR³;

wherein one but not both of Y and Z is NR³;

Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵—, —OCR⁴R⁵CR⁶R⁷— and —CR⁴R⁵O—;

R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring; R² is H, halo, CN, C₁₋₆ alkyl optionally substituted with OH or 1 to 6 fluorine atoms, C₁₋₆ alkoxy optionally substituted with OH or 1 to 6 fluorine atoms, or C₃₋₆ cycloalkyl optionally substituted with OH or 1 to 6 fluorine atoms wherein one atom of the C₁₋₆ alkyl, C₁₋₆ alkoxy or C₃₋₆ cycloalkyl group may be optionally replaced by a heteroatom selected from O, N, S and oxidised forms thereof;

R³ is a group -V-L-W;

R⁴, R⁵, R⁶, R⁷, R⁸ and R⁹ are independently selected from H and C₁₋₃ alkyl; wherein V is a 6-membered optionally substituted aryl or heteroaryl ring substituted with L at the meta-position relative to the position of attachment of R³ to the remainder of the molecule;

L is selected from CH₂, CHD, CD₂, CHF, CF₂, C═O, CHOH, O and NH;

and W is an optionally substituted monocyclic or polycyclic ring system.

Compounds of the present invention may be used as GPR52 modulators. Compounds of the present invention may be used as GPR52 agonists. Compounds of the present invention may be used in the manufacture of medicaments. The compounds or medicaments may be for use in treating, preventing, ameliorating, controlling or reducing the risk of diseases or disorders in which GPR52 receptors are involved. The compounds or medicaments may be for use in treating, preventing, ameliorating, controlling or reducing the risk of diseases or disorders in which modulation of GPR52 receptors may be beneficial. Compounds of the present invention may be useful in the treatment of psychiatric disorders; neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; depressive disorders; hypothalamic disorders; pituitary disorders; prolactin-related disorders; trauma- or stressor-related disorders; disruptive, impulse-control or conduct disorders; sleep-wake disorders; substance-related disorders; addictive disorders; behavioral disorders; hypofrontality; abnormalities in the tuberoinfundibular, mesolimbic, mesocortical, or nigrostriatal pathway; decreased activity in the striatum; cortical dysfunction; neurocognitive dysfunction or conditions or symptoms related thereto.

Compounds of the present invention may be useful in the treatment of schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, anhedonia, agitation, Alzheimer's disease, Parkinson's disease, Huntington's disease, vascular dementia, Lewy body disease, frontotemporal dementia, Tourette's syndrome, hyperprolactinemia, pituitary adenoma, prolactinoma, craniopharyngioma, Cushing's disease, diabetes insipidus, non-functioning tumours, obesity, posttraumatic stress disorder (PTSD), akathisia and associated movements, athetosis, ataxia, ballismus, hemiballismus, chorea, choreoathetosis, dyskinesia, tardive dyskinesia, neuroleptic-induced dyskinesia, myoclonus, mirror movement disorder, paroxysmal kinesigenic dyskinesia, restless legs syndrome, spasms, stereotypic movement disorder, sterotypy, Tic disorder, tremor, Wilson's disease, schizotypal personality disorder, delusional disorder, brief psychotic disorder, schizophreniform disorder, schizoaffective disorder, substance- or medication-induced psychotic disorder, delusions, hallucinations, disorganized thinking, grossly disorganized or abnormal motor behavior, catatonia, major depressive disorder, bipolar I disorder, bipolar II disorder, cyclothymic disorder, substance- or medication-induced bipolar and related disorders, bipolar and related disorders due to another medical condition, separation anxiety disorder, selective mutism, specific phobia, social anxiety disorder, panic disorder, agoraphobia, generalized anxiety disorder, substance- or medication-induced anxiety disorder, anxiety disorders due to another medical condition, delirium, major neurocognitive disorder, minor neurocognitive disorder, amnesia, dementia, developmental coordination disorder, stereotypic movement disorder, a post-stroke effect, dentatorubral-pallidoluysian atrophy, diminished emotional expression, avolition, alogia and asociality.

### DETAILED DESCRIPTION OF THE INVENTION

The invention relates to novel compounds. The invention also relates to the use of novel compounds as modulators of the GPR52 receptor. The invention further relates to the use of novel compounds in the manufacture of medicaments for use as GPR52 modulators. Compounds of the present invention may be used as GPR52 agonists. The compounds or medicaments may be for use in treating, preventing, ameliorating, controlling or reducing the risk of diseases or disorders in which GPR52 receptors are involved. The compounds or medicaments may be for use in treating, preventing, ameliorating, controlling or reducing the risk of diseases or disorders in which modulation of GPR52 receptors may be beneficial.

The invention further relates to compounds, compositions and medicaments that may be useful in the treatment of psychiatric disorders; neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; depressive disorders; prolactin-related disorders; trauma- or stressor-related disorders; disruptive, impulse-control or conduct disorders; sleep-wake disorders; substance-related disorders; addictive disorders; behavioral disorders; hypofrontality; abnormalities in the tuberoinfundibular, mesolimbic, mesocortical, or nigrostriatal pathway; decreased activity in the striatum; cortical dysfunction; neurocognitive dysfunction or conditions or symptoms related thereto.

Provided is a compound of Formula (1′):

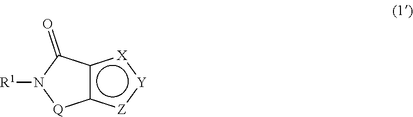

or a salt thereof, wherein;

X is N or CR²;

Y is N, NR³ or CR²;

Z is N or NR³;

wherein one but not both of Y and Z is NR³;

Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵—, —OCR⁴R⁵CR⁶R⁷— and —CR⁴R⁵O—;

R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring;

R² is H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof;

R³ is a group -V-L-W;

R⁴, R⁵, R⁶, R⁷, R⁸ and R⁹ are independently selected from H and C₁₋₃ alkyl; wherein V is a 6-membered optionally substituted aryl or heteroaryl ring substituted with L at the meta-position relative to the position of attachment of R³ to the remainder of the molecule; L is selected from CH₂, CHD, CD₂, CHF, CF₂, C═O, CHOH, O and NH;

and W is an optionally substituted monocyclic or polycyclic ring system.

Also provided is a compound of Formula (1):

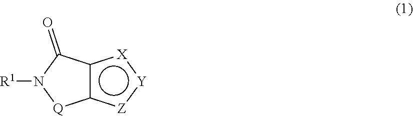

or a salt thereof, wherein;

X is N or CR²;

Y is N, NR³ or CR²;

Z is N or NR³;

wherein one but not both of Y and Z is NR³;

Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵— and —OCR⁴R⁵CR⁶R⁷—;

R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring;

R² is H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof;

R³ is a group -V-L-W;

R⁴, R⁵, R⁶, R⁷, R⁸ and R⁹ are independently selected from H and C₁₋₃ alkyl;

wherein V is a 6-membered optionally substituted aryl or heteroaryl ring substituted with L at the meta-position relative to the position of attachment of R³ to the remainder of the molecule;

L is selected from CH₂, CHD, CD₂, CHF, CF₂, C═O, CHOH, O and NH;

and W is an optionally substituted monocyclic or polycyclic ring system.

Also provided is a compound of Formula (1a):

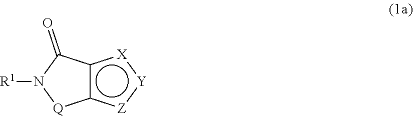

or a salt thereof, wherein;

X is N or CR²;

Y is N, NR³ or CR²;

Z is N or NR³;

wherein one but not both of Y and Z is NR³;

Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵—, —OCR⁴R⁵CR⁶R⁷— and —CR⁴R⁵O—;

R¹ is H, C₁₋₃ alkyl or C₃₋₆ cycloalkyl;

R² is H, halo, C₁₋₃ alkyl or C₃₋₆ cycloalkyl;

R³ is a group -V-L-W;

R⁴, R⁵, R⁶, R⁷, R⁸ and R⁹ are independently selected from H and C₁₋₃ alkyl; wherein V is a 6-membered optionally substituted aryl or heteroaryl ring substituted with L at the meta-position;

L is selected from CH₂, CHD, CD₂, CHF, CF₂, C═O, CHOH, O and NH;

and W is an optionally substituted monocyclic or polycyclic ring system.

Also provided is a compound of Formula (1b):

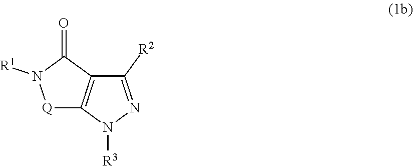

or a salt thereof, wherein Q, R¹, R² and R³ are as defined herein.

Also provided is a compound of Formula (1b):

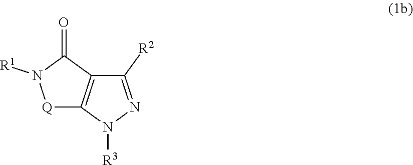

or a salt thereof, wherein;

Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵— and —OCR⁴R⁵CR⁶R⁷—

R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring; R² is H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof;

R⁴, R⁵, R⁶, R⁷, R⁸ and R⁹ are independently selected from H and C₁₋₃ alkyl; R³ is a group of the formula:

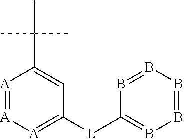

wherein, each A is independently N or CR¹⁰;

L is selected from CH₂, CHD, CD₂, CHF, CF₂, C═O, CHOH, O and NH;

each B is independently N, CR¹¹, CR¹², CR¹³, CR¹⁵ or CR¹⁶;

R¹⁰ is selected from H, halo and C₁₋₃ alkyl optionally substituted with 1 to 6 fluorine atoms;

R¹¹, R¹², R¹³, R¹⁵ and R¹⁶ are independently selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃.

Also provided is a compound of Formula (1b):

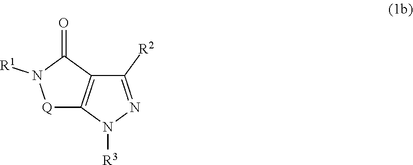

or a salt thereof, wherein;

Q is a C₁₋₃ alkyl linker;

R¹ is H or C₁₋₃ alkyl;

R² is selected from CN, halo, C₁₋₆ alkyl optionally substituted with OH or 1 to 6 fluorine atoms and C₁₋₆ alkoxy optionally substituted with OH or 1 to 6 fluorine atoms, wherein one atom of the C₁₋₆ alkyl or C₁₋₆ alkoxy group may be optionally replaced by a heteroatom selected from O, N, S and oxidised forms thereof;

R³ is a group of the formula:

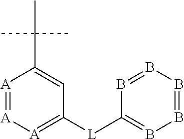

wherein, A is N or CR¹⁰;

L is selected from CH₂, CHD, CD₂, CHF, CF₂, C═O, CHOH, O and NH;

B is N, CR¹¹, CR¹² or CR¹³;

R¹⁰ is selected from H, halo and C₁₋₃ alkyl optionally substituted with 1 to 6 fluorine atoms;

R¹¹, R¹² and R¹³ are independently selected from H, CN, halo, C₁₋₆ alkyl optionally substituted with 1 to 6 fluorine atoms and C₁₋₆ alkoxy optionally substituted with 1 to 6 fluorine atoms, wherein one atom of the C₁₋₆ alkyl or C₁₋₆ alkoxy group may be optionally replaced by a heteroatom selected from O, N, S and oxidised forms thereof.

In the compounds herein, X can be N or CR². X can be N. X can be CR².

In the compounds herein, Y can be N, NR³ or CR². Y can be N. Y can be NR³. Y can be CR². When Y is NR³, Z is N.

In the compounds herein, Z can be N or NR³. Z can be N. Z can be NR³. When Z is NR³, Y is N or CR².

In the compounds herein, one but not both of Y and Z is NR³. Y can be NR³ and Z can be N. Z can be NR³ and Y can be N. Z can be NR³ and Y can be CR².

In the compounds herein, R¹ can be H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring. R¹ can be H, C₁₋₃ alkyl or C₃₋₆ cycloalkyl. R¹ can be H or a C₁₋₃ alkyl group. R¹ can be H. R¹ can be a C₁₋₆ alkyl group optionally substituted with 1 to 6 fluorine atoms. R¹ can be a C₁₋₃ alkyl group optionally substituted with 1 to 6 fluorine atoms. R¹ can be a C₃₋₆ cycloalkyl group optionally substituted with 1 to 6 fluorine atoms. R¹ can be a C₁₋₆ alkyl group. R¹ can be a C₁₋₃ alkyl group. R¹ can be a C₃₋₆ cycloalkyl group. R¹ can be —C(R¹⁴)₂C(R¹⁸)₂OR¹ or —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring. R¹ can be H, methyl, —CH₂CH₂OCH₃, —CH₂CH₂OH, CH(CH₃)CH₂OCH₃, —CH(CH₃)CH₂OH, —CH₂CH(CH₃)OCH₃ or —CH₂CH(CH₃)OH. R¹ can be H, methyl, —CH₂CH₂OCH₃ or —CH₂CH₂OH. R¹ can be H or methyl. R¹ can be methyl. R¹ can be an oxolane ring. R¹ can be a tetrahydrofuran ring. R¹ can be an oxetane ring.

R¹ can be

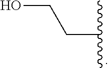 [SMILES: *CCO]

### R¹ can be

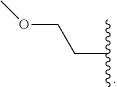 [SMILES: *CCOC]

### R¹ can be

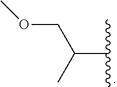 [SMILES: *C(C)COC]

### R¹ can be

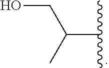 [SMILES: *C(C)CO]

### R¹ can be

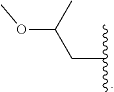 [SMILES: *CC(C)OC]

### R¹ can be

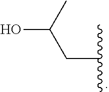 [SMILES: *CC(C)O]

### R¹ can be

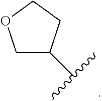 [SMILES: *C1CCOC1]

### R¹ can be

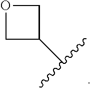 [SMILES: *C1COC1]

In the compounds herein, R² can be H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof.

R² can be H, halo, CN, C₁₋₆ alkyl optionally substituted with OH or 1 to 6 fluorine atoms, C₁₋₆ alkoxy optionally substituted with OH or 1 to 6 fluorine atoms, or C₃₋₆ cycloalkyl optionally substituted with OH or 1 to 6 fluorine atoms.

R² can be H, halo, C₁₋₃ alkyl or C₃₋₆ cycloalkyl. R² can be H or a C₁₋₃ alkyl group. R² can be H.

R² can be halo. R² can be a C₁₋₃ alkyl group. R² can be a C₃₋₆ cycloalkyl group. R² can be H or methyl. R² can be methyl. R² can be F. R² can be Cl. R² can be Br. R² can be selected from CN, halo, C₁₋₆ alkyl optionally substituted with OH or 1 to 6 fluorine atoms and C₁₋₆ alkoxy optionally substituted with OH or 1 to 6 fluorine atoms, wherein one atom of the C₁₋₆ alkyl or C₁₋₆ alkoxy group may be optionally replaced by a heteroatom selected from O, N, S and oxidised forms thereof. R² can be selected from methyl, F, Cl, CN, CF₃ and CH₂OH. R² can be C₁₋₆ alkyl optionally substituted with OH. R² can be C₁₋₆ alkyl optionally substituted with 1 to 6 fluorine atoms. R² can be C₁₋₃ alkyl optionally substituted with OH. R² can be C₁₋₃ alkyl optionally substituted with 1 to 6 fluorine atoms.

In the compounds herein, R³ can be a group -V-L-W.

In the compounds herein, V can be a 6-membered optionally substituted aryl or heteroaryl ring substituted with L at the meta-position relative to the position of attachment of R³ to the remainder of the molecule. V can be a 6-membered optionally substituted aryl or heteroaryl ring substituted with L at the meta-position. V can be a 6-membered optionally substituted aryl ring substituted with L at the meta-position. V can be a 6-membered optionally substituted heteroaryl ring substituted with L at the meta-position. V can be a phenyl ring substituted with L at the meta-position. V can be a pyridine ring substituted with L at the meta-position. V can be a pyrimidine ring substituted with L at the meta-position. V can be a pyridazine ring substituted with L at the meta-position. The term “meta-position” as used herein in relation to L should be interpreted to mean that substituent -L-W is positioned at the meta-position (or 3-position) of ring V relative to the point of attachment of ring V to Z or Y, i.e. relative to the position of attachment of R³ to the remainder of the molecule.

In the compounds herein, L can be selected from CH₂, CHD, CD₂, CHF, CF₂, C═O, CHOH, O or NH. L can be CH₂. L can be CHD. L can be CD₂. L can be CHF. L can be CF₂. L can be C═O. L can be CHOH. L can be O. L can be NH.

In the compounds herein, W can be an optionally substituted monocyclic or polycyclic ring system. W can be an optionally substituted monocyclic ring system. W can be an optionally substituted polycyclic ring system. W can be a 6-membered optionally substituted aryl or heteroaryl ring. W can be a 6-membered optionally substituted aryl ring. W can be a 6-membered optionally substituted heteroaryl ring. W can be an optionally substituted phenyl ring. W can be an optionally substituted pyridine ring. W can be a 9-10-membered optionally substituted heterobicyclic ring system. W can be a 9-10-membered heterobicyclic ring system. W can be a 9-10-membered optionally substituted heterobicyclic ring system, where one or more of the rings is aromatic. W can be a 9-membered optionally substituted heterobicyclic ring system, where one or more of the rings is aromatic. W can be a 10-membered optionally substituted heterobicyclic ring system, where one or more of the rings is aromatic.

W can be a 9-10-membered optionally substituted heterobicyclic ring system selected from:

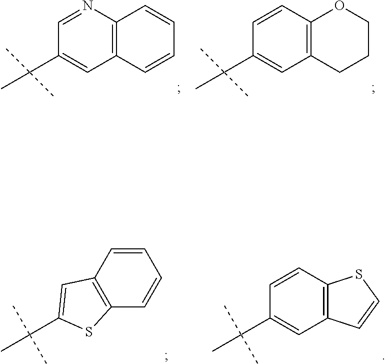

W can be an optionally substituted polycyclic ring selected from:

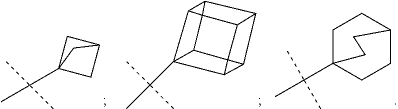

W can be an optionally substituted polycyclic ring system selected from: quinoline, 3,4-dihydro-2H-1-benzopyran, 1-benzothiophene, bicyclo[1.1.1]pentane, cubane and bicyclo[2.2.2]octane.

In the compounds herein, R³ can be a group of the formula:

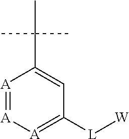

wherein, A is N or CR¹⁰;

L is selected from CH₂, CHD, CD₂, CHF, CF₂, C═O, CHOH, O and NH;

R¹⁰ is selected from H, halo and C₁₋₃ alkyl optionally substituted with 1 to 6 fluorine atoms; and W is either:

(i) a 6-membered optionally substituted aryl or heteroaryl ring;

(ii) a 9-10-membered optionally substituted heterobicyclic ring system, where one or more of the rings is aromatic;

or

(iii) an optionally substituted polycyclic ring system selected from:

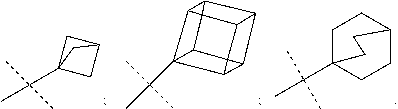

In the compounds herein R³ can be a group of the formula:

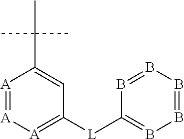

wherein, A is N or CR¹⁰;

L is selected from CH₂, CHD, CD₂, CHF, CF₂, C═O, CHOH, O and NH;

B is N, CR¹¹, CR¹² or CR¹³;

R¹⁰ is selected from H, halo and C₁₋₃ alkyl optionally substituted with 1 to 6 fluorine atoms; R¹¹, R¹² and R¹³ are independently selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃.

In the compounds herein R³ can be a group of the formula:

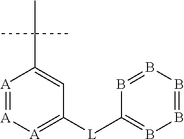

wherein, each A is independently N or CR¹⁰;

L is selected from CH₂, CHD, CD₂, CHF, CF₂, C═O, CHOH, O and NH;

each B is independently N, CR¹¹, CR¹², CR¹³, CR¹⁵ or CR¹⁶;

R¹⁰ is selected from H, halo and C₁₋₃ alkyl optionally substituted with 1 to 6 fluorine atoms;

R¹¹, R¹², R¹³, R¹⁵ and R¹⁶ are independently selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃.

In the compounds herein, A can be N or CR¹⁰. A can be N. A can be CR¹⁰. Each A can independently be N or CR¹⁰.

In the compounds herein, R¹⁰ can be H, halo or C₁₋₃ alkyl optionally substituted with 1 to 6 fluorine atoms. R¹⁰ can be H. R¹⁰ can be halo. R¹⁰ can be F. R¹⁰ can be Cl. R¹⁰ can be Br. R¹⁰ can be a C₁₋₃ alkyl group optionally substituted with 1 to 6 fluorine atoms. R¹⁰ can be a C₁₋₃ alkyl group. R¹⁰ can be methyl. R¹⁰ can be CF₃.

In the compounds herein, B can be selected from N, CR¹¹, CR¹² or CR¹³. B can be N. B can be CR¹¹. B can be CR¹². B can be CR¹³. Each B can independently be N, CR¹¹, CR¹² or CR¹³. B can be selected from N, CR¹¹, CR¹², CR¹³, CR¹⁵ or CR¹⁶. B can be N. B can be CR¹¹. B can be CR¹². B can be CR¹³. B can be CR¹⁵. B can be CR¹⁶. Each B can independently be N, CR¹¹, CR¹², CR¹³, CR¹⁵ or CR¹⁶.

In the compounds herein, R³ can be selected from group consisting of:

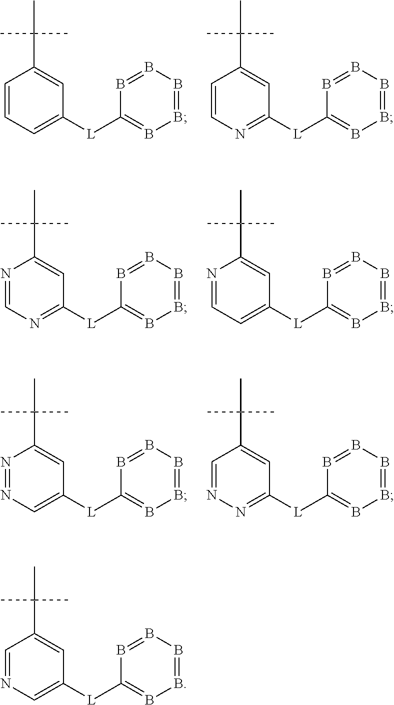

In the compounds herein, the group:

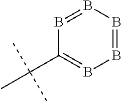

can be selected from the group consisting of:

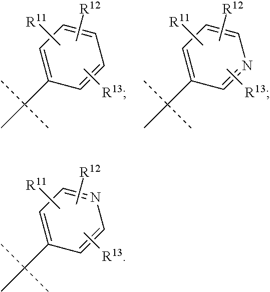

In the compounds herein, the group:

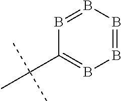

can be selected from the group consisting of:

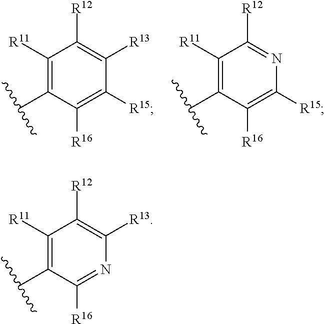

In the compounds herein, W can be selected from the group consisting of:

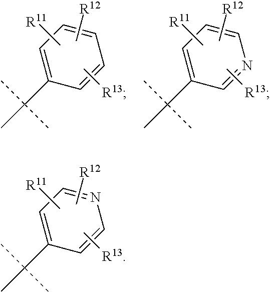

In the compounds herein, W can be selected from the group consisting of:

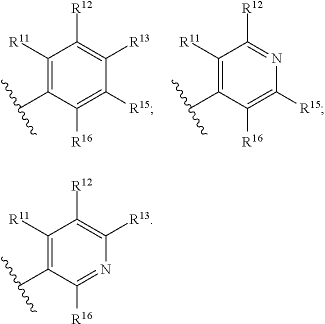

In the compounds herein, R¹¹, R¹² and R¹³ can independently be selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃. R¹¹, R¹² and R¹³ can independently be H, CN, halo, C₁₋₆ alkyl optionally substituted with 1 to 6 fluorine atoms wherein one atom of the C₁₋₆ alkyl group may be optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms. R¹¹, R¹² and R¹³ can be independently selected from H, CN, halo, C₁₋₆ alkyl optionally substituted with 1 to 6 fluorine atoms and C₁₋₆ alkoxy optionally substituted with 1 to 6 fluorine atoms, wherein one atom of the C₁₋₆ alkyl or C₁₋₆ alkoxy group may be optionally replaced by a heteroatom selected from O, N, S and oxidised forms thereof. R¹¹, R¹² and R¹³ can independently be selected from H, CN, SF₅, halo, a C₁₋₆ saturated hydrocarbon group optionally substituted with 1 to 6 fluorine atoms or OC₁₋₆ alkyl optionally substituted with 1 to 6 fluorine atoms.

In the compounds herein, R¹¹, R¹², R¹³, R¹⁵ and R¹⁶ can independently be selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃. R¹¹, R¹², R¹³, R¹⁵ and R¹⁶ can independently be H, CN, halo, C₁₋₆ alkyl optionally substituted with 1 to 6 fluorine atoms wherein one atom of the C₁₋₆ alkyl group may be optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms. R¹¹, R¹², R¹³, R¹⁵ and R¹⁶ can be independently selected from H, CN, halo, C₁₋₆ alkyl optionally substituted with 1 to 6 fluorine atoms and C₁₋₆ alkoxy optionally substituted with 1 to 6 fluorine atoms, wherein one atom of the C₁₋₆ alkyl or C₁₋₆ alkoxy group may be optionally replaced by a heteroatom selected from O, N, S and oxidised forms thereof. R¹¹, R¹², R¹³, R¹⁵ and R¹⁶ can independently be selected from H, CN, SF₅, halo, a C₁₋₆ saturated hydrocarbon group optionally substituted with 1 to 6 fluorine atoms or OC₁₋₆ alkyl optionally substituted with 1 to 6 fluorine atoms.

In the compounds herein, R¹¹, R¹² and R¹³ can independently be H, CN, SF₅, F, Cl, methyl, ethyl, isopropyl, cyclopropyl, CF₃, CF₂H, OCF₂H, OMe and SO₂Me. R¹¹, R¹² and R¹³ can be independently selected from H, F and CF₃.

In the compounds herein, R¹¹, R¹², R¹³, R¹⁵ and R¹⁶ can independently be H, CN, SF₅, F, Cl, methyl, ethyl, isopropyl, cyclopropyl, CF₃, CF₂H, OCF₂H, OMe and SO₂Me. R¹¹, R¹², R¹³, R¹⁵ and R¹⁶ can be independently selected from H, F and CF₃.

In the compounds herein, R¹¹ can be H. R¹¹ can be CN. R¹¹ can be halo. R¹¹ can be F or Cl. R¹¹ can be F. R¹¹ can be SF₅. R¹¹ can be a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof. R¹¹ can be a C₁₋₆ alkyl group which is optionally substituted with 1 to 6 fluorine atoms wherein one atom of the C₁₋₆ alkyl group may be optionally replaced by a heteroatom selected from O, N, S and oxidised forms thereof. R¹¹ can be a C₁₋₆ alkyl group which is optionally substituted with 1 to 6 fluorine atoms. R¹¹ can be a C₁₋₆ alkyl group. R¹¹ can be a OC₁₋₆ alkyl group which is optionally substituted with 1 to 6 fluorine atoms. R¹¹ can be a OC₁₋₆ alkyl group. R¹¹ can be a C₁₋₆ alkoxy group which is optionally substituted with 1 to 6 fluorine atoms, R¹¹ can be a C₁₋₆ alkoxy group. R¹¹ can be a SO₂C₁₋₆ alkyl group which is optionally substituted with 1 to 6 fluorine atoms. R¹¹ can be a SO₂C₁₋₆ alkyl group. R¹¹ can be a C₃₋₆ cycloalkyl group which is optionally substituted with 1 to 6 fluorine atoms or optionally substituted with CF₃. R¹¹ can be a C₃₋₆ cycloalkyl group. R¹¹ can be H. R¹¹ can be CN. R¹¹ can be F. R¹¹ can be Cl. R¹¹ can be methyl. R¹¹ can be ethyl. R¹¹ can be isopropyl. R¹¹ can be cyclopropyl. R¹¹ can be CF₃. R¹¹ can be OCF₂H. R¹¹ can be SO₂Me. R¹¹ can be CF₂H. R¹¹ can be OMe. R¹¹ can be

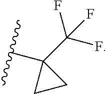 [SMILES: *C1(C(F)(F)F)CC1]

In the compounds herein, R¹² can be H. R¹² can be CN. R¹² can be halo. R¹² can be F or Cl. R¹² can be F. R¹² can be SF₅. R¹² can be a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof. R¹² can be a C₁₋₆ alkyl group which is optionally substituted with 1 to 6 fluorine atoms wherein one atom of the C₁₋₆ alkyl group may be optionally replaced by a heteroatom selected from O, N, S and oxidised forms thereof. R¹² can be a C₁₋₆ alkyl group which is optionally substituted with 1 to 6 fluorine atoms. R¹² can be a C₁₋₆ alkyl group. R¹² can be a OC₁₋₆ alkyl group which is optionally substituted with 1 to 6 fluorine atoms. R¹² can be a OC₁₋₆ alkyl group. R¹² can be a C₁₋₆ alkoxy group which is optionally substituted with 1 to 6 fluorine atoms, R¹² can be a C₁₋₆ alkoxy group. R¹² can be a SO₂C₁₋₆ alkyl group which is optionally substituted with 1 to 6 fluorine atoms. R¹² can be a SO₂C₁₋₆ alkyl group. R¹² can be a C₃₋₆ cycloalkyl group which is optionally substituted with 1 to 6 fluorine atoms or optionally substituted with CF₃. R¹² can be a C₃₋₆ cycloalkyl group. R¹² can be H. R¹² can be CN. R¹² can be F. R¹² can be Cl. R¹² can be methyl. R¹² can be ethyl. R¹² can be isopropyl. R¹² can be cyclopropyl. R¹² can be CF₃. R¹² can be OCF₂H. R¹² can be SO₂Me. R¹² can be CF₂H. R¹² can be OMe. R¹² can be

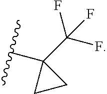 [SMILES: *C1(C(F)(F)F)CC1]

In the compounds herein, R¹³ can be H. R¹³ can be CN. R¹³ can be halo. R¹³ can be F or Cl. R¹³ can be F. R¹³ can be SF₅. R¹³ can be a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof. R¹³ can be a C₁₋₆ alkyl group which is optionally substituted with 1 to 6 fluorine atoms wherein one atom of the C₁₋₆ alkyl group may be optionally replaced by a heteroatom selected from O, N, S and oxidised forms thereof. R¹³ can be a C₁₋₆ alkyl group which is optionally substituted with 1 to 6 fluorine atoms. R¹³ can be a C₁₋₆ alkyl group. R¹³ can be a OC₁₋₆ alkyl group which is optionally substituted with 1 to 6 fluorine atoms. R¹³ can be a OC₁₋₆ alkyl group. R¹³ can be a C₁₋₆ alkoxy group which is optionally substituted with 1 to 6 fluorine atoms, R¹³ can be a C₁₋₆ alkoxy group. R¹³ can be a SO₂C₁₋₆ alkyl group which is optionally substituted with 1 to 6 fluorine atoms. R¹³ can be a SO₂C₁₋₆ alkyl group. R¹³ can be a C₃₋₆ cycloalkyl group which is optionally substituted with 1 to 6 fluorine atoms or optionally substituted with CF₃. R¹³ can be a C₃₋₆ cycloalkyl group. R¹³ can be H. R¹³ can be CN. R¹³ can be F. R¹³ can be Cl. R¹³ can be methyl. R¹³ can be ethyl. R¹³ can be isopropyl. R¹³ can be cyclopropyl. R¹³ can be CF₃. R¹³ can be OCF₂H. R¹³ can be SO₂Me. R¹³ can be CF₂H. R¹³ can be OMe. R¹³ can be

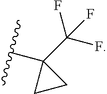 [SMILES: *C1(C(F)(F)F)CC1]

In the compounds herein, R¹⁵ can be H. R¹⁵ can be CN. R¹⁵ can be halo. R¹⁵ can be F or Cl. R¹⁵ can be F. R¹⁵ can be SF₅. R¹⁵ can be a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof. R¹⁵ can be a C₁₋₆ alkyl group which is optionally substituted with 1 to 6 fluorine atoms wherein one atom of the C₁₋₆ alkyl group may be optionally replaced by a heteroatom selected from O, N, S and oxidised forms thereof. R¹⁵ can be a C₁₋₆ alkyl group which is optionally substituted with 1 to 6 fluorine atoms. R¹⁵ can be a C₁₋₆ alkyl group. R¹⁵ can be a OC₁₋₆ alkyl group which is optionally substituted with 1 to 6 fluorine atoms. R¹⁵ can be a OC₁₋₆ alkyl group. R¹⁵ can be a C₁₋₆ alkoxy group which is optionally substituted with 1 to 6 fluorine atoms, R¹⁵ can be a C₁₋₆ alkoxy group. R¹⁵ can be a SO₂C₁₋₆ alkyl group which is optionally substituted with 1 to 6 fluorine atoms. R¹⁵ can be a SO₂C₁₋₆ alkyl group. R¹⁵ can be a C₃₋₆ cycloalkyl group which is optionally substituted with 1 to 6 fluorine atoms or optionally substituted with CF₃. R¹⁵ can be a C₃₋₆ cycloalkyl group. R¹⁵ can be H. R¹⁵ can be CN. R¹⁵ can be F. R¹⁵ can be Cl. R¹⁵ can be methyl. R¹⁵ can be ethyl. R¹⁵ can be isopropyl. R¹⁵ can be cyclopropyl. R¹⁵ can be CF₃. R¹⁵ can be OCF₂H. R¹⁵ can be SO₂Me. R¹⁵ can be CF₂H. R¹⁵ can be OMe. R¹⁵ can be

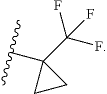 [SMILES: *C1(C(F)(F)F)CC1]

In the compounds herein, R¹⁶ can be H. R¹⁶ can be CN. R¹⁶ can be halo. R¹⁶ can be F or Cl. R¹⁶ can be F. R¹⁶ can be SF₅. R¹⁶ can be a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof. R¹⁶ can be a C₁₋₆ alkyl group which is optionally substituted with 1 to 6 fluorine atoms wherein one atom of the C₁₋₆ alkyl group may be optionally replaced by a heteroatom selected from O, N, S and oxidised forms thereof. R¹⁶ can be a C₁₋₆ alkyl group which is optionally substituted with 1 to 6 fluorine atoms. R¹⁶ can be a C₁₋₆ alkyl group. R¹⁶ can be a OC₁₋₆ alkyl group which is optionally substituted with 1 to 6 fluorine atoms. R¹⁶ can be a OC₁₋₆ alkyl group. R¹⁶ can be a C₁₋₆ alkoxy group which is optionally substituted with 1 to 6 fluorine atoms, R¹⁶ can be a C₁₋₆ alkoxy group. R¹⁶ can be a SO₂C₁₋₆ alkyl group which is optionally substituted with 1 to 6 fluorine atoms. R¹⁶ can be a SO₂C₁₋₆ alkyl group. R¹⁶ can be a C₃₋₆ cycloalkyl group which is optionally substituted with 1 to 6 fluorine atoms or optionally substituted with CF₃. R¹⁶ can be a C₃₋₆ cycloalkyl group. R¹⁶ can be H. R¹⁶ can be CN. R¹⁶ can be F. R¹⁶ can be Cl. R¹⁶ can be methyl. R¹⁶ can be ethyl. R¹⁶ can be isopropyl. R¹⁶ can be cyclopropyl. R¹⁶ can be CF₃. R¹⁶ can be OCF₂H. R¹⁶ can be SO₂Me. R¹⁶ can be CF₂H. R¹⁶ can be OMe. R¹⁶ can be

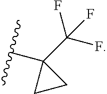 [SMILES: *C1(C(F)(F)F)CC1]

In the compounds herein, each R¹⁴ can independently be H, F or C₁₋₃ alkyl optionally substituted with 1 to 6 fluorine atoms. R¹⁴ can be H, F or C₁₋₃ alkyl optionally substituted with 1 to 6 fluorine atoms. R¹⁴ can be H. R¹⁴ can be F. R¹⁴ can be methyl. One R¹⁴ can be joined to R¹⁷ to form an oxolane or oxetane ring. R¹⁴ can be joined to R¹⁷ to form an oxolane or oxetane ring.

In the compounds herein, R¹⁷ can be C₁₋₃ alkyl optionally substituted with 1 to 6 fluorine atoms. R¹⁷ can be methyl. R¹⁷ can be joined to one R¹⁴ to form an oxolane or oxetane ring. R¹⁷ can be joined to R¹⁴ to form an oxolane or oxetane ring.

In the compounds herein, each R¹⁸ can independently be H, F or C₁₋₃ alkyl optionally substituted with 1 to 6 fluorine atoms. R¹⁸ can be H, F or C₁₋₃ alkyl optionally substituted with 1 to 6 fluorine atoms. R¹⁸ can be H. R¹⁸ can be F. R¹⁸ can be methyl.

In the compounds herein, each R¹⁹ can independently be H or C₁₋₃ alkyl optionally substituted with 1 to 6 fluorine atoms. R¹⁹ can be H or C₁₋₃ alkyl optionally substituted with 1 to 6 fluorine atoms. R¹⁹ can be H. R¹⁹ can be methyl.

In the compounds herein, R³ can be selected from the group consisting of:

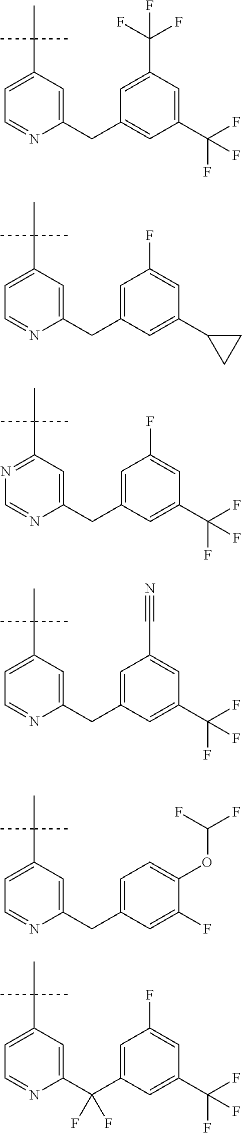

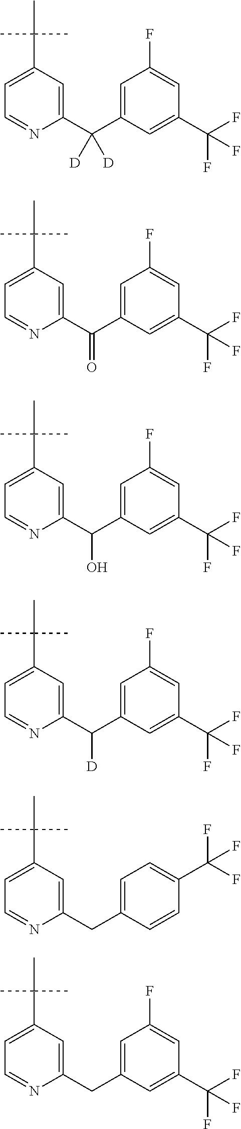

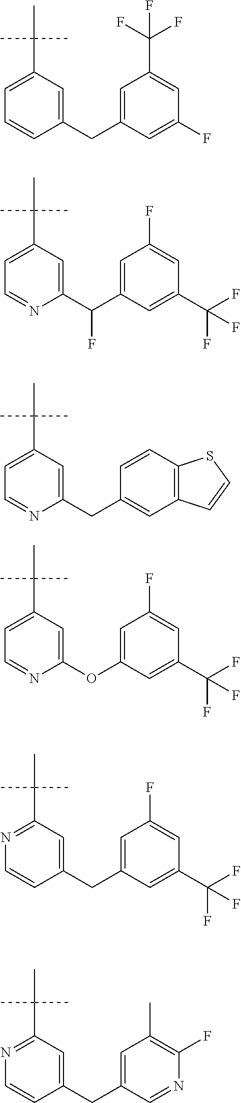

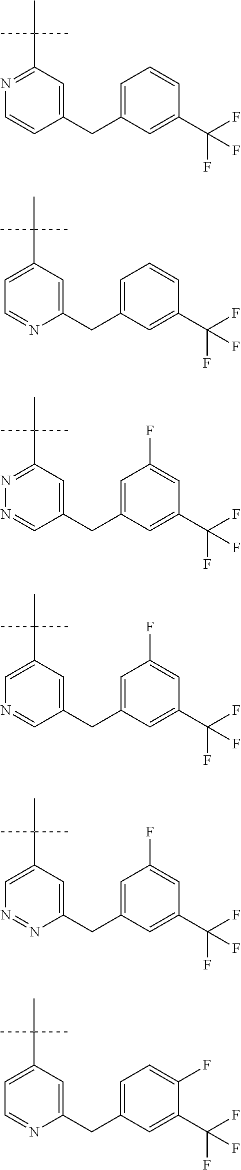

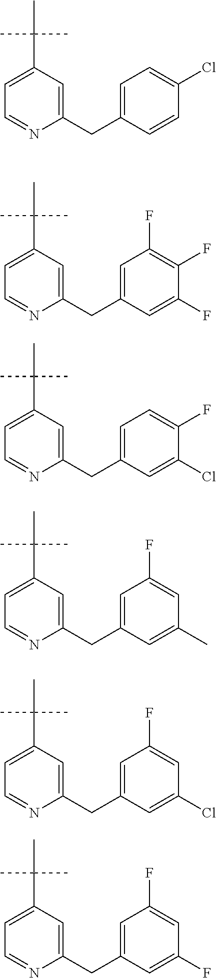

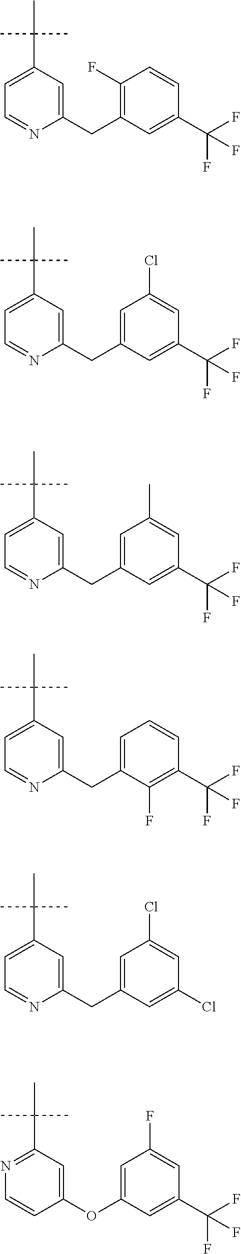

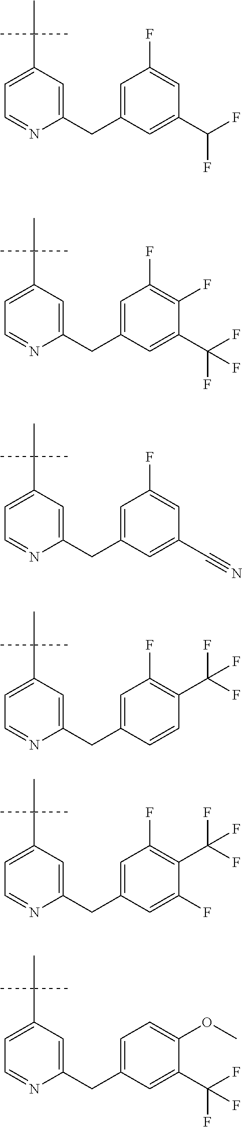

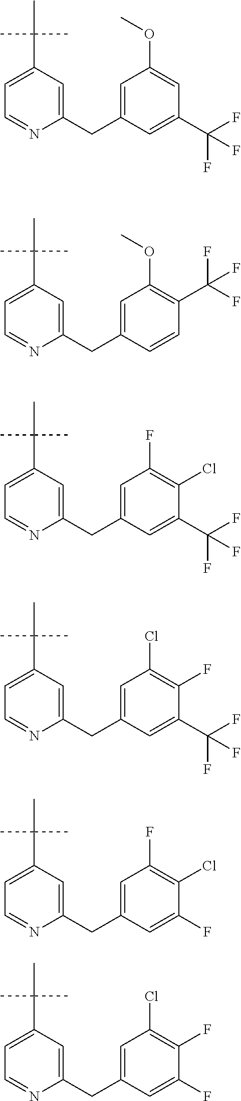

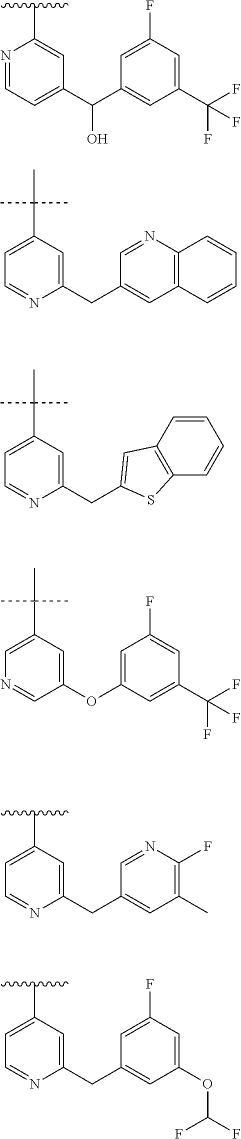

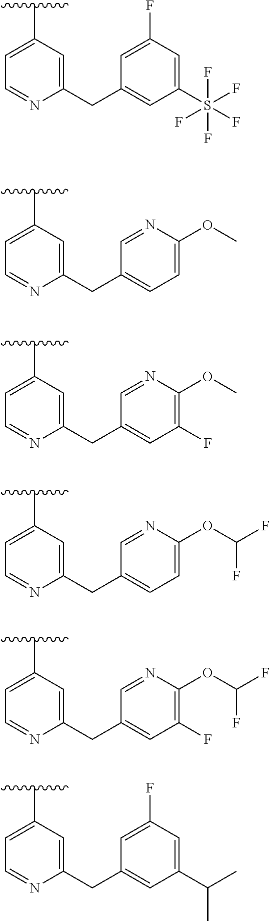 [SMILES: *c1ccnc(Cc2cc(F)cc(S(F)(F)(F)(F)F)c2)c1 ; *c1ccnc(Cc2ccc(OC)nc2)c1 ; *c1ccnc(Cc2cnc(OC)c(F)c2)c1 ; *c1ccnc(Cc2ccc(OC(F)F)nc2)c1 ; *c1ccnc(Cc2cnc(OC(F)F)c(F)c2)c1 ; *c1ccnc(Cc2cc(F)cc(C(C)C)c2)c1]

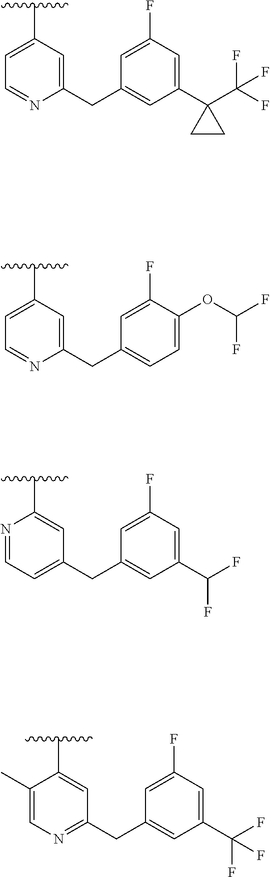 [SMILES: *c1ccnc(Cc2cc(F)cc(C3(C(F)(F)F)CC3)c2)c1 ; *c1ccnc(Cc2ccc(OC(F)F)c(F)c2)c1 ; *c1cc(Cc2cc(F)cc(C(F)F)c2)ccn1 ; *c1cc(Cc2cc(F)cc(C(F)(F)F)c2)ncc1C]

In the compounds herein, Q can be selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵—, —OCR⁴R⁵CR⁶R⁷— and —CR⁴R⁵O—. Q can be selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵— and —OCR⁴R⁵CR⁶R⁷—. Q can be —CR⁴R⁵—. Q can be —CR⁴R⁵CR⁶R⁷—. Q can be —CR⁴R⁵CR⁶R⁷CR⁸R⁹—. Q can be —CR⁴R⁵CR⁶R⁷O—. Q can be —OCR⁴R⁵—. Q can be —OCR⁴R⁵CR⁶R⁷—. Q can be —CR⁴R⁵O—.

In the compounds herein, Q can be selected from —CH₂CH₂—, —CH₂CH₂CH₂—, —CH₂—, —OCH₂—, —CH₂O—, —CH₂CH₂O—, —OCH₂CH₂—, —CH(CH₃)CH₂— and —CH₂CH(CH₃)—. Q can be selected from the group consisting of —CH₂CH₂—, —CH₂CH₂CH₂—, —CH₂—, —OCH₂—, —CH₂CH₂O—, —CH(CH₃)CH₂— and —CH₂CH(CH₃)—. Q can be —CH₂CH₂—. Q can be —CH₂CH₂CH₂—. Q can be —CH₂—. Q can be —OCH₂—. Q can be —CH₂O—. Q can be —CH₂CH₂O—. Q can be CH₂CH(CH₃)—. Q can be —OCH₂CH₂—. Q can be —CH(CH₃)CH₂—. Q can be a C₁₋₃ alkyl linker. Q can be selected from —CH₂—, —CH₂CH₂— and —CH₂CH₂CH₂—. Q can be —CH₂—. Q can be —CH₂CH₂—. Q can be —CH₂CH₂CH₂—.

In the compounds herein, R⁴, R⁵, R⁶, R⁷, R⁸ and R⁹ can independently be H or a C₁₋₃ alkyl group. R⁴, R⁵, R⁶, R⁷, R⁸ and R⁹ can independently be H or methyl.

In the compounds herein, R⁴ can be H. R⁴ can be a C₁₋₃ alkyl group. R⁴ can be methyl.

In the compounds herein, R⁵ can be H. R⁵ can be a C₁₋₃ alkyl group. R⁵ can be methyl.

In the compounds herein, R⁶ can be H. R⁶ can be a C₁₋₃ alkyl group. R⁶ can be methyl.

In the compounds herein, R⁷ can be H. R⁷ can be a C₁₋₃ alkyl group. R⁷ can be methyl.

In the compounds herein, R⁸ can be H. R⁸ can be a C₁₋₃ alkyl group. R⁸ can be methyl.

In the compounds herein, R⁹ can be H. R⁹ can be a C₁₋₃ alkyl group. R⁹ can be methyl.

Particular compounds include compounds of Formula (2a), (2b), (2c), (2d), (2e), (2f) and (2g):

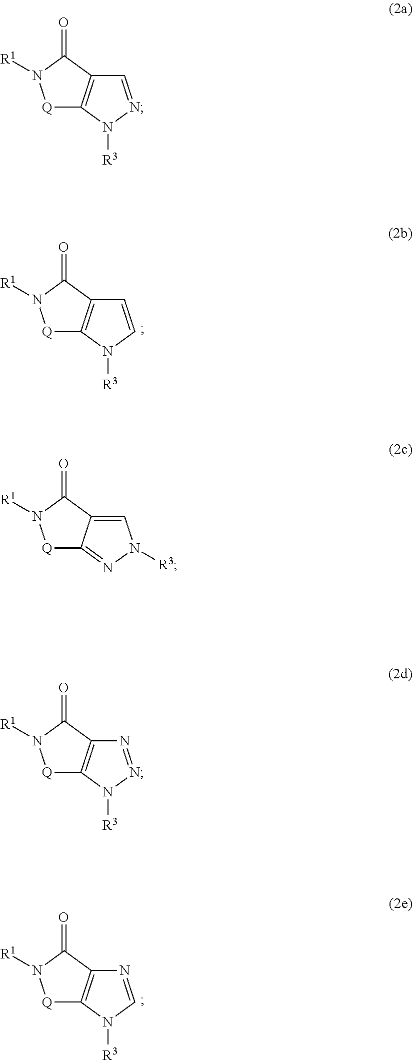

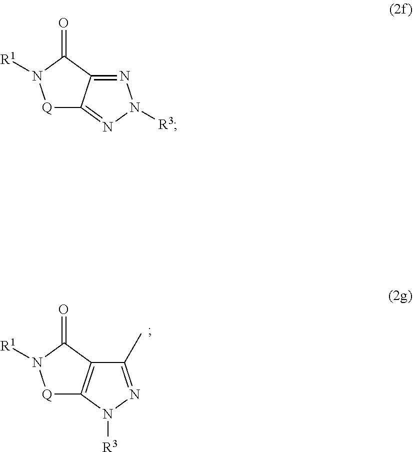

or a salt thereof, wherein R¹, R³ and Q are as defined above.

Particular compounds include compounds of Formula (3a), (3b), (3c), (3d), (3e), (3f) and (3g):

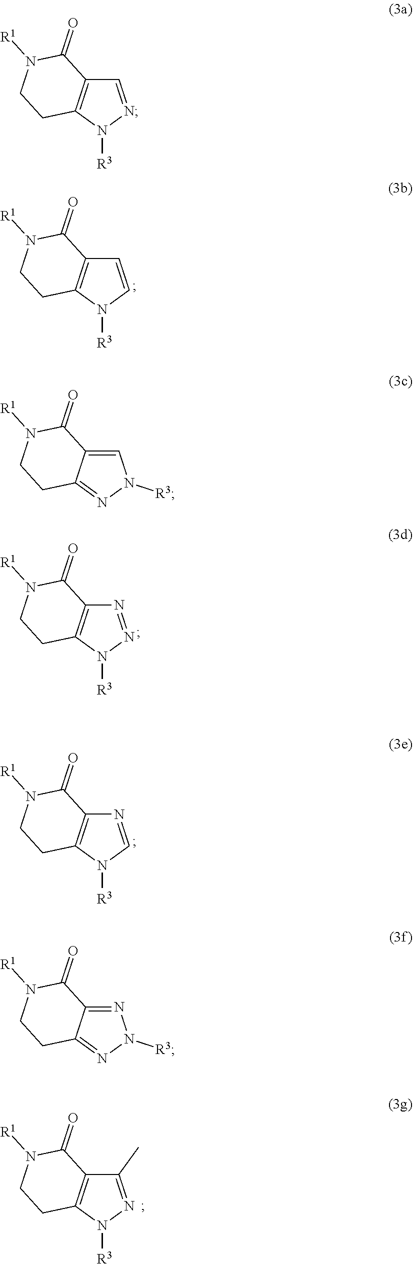

or a salt thereof, wherein R¹ and R³ are as defined above.

Particular compounds include compounds of formula (4a), (4b), (4c), (4d), (4e), (4f), (4g), (4h), (4i) or (4j):

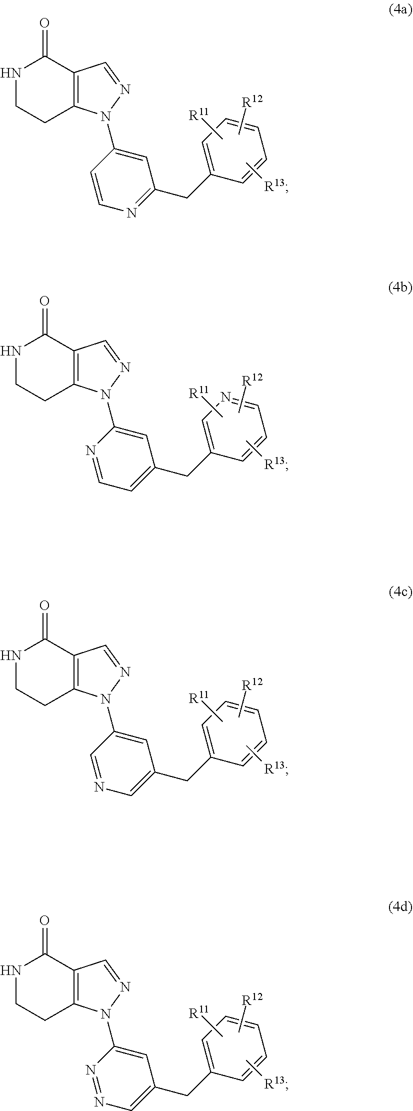

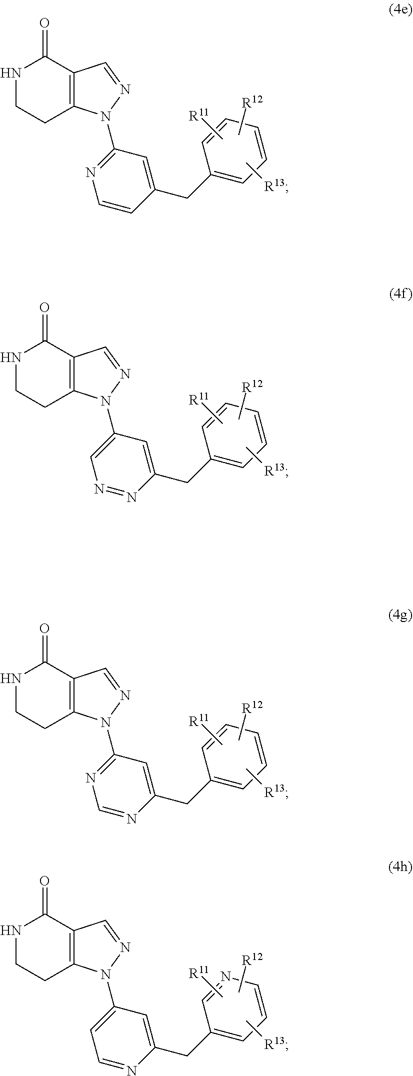

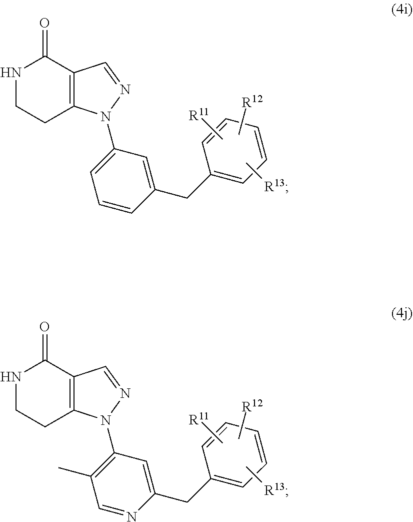

or a salt thereof, wherein R¹¹, R¹² and R¹³ are as defined above.

Particular compounds include compounds of formula (5a):

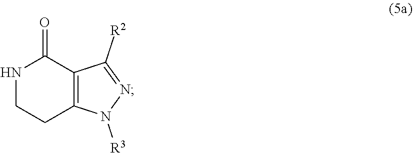

or a salt thereof, wherein R² and R³ are as defined above.

The compound can be a compound of formula (6a), (6b), (6c), (6d), (6e), (6f), (6g), (6h), (6i) or (6j):

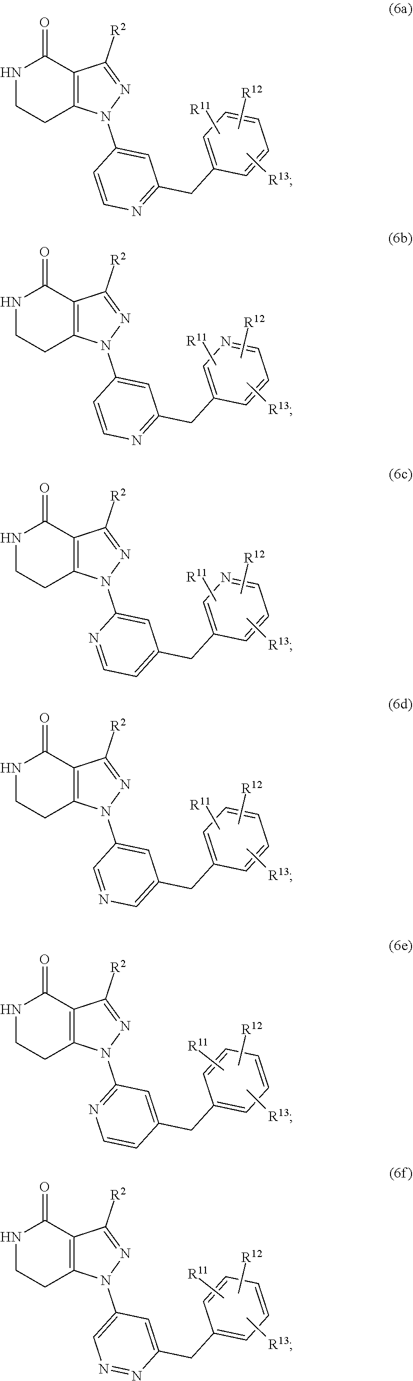

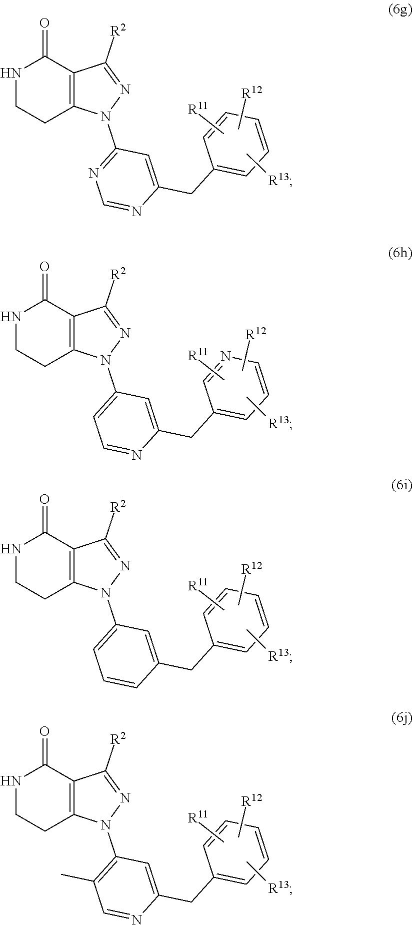

or a salt thereof, wherein R², R¹¹, R¹² and R¹³ are as defined above.

The compound can be a compound of formula (6a):

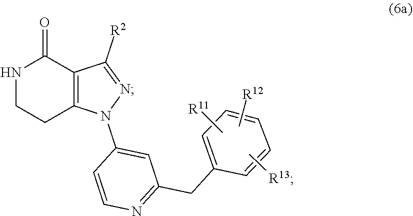

or a salt thereof, wherein R², R¹¹, R¹² and R¹³ are as defined above.

The compound can be a compound of formula (8a), (8b) or (8c):

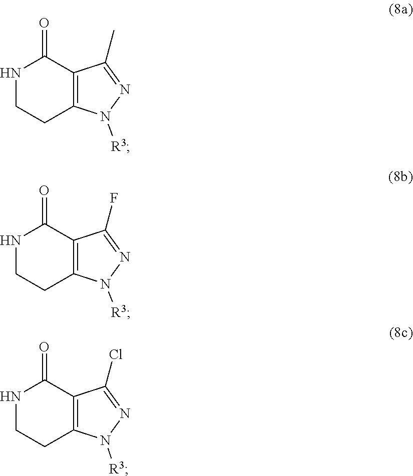

or a salt thereof, wherein R³ is as defined above.

Particular compounds include compounds of formula (9a), (9b), (9c), (9d), (9e), (9f), (9g), (9h), (9i) or (9j):

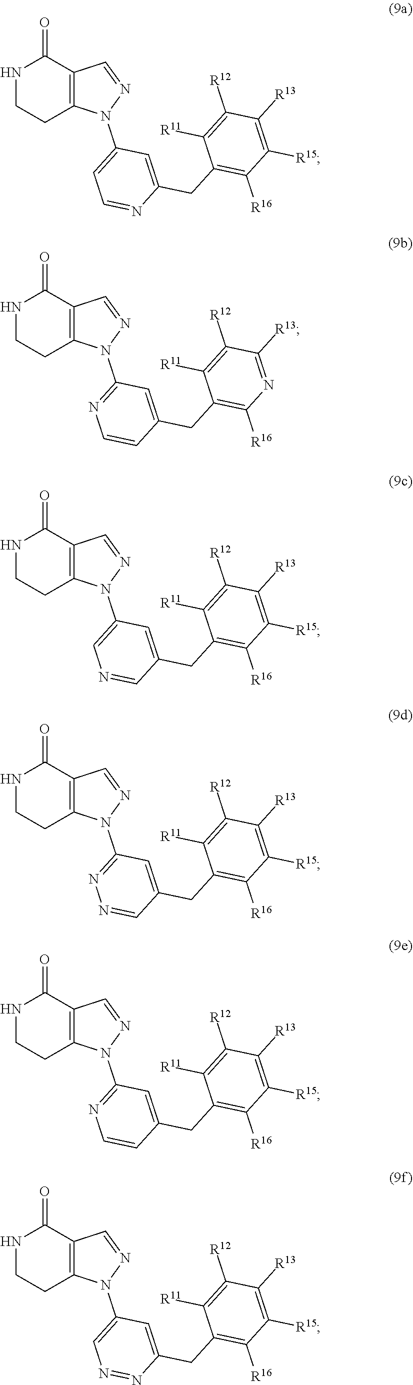

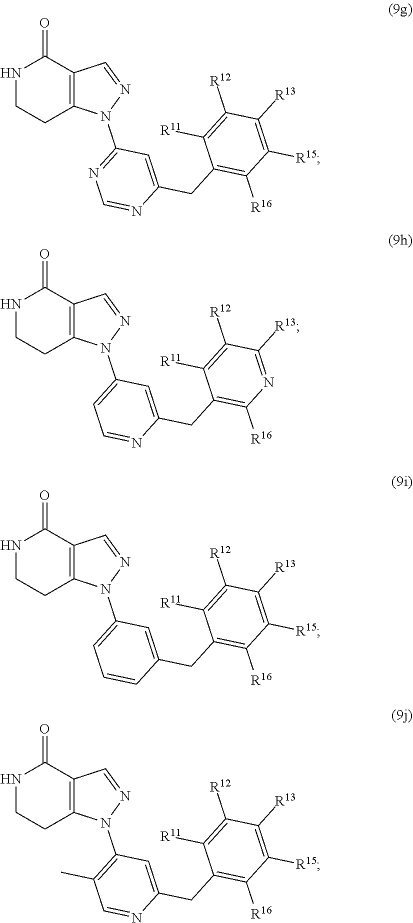

or a salt thereof, wherein R¹¹, R¹², R¹³, R¹⁵ and R¹⁶ are as defined above.

The compound can be a compound of formula (10a), (10b), (10c), (10d), (10e), (10f), (10g), (10h), (10i) or (10j):

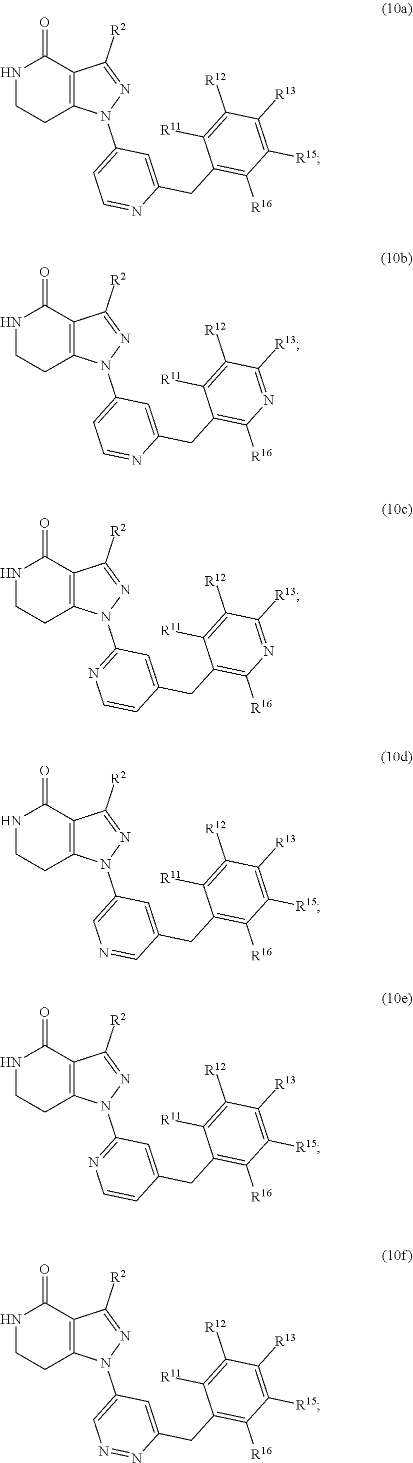

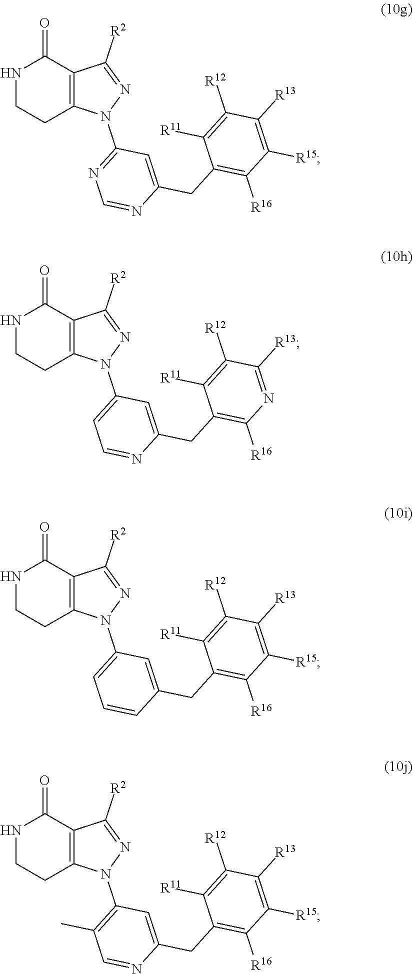

or a salt thereof, wherein R², R¹¹, R¹², R¹³, R¹⁵ and R¹⁶ are as defined above.

The compound can be a compound of formula (10a):

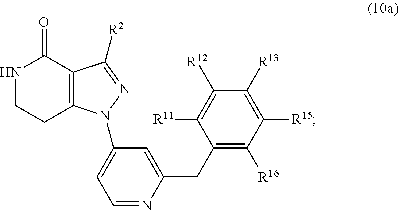

or a salt thereof, wherein R², R¹¹, R¹², R¹³, R¹⁵ and R¹⁶ are as defined above.

Particular compounds include compounds of Formula (11a), (11b) and (11c):

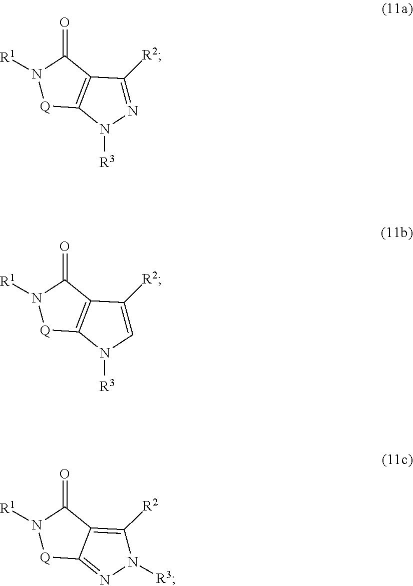

or a salt thereof, wherein R¹, R², R³ and Q are as defined above.

Particular compounds include compounds of Formula (12a), (12b) and (12c):

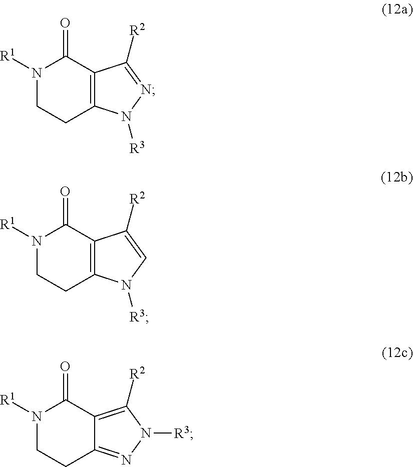

or a salt thereof, wherein R¹, R² and R³ are as defined above.

The compound can be a compound of formula (13a), (13b), (13c), (13d), (13e), (13f), (13g), (13h), (13i) or (13j):

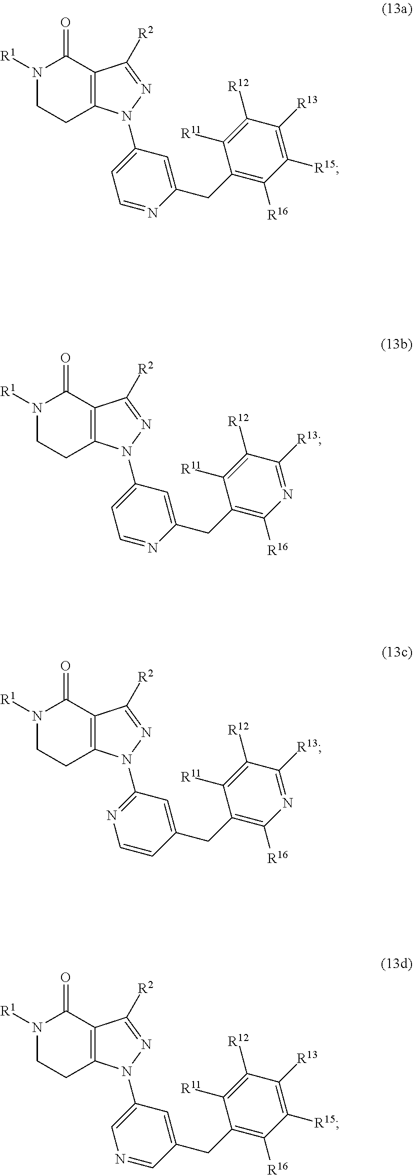

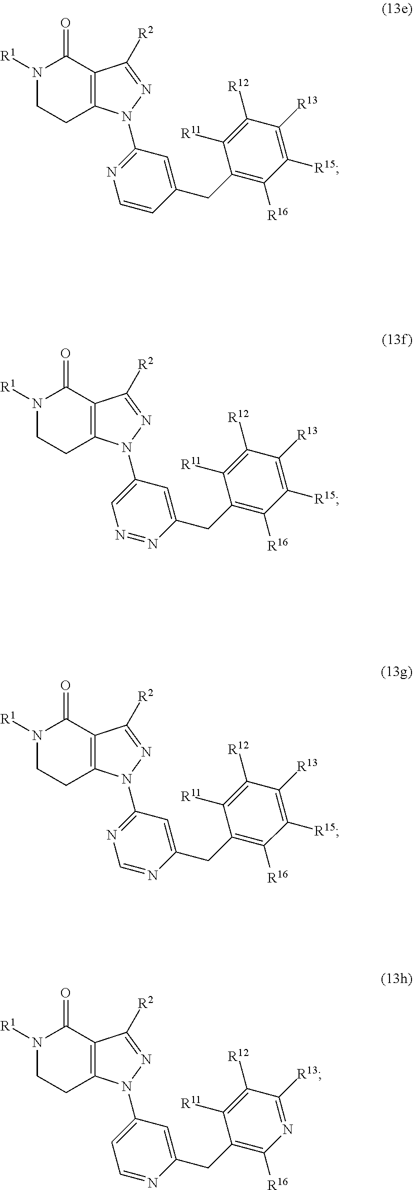

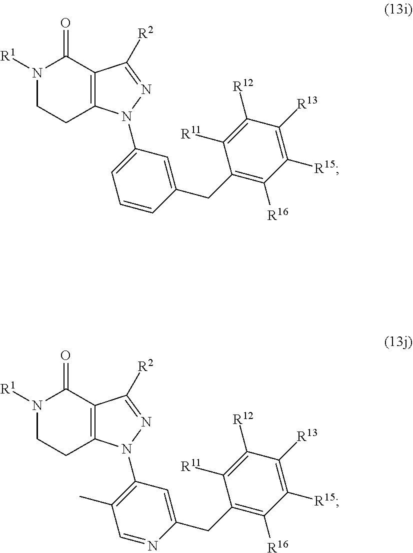

or a salt thereof, wherein R¹, R², R¹¹, R¹², R¹³, R¹⁵ and R¹⁶ are as defined above.

The compound can be a compound of formula (14a), (14b), (14c), (14d), (14e), (14f), (14g), (14h), (14i) or (14j):

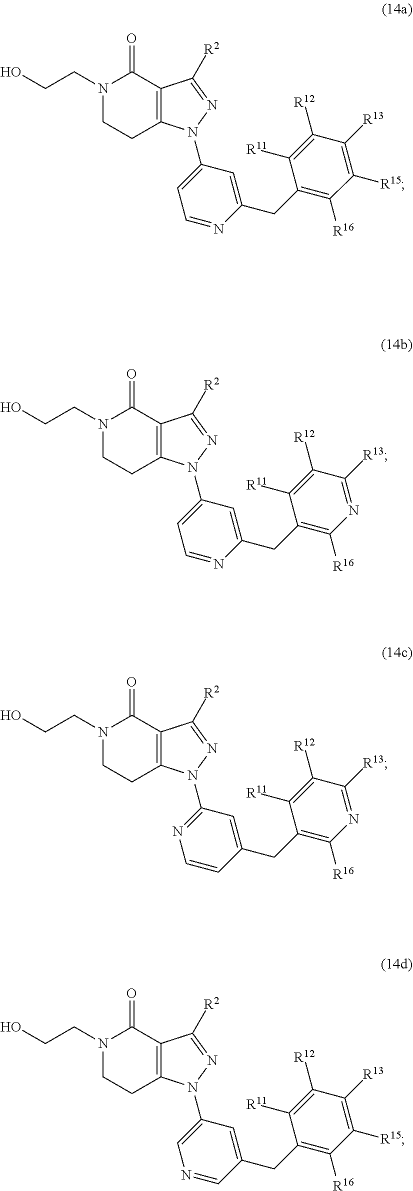

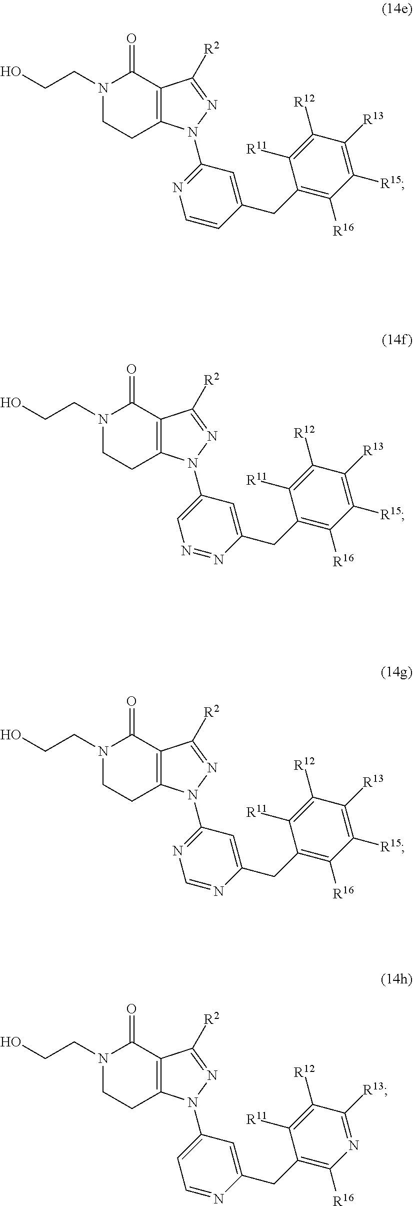

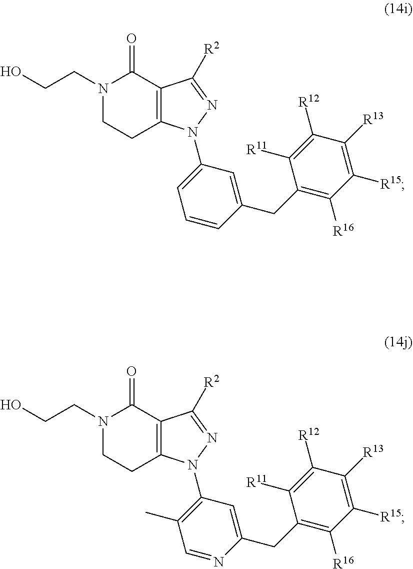

or a salt thereof, wherein R², R¹¹, R¹², R¹³, R¹⁵ and R¹⁶ are as defined above.

The compound can be selected from any one of Examples 1 to 118 as shown in Table 1 or a salt thereof.

The compound can be selected from the group consisting of:
1-(2-(3-fluoro-5-(trifluoromethyl)benzyl)pyridin-4-yl)-1,5,6,7-tetrahydro-4H-pyrazolo[4,3-c]pyridin-4-one;
1-(4-(3-(trifluoromethyl)benzyl)pyridin-2-yl)-1,5,6,7-tetrahydro-4H-pyrazolo[4,3-c]pyridin-4-one;
5-methyl-1-(4-(3-(trifluoromethyl)benzyl)pyridin-2-yl)-1,5,6,7-tetrahydro-4H-pyrazolo[4,3-c]pyridin-4-one;
1-(4-(3-fluoro-5-(trifluoromethyl)benzyl)pyridin-2-yl)-1,5,6,7-tetrahydro-4H-pyrazolo[4,3-c]pyridin-4-one;
1-(4-(3-fluoro-5-(trifluoromethyl)benzyl)pyridin-2-yl)-5-methyl-1,5,6,7-tetrahydro-4H-pyrazolo[4,3-c]pyridin-4-one;
1-(4-((6-fluoro-5-methylpyridin-3-yl)methyl)pyridin-2-yl)-1,5,6,7-tetrahydro-4H-pyrazolo[4,3-c]pyridin-4-one;
1-(4-((6-fluoro-5-methylpyridin-3-yl)methyl)pyridin-2-yl)-5-methyl-1,5,6,7-tetrahydro-4H-pyrazolo[4,3-c]pyridin-4-one;
2-(4-(3-(trifluoromethyl)benzyl)pyridin-2-yl)-2,5,6,7-tetrahydro-4H-pyrazolo[4,3-c]pyridin-4-one;
2-(2-(3-fluoro-5-(trifluoromethyl)benzyl)pyridin-4-yl)-2,5,6,7-tetrahydro-4H-pyrazolo[4,3-c]pyridin-4-one;
1-(4-(3-fluoro-5-(trifluoromethyl)benzyl)pyridin-2-yl)-5,6,7,8-tetrahydropyrazolo[4,3-c]azepin-4(1H)-one;
1-(4-(3-(trifluoromethyl)benzyl)pyridin-2-yl)-5,6,7,8-tetrahydropyrazolo[4,3-c]azepin-4(1H)-one;
1-(2-(3-fluoro-5-(trifluoromethyl)benzyl)pyridin-4-yl)-5,6,7,8-tetrahydropyrazolo[4,3-c]azepin-4(1H)-one;
1-(4-(3-(trifluoromethyl)benzyl)pyridin-2-yl)-6,7-dihydro-1H-pyrazolo[4,3-f][1,4]oxazepin-4(5H)-one;
1-(4-(3-fluoro-5-(trifluoromethyl)benzyl)pyridin-2-yl)-6,7-dihydro-1H-pyrazolo[4,3-f][1,4]oxazepin-4(5H)-one;
1-(5-(3-fluoro-5-(trifluoromethyl)benzyl)pyridazin-3-yl)-1,5,6,7-tetrahydro-4H-pyrazolo[4,3-c]pyridin-4-one;
1-(5-(3-fluoro-5-(trifluoromethyl)benzyl)pyridin-3-yl)-1,5,6,7-tetrahydro-4H-pyrazolo[4,3-c]pyridin-4-one;
1-(6-(3-fluoro-5-(trifluoromethyl)benzyl)pyridazin-4-yl)-1,5,6,7-tetrahydro-4H-pyrazolo[4,3-c]pyridin-4-one;
1-{4-[3-fluoro-5-(trifluoromethyl)phenoxy]pyridin-2-yl}-1,5,6,7-tetrahydro-4H-pyrazolo[4,3-c]pyridin-4-one;
1-(2-(4-chlorobenzyl)pyridin-4-yl)-1,5,6,7-tetrahydro-4H-pyrazolo[4,3-c]pyridin-4-one;
1-(2-(3-chloro-5-fluorobenzyl)pyridin-4-yl)-1,5,6,7-tetrahydro-4H-pyrazolo[4,3-c]pyridin-4-one;
1-(2-(3,5-difluorobenzyl)pyridin-4-yl)-1,5,6,7-tetrahydro-4H-pyrazolo[4,3-c]pyridin-4-one;
1-(2-(3-(trifluoromethyl)benzyl)pyridin-4-yl)-1,5,6,7-tetrahydro-4H-pyrazolo[4,3-c]pyridin-4-one;
1-(2-(4-fluoro-3-(trifluoromethyl)benzyl)pyridin-4-yl)-1,5,6,7-tetrahydro-4H-pyrazolo[4,3-c]pyridin-4-one;
1-(2-(3,4,5-trifluorobenzyl)pyridin-4-yl)-1,5,6,7-tetrahydro-4H-pyrazolo[4,3-c]pyridin-4-one;
1-(2-(3-chloro-4-fluorobenzyl)pyridin-4-yl)-1,5,6,7-tetrahydro-4H-pyrazolo[4,3-c]pyridin-4-one;
1-(2-(3-fluoro-5-methylbenzyl)pyridin-4-yl)-1,5,6,7-tetrahydro-4H-pyrazolo[4,3-c]pyridin-4-one;
1-(2-(3-chloro-5-(trifluoromethyl)benzyl)pyridin-4-yl)-1,5,6,7-tetrahydro-4H-pyrazolo[4,3-c]pyridin-4-one;
1-(2-(3-methyl-5-(trifluoromethyl)benzyl)pyridin-4-yl)-1,5,6,7-tetrahydro-4H-pyrazolo[4,3-c]pyridin-4-one;
1-(2-(2-fluoro-3-(trifluoromethyl)benzyl)pyridin-4-yl)-1,5,6,7-tetrahydro-4H-pyrazolo[4,3-c]pyridin-4-one;
1-(2-(2-fluoro-5-(trifluoromethyl)benzyl)pyridin-4-yl)-1,5,6,7-tetrahydro-4H-pyrazolo[4,3-c]pyridin-4-one;
1-(2-(3,5-dichlorobenzyl)pyridin-4-yl)-1,5,6,7-tetrahydro-4H-pyrazolo[4,3-c]pyridin-4-one;
1-(2-(4-(trifluoromethyl)benzyl)pyridin-4-yl)-1,5,6,7-tetrahydro-4H-pyrazolo[4,3-c]pyridin-4-one:
3-((4-(4-oxo-4,5,6,7-tetrahydro-1H-pyrazolo[4,3-c]pyridin-1-yl)pyridin-2-yl)methyl)-5-(trifluoromethyl)benzonitrile;
1-(2-(3,5-bis(trifluoromethyl)benzyl)pyridin-4-yl)-1,5,6,7-tetrahydro-4H-pyrazolo[4,3-c]pyridin-4-one;
1-(2-(3-fluoro-5-(trifluoromethyl)benzoyl)pyridin-4-yl)-1,5,6,7-tetrahydro-4H-pyrazolo[4,3-c]pyridin-4-one;
1-(2-((3-fluoro-5-(trifluoromethyl)phenyl)(hydroxy)methyl)pyridin-4-yl)-1,5,6,7-tetrahydro-4H-pyrazolo[4,3-c]pyridin-4-one;
1-(2-(fluoro(3-fluoro-5-(trifluoromethyl)phenyl)methyl)pyridin-4-yl)-1,5,6,7-tetrahydro-4H-pyrazolo[4,3-c]pyridin-4-one;
1-(2-(difluoro(3-fluoro-5-(trifluoromethyl)phenyl)methyl)pyridin-4-yl)-1,5,6,7-tetrahydro-4H-pyrazolo[4,3-c]pyridin-4-one;
1-(2-((3-fluoro-5-(trifluoromethyl)phenyl)methyl-d₂)pyridin-4-yl)-1,5,6,7-tetrahydro-4H-pyrazolo[4,3-]pyridin-4-one;
1-(2-(3-fluoro-5-(trifluoromethyl)benzyl)pyridin-4-yl)-6-methyl-1,5,6,7-tetrahydro-4H-pyrazolo[4,3-c]pyridin-4-one;
1-(4-(3-(trifluoromethyl)benzyl)pyridin-2-yl)-5,6,7,8-tetrahydro-[1,2,3]triazolo[4,5-c]azepin-4(1H)-one;
1-(4-(3-(trifluoromethyl)benzyl)pyridin-2-yl)-1,5,6,7-tetrahydro-4H-[1,2,3]triazolo[4,5-c]pyridin-4-one;
2-(4-(3-(trifluoromethyl)benzyl)pyridin-2-yl)-2,5,6,7-tetrahydro-4H-[1,2,3]triazolo[4,5-c]pyridin-4-one;
5-methyl-2-(4-(3-(trifluoromethyl)benzyl)pyridin-2-yl)-2,5,6,7-tetrahydro-4H-[1,2,3]triazolo[4,5-c]pyridin-4-one;
2-(4-(3-(trifluoromethyl)benzyl)pyridin-2-yl)-5,6,7,8-tetrahydro-[1,2,3]triazolo[4,5-c]azepin-4(2H)-one;
1-(4-(3-fluoro-5-(trifluoromethyl)benzyl)pyridin-2-yl)-5,6,7,8-tetrahydro-[1,2,3]triazolo[4,5-c]azepin-4(1H)-one;
1-(4-(3-fluoro-5-(trifluoromethyl)benzyl)pyridin-2-yl)-1,7-dihydropyrazolo[4,3-d][1,2]oxazin-4(5H)-one;
1-(2-(3-fluoro-5-(trifluoromethyl)benzyl)pyridin-4-yl)-5,6-dihydropyrrolo[3,4-c]pyrazol-4(1H)-one;
1-(2-((3-fluoro-5-(trifluoromethyl)phenyl)methyl-d)pyridin-4-yl)-1,5,6,7-tetrahydro-4H-pyrazolo[4,3-c]pyridin-4-one;
1-(2-(3-fluoro-5-(trifluoromethyl)benzyl)pyridin-4-yl)-7-methyl-1,5,6,7-tetrahydro-4H-pyrazolo[4,3-c]pyridin-4-one;
1-(2-(3-fluoro-5-(trifluoromethyl)benzyl)pyridin-4-yl)-1,5,6,7-tetrahydro-4H-pyrrolo[3,2-c]pyridin-4-one;
1-(2-(3-fluoro-5-(trifluoromethyl)phenoxy)pyridin-4-yl)-1,5,6,7-tetrahydro-4H-pyrazolo[4,3-c]pyridin-4-one;
1-(2-(3-cyclopropyl-5-fluorobenzyl)pyridin-4-yl)-1,5,6,7-tetrahydro-4H-pyrazolo[4,3-c]pyridin-4-one;
1-(2-(3-fluoro-4-(trifluoromethyl)benzyl)pyridin-4-yl)-1,5,6,7-tetrahydro-4H-pyrazolo[4,3-c]pyridin-4-one;
1-(2-(4-methoxy-3-(trifluoromethyl)benzyl)pyridin-4-yl)-1,5,6,7-tetrahydro-4H-pyrazolo[4,3-c]pyridin-4-one;
1-(6-(3-fluoro-5-(trifluoromethyl)benzyl)pyrimidin-4-yl)-1,5,6,7-tetrahydro-4H-pyrazolo[4,3-c]pyridin-4-one;
1-(2-(3-methoxy-5-(trifluoromethyl)benzyl)pyridin-4-yl)-1,5,6,7-tetrahydro-4H-pyrazolo[4,3-c]pyridin-4-one;
1-(2-(4-chloro-3,5-difluorobenzyl)pyridin-4-yl)-1,5,6,7-tetrahydro-4H-pyrazolo[4,3-c]pyridin-4-one;
1-(2-(benzo[b]thiophen-5-ylmethyl)pyridin-4-yl)-1,5,6,7-tetrahydro-4H-pyrazolo[4,3-c]pyridin-4-one;
1-(2-(3-methoxy-4-(trifluoromethyl)benzyl)pyridin-4-yl)-1,5,6,7-tetrahydro-4H-pyrazolo[4,3-c]pyridin-4-one;
1-(2-(3-fluoro-5-(pentafluoro-λ⁶-sulfaneyl)benzyl)pyridin-4-yl)-1,5,6,7-tetrahydro-4H-pyrazolo[4,3-c]pyridin-4-one;
1-(2-(3-(difluoromethoxy)-5-fluorobenzyl)pyridin-4-yl)-1,5,6,7-tetrahydro-4H-pyrazolo[4,3-c]pyridin-4-one;
1-(2-(4-(difluoromethoxy)-3-fluorobenzyl)pyridin-4-yl)-1,5,6,7-tetrahydro-4H-pyrazolo[4,3-c]pyridin-4-one;
1-(2-(3,4-difluoro-5-(trifluoromethyl)benzyl)pyridin-4-yl)-1,5,6,7-tetrahydro-4H-pyrazolo[4,3-c]pyridin-4-one;
1-(2-(3-(difluoromethyl)-5-fluorobenzyl)pyridin-4-yl)-1,5,6,7-tetrahydro-4H-pyrazolo[4,3-c]pyridin-4-one;
1-(2-(3-chloro-4,5-difluorobenzyl)pyridin-4-yl)-1,5,6,7-tetrahydro-4H-pyrazolo[4,3-c]pyridin-4-one;
1-(2-(quinolin-3-ylmethyl)pyridin-4-yl)-1,5,6,7-tetrahydro-4H-pyrazolo[4,3-c]pyridin-4-one;
1-(2-(4-chloro-3-fluoro-5-(trifluoromethyl)benzyl)pyridin-4-yl)-1,5,6,7-tetrahydro-4H-pyrazolo[4,3-c]pyridin-4-one;
1-(2-(benzo[b]thiophen-2-ylmethyl)pyridin-4-yl)-1,5,6,7-tetrahydro-4H-pyrazolo[4,3-c]pyridin-4-one;
1-(2-(3,5-difluoro-4-(trifluoromethyl)benzyl)pyridin-4-yl)-1,5,6,7-tetrahydro-4H-pyrazolo[4,3-c]pyridin-4-one:
1-(2-(3-fluoro-5-isopropylbenzyl)pyridin-4-yl)-1,5,6,7-tetrahydro-4H-pyrazolo[4,3-c]pyridin-4-one;
1-(2-((5-fluoro-6-methoxypyridin-3-yl)methyl)pyridin-4-yl)-1,5,6,7-tetrahydro-4H-pyrazolo[4,3-c]pyridin-4-one;
1-(2-(3-chloro-4-fluoro-5-(trifluoromethyl)benzyl)pyridin-4-yl)-1,5,6,7-tetrahydro-4H-pyrazolo[4,3-c]pyridin-4-one;
1-(2-(3-fluoro-5-(1-(trifluoromethyl)cyclopropyl)benzyl)pyridin-4-yl)-1,5,6,7-tetrahydro-4H-pyrazolo[4,3-c]pyridin-4-one;
1-(2-(3-fluoro-5-(trifluoromethyl)benzyl)pyridin-4-yl)-3-methyl-1,5,6,7-tetrahydro-4H-pyrazolo[4,3-c]pyridin-4-one;
1-(2-((6-fluoro-5-methylpyridin-3-yl)methyl)pyridin-4-yl)-3-methyl-1,5,6,7-tetrahydro-4H-pyrazolo[4,3-c]pyridin-4-one;
3-methyl-1-(2-(3,4,5-trifluorobenzyl)pyridin-4-yl)-1,5,6,7-tetrahydro-4H-pyrazolo[4,3-c]pyridin-4-one;
3-methyl-1-(2-(3-(trifluoromethyl)benzyl)pyridin-4-yl)-1,5,6,7-tetrahydro-4H-pyrazolo[4,3-c]pyridin-4-one;
1-(2-(3-chloro-5-fluorobenzyl)pyridin-4-yl)-3-methyl-1,5,6,7-tetrahydro-4H-pyrazolo[4,3-c]pyridin-4-one;
1-(2-(3-chloro-4-fluorobenzyl)pyridin-4-yl)-3-methyl-1,5,6,7-tetrahydro-4H-pyrazolo[4,3-c]pyridin-4-one;
1-(2-(4-(difluoromethoxy)-3-fluorobenzyl)pyridin-4-yl)-3-methyl-1,5,6,7-tetrahydro-4H-pyrazolo[4,3-c]pyridin-4-one;
1-(5-(3-fluoro-5-(trifluoromethyl)benzyl)pyridin-3-yl)-3-methyl-1,5,6,7-tetrahydro-4H-pyrazolo[4,3-c]pyridin-4-one;
1-(4-((6-fluoro-5-methylpyridin-3-yl)methyl)pyridin-2-yl)-3-methyl-1,5,6,7-tetrahydro-4H-pyrazolo[4,3-c]pyridin-4-one;
1-(2-(3-chloro-4,5-difluorobenzyl)pyridin-4-yl)-3-methyl-1,5,6,7-tetrahydro-4H-pyrazolo[4,3-c]pyridin-4-one;
3-fluoro-5-((4-(3-methyl-4-oxo-4,5,6,7-tetrahydro-1H-pyrazolo[4,3-c]pyridin-1-yl)pyridin-2-yl)methyl)benzonitrile;
1-(2-(3,4-difluoro-5-(trifluoromethyl)benzyl)pyridin-4-yl)-3-methyl-1,5,6,7-tetrahydro-4H-pyrazolo[4,3-c]pyridin-4-one;
1-(2-(4-chloro-3-fluoro-5-(trifluoromethyl)benzyl)pyridin-4-yl)-3-methyl-1,5,6,7-tetrahydro-4H-pyrazolo[4,3-c]pyridin-4-one;
1-(2-(3-chloro-4-fluoro-5-(trifluoromethyl)benzyl)pyridin-4-yl)-3-methyl-1,5,6,7-tetrahydro-4H-pyrazolo[4,3-c]pyridin-4-one;
1-(2-(3-(difluoromethoxy)-5-fluorobenzyl)pyridin-4-yl)-3-methyl-1,5,6,7-tetrahydro-4H-pyrazolo[4,3-c]pyridin-4-one;
1-(2-(4-chloro-3,5-difluorobenzyl)pyridin-4-yl)-3-methyl-1,5,6,7-tetrahydro-4H-pyrazolo[4,3-c]pyridin-4-one;
1-(2-(3-(difluoromethyl)-5-fluorobenzyl)pyridin-4-yl)-3-methyl-1,5,6,7-tetrahydro-4H-pyrazolo[4,3-c]pyridin-4-one;
1-(2-((6-methoxypyridin-3-yl)methyl)pyridin-4-yl)-3-methyl-1,5,6,7-tetrahydro-4H-pyrazolo[4,3-c]pyridin-4-one;
1-(2-((5-fluoro-6-methoxypyridin-3-yl)methyl)pyridin-4-yl)-3-methyl-1,5,6,7-tetrahydro-4H-pyrazolo[4,3-c]pyridin-4-one;
1-(2-((6-(difluoromethoxy)pyridin-3-yl)methyl)pyridin-4-yl)-3-methyl-1,5,6,7-tetrahydro-4H-pyrazolo[4,3-c]pyridin-4-one;
1-(4-(3-fluoro-5-(trifluoromethyl)benzyl)pyridin-2-yl)-3-methyl-1,5,6,7-tetrahydro-4H-pyrazolo[4,3-c]pyridin-4-one;
3-fluoro-5-((4-(4-oxo-4,5,6,7-tetrahydro-1H-pyrazolo[4,3-c]pyridin-1-yl)pyridin-2-yl)methyl)benzonitrile;
1-(2-(3-fluoro-5-(trifluoromethyl)benzyl)pyridin-4-yl)-5,6,7,8-tetrahydro-[1,2,3]triazolo[4,5-c]azepin-4(1H)-one;
2-(4-(3-fluoro-5-(trifluoromethyl)benzyl)pyridin-2-yl)-2,5,6,7-tetrahydro-4H-[1,2,3]triazolo[4,5-c]pyridin-4-one;
1-(4-(3-fluoro-5-(trifluoromethyl)benzyl)pyridin-2-yl)-1,5,6,7-tetrahydro-4H-[1,2,3]triazolo[4,5-c]pyridin-4-one;
1-(2-(3-fluoro-5-(trifluoromethyl)benzyl)pyridin-4-yl)-1,5,6,7-tetrahydro-4H-[1,2,3]triazolo[4,5-c]pyridin-4-one;
2-(2-(3-fluoro-5-(trifluoromethyl)benzyl)pyridin-4-yl)-2,5,6,7-tetrahydro-4H-[1,2,3]triazolo[4,5-c]pyridin-4-one;
1-(4-(3-(difluoromethyl)-5-fluorobenzyl)pyridin-2-yl)-1,5,6,7-tetrahydro-4H-pyrazolo[4,3-c]pyridin-4-one;
1-(5-(3-fluoro-5-(trifluoromethyl)phenoxy)pyridin-3-yl)-1,5,6,7-tetrahydro-4H-pyrazolo[4,3-c]pyridin-4-one;
1-(2-(3-fluoro-5-(trifluoromethyl)benzyl)pyridin-4-yl)-5-(2-methoxyethyl)-1,5,6,7-tetrahydro-4H-pyrazolo[4,3-c]pyridin-4-one;
1-(2-(3-fluoro-5-(trifluoromethyl)benzyl)pyridin-4-yl)-5-(2-hydroxyethyl)-1,5,6,7-tetrahydro-4H-pyrazolo[4,3-c]pyridin-4-one;
1-(4-(3-fluoro-5-(trifluoromethyl)benzyl)pyridin-2-yl)-5-(2-methoxyethyl)-1,5,6,7-tetrahydro-4H-pyrazolo[4,3-c]pyridin-4-one;
1-(4-(3-fluoro-5-(trifluoromethyl)benzyl)pyridin-2-yl)-5-(2-hydroxyethyl)-1,5,6,7-tetrahydro-4H-pyrazolo[4,3-c]pyridin-4-one;
1-(2-(3-fluoro-5-(trifluoromethyl)benzyl)-5-methylpyridin-4-yl)-1,5,6,7-tetrahydro-4H-pyrazolo[4,3-c]pyridin-4-one;
1-(3-(3-fluoro-5-(trifluoromethyl)benzyl)phenyl)-1,5,6,7-tetrahydro-4H-pyrazolo[4,3-c]pyridin-4-one;
1-(2-((6-(difluoromethoxy)-5-fluoropyridin-3-yl)methyl)pyridin-4-yl)-1,5,6,7-tetrahydro-4H-pyrazolo[4,3-c]pyridin-4-one;
1-(2-((6-(difluoromethoxy)-5-fluoropyridin-3-yl)methyl)pyridin-4-yl)-3-methyl-1,5,6,7-tetrahydro-4H-pyrazolo[4,3-c]pyridin-4-one;
2-(2-(3-fluoro-5-(trifluoromethyl)benzyl)pyridin-4-yl)-6,7-dihydro-2H-pyrazolo[4,3-][1,4]oxazepin-4(5H)-one;
1-(4-(3-fluoro-5-(trifluoromethyl)benzyl)pyridin-2-yl)-3-(hydroxymethyl)-1,5,6,7-tetrahydro-4H-pyrazolo[4,3-c]pyridin-4-one;
1-(2-(3-fluoro-5-(trifluoromethyl)benzyl)pyridin-4-yl)-3-(hydroxymethyl)-1,5,6,7-tetrahydro-4H-pyrazolo[4,3-c]pyridin-4-one;
1-(2-(3-fluoro-5-(trifluoromethyl)benzyl)pyridin-4-yl)-4-oxo-4,5,6,7-tetrahydro-1H-pyrazolo[4,3-c]pyridine-3-carbonitrile;
1-(2-(3-fluoro-5-(trifluoromethyl)benzyl)pyridin-4-yl)-3-(trifluoromethyl)-1,5,6,7-tetrahydro-4H-pyrazolo[4,3-c]pyridin-4-one;
1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one;
1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one;
   or a salt thereof.

Further embodiments of the invention include the use of a compound of Formula (1) or a salt thereof or a pharmaceutical composition comprising a compound of Formula (1) as a GPR52 receptor modulator or a GPR52 receptor agonist. Compounds of the present invention may be used as GPR52 modulators. Compounds of the present invention may be used as GPR52 agonists. General references to Formula 1 throughout the specification include all compounds of Formula (1x) including (1′), (1a), (1b).

Compounds of the present invention may be used in the treatment of psychiatric disorders; neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; depressive disorders; hypothalamic disorders; pituitary disorders; prolactin-related disorders; trauma- or stressor-related disorders; disruptive, impulse-control or conduct disorders; sleep-wake disorders; substance-related disorders; addictive disorders; behavioral disorders; hypofrontality; abnormalities in the tuberoinfundibular, mesolimbic, mesocortical, or nigrostriatal pathway; decreased activity in the striatum; cortical dysfunction; neurocognitive dysfunction or conditions or symptoms related thereto.

Compounds of the present invention may be used in the treatment of schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, anhedonia, agitation, Alzheimer's disease, Parkinson's disease, Huntington's disease, vascular dementia, Lewy body disease, frontotemporal dementia, Tourette's syndrome, hyperprolactinemia, pituitary adenoma, prolactinoma, craniopharyngioma, Cushing's disease, diabetes insipidus, non-functioning tumours, obesity, posttraumatic stress disorder (PTSD), akathisia and associated movements, athetosis, ataxia, ballismus, hemiballismus, chorea, choreoathetosis, dyskinesia, tardive dyskinesia, neuroleptic-induced dyskinesia, myoclonus, mirror movement disorder, paroxysmal kinesigenic dyskinesia, restless legs syndrome, spasms, stereotypic movement disorder, sterotypy, Tic disorder, tremor, Wilson's disease, schizotypal personality disorder, delusional disorder, brief psychotic disorder, schizophreniform disorder, schizoaffective disorder, substance- or medication-induced psychotic disorder, delusions, hallucinations, disorganized thinking, grossly disorganized or abnormal motor behavior, catatonia, major depressive disorder, bipolar I disorder, bipolar II disorder, cyclothymic disorder, substance- or medication-induced bipolar and related disorders, bipolar and related disorders due to another medical condition, separation anxiety disorder, selective mutism, specific phobia, social anxiety disorder, panic disorder, agoraphobia, generalized anxiety disorder, substance- or medication-induced anxiety disorder, anxiety disorders due to another medical condition, delirium, major neurocognitive disorder, minor neurocognitive disorder, amnesia, dementia, developmental coordination disorder, stereotypic movement disorder, a post-stroke effect, dentatorubral-pallidoluysian atrophy, diminished emotional expression, avolition, alogia and asociality.

Compounds of the present invention may be used in the treatment of schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, neurocognitive disorder, delirium, anhedonia, agitation, Alzheimer's disease, Parkinson's disease, Huntington's disease, vascular dementia, Lewy body disease, frontotemporal dementia, Tourette's syndrome, hyperprolactinemia, obesity, and posttraumatic stress disorder (PTSD). Compounds of the present invention may be used in the treatment of schizophrenia.

### Definitions

In this application, the following definitions apply, unless indicated otherwise.

The term “GPR52 modulator” as used herein refers to any compound which binds to and modulates the function of the GPR52 receptor. The term “modulator” should be interpreted to include modulation by modalities including, but not limited to, agonists, partial agonists and inverse agonists.

The term “treatment”, in relation to the uses of any of the compounds described herein, including those of Formula (1), (1′), (1a), (1b), is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” (for example in relation to methods of treatment of a disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain.

Terms such as “alkyl”, “hydrocarbon”, “alkoxy”, “halo”, “aryl”, “heteroaryl”, “monocyclic”, “polycyclic” and “cycloalkyl” are all used in their conventional sense (e.g. as defined in the IUPAC Gold Book), unless indicated otherwise. “optionally substituted” as applied to any group means that the said group may if desired be substituted with one or more substituents, which may be the same or different.

Examples of heteroatom replacements for carbon atoms include replacement of a carbon atom in a —CH₂—CH₂—CH₂— chain with oxygen or sulfur to give an ether —CH₂—O—CH₂— or thioether —CH₂—S—CH₂—, replacement of a carbon atom in a group CH₂—C═C—H with nitrogen to give a nitrile (cyano) group CH₂—C═N, replacement of a carbon atom in a group —CH₂—CH₂—CH₂— with C═O to give a ketone —CH₂—C(O)—CH₂—, replacement of a carbon atom in a group —CH₂—CH═CH₂ with C═O to give an aldehyde —CH₂—C(O)H, replacement of a carbon atom in a group —CH₂—CH₂—CH₃ with O to give an alcohol —CH₂—CH₂—CH₂OH, replacement of a carbon atom in a group —CH₂—CH₂—CH₃ with O to give an ether —CH₂—O—CH₃, replacement of a carbon atom in a group —CH₂—CH₂—CH₃ with S to give an thiol —CH₂—CH₂—CH₂SH, replacement of a carbon atom in a group —CH₂—CH₂—CH₂— with S═O or SO₂ to give a sulfoxide —CH₂—S(O)—CH₂— or sulfone —CH₂—S(O)₂—CH₂—, replacement of a carbon atom in a —CH₂—CH₂—CH₂— chain with C(O)NH to give an amide —CH₂—CH₂—C(O)—NH—, replacement of a carbon atom in a —CH₂—CH₂—CH₂— chain with nitrogen to give an amine —CH₂—NH—CH₂—, and replacement of a carbon atom in a —CH₂—CH₂—CH₂— chain with C(O)O to give an ester (or carboxylic acid) —CH₂—CH₂—C(O)—O—. In each such replacement, at least one carbon atom of the alkyl group must remain.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, Ind., USA, 1999, ISBN 0-967-06710-3.

The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### Pharmaceutical Formulations

While it is possible for the active compound to be administered alone, it is preferable to present it as a pharmaceutical composition (e.g. formulation).

Accordingly, in one embodiment of the invention, there is provided a pharmaceutical composition comprising at least one compound of Formula (1), (1′), (1a), (1b) as defined above together with at least one pharmaceutically acceptable excipient.

The composition may be a tablet composition. The composition may be a capsule composition.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the Formula (1), (1′), (1a), (1b) can be formulated in accordance with known techniques, see for example, Remington's Pharmaceutical Sciences, Mack Publishing Company, Easton, Pa., USA. The pharmaceutical compositions can be in any form suitable for oral, parenteral, topical, intranasal, intrabronchial, sublingual, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably % (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragees, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compounds of the Formula (1) (1′), (1a), (1b) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### EXAMPLES

The invention will now be illustrated, but not limited, by reference to the following examples shown in Table 1. NMR and LCMS properties are set out in Table 3. Intermediates used are listed in Table 2.

TABLE 1
Examples

| | |
|---|---|
| 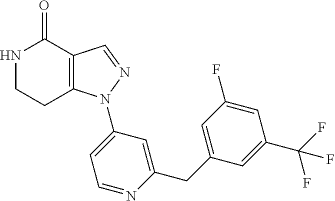 [SMILES: O=C1NCCc2c1cnn2-c1ccnc(Cc2cc(F)cc(C(F)(F)F)c2)c1] | Example 1 |
| 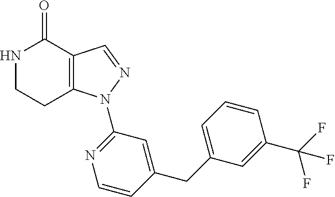 [SMILES: O=C1NCCc2c1cnn2-c1cc(Cc2cccc(C(F)(F)F)c2)ccn1] | Example 2 |
| 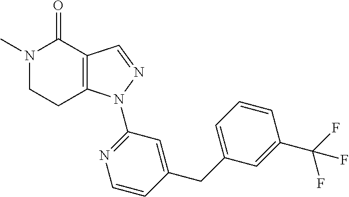 [SMILES: CN1CCc2c(cnn2-c2cc(Cc3cccc(C(F)(F)F)c3)ccn2)C1=O] | Example 3 |
| 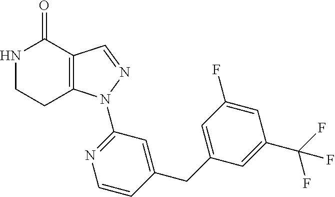 [SMILES: O=C1NCCc2c1cnn2-c1cc(Cc2cc(F)cc(C(F)(F)F)c2)ccn1] | Example 4 |
| 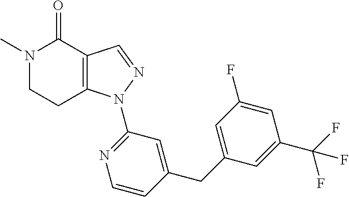 [SMILES: CN1CCc2c(cnn2-c2cc(Cc3cc(F)cc(C(F)(F)F)c3)ccn2)C1=O] | Example 5 |
| 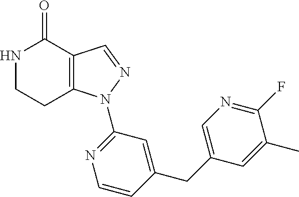 [SMILES: Cc1cc(Cc2ccnc(-n3ncc4c3CCNC4=O)c2)cnc1F] | Example 6 |
| 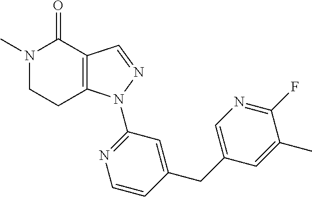 | Example 7 |
| 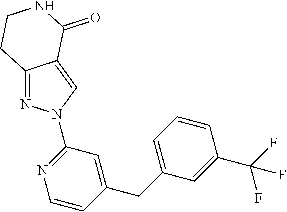 [SMILES: O=C1NCCc2nn(-c3cc(Cc4cccc(C(F)(F)F)c4)ccn3)cc21] | Example 8 |
| 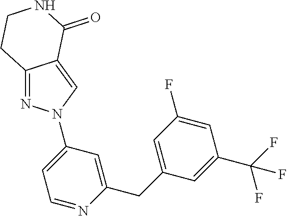 [SMILES: O=C1NCCc2nn(-c3ccnc(Cc4cc(F)cc(C(F)(F)F)c4)c3)cc21] | Example 9 |
| 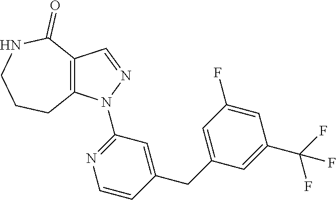 [SMILES: O=C1NCCCc2c1cnn2-c1cc(Cc2cc(F)cc(C(F)(F)F)c2)ccn1] | Example 10 |
| 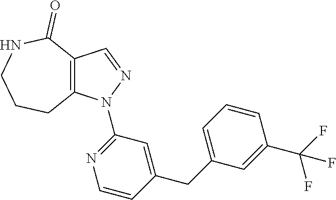 [SMILES: O=C1NCCCc2c1cnn2-c1cc(Cc2cccc(C(F)(F)F)c2)ccn1] | Example 11 |
| 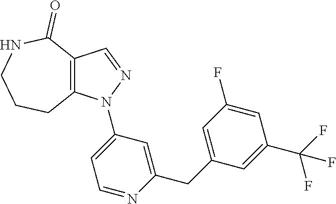 [SMILES: O=C1NCCCc2c1cnn2-c1ccnc(Cc2cc(F)cc(C(F)(F)F)c2)c1] | Example 12 |
| 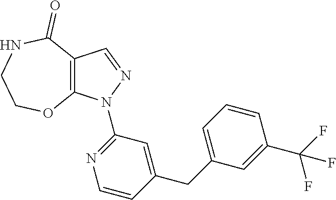 [SMILES: O=C1NCCOc2c1cnn2-c1cc(Cc2cccc(C(F)(F)F)c2)ccn1] | Example 13 |
| 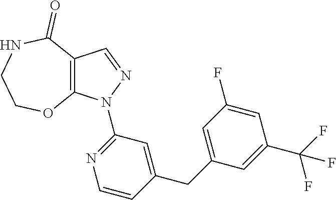 [SMILES: O=C1NCCOc2c1cnn2-c1cc(Cc2cc(F)cc(C(F)(F)F)c2)ccn1] | Example 14 |
| 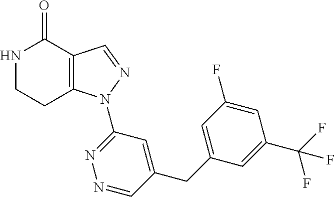 [SMILES: O=C1NCCc2c1cnn2-c1cc(Cc2cc(F)cc(C(F)(F)F)c2)cnn1] | Example 15 |
| 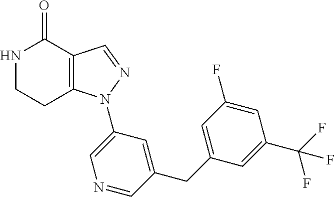 [SMILES: O=C1NCCc2c1cnn2-c1cncc(Cc2cc(F)cc(C(F)(F)F)c2)c1] | Example 16 |
| 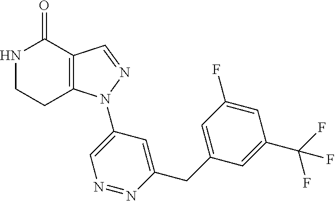 [SMILES: O=C1NCCc2c1cnn2-c1cnnc(Cc2cc(F)cc(C(F)(F)F)c2)c1] | Example 17 |
| 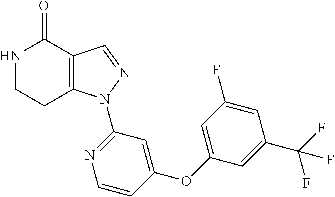 [SMILES: O=C1NCCc2c1cnn2-c1cc(Oc2cc(F)cc(C(F)(F)F)c2)ccn1] | Example 18 |
| 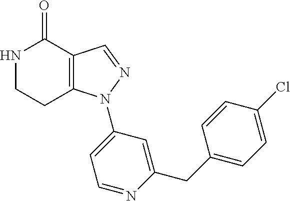 [SMILES: O=C1NCCc2c1cnn2-c1ccnc(Cc2ccc(Cl)cc2)c1] | Example 19 |
| 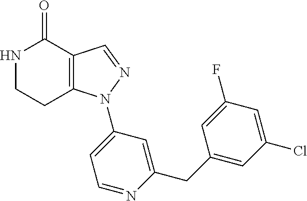 [SMILES: O=C1NCCc2c1cnn2-c1ccnc(Cc2cc(F)cc(Cl)c2)c1] | Example 20 |
| 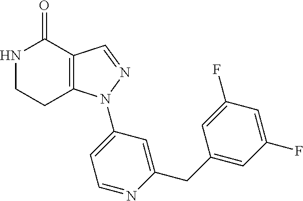 [SMILES: O=C1NCCc2c1cnn2-c1ccnc(Cc2cc(F)cc(F)c2)c1] | Example 21 |
| 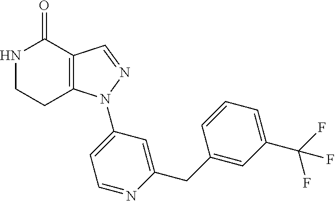 [SMILES: O=C1NCCc2c1cnn2-c1ccnc(Cc2cccc(C(F)(F)F)c2)c1] | Example 22 |
| 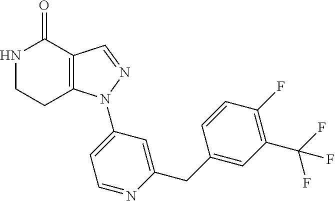 [SMILES: O=C1NCCc2c1cnn2-c1ccnc(Cc2ccc(F)c(C(F)(F)F)c2)c1] | Example 23 |
| 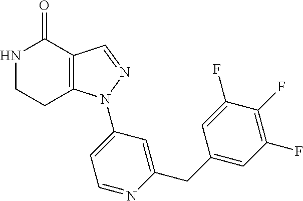 [SMILES: O=C1NCCc2c1cnn2-c1ccnc(Cc2cc(F)c(F)c(F)c2)c1] | Example 24 |
| 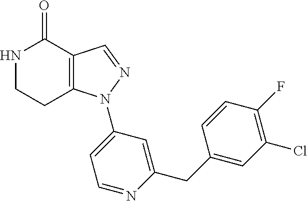 [SMILES: O=C1NCCc2c1cnn2-c1ccnc(Cc2ccc(F)c(Cl)c2)c1] | Example 25 |
| 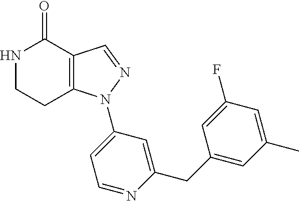 [SMILES: Cc1cc(F)cc(Cc2cc(-n3ncc4c3CCNC4=O)ccn2)c1] | Example 26 |
| 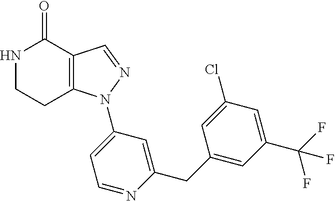 [SMILES: O=C1NCCc2c1cnn2-c1ccnc(Cc2cc(Cl)cc(C(F)(F)F)c2)c1] | Example 27 |
| 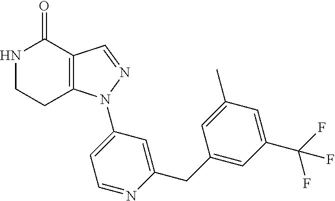 [SMILES: Cc1cc(Cc2cc(-n3ncc4c3CCNC4=O)ccn2)cc(C(F)(F)F)c1] | Example 28 |
| 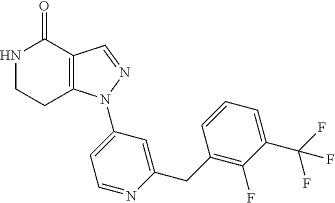 [SMILES: O=C1NCCc2c1cnn2-c1ccnc(Cc2cccc(C(F)(F)F)c2F)c1] | Example 29 |
| 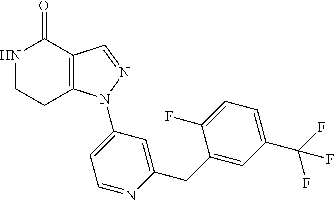 [SMILES: O=C1NCCc2c1cnn2-c1ccnc(Cc2cc(C(F)(F)F)ccc2F)c1] | Example 30 |
| 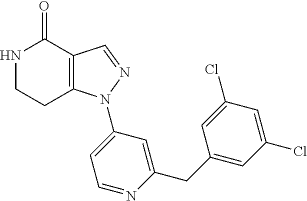 [SMILES: O=C1NCCc2c1cnn2-c1ccnc(Cc2cc(Cl)cc(Cl)c2)c1] | Example 31 |
| 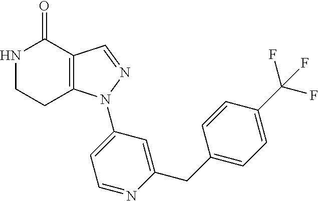 [SMILES: O=C1NCCc2c1cnn2-c1ccnc(Cc2ccc(C(F)(F)F)cc2)c1] | Example 32 |
| 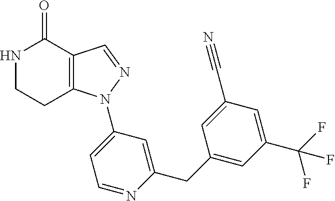 [SMILES: N#Cc1cc(Cc2cc(-n3ncc4c3CCNC4=O)ccn2)cc(C(F)(F)F)c1] | Example 33 |
| 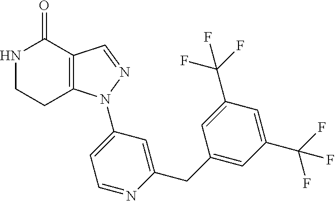 [SMILES: O=C1NCCc2c1cnn2-c1ccnc(Cc2cc(C(F)(F)F)cc(C(F)(F)F)c2)c1] | Example 34 |
| 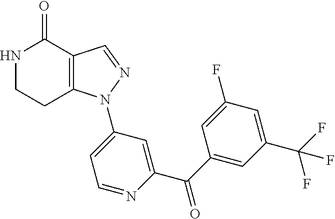 [SMILES: O=C(c1cc(F)cc(C(F)(F)F)c1)c1cc(-n2ncc3c2CCNC3=O)ccn1] | Example 35 |
| 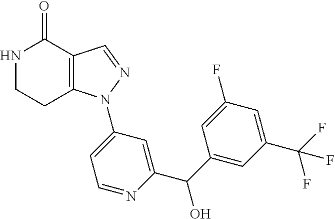 [SMILES: O=C1NCCc2c1cnn2-c1ccnc(C(O)c2cc(F)cc(C(F)(F)F)c2)c1] | Example 36 |
| 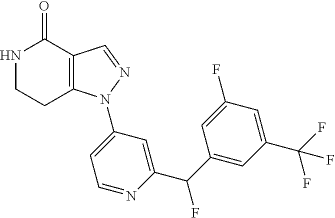 [SMILES: O=C1NCCc2c1cnn2-c1ccnc(C(F)c2cc(F)cc(C(F)(F)F)c2)c1] | Example 37 |
| 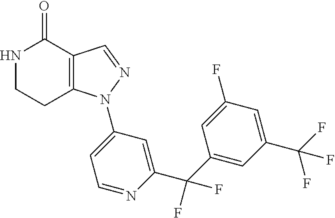 [SMILES: O=C1NCCc2c1cnn2-c1ccnc(C(F)(F)c2cc(F)cc(C(F)(F)F)c2)c1] | Example 38 |
| 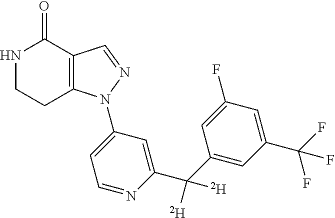 [SMILES: [2H]C([2H])(c1cc(F)cc(C(F)(F)F)c1)c1cc(-n2ncc3c2CCNC3=O)ccn1] | Example 39 |
| 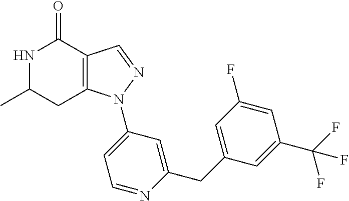 [SMILES: CC1Cc2c(cnn2-c2ccnc(Cc3cc(F)cc(C(F)(F)F)c3)c2)C(=O)N1] | Example 40 |
| 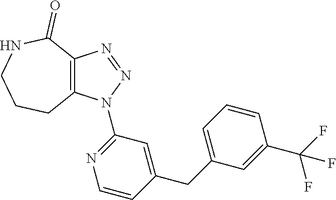 [SMILES: O=C1NCCCc2c1nnn2-c1cc(Cc2cccc(C(F)(F)F)c2)ccn1] | Example 41 |
| 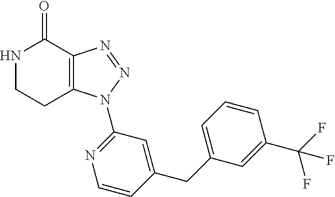 [SMILES: O=C1NCCc2c1nnn2-c1cc(Cc2cccc(C(F)(F)F)c2)ccn1] | Example 42 |
| 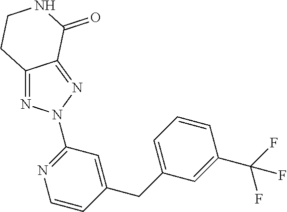 [SMILES: O=C1NCCc2nn(-c3cc(Cc4cccc(C(F)(F)F)c4)ccn3)nc21] | Example 43 |
| 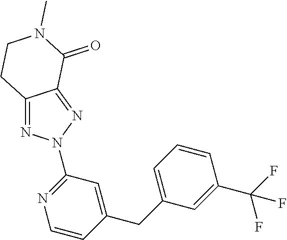 [SMILES: CN1CCc2nn(-c3cc(Cc4cccc(C(F)(F)F)c4)ccn3)nc2C1=O] | Example 44 |
| 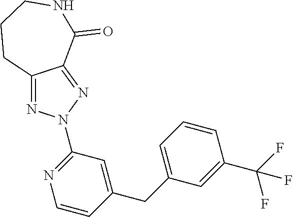 [SMILES: O=C1NCCCc2nn(-c3cc(Cc4cccc(C(F)(F)F)c4)ccn3)nc21] | Example 45 |
| 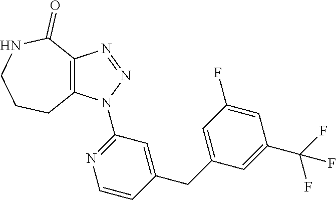 [SMILES: O=C1NCCCc2c1nnn2-c1cc(Cc2cc(F)cc(C(F)(F)F)c2)ccn1] | Example 46 |
| 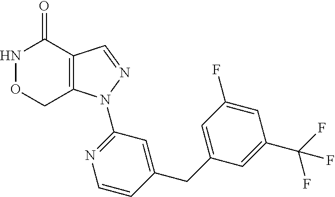 [SMILES: O=C1NOCc2c1cnn2-c1cc(Cc2cc(F)cc(C(F)(F)F)c2)ccn1] | Example 47 |
| 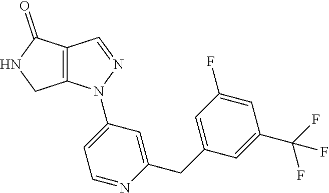 [SMILES: O=C1NCc2c1cnn2-c1ccnc(Cc2cc(F)cc(C(F)(F)F)c2)c1] | Example 48 |
| 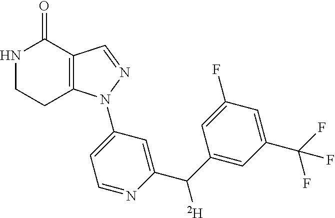 [SMILES: [2H]C(c1cc(F)cc(C(F)(F)F)c1)c1cc(-n2ncc3c2CCNC3=O)ccn1] | Example 49 |
| 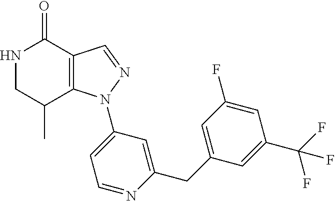 [SMILES: CC1CNC(=O)c2cnn(-c3ccnc(Cc4cc(F)cc(C(F)(F)F)c4)c3)c21] | Example 50 |
| 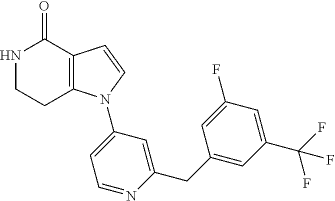 [SMILES: O=C1NCCc2c1ccn2-c1ccnc(Cc2cc(F)cc(C(F)(F)F)c2)c1] | Example 51 |
| 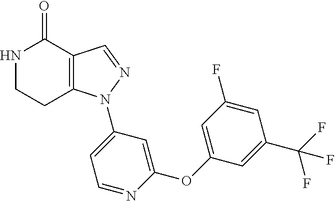 [SMILES: O=C1NCCc2c1cnn2-c1ccnc(Oc2cc(F)cc(C(F)(F)F)c2)c1] | Example 52 |
| 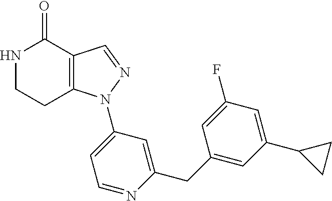 [SMILES: O=C1NCCc2c1cnn2-c1ccnc(Cc2cc(F)cc(C3CC3)c2)c1] | Example 53 |
| 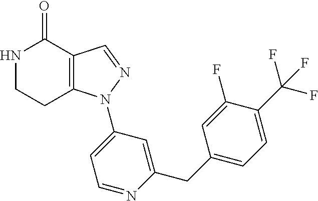 [SMILES: O=C1NCCc2c1cnn2-c1ccnc(Cc2ccc(C(F)(F)F)c(F)c2)c1] | Example 54 |
| 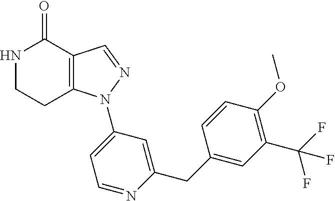 [SMILES: COc1ccc(Cc2cc(-n3ncc4c3CCNC4=O)ccn2)cc1C(F)(F)F] | Example 55 |
| 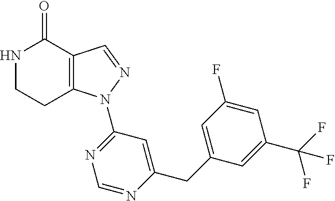 [SMILES: O=C1NCCc2c1cnn2-c1cc(Cc2cc(F)cc(C(F)(F)F)c2)ncn1] | Example 56 |
| 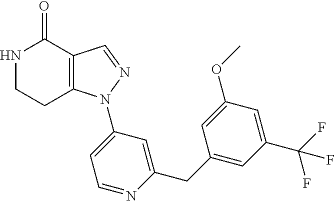 [SMILES: COc1cc(Cc2cc(-n3ncc4c3CCNC4=O)ccn2)cc(C(F)(F)F)c1] | Example 57 |
| 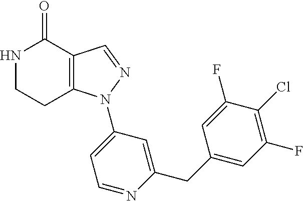 [SMILES: O=C1NCCc2c1cnn2-c1ccnc(Cc2cc(F)c(Cl)c(F)c2)c1] | Example 58 |
| 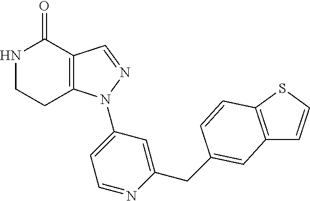 [SMILES: O=C1NCCc2c1cnn2-c1ccnc(Cc2ccc3sccc3c2)c1] | Example 59 |
| 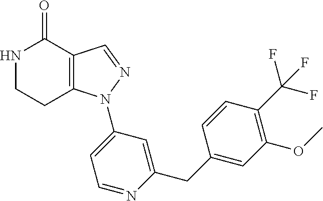 [SMILES: COc1cc(Cc2cc(-n3ncc4c3CCNC4=O)ccn2)ccc1C(F)(F)F] | Example 60 |
| 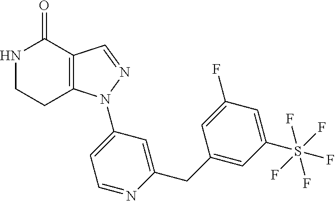 [SMILES: O=C1NCCc2c1cnn2-c1ccnc(Cc2cc(F)cc(S(F)(F)(F)(F)F)c2)c1] | Example 61 |
| 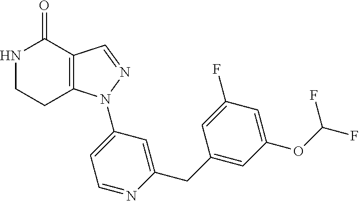 [SMILES: O=C1NCCc2c1cnn2-c1ccnc(Cc2cc(F)cc(OC(F)F)c2)c1] | Example 62 |
| 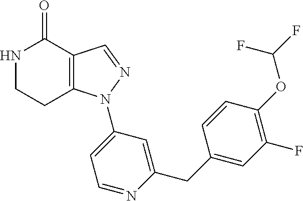 [SMILES: O=C1NCCc2c1cnn2-c1ccnc(Cc2ccc(OC(F)F)c(F)c2)c1] | Example 63 |
| 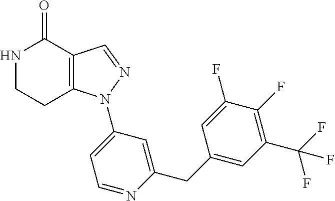 [SMILES: O=C1NCCc2c1cnn2-c1ccnc(Cc2cc(F)c(F)c(C(F)(F)F)c2)c1] | Example 64 |
| 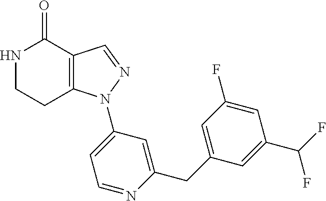 [SMILES: O=C1NCCc2c1cnn2-c1ccnc(Cc2cc(F)cc(C(F)F)c2)c1] | Example 65 |
| 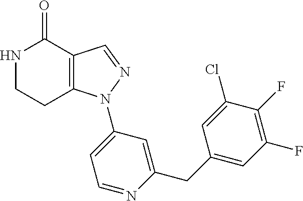 [SMILES: O=C1NCCc2c1cnn2-c1ccnc(Cc2cc(F)c(F)c(Cl)c2)c1] | Example 66 |
| 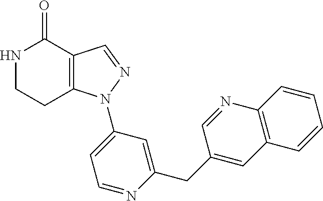 [SMILES: O=C1NCCc2c1cnn2-c1ccnc(Cc2cnc3ccccc3c2)c1] | Example 67 |
| 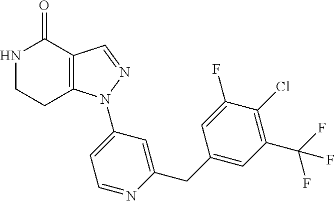 [SMILES: O=C1NCCc2c1cnn2-c1ccnc(Cc2cc(F)c(Cl)c(C(F)(F)F)c2)c1] | Example 68 |
| 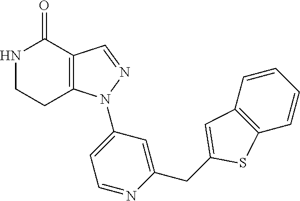 [SMILES: O=C1NCCc2c1cnn2-c1ccnc(Cc2cc3ccccc3s2)c1] | Example 69 |
| 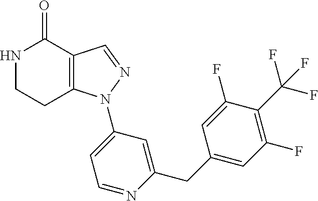 [SMILES: O=C1NCCc2c1cnn2-c1ccnc(Cc2cc(F)c(C(F)(F)F)c(F)c2)c1] | Example 70 |
| 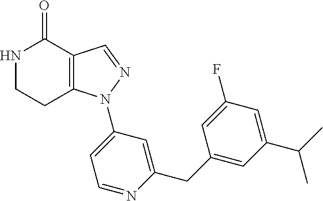 [SMILES: CC(C)c1cc(F)cc(Cc2cc(-n3ncc4c3CCNC4=O)ccn2)c1] | Example 71 |
| 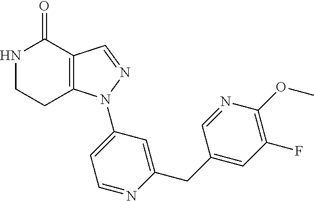 [SMILES: COc1ncc(Cc2cc(-n3ncc4c3CCNC4=O)ccn2)cc1F] | Example 72 |
| 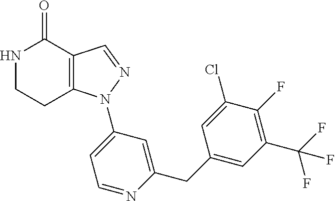 [SMILES: O=C1NCCc2c1cnn2-c1ccnc(Cc2cc(Cl)c(F)c(C(F)(F)F)c2)c1] | Example 73 |
| 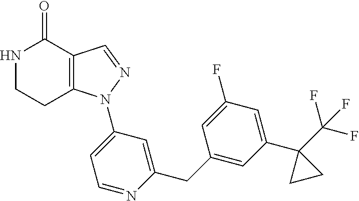 [SMILES: O=C1NCCc2c1cnn2-c1ccnc(Cc2cc(F)cc(C3(C(F)(F)F)CC3)c2)c1] | Example 74 |
| 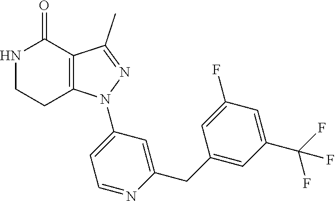 [SMILES: Cc1nn(-c2ccnc(Cc3cc(F)cc(C(F)(F)F)c3)c2)c2c1C(=O)NCC2] | Example 75 |
| 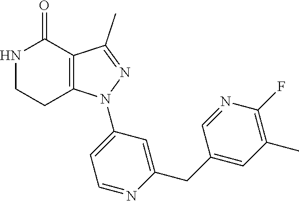 [SMILES: Cc1cc(Cc2cc(-n3nc(C)c4c3CCNC4=O)ccn2)cnc1F] | Example 76 |
| 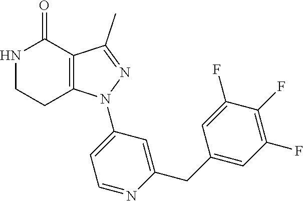 [SMILES: Cc1nn(-c2ccnc(Cc3cc(F)c(F)c(F)c3)c2)c2c1C(=O)NCC2] | Example 77 |
| 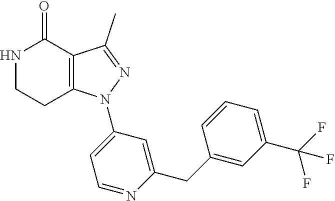 [SMILES: Cc1nn(-c2ccnc(Cc3cccc(C(F)(F)F)c3)c2)c2c1C(=O)NCC2] | Example 78 |
| 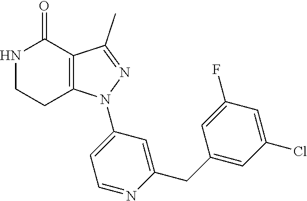 [SMILES: Cc1nn(-c2ccnc(Cc3cc(F)cc(Cl)c3)c2)c2c1C(=O)NCC2] | Example 79 |
| 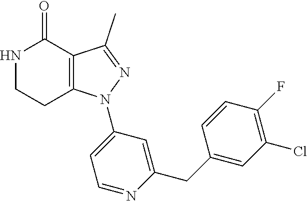 [SMILES: Cc1nn(-c2ccnc(Cc3ccc(F)c(Cl)c3)c2)c2c1C(=O)NCC2] | Example 80 |
| 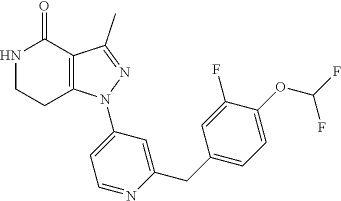 [SMILES: Cc1nn(-c2ccnc(Cc3ccc(OC(F)F)c(F)c3)c2)c2c1C(=O)NCC2] | Example 81 |
| 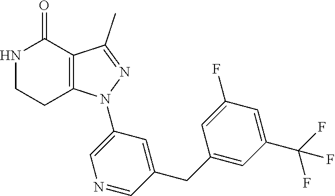 [SMILES: Cc1nn(-c2cncc(Cc3cc(F)cc(C(F)(F)F)c3)c2)c2c1C(=O)NCC2] | Example 82 |
| 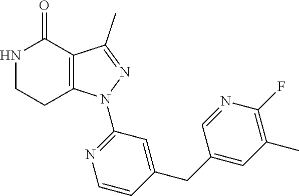 [SMILES: Cc1cc(Cc2ccnc(-n3nc(C)c4c3CCNC4=O)c2)cnc1F] | Example 83 |
| 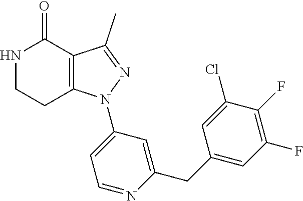 [SMILES: Cc1nn(-c2ccnc(Cc3cc(F)c(F)c(Cl)c3)c2)c2c1C(=O)NCC2] | Example 84 |
| 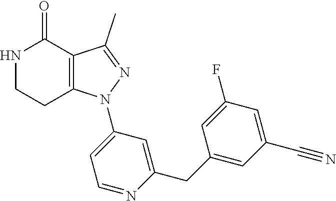 [SMILES: Cc1nn(-c2ccnc(Cc3cc(F)cc(C#N)c3)c2)c2c1C(=O)NCC2] | Example 85 |
| 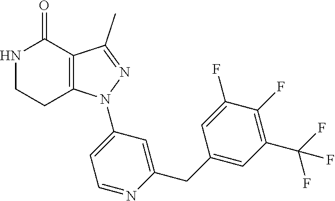 [SMILES: Cc1nn(-c2ccnc(Cc3cc(F)c(F)c(C(F)(F)F)c3)c2)c2c1C(=O)NCC2] | Example 86 |
| 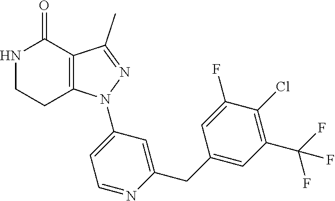 [SMILES: Cc1nn(-c2ccnc(Cc3cc(F)c(Cl)c(C(F)(F)F)c3)c2)c2c1C(=O)NCC2] | Example 87 |
| 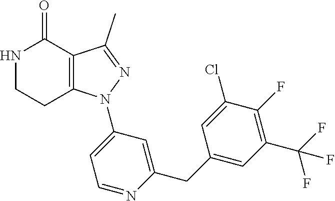 [SMILES: Cc1nn(-c2ccnc(Cc3cc(Cl)c(F)c(C(F)(F)F)c3)c2)c2c1C(=O)NCC2] | Example 88 |
| 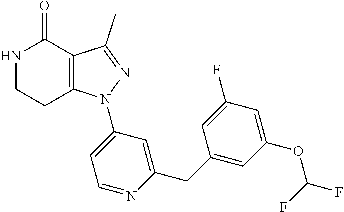 [SMILES: Cc1nn(-c2ccnc(Cc3cc(F)cc(OC(F)F)c3)c2)c2c1C(=O)NCC2] | Example 89 |
| 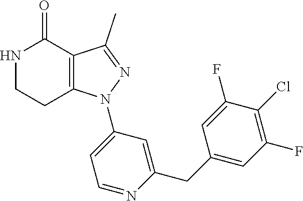 [SMILES: Cc1nn(-c2ccnc(Cc3cc(F)c(Cl)c(F)c3)c2)c2c1C(=O)NCC2] | Example 90 |
| 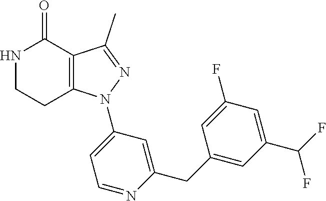 [SMILES: Cc1nn(-c2ccnc(Cc3cc(F)cc(C(F)F)c3)c2)c2c1C(=O)NCC2] | Example 91 |
| 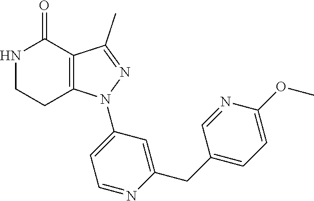 [SMILES: COc1ccc(Cc2cc(-n3nc(C)c4c3CCNC4=O)ccn2)cn1] | Example 92 |
| 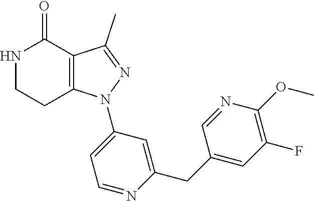 [SMILES: COc1ncc(Cc2cc(-n3nc(C)c4c3CCNC4=O)ccn2)cc1F] | Example 93 |
| 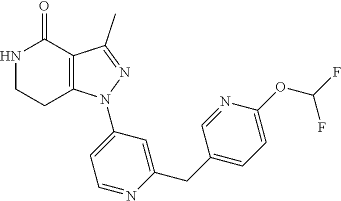 [SMILES: Cc1nn(-c2ccnc(Cc3ccc(OC(F)F)nc3)c2)c2c1C(=O)NCC2] | Example 94 |
| 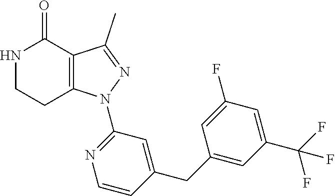 [SMILES: Cc1nn(-c2cc(Cc3cc(F)cc(C(F)(F)F)c3)ccn2)c2c1C(=O)NCC2] | Example 95 |
| 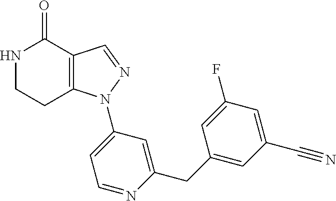 [SMILES: N#Cc1cc(F)cc(Cc2cc(-n3ncc4c3CCNC4=O)ccn2)c1] | Example 96 |
| 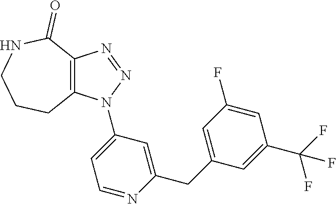 [SMILES: O=C1NCCCc2c1nnn2-c1ccnc(Cc2cc(F)cc(C(F)(F)F)c2)c1] | Example 97 |
| 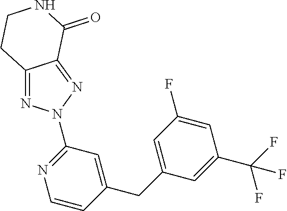 [SMILES: O=C1NCCc2nn(-c3cc(Cc4cc(F)cc(C(F)(F)F)c4)ccn3)nc21] | Example 98 |
| 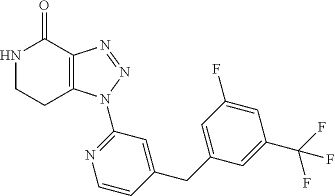 [SMILES: O=C1NCCc2c1nnn2-c1cc(Cc2cc(F)cc(C(F)(F)F)c2)ccn1] | Example 99 |
| 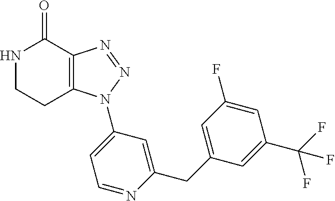 [SMILES: O=C1NCCc2c1nnn2-c1ccnc(Cc2cc(F)cc(C(F)(F)F)c2)c1] | Example 100 |
| 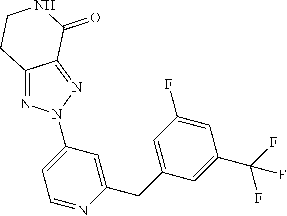 [SMILES: O=C1NCCc2nn(-c3ccnc(Cc4cc(F)cc(C(F)(F)F)c4)c3)nc21] | Example 101 |
| 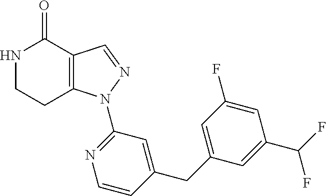 [SMILES: O=C1NCCc2c1cnn2-c1cc(Cc2cc(F)cc(C(F)F)c2)ccn1] | Example 102 |
| 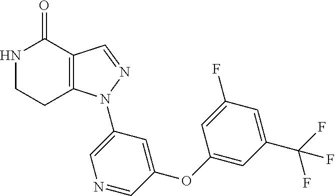 [SMILES: O=C1NCCc2c1cnn2-c1cncc(Oc2cc(F)cc(C(F)(F)F)c2)c1] | Example 103 |
| 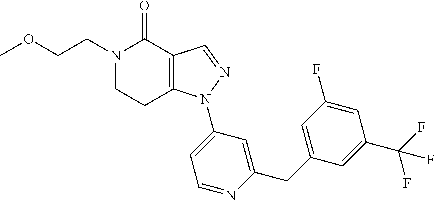 [SMILES: COCCN1CCc2c(cnn2-c2ccnc(Cc3cc(F)cc(C(F)(F)F)c3)c2)C1=O] | Example 104 |
| 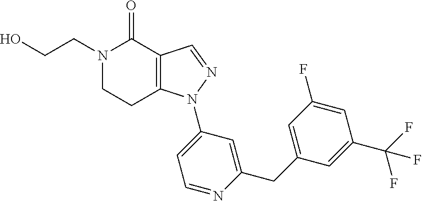 [SMILES: O=C1c2cnn(-c3ccnc(Cc4cc(F)cc(C(F)(F)F)c4)c3)c2CCN1CCO] | Example 105 |
| 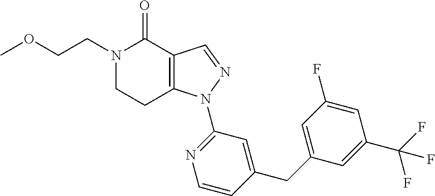 [SMILES: COCCN1CCc2c(cnn2-c2cc(Cc3cc(F)cc(C(F)(F)F)c3)ccn2)C1=O] | Example 106 |
| 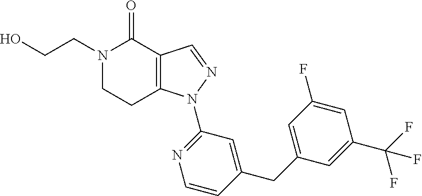 [SMILES: O=C1c2cnn(-c3cc(Cc4cc(F)cc(C(F)(F)F)c4)ccn3)c2CCN1CCO] | Example 107 |
| 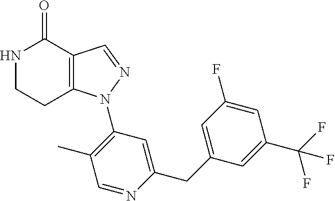 [SMILES: Cc1cnc(Cc2cc(F)cc(C(F)(F)F)c2)cc1-n1ncc2c1CCNC2=O] | Example 108 |
| 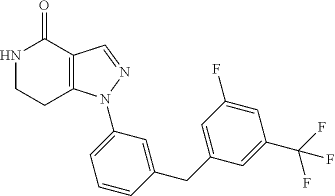 [SMILES: O=C1NCCc2c1cnn2-c1cccc(Cc2cc(F)cc(C(F)(F)F)c2)c1] | Example 109 |
| 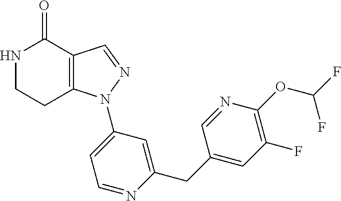 [SMILES: O=C1NCCc2c1cnn2-c1ccnc(Cc2cnc(OC(F)F)c(F)c2)c1] | Example 110 |
| 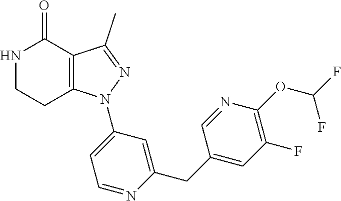 [SMILES: Cc1nn(-c2ccnc(Cc3cnc(OC(F)F)c(F)c3)c2)c2c1C(=O)NCC2] | Example 111 |
| 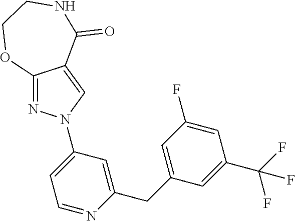 [SMILES: O=C1NCCOc2nn(-c3ccnc(Cc4cc(F)cc(C(F)(F)F)c4)c3)cc21] | Example 112 |
| 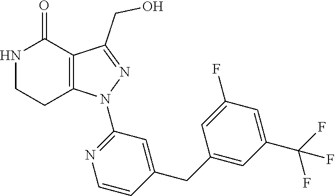 [SMILES: O=C1NCCc2c1c(CO)nn2-c1cc(Cc2cc(F)cc(C(F)(F)F)c2)ccn1] | Example 113 |
| 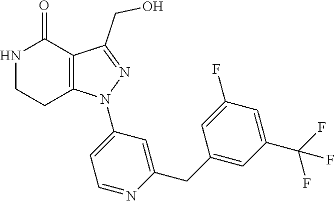 [SMILES: O=C1NCCc2c1c(CO)nn2-c1ccnc(Cc2cc(F)cc(C(F)(F)F)c2)c1] | Example 114 |
| 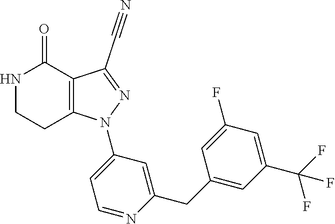 [SMILES: N#Cc1nn(-c2ccnc(Cc3cc(F)cc(C(F)(F)F)c3)c2)c2c1C(=O)NCC2] | Example 115 |
| 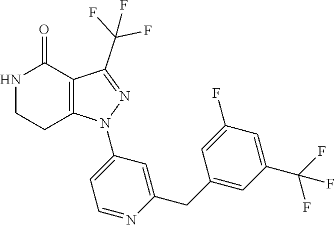 [SMILES: O=C1NCCc2c1c(C(F)(F)F)nn2-c1ccnc(Cc2cc(F)cc(C(F)(F)F)c2)c1] | Example 116 |
| 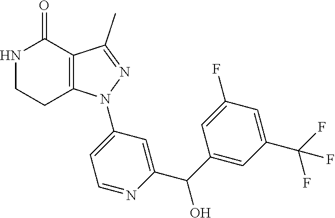 [SMILES: Cc1nn(-c2ccnc(C(O)c3cc(F)cc(C(F)(F)F)c3)c2)c2c1C(=O)NCC2] | Example 117 |
| 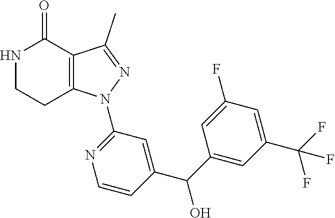 [SMILES: Cc1nn(-c2cc(C(O)c3cc(F)cc(C(F)(F)F)c3)ccn2)c2c1C(=O)NCC2] | Example 118 |

### Methods for the Preparation of Compounds of the Formula (1), (1′), (1a), (1b)

Compounds of Formula (1) (1′), (1a), (1b) can be prepared in accordance with synthetic methods well known to the skilled person. Provided is a process for the preparation of a compound as defined in Formula (1) above. Compounds of Formula (1) and associated intermediates can be prepared according to the procedures and schemes described herein. Disclosed intermediates may be applicable to the synthesis of one or more compounds of Formula (1). Procedures described herein may be applicable to the synthesis of one or more intermediates or compounds of Formula (1). Compounds of Formula (1) may be prepared by reaction of another compound of Formula (1). Intermediates may be used in subsequent synthetic steps without isolation or full characterisation per se. Certain compounds of the invention may be prepared according to the below general scheme:

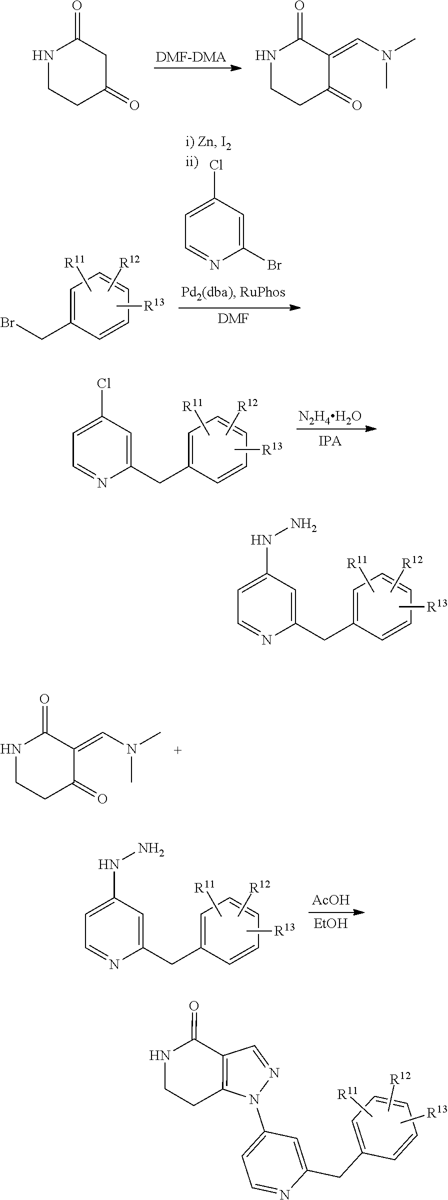

Certain compounds of the invention may be prepared according to the below general scheme:

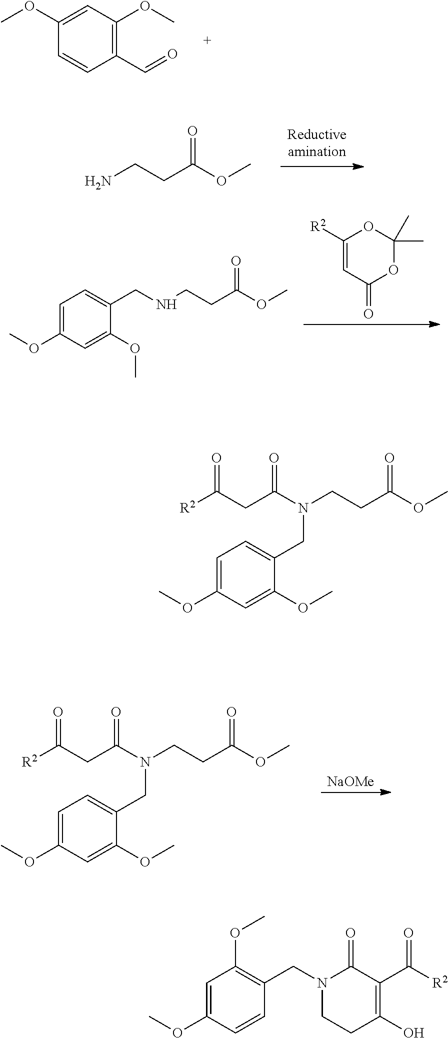

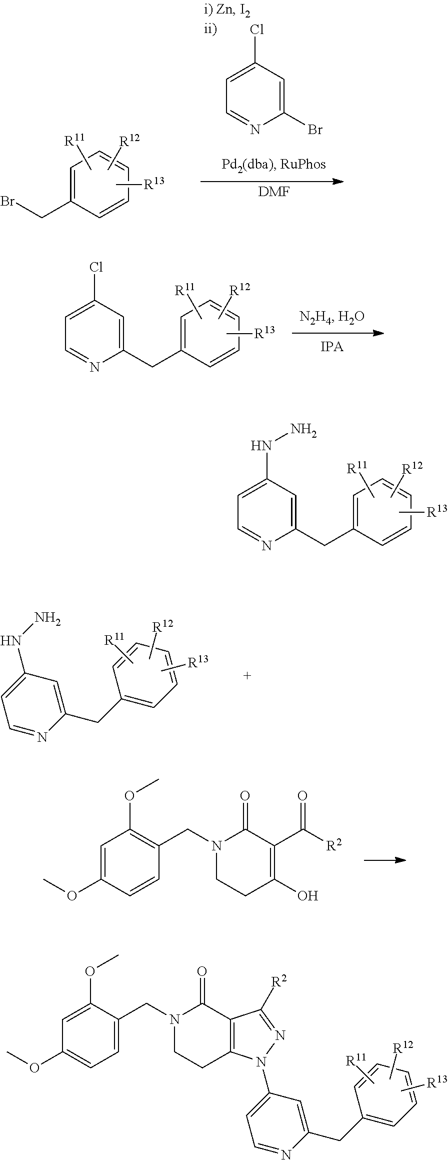

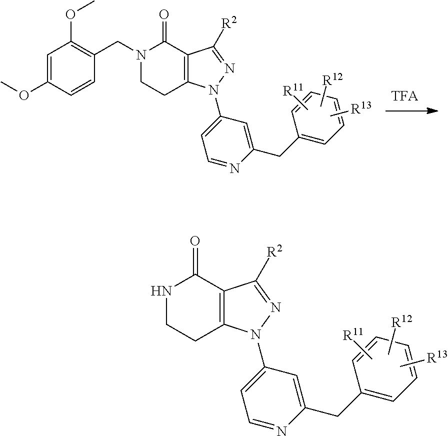

Where intermediates are commercially available they are identified by their chemical abstracts service (CAS) reference number in Table 3, where not commercially available the synthesis of the intermediates using standard transformations is detailed herein. Commercial reagents were utilized without further purification.

### General Procedures

Room temperature (RT) refers to approximately 20-27° C. ¹H NMR spectra were typically recorded at 400 MHz at ambient temperature unless otherwise specified. Chemical shift values are expressed in parts per million (ppm), i.e. (δ)-values. Standard abbreviations, or their combinations, are used for the multiplicity of the NMR signals, for example: s=singlet, br=broad, d=doublet, t=triplet, q=quartet, quin=quintet or p=pentet, h=heptet, dd=doublet of doublets, dt=doublet of triplets, m=multiplet. Coupling constants are listed as J values, measured in Hz. NMR and mass spectroscopy results were corrected to account for background peaks. Chromatography refers to column chromatography performed using silica or C18 silica and executed under positive pressure (flash chromatography) conditions.

### LCMS Methods

LCMS experiments were carried out using electrospray conditions under the conditions below (Solvents: A1=0.1% TFA in H₂O:MeCN (95:5); A2=5 mM ammonium acetate in H₂O; A3=2.5 L H₂O+2.5 mL 28% ammonia in H₂O solution; A4=0.1% HCO₂H in H₂O:MeCN (95:5); A5=10 mM NH₄HCO₃ in H₂O; A6=0.2% of 28% ammonia solution in H₂O; A7=0.1% TFA in H₂O; A8=5 mM NH₄HCO₃ in H₂O; A9=10 mM ammonium acetate in H₂O; 1=0.1% TFA in MeCN; B2=MeCN; B3=2.5 L MeCN+135 mL H₂O+2.5 mL 28% ammonia in H₂O solution.) LCMS data are given in the format: Mass ion, electrospray mode (positive or negative), retention time (experimental text and Table 2); Mass ion, electrospray mode (positive or negative), retention time, approximate purity (Table 3).

Method 1. Instruments: Hewlett Packard 1100 with G1315A DAD, Micromass ZQ; Column: Phenomenex Gemini-NX C18, 3 micron, 2.0×30 mm; Gradient [time (min)/solvent B3 in A3(%)]: 0.00/2, 0.10/2, 8.40/95, 10.00/95; Injection volume 1 μL; UV detection 230 to 400 nM; Column temperature 45° C.; Flow rate 1.5 mL/min.

Method 2. Instruments: Agilent Technologies 1290 Infinity II Series LC, 6125 Quadrupole MSD SL; Column: Waters XBridgeC8 3.5 micron, 4.6×50 mm; Gradient [time (min)/solvent B1 in A1(%)]: 0.0/5, 2.5/95, 4.0/95, 4.5/5, 6.0/5; Injection volume 1 μL; UV detection 210 to 400 nM; Column temperature 25° C.; 1.5 mL/min.

Method 3. Instruments: Agilent Technologies 1290 Infinity II Series LC, 6125 Quadrupole MSD SL; Column: Zorbax XDB C18, 5 micron; Gradient [time (min)/solvent B2 in A4(%)]: 0.00/5, 2.50/95, 4.00/95, 4.50/5, 6.00/5; Injection volume 1 μL; UV detection 210-400 nm; Column temperature 25° C.; Flow rate 1.5 mL/min.

Method 4. Instruments: Agilent Technologies 1260 LC with Chemstation software, Diode Array Detector, Agilent 6120 Quadrupole MS with APCI and ES Source; Column: Phenomenex Gemini-NX C18, 3 micron, 2×30 mm; Gradient [time (min)/solvent B3 in A3(%)]: 0.00/5, 2.00/95, 2.50/95, 2.60/5, 3.00/5; Injection volume 0.5 μL; UV detection 190-400 nm; column temperature 40° C.; Flow rate 1.5 mL/min.

Method 5. Instruments: Waters Acquity UPLC, Waters 3100 PDA Detector, SQD; Column: Acquity BEH C-18, 1.7 micron, 2.1×100 mm; Gradient [time (min)/solvent B2 in A2(%)]: 0.00/2, 2.00/2, 7.00/50, 8.50/80, 9.50/2, 10.0/2; Injection volume 1 μL; Detection wavelength 214 nm; Column temperature 30° C.; Flow rate 0.3 mL per min.

Method 6. Instruments: Agilent Technologies 1290 Infinity II Series LC, 6125 Quadrupole MSD SL; Column: Waters XBridgeC8 3.5 micron, 4.6×50 mm; Gradient [time (min)/solvent B2 in A5(%)]: 0.0/10, 4.0/95, 5.0/95, 5.5/10, 7.0/10; Injection volume 1 μL; UV detection 210 to 400 nM; Column temperature 25° C.; Flow rate 1.2 mL/min.

Method 7. Instruments: Agilent Technologies 1290 Infinity II Series LC, 6125 Quadrupole MSD SL; Column: Zorbax eclipse plus C18, 1.8 micron, 2.1×50 mm; Gradient [time (min)/solvent B2 in A4(%)]: 0.0/5, 0.25/5, 2.5/100, 3.0/100, 3.1/5, 4.0/5; Injection volume 1 μL; UV detection 210-400 nm; Column temperature 25° C.; Flow rate 0.8 mL/min.

Method 8. Instruments: Waters Acquity UPLC, Waters 3100 PDA Detector, SQD; Column: Acquity HSS-T3, 1.8 micron, 2.1×100 mm; Gradient [time (min)/solvent B2 in A7(%)]: 0.0/10, 1.00/10, 2.00/15, 4.50/55, 6.00/90, 8.00/90, 9.00/10, 10.00/10; Injection volume 1 μL; Detection wavelength 214 nm; Column temperature 30° C.; Flow rate 0.3 mL per min.

Method 9. Instruments: Waters Acquity UPLC, Waters 3100 PDA Detector, SQD; Column: Acquity BEH C-18, 1.7 micron, 2.1×100 mm; Gradient [time (min)/solvent B2 in A2(%)]: 0.00/5, 0.25/5, 1.50/35, 2.50/95, 3.20/95 3.60/5, 4.00/5; Injection volume 1 μL; Detection wavelength 214 nm; Column temperature 35° C.; Flow rate 0.6 mL per min to 3.20 min then 0.8 mL per min.

Method 10. Instruments: Agilent Technologies 1290 Infinity II Series LC, 6125 Quadrupole MSD SL; Column: Zorbax extend C18, 5 micron, 4.6×50 mm; Gradient [time (min)/solvent B2 in A9(%)]: 0.0/10, 4.0/95, 5.0/95, 5.5/5, 6.0/5; Injection volume 1 μL; UV detection 210-400 nm; Column temperature 25° C.; Flow rate 1.2 mL/min.

Method 11. Instruments: Agilent Technologies 1290 Infinity II Series LC, 6125 Quadrupole MSD SL; Column: Acquity BEH C18, 1.7 micron, 2.1×50 mm; Gradient [time (min)/solvent B2 in A9(%)]: 0.0/5, 0.25/5, 2.5/100, 3.0/100, 3.1/5, 4.0/5; Injection volume 1 μL; UV detection 210-400 nm; Column temperature 25° C.; Flow rate 0.8 mL/min.

Method 12. Instruments: Agilent Technologies 1290 Infinity II Series LC, 6125 Quadrupole MSD SL; Column: Atlantis dC18, 5 micron, 4.6×50 mm; Gradient [time (min)/solvent B2 in A4(%)]: 0.0/5, 2.5/95, 4.0/95, 4.5/5, 6.0/5; Injection volume 1 μL; UV detection 210-400 nm; Column temperature 25° C.; Flow rate 1.5 mL/min.

### GCMS Methods

GCMS data are given in the format: Mass ion, electrospray mode (positive or negative), retention time.

Method 1. Instrument: Agilent GCMS 7890B; Column: HP-5 ms UI (30 m×250 μm×0.25 μm); Inlet temp: 250° C.; Split ratio: 75:1; Oven temp: 50° C., hold time 3 min; Ramp 1: 40° C./min to 300° C., hold time 2 min; Detector temperature: 310° C.; Column flow: 2 mL/min; Air flow: 300 mL/min; H₂ flow: 40 mL/min; Make up flow (He): 25 mL/min; Source temp: 230° C.

Method 2. Instrument: Agilent GCMS 7890B; Column: HP-5 ms UI (30 m×250 μm×0.25 μm); Inlet temp: 250° C.; Split ratio: 75:1; Oven temp: 120° C., hold time 1 min; Ramp 1: 40° C./min to 300° C., hold time 4 min; Detector temperature: 310° C.; Column flow: 2 mL/min; Air flow: 300 mL/min; H₂ flow: 40 mL/min; Make up flow (He): 25 mL/min; Source temp: 230° C.

### MS Methods

Method 1. Data acquired on either a Waters QDA or Waters SQD instrument after a 4-6 minute run through a UPLC column using buffer.

### Prep HPLC Methods

See LCMS methods section for solvent conditions.

Method 1. Instruments: Agilent Technologies 1260 Infinity II Series LC/6125 Quadrupole MSD; Column: Waters XBridge C8 5 micron 19×150 mm; Gradient [time (min)/solvent B2 in A5(%)]: 0.0/10, 15/95, 18/95, 19/10, 21/10.

Method 2. Instruments: Gilson Semi Preparative HPLC System—321 Pump/171 Diode Array Detector/GX-271 Liquid Handler; Column: Phenomenex Gemini-NX C18 5 micron 30×100 mm; Gradient 12.5 min, solvent B2 in A6(%) varies on individual run basis (see exemplified procedures for details).

Method 3. Instruments: Waters 2767 Auto purification; Column: Reprosil Gold C18 5 micron 19×250 mm; Gradient 15 min, solvent B2 in A2(%) varies on individual run basis (see exemplified procedures for details).

Method 4. Instruments: Agilent Technologies 1260 Infinity II Series LC/6125 Quadrupole MSD; Column: Waters XBridge C8 5 micron 19×150 mm; Gradient [time (min)/solvent B2 in A7(%)]: 0.0/10, 15/95, 18/95, 19/10, 21/10.

Method 5. Instruments: Waters 2767 Auto purification; Column: Xtimate hexyl phenyl 10 micron 19×250 mm; Gradient 18 min, solvent B2 in A8(%) varies on individual run basis (see exemplified procedures for details).

Method 6. Instruments: Waters 2767 Auto purification; Column: X Select C18 10 micron 19×250 mm; Gradient 20 min, solvent B2 in A8(%) varies on individual run basis (see exemplified procedures for details).

### Chiral SFC Methods

Method 1. Instruments: PIC Solution PIC-100, PIC-150, PIC-175 and PIC-400; Column: Lux A1 5 micron, 21.2×250 mm; Co-solvent 0.5% iso-propyl amine in MeOH; Column temperature 35° C.; 20 mL/min.

Method 2. Instruments: Sepiatec Prep SFC 100 with Prep SFC 100 control software and UV/Vis detector; Column: Lux C1 5 micron, 21.2×250 mm; Co-solvent 0.2% NH₃ in IPA; Column temperature 40° C.; 50 mL/min.

Method 3. Instruments: PIC Solution PIC 10-20 and PIC-10; Column: Lux A1 3 micron, 2×50 mm; Co-solvent 0.5% iso-propyl amine in MeOH; Column temperature 35° C.; 3 mL/min.

Method 4. Instruments: Waters Acquity UPC2 with Masslynx software, PDA detector and a QDa mass detector; Column: Lux C1 3 micron, 2×50 mm; Co-solvent IPA; Column temperature 45° C.; 1.5 mL/min.

Method 5. Instruments: PIC Solution PIC-100, PIC-150, PIC-175 and PIC-400; Column: Lux A1 5 micron, 21.2×250 mm; Co-solvent MeOH/MeCN (1:1); Column temperature 35° C.; 30 mL/min.

Method 6. Instruments: PIC Solution PIC 10-20 and PIC-10; Column: Lux A1 3 micron, 2×50 mm; Co-solvent MeOH/MeCN (1:1); Column temperature 35° C.; 3 mL/min.

Method 7. Instruments: PIC Solution PIC-100, PIC-150, PIC-175 and PIC-400; Column: Chiralpak AS-H, 30×250 mm; Co-solvent 0.5% iso-propyl amine in MeOH; Column temperature 35° C.; 30 mL/min.

### Abbreviations

aq=aqueous
Boc=tert-butoxycarbonyl
DAST=(diethylamino)sulfur trifluoride
Davis reagent=2-(phenylsulfonyl)-3-phenyl-oxaziridine
dba=dibenzylideneacetone
DCM=dichloromethane
Dess-Martin=1,1,1-tris(acetyloxy)-1,1-dihydro-1,2-benziodoxol-3-(1H)-one

### DIPEA=N,N-diisopropylethylamine

### DMA=N,N-Dimethylacetamide

### DMAP=4-(dimethylamino)pyridine

DME=1,2-dimethoxyethane

### DMF=N,N-dimethylformamide

DMF-DMA=N,N-dimethylformamide dimethyl acetal
DMS=dimethyl sulfide
DMSO=dimethylsulfoxide
dppf=1,1′-ferrocenediyl-bis(diphenylphosphine)
EDCI=N-(3-dimethylaminopropyl)-N′-ethylcarbodiimide hydrochloride
ES=electrospray
EtOAc=ethyl acetate
EtOH=ethanol
h=hour(s)
HATU=N-[(Dimethylamino)-1H-1,2,3-triazolo-[4,5-b]pyridin-1-ylmethylene]-N-methylmethanaminium hexafluorophosphate N-oxide
HMDS=hexamethyldisilazane
IPA=i-propyl alcohol
L=litre
LC=liquid chromatography
LCMS=liquid chromatography mass spectrometry
LiAlH₄=lithium aluminum hydride
MeCN=acetonitrile
MeOH=methanol
min=minute(s)
MS=mass spectrometry
NMP=1-methyl-2-pyrrolidinone
NMR=nuclear magnetic resonance
MTBE=methyl tert-butyl ether

### NIS=N-iodosuccinimide

Pet-ether=petroleum ether
pin=pinacol
RT=room temperature
RuPhos=2-dicyclohexylphosphino-2′,6′-diisopropoxybiphenyl
SFC=supercritical fluid chromatography
SPhos=2-dicyclohexylphosphino-2′,6′-dimethoxybiphenyl
TFA=trifluoroacetic acid
THF=tetrahydrofuran
TMSI=iodotrimethylsilane
TrixiePhos=rac-2-(di-tert-butylphosphino)-1,1′-binaphthyl
Ts=para-toluenesulfonyl
XPhos=2-dicyclohexylphosphino-2′,4′,6′-triisopropylbiphenyl

Prefixes n-, s-, i-, t- and tert- have their usual meanings: normal, secondary, iso, and tertiary.

### Synthesis of Intermediates

### Preparation of Substituted (Dimethylamino)Methylene Intermediates

### Typical Procedure 1, Exemplified by the Preparation of Intermediate 1, (E)-3-((dimethylamino)methylene)piperidine-2,4-dione

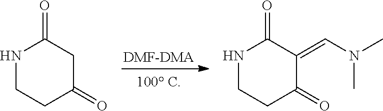

A solution of piperidine-2,4-dione (10 g, 88.5 mmol) in DMF-DMA (100 mL) was stirred at 100° C. for 3 h and then at RT for 15 h. Precipitated solid was filtered, rinsed with Et₂O (2×30 mL) and dried in vacuo to afford (E)-3-((dimethylamino)methylene)piperidine-2,4-dione as a brown solid (5 g, 33%). Data in table 2.

### Intermediate 2, 2-((dimethylamino)methylene)cyclohexane-1,3-dione

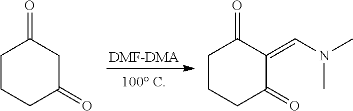

The title compound (1.3 g, 88%) was prepared from cyclohexane-1,3-dione (1 g, 8.8 mmol) in DMF-DMA (10 mL) heated at 100° C. for 3 h using the method of Intermediate 1. Data in table 2.

### Intermediate 17, (E)-3-((dimethylamino)methylene)-6-methylpiperidine-2,4-dione

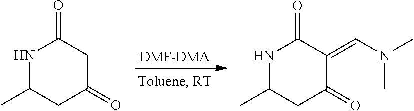

The title compound (390 mg, 91%) was prepared from 6-methylpiperidine-2,4-dione (300 mg, 2.36 mmol) in DMF-DMA (0.5 mL) and toluene (5 mL) stirred at RT for 2 h using the method of Intermediate 1. Data in table 2.

### Intermediate 20, (E)-3-((dimethylamino)methylene)-5-methylpiperidine-2,4-dione

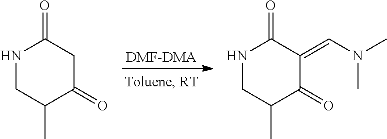

The title compound (257 mg, 90%) was prepared from 5-methylpiperidine-2,4-dione (200 mg, 1.56 mmol) in DMF-DMA (0.3 mL) and toluene (2 mL) stirred at RT for 2 h using the method of Intermediate 1. Data in table 2.

### Typical Procedure 2, Exemplified by the Preparation of Intermediate 19, ethyl (Z)-4-(tert-butoxy)-2-((dimethylamino)methylene)-3-oxobutanoate

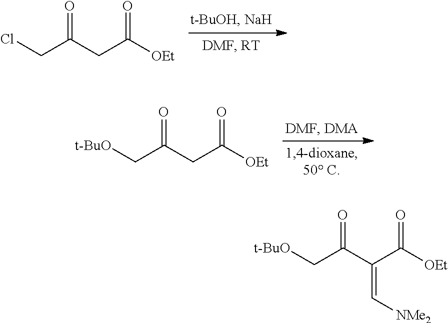 [SMILES: CCOC(=O)CC(=O)CCl ; CCOC(=O)CC(=O)COC(C)(C)C ; CCOC(=O)/C(=C\N(C)C)C(=O)COC(C)(C)C]

Step 1. Ethyl 4-chloro-3-oxobutanoate (2.00 g, 12.2 mmol) and t-BuOH (1.80 g, 24.3 mmol) were added to a suspension of sodium hydride (60% in mineral oil, 1.46 g, 36.5 mmol) in DMF (5 mL) and the reaction mixture was stirred at RT for 16 h. The reaction mixture was quenched with 1N HCl (20 mL) and the aqueous layer was extracted with EtOAc (3×50 mL). The organic layers were combined, dried (Na₂SO₄) and the solvent removed in vacuo to afford ethyl 4-(tert-butoxy)-3-oxobutanoate as a yellow liquid (1.60 g, 65%).

¹H NMR: (400 MHz, CDCl₃) δ: 4.14 (q, J=6.8 Hz, 2H), 3.96 (s, 2H), 3.49 (s, 2H), 1.26-1.20 (m, 3H), 1.14 (s, 9H).

Step 2. DMF-DMA (1.27 mL) was added dropwise to a solution of ethyl 4-(tert-butoxy)-3-oxobutanoate (1.60 g, 7.91 mmol) in 1,4-dioxane (10 mL). The reaction mixture was heated at 50° C. for 2 h. The reaction mixture was concentrated in vacuo to afford ethyl (Z)-4-(tert-butoxy)-2-((dimethylamino)methylene)-3-oxobutanoate as a brown liquid (1.60 g, 79%). Data in table 2.

### Preparation of Substituted Dihydropyridinone and Equivalent Intermediates

### Typical Procedure 3, Exemplified by the Preparation of Intermediate 35, 3-acetyl-1-(2,4-dimethoxybenzyl)-4-hydroxy-5,6-dihydropyridin-2(1H)-one

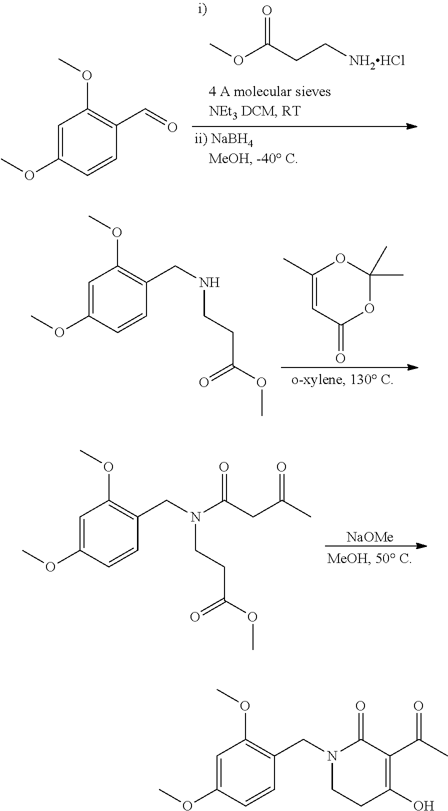

Step 1. 4 Å Molecular sieves (10 g) were added to a suspension of 2,4-dimethoxybenzaldehyde (5 g, 30.1 mmol) and methyl 3-aminopropanoate hydrochloride (5.38 g, 38.7 mmol) in DCM (100 mL) at RT followed by the addition of triethyl amine (15 mL, 107 mmol) and the reaction mixture was stirred at RT for 15 h. The reaction mixture was filtered through celite and washed with DCM (100 mL). The filtrate was washed with 10% NaHCO₃ solution (100 mL) and brine (100 mL). The organic layer was separated, dried (Na₂SO₄) and the solvent removed in vacuo. The residue was dissolved MeOH (50 mL), cooled to −40° C. and NaBH₄ (1.71 g, 45.2 mmol) was added portion-wise and the reaction mixture was stirred at −40° C. for 1 h. The solvent was removed in vacuo and the residue obtained was dissolved in EtOAc (200 mL) and washed with H₂O (200 mL). The organic layer was separated, dried (Na₂SO₄) and the solvent removed in vacuo to afford methyl 3-((2,4-dimethoxybenzyl)amino)propanoate as a pale yellow gum (6.8 g, 89%).

LCMS (Method 3): m/z 254.1 (ES+), at 1.02 min.

¹H NMR: (400 MHz, CDCl₃) δ: 7.15 (d, J=8.0 Hz, 1H), 6.48-6.44 (m, 2H), 3.84 (s, 3H), 3.82 (s, 3H), 3.75 (s, 2H), 3.70 (s, 3H), 2.87 (t, J=6.4 Hz, 2H), 2.55 (t, J=6.8 Hz, 2H). 1 exchangeable proton not observed.

Step 2. A solution of methyl 3-((2,4-dimethoxybenzyl)amino)propanoate (6.8 g, 26.8 mmol) and 2,2,6-trimethyl-4H-1,3-dioxin-4-one (7.6 g, 53.7 mmol) in o-xylene (100 mL) was stirred at 130° C. for 4 h. The solvent was removed in vacuo and the residue was purified by gradient flash column chromatography eluting with 0-50% EtOAc in pet-ether to afford methyl 3-(N-(2,4-dimethoxybenzyl)-3-oxobutanamido)propanoate as a yellow gum (6.0 g, 66%).

LCMS (Method 7): m/z 338.2 (ES+), at 1.82 min.

¹H NMR: (400 MHz, DMSO-d₆) δ: 7.06 (d, J=11.2 Hz, 1H), 6.59-6.47 (m, 2H), 4.42-4.34 (m, 2H), 3.79-3.75 (m, 9H), 3.64 (s, 2H), 3.58-3.56 (m, 4H), 2.12 (s, 3H).

Step 3. Sodium methoxide (0.6 g, 11.3 mmol) was added to a stirred solution of methyl 3-(N-(2,4-dimethoxybenzyl)-3-oxobutanamido)propanoate (2.5 g, 7.42 mmol) in MeOH (50 mL) and the reaction mixture was heated at 50° C. for 4 h. The solvent was removed in vacuo and the residue was dissolved in EtOAc (50 mL) and washed with water (50 mL). The organic layer was separated, washed with brine solution (50 mL), dried (Na₂SO₄) and the solvent removed in vacuo. The residue was purified by gradient flash column chromatography eluting with 0-40% EtOAc in pet-ether to afford 3-acetyl-1-(2,4-dimethoxybenzyl)-4-hydroxy-5,6-dihydropyridin-2(1H)-one as an off-white solid (450 mg, 20%). Data in table 2.

### Typical Procedure 4, Exemplified by the Preparation of Intermediate 44, tert-butyl 3-(2-methoxyacetyl)-2,4-dioxopiperidine-1-carboxylate

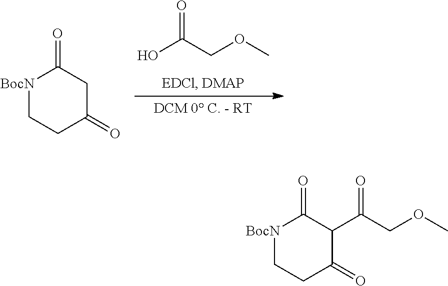

N-(3-Dimethylaminopropyl)-N′-ethylcarbodiimide hydrochloride (0.180 g, 0.94 mmol) and DMAP (0.126 g, 1.03 mmol) were added to a stirred solution of tert-butyl 2,4-dioxopiperidine-1-carboxylate (0.2 g, 0.94 mmol) and 2-methoxyacetic acid (0.084 g, 0.94 mmol) in DCM (10 mL) at 0° C. and the reaction mixture was stirred at RT for 2 h. The reaction mixture was partitioned between water (30 mL) and DCM (2×30 mL). The combined organic layers were washed with brine solution (30 mL), dried (Na₂SO₄) and the solvent removed in vacuo. The residue was purified by gradient flash column chromatography eluting with 0-20% EtOAc in pet-ether to afford tert-butyl 3-(2-methoxyacetyl)-2,4-dioxopiperidine-1-carboxylate as a yellow liquid (0.2 g, 75%). Data in table 2.

### Preparation of Substituted Fluoropyridine Intermediates

### Typical Procedure 5, Exemplified by the Preparation of Intermediate 3, 4-fluoro-2-(3-fluoro-5-(trifluoromethyl)benzyl)pyridine

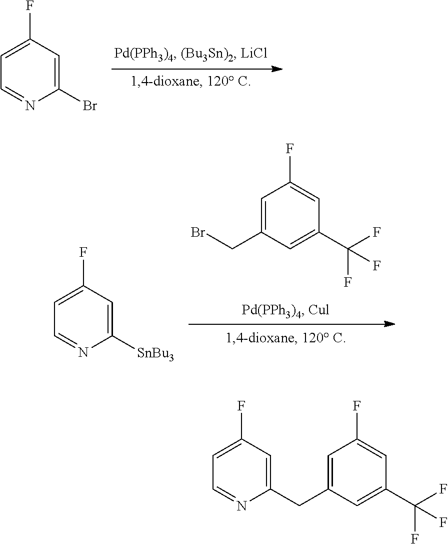

Step 1. A solution of 2-bromo-4-fluoropyridine (4.0 g, 22.7 mmol) in 1,4-dioxane (60 mL) was degassed with argon for 10 min and bis(tributyltin) (17.3 mL, 34.0 mmol), LiCl (2.88 g, 68.1 mmol) and Pd(PPh₃)₄ (1.31 g, 1.13 mmol) were added. The reaction mixture was heated at 120° C. for 16 h. The reaction was quenched with water (100 mL) and the aqueous layer was extracted with EtOAc (2×100 mL). The organic layers were combined, dried (Na₂SO₄) and the solvent removed in vacuo to afford 4-fluoro-2-(tributylstannyl)pyridine as a yellow liquid (14.3 g crude). The crude product was used in the next step without further purification.

MS (Method 1): m/z 388 (ES+)

Step 2. 1-(Bromomethyl)-3-fluoro-5-(trifluoromethyl)benzene (1.33 g, 5.18 mmol) was added to a solution of 4-fluoro-2-(tributylstannyl)pyridine (14.3 g, 5.18 mmol) in 1,4-dioxane (30 mL). The reaction mixture was degassed with argon for 10 min and CuI (98 mg, 0.51 mmol), Pd(PPh₃)₄ (299 mg, 0.26 mmol) were added. The reaction mixture was heated at 120° C. for 16 h. The reaction was quenched with water (30 mL) and the aqueous layer was extracted EtOAc (2×100 mL). The combined organic layers were washed with brine, dried (Na₂SO₄) and the solvent removed in vacuo. The residue was purified by gradient flash column chromatography eluting with 5-10% EtOAc in hexane to afford 4-fluoro-2-(3-fluoro-5-(trifluoromethyl)benzyl)pyridine as a light yellow liquid (400 mg, 6.4% over two steps). Data in table 2.

### Typical Procedure 6, Exemplified by the Preparation of Intermediate 4, 2-fluoro-4-(3-(trifluoromethyl)benzyl)pyridine

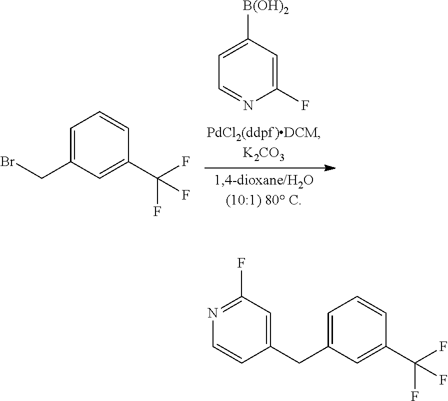 [SMILES: OB(O)c1ccnc(F)c1 ; FC(F)(F)c1cccc(CBr)c1 ; Fc1cc(Cc2cccc(C(F)(F)F)c2)ccn1]

1-(Bromomethyl)-3-(trifluoromethyl)benzene (0.14 mL, 0.88 mmol) was added to a suspension of 2-fluoropyridine-4-boronic acid (150 mg, 1.06 mmol), K₂CO₃ (146 mg, 1.06 mmol) and PdCl₂(dppf).DCM (129 mg, 0.18 mmol) in 1,4-dioxane (4 mL)/water (0.4 mL) and the reaction mixture heated at 80° C. for 2 hours. The reaction mixture was partitioned between water (6 mL) and EtOAc (6 mL) and the organic layer removed. The aqueous layer was extracted with EtOAc (2×6 mL), the combined organic layers dried (phase separator) and the solvent removed in vacuo. The residue was purified by gradient flash column chromatography eluting with 0-50% EtOAc in i-hexane to afford 2-fluoro-4-(3-(trifluoromethyl)benzyl)pyridine as a yellow liquid (167 mg, 74%). Data in table 2.

### Intermediate 5, 2-fluoro-4-(3-fluoro-5-(trifluoromethyl)benzyl)pyridine

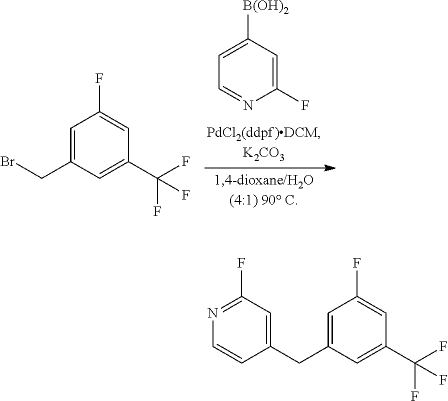 [SMILES: Fc1cc(CBr)cc(C(F)(F)F)c1 ; OB(O)c1ccnc(F)c1 ; Fc1cc(Cc2ccnc(F)c2)cc(C(F)(F)F)c1]

The title compound (1.8 g, 86%) was prepared from (2-fluoropyridin-4-yl)boronic acid (1.3 g, 7.77 mmol), 1-(bromomethyl)-3-fluoro-5-(trifluoromethyl)benzene (1.3 g, 9.30 mmol), PdCl₂(dppf).DCM (284 mg, 0.38 mmol) and K₂CO₃ (3.2 g, 22.3 mmol) in 1,4-dioxane (20 mL)/water (5 mL) heated at 90° C. for 1 h using the method of Intermediate 4. The title compound was isolated as a brown oil by partitioning between EtOAc (100 mL) and water (70 mL). The organic layer was separated, washed with brine (50 mL), dried (Na₂SO₄) and the solvent removed in vacuo. The residue was purified by gradient flash column chromatography eluting with 0-30% EtOAc in pet-ether. Data in table 2.

### Intermediate 18, 2-chloro-4-(3-fluoro-5-(trifluoromethyl)benzyl)pyridine

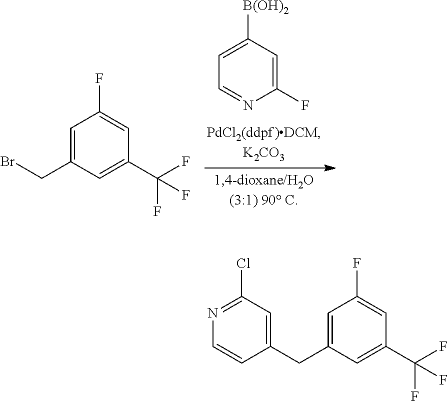 [SMILES: Fc1cc(CBr)cc(C(F)(F)F)c1 ; OB(O)c1ccnc(F)c1 ; Fc1cc(Cc2ccnc(Cl)c2)cc(C(F)(F)F)c1]

The title compound (3.00 g, 88%) was prepared from (2-chloropyridin-4-yl)boronic acid (1.84 g, 11.7 mmol), 1-(bromomethyl)-3-fluoro-5-(trifluoromethyl)benzene (3.00 g, 11.7 mmol), PdCl₂(dppf).DCM (476 mg, 0.58 mmol) and K₂CO₃ (4.84 g, 35.0 mmol) in 1,4-dioxane (33.7 mL)/water (11.3 mL) heated at 90° C. for 2 h using the method of Intermediate 4. The title compound was isolated as a colourless oil by partitioning between EtOAc (50 mL) and water (50 mL). The organic layer was separated and the aqueous layer extracted with EtOAc (2×50 mL). The combined organic layers were dried (Na₂SO₄) and the solvent removed in vacuo. The residue was purified by gradient flash column chromatography eluting with 10-12% EtOAc in hexane. Data in table 2.

### Preparation of Substituted Hydrazineyl Intermediates

### Typical Procedure 7, Exemplified by the Preparation of Intermediate 6, 2-(3-fluoro-5-(trifluoromethyl)benzyl)-4-hydrazineylpyridine

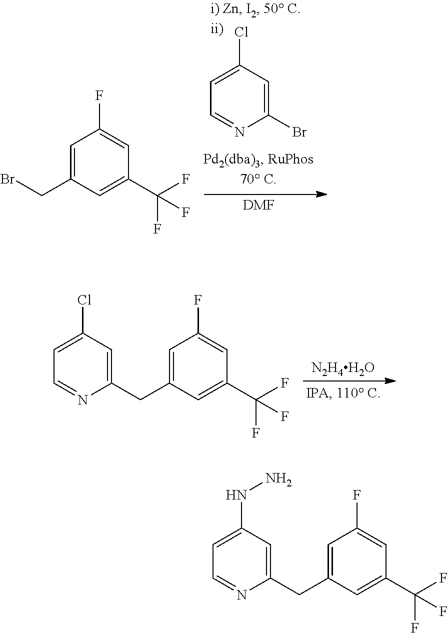

Step 1. A pinch of iodine was added to a stirred solution of activated zinc (35 g, 583 mmol) in DMF (300 mL) and the solution heated at 50° C. for 5 min followed by the addition of 1-(bromomethyl)-3-fluoro-5-(trifluoromethyl)benzene (32 g, 124 mmol) in DMF (50 mL). The reaction mixture was heated at 50° C. for 1 h and then allowed to cool to RT. The residual zinc was allowed to settle and the supernatant pale green DMF layer was transferred via cannula to a degassed suspension of 2-bromo-4-chloropyridine (16 g, 83.3 mmol) and RuPhos (2.3 g, 4.99 mmol) in DMF (50 mL) followed by the addition of tris(dibenzylideneacetone)dipalladium(0) (3.8 g, 4.16 mmol). The reaction mixture was heated at 70° C. for 16 h and then filtered through Celite and washed with EtOAc (600 mL). The filtrate was washed with brine (3×300 mL). The organic layer was separated, dried (Na₂SO₄) and the solvent removed in vacuo. The residue was purified by gradient flash column chromatography eluting with 0-5% EtOAc in pet-ether to afford 4-chloro-2-(3-fluoro-5-(trifluoromethyl)benzyl)pyridine as a yellow semi solid (8 g, 33%).

LCMS (Method 3): m/z 290.1 (ES+), at 2.65 min.

¹H NMR: (400 MHz, DMSO-d₆) δ: 8.49 (d, J=5.2 Hz, 1H), 7.83-7.79 (m, 1H), 7.61-7.41 (m, 4H), 4.23 (s, 2H).

Step 2. Hydrazine hydrate (20 g, 415 mmol) was added to a stirred solution of 4-chloro-2-(3-fluoro-5-(trifluoromethyl)benzyl)pyridine (8 g, 27.68 mmol) in IPA (100 mL) in a sealed tube and the reaction mixture was heated at 110° C. for 72 h. The solvent was removed in vacuo and the residue partitioned between water (200 mL) and EtOAc (200 mL). The organic layer was separated, washed with brine (200 mL), dried (Na₂SO₄) and the solvent removed in vacuo to afford 2-(3-fluoro-5-(trifluoromethyl)benzyl)-4-hydrazineylpyridine as a yellow gum (5 g, 63%). Data in table 2.

### Intermediate 7, 2-hydrazinyl-4-(3-(trifluoromethyl)benzyl)pyridine

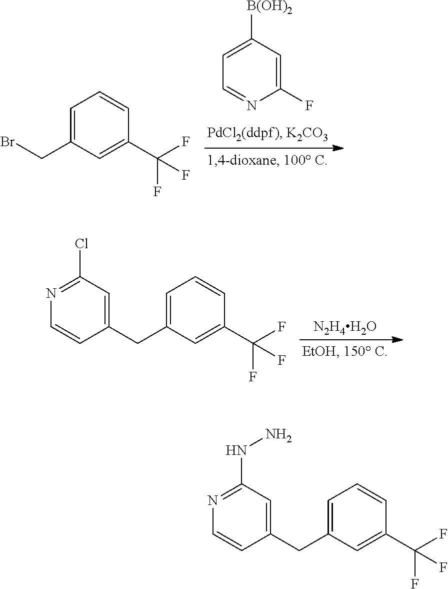

The title compound (486 mg, 80%) was prepared in two steps from (2-chloropyridin-4-yl)boronic acid (3.6 g, 23.2 mmol), 1-(bromomethyl)-3-(trifluoromethyl)benzene (5 g, 21.09 mmol), PdCl₂(dppf).DCM (3.4 g, 4.21 mmol, 20 mol %) and K₂CO₃ (3.4 g, 25.3 mmol) in 1,4-dioxane (40 mL) heated at 100° C. for 1 h; and step 1 product (500 mg, 1.84 mmol), hydrazine hydrate (0.45 mL, 9.20 mmol) in EtOH (4 mL) heated at 150° C. for 12 h using the methods of Intermediate 4 and Intermediate 6, step 2. After completion of step 2, the title compound was isolated as an orange oil by partitioning between EtOAc (6 mL) and water (6 mL). The aqueous layer was washed with EtOAc (2×6 mL), dried (phase separator) and the solvent removed in vacuo. Data in table 2.

### Intermediate 39, 4-(3-(difluoromethyl)-5-fluorobenzyl)-2-hydrazineylpyridine

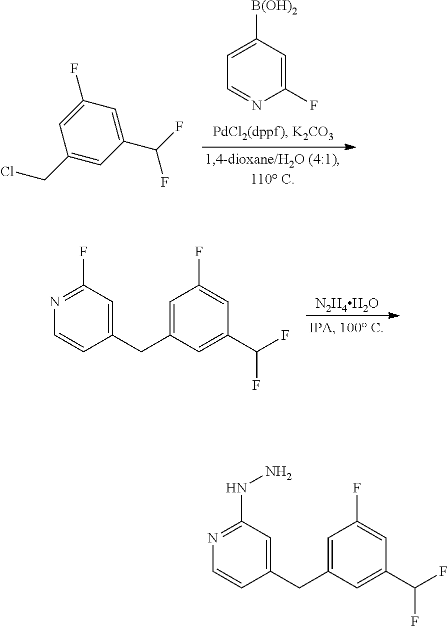 [SMILES: Fc1cc(CCl)cc(C(F)F)c1 ; OB(O)c1ccnc(F)c1 ; Fc1cc(Cc2ccnc(F)c2)cc(C(F)F)c1 ; "N2H4·H2O" ; NNc1cc(Cc2cc(F)cc(C(F)F)c2)ccn1]

The title compound (110 mg, crude) was prepared in two steps from (2-fluoropyridin-4-yl)boronic acid (145 mg, 1.03 mmol), 1-(chloromethyl)-3-(difluoromethyl)-5-fluorobenzene (Intermediate 26, 200 mg, 1.03 mmol), PdCl₂(dppf).DCM (84 mg, 0.103 mmol) and K₂CO₃ (426 mg, 3.09 mmol) in 1,4-dioxane (8 mL)/water (2 mL) heated at 110° C. for 16 h; and hydrazine hydrate (0.5 mL, 9.77 mmol) in IPA (10 mL) heated at 100° C. for 48 h using the methods of Intermediate 4 and Intermediate 6, step 2. After completion of step 2, the title compound was isolated as a yellow gum by partitioning between EtOAc (10 mL) and water (10 mL). The aqueous layer was extracted with EtOAc (10 mL). The combined organic layers were washed with brine solution (10 mL), dried (Na₂SO₄) and the solvent removed in vacuo. The crude product was used in the next step without further purification. Data in table 2.

### Intermediate 8, 4-(3-fluoro-5-(trifluoromethyl)benzyl)-2-hydrazineylpyridine

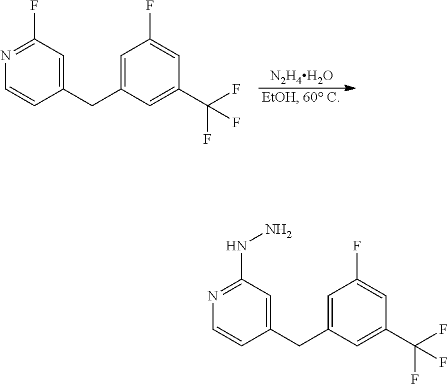

The title compound (350 mg, 100%) was prepared from 2-fluoro-4-(3-fluoro-5-(trifluoromethyl)benzyl)pyridine (Intermediate 5, 300 mg, 1.17 mmol) and hydrazine hydrate (0.17 mL, 3.52 mmol) in EtOH (10 mL) heated at 60° C. for 16 h using the method of Intermediate 6, step 2. The title compound was isolated as a brown oil by partitioning between EtOAc (50 mL) and water (50 mL). The organic layer was separated, washed with brine (50 mL), dried (Na₂SO₄) and the solvent removed in vacuo. Data in table 2.

### Typical Procedure 8, Exemplified by the Preparation of Intermediate 9, 5-(3-fluoro-5-(trifluoromethyl)benzyl)-3-hydrazineylpyridazine

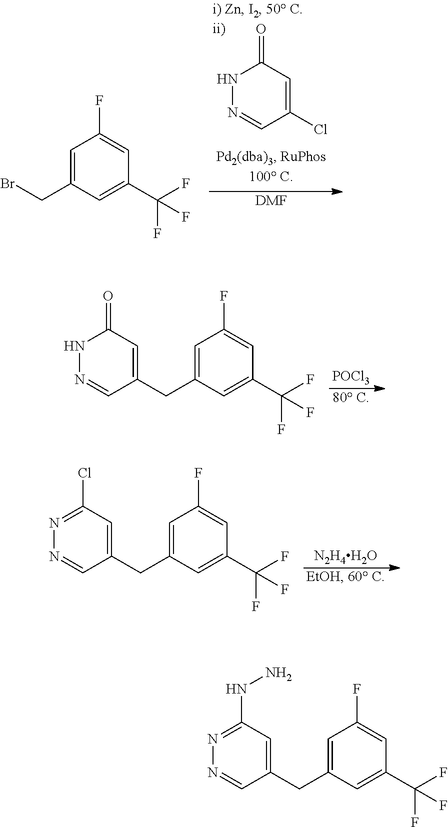

Step 1. A pinch of iodine was added to a stirred solution of activated zinc (1.49 g, 23.0 mmol) in dry DMF (30 mL) and heated to 50° C. for 5 min followed by the addition of 1-(bromomethyl)-3-fluoro-5-(trifluoromethyl)benzene (1.19 g, 4.61 mmol) in DMF (10 mL). The reaction mixture was heated at 50° C. for 1 h and then allowed to cool to RT. The residual zinc was allowed to settle and the supernatant pale green DMF layer was transferred via cannula to a degassed suspension of 5-chloropyridazin-3(2H)-one (200 mg, 1.53 mmol), RuPhos (70 mg, 0.15 mmol) and tris(dibenzylideneacetone)dipalladium(0) (140 mg, 0.15 mmol) in DMF (10 mL) and the reaction mixture was heated to 100° C. for 16 h and then filtered through Celite and washed with EtOAc (2×50 mL). The filtrate was washed with water (2×70 mL). The organic layer was separated, dried (Na₂SO₄) and the solvent removed in vacuo. The residue was purified by gradient flash chromatography eluting with 0-20% EtOAc in pet-ether to afford 5-(3-fluoro-5-(trifluoromethyl)benzyl)pyridazin-3(2H)-one as a pale yellow liquid (95 mg, 21%).

LCMS (Method 3): m/z 273.1 (ES+), at 1.77 min.

¹H NMR: (400 MHz, DMSO-d₆) δ: 12.94 (s, 1H), 7.86 (d, J=2.8 Hz, 1H), 7.63-7.57 (m, 3H), 6.71 (s, 1H), 3.96 (s, 2H).

Step 2. A suspension of 5-(3-fluoro-5-(trifluoromethyl)benzyl)pyridazin-3(2H)-one (90 mg) in POCl₃ (2 mL) was heated at 80° C. for 1 h. The reaction mixture was quenched with ice-water (30 mL) and extracted with EtOAc (30 mL). Organic layer was separated, washed with brine (20 mL), dried (Na₂SO₄) and the solvent removed in vacuo to afford 3-chloro-5-(3-fluoro-5-(trifluoromethyl)benzyl)pyridazine as a brown liquid (120 mg, crude). The crude product was used in the next step without further purification.

LCMS (Method 3): m/z 291.0 (ES+), at 2.26 min.

Step 3. Hydrazine hydrate (0.2 ml, 0.68 mmol) was added to a stirred solution of 3-chloro-5-(3-fluoro-5-(trifluoromethyl)benzyl)pyridazine (100 mg) in ethanol (20 mL) and the reaction mixture was heated at 80° C. for 16 h. The solvent was removed in vacuo and the residue partitioned between water (50 mL) and EtOAc (50 mL). The organic layer was separated, washed with brine (20 mL), dried (Na₂SO₄) and the solvent removed in vacuo to afford 5-(3-fluoro-5-(trifluoromethyl)benzyl)-3-hydrazineylpyridazine as a pale yellow liquid (90 mg, crude). The crude product was used in the next step without further purification. Data in table 2.

### Typical Procedure 9, Exemplified by the Preparation of Intermediate 42, tert-butyl 2-(3-(3-fluoro-5-(trifluoromethyl)benzyl)phenyl)hydrazine-1-carboxylate

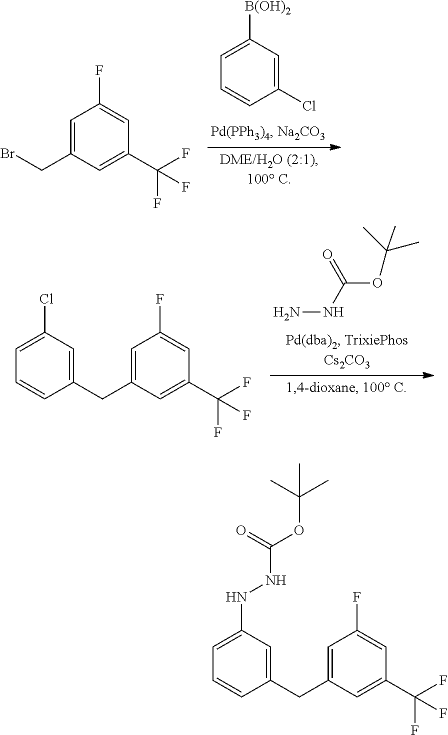

Step 1. (3-Chlorophenyl)boronic acid (188 mg, 1.2 mmol), 3-fluoro-5-(trifluoromethyl)benzyl bromide (0.16 mL, 1.00 mmol), Pd(PPh₃)₄ (23 mg, 0.02 mmol) and sodium carbonate (223 mg, 2.1 mmol) were dissolved in DME/water (2 mL:1 mL) and the reaction mixture degassed with N₂ for 5 min. The reaction mixture was then heated at 100° C. in a microwave for 3 h. The reaction mixture was diluted with water (3 mL) and extracted with DCM (3×3 mL). The combined organic layers were dried (phase separator) and the solvent removed in vacuo. The residue was purified by gradient flash column chromatography eluting with 0-100% Et₂O in i-hexane to give 1-[(3-chlorophenyl)methyl]-3-fluoro-5-(trifluoromethyl)benzene as a clear oil (303 mg, quant.).

LCMS (Method 1): m/z No ionisation observed (ES+), at 6.15 min.

¹H NMR: (400 MHz, CDCl₃) δ: 7.28-7.26 (m, 2H), 7.23 (tq, J=1.7, 0.6 Hz, 1H), 7.20 (ddq, J=2.2, 1.7, 0.6 Hz, 1H), 7.19 (ddd, J=2.3, 1.4, 0.8 Hz, 1H), 7.10-7.03 (m, 2H), 4.02 (d, J=0.8 Hz, 2H).

Step 2. tert-Butyl carbazate (166 mg, 1.26 mmol), Pd(dba)₂ (19 mg, 0.03 mmol), rac-2-(di-tert-butylphosphino)-1,1′-binaphthyl (13 mg, 0.03 mmol) and cesium carbonate (514 mg, 1.57 mmol) were placed in a microwave vial which was evacuated and backfilled with N₂ three times. 1-[(3-chlorophenyl)methyl]-3-fluoro-5-(trifluoromethyl)benzene (303 mg, 1.05 mmol) dissolved in 1,4-dioxane (3 mL) was added and the reaction mixture heated at 100° C. in a microwave for 8 h. The reaction mixture was partitioned between EtOAc (3 mL) and water (3 mL) and the organic layer removed. The aqueous layer was extracted with EtOAc (2×3 mL), the combined organic layers dried (phase separator) and the solvent removed in vacuo. The residue was purified by gradient flash column chromatography eluting with 0-20% Et₂O in i-hexane to give tert-butyl 2-(3-(3-fluoro-5-(trifluoromethyl)benzyl)phenyl)hydrazine-1-carboxylate as a clear oil (181 mg, 45%). Data in table 2.

### Preparation of 1-substituted 1,5,6,7-tetrahydro-4H-pyrazolo[4,3-c]pyridin-4-one and 1,5,6,7-tetrahydro-4H-pyrrolo[3,2-c]pyridin-4-one intermediates

### Typical Procedure 10, Exemplified by the Preparation of Intermediate 10, 1-(6-chloropyridazin-4-yl)-1,5,6,7-tetrahydro-4H-pyrazolo[4,3-c]pyridin-4-one

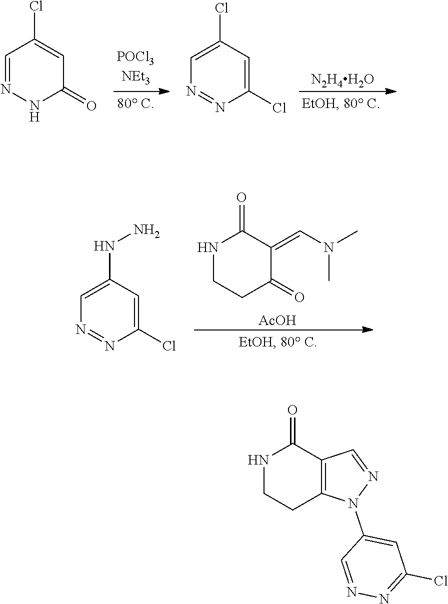

Step 1. Et₃N (2 mL) was added to suspension of 5-chloropyridazin-3(2H)-one (5 g, 38.3 mmol) in POCl₃ (50 mL) at 0° C. and the reaction mixture heated at 100° C. for 12 h. The solvent was removed in vacuo and the residue was purified by gradient flash chromatography eluting with 0-20% EtOAc in pet-ether to afford 3,5-dichloropyridazine as an off-white solid (2 g, 35%).

LCMS (Method 3): m/z 149.0 (ES+), at 1.24 min.

¹H NMR: (400 MHz, CDCl₃) δ: 9.15 (d, J=2.0 Hz, 1H), 7.62 (d, J=2.0 Hz, 1H).

Step 2. Hydrazine hydrate (0.5 mL) was added to a solution of 3,5-dichloropyridazine (2 g, 13.4 mmol) in EtOH (20 mL) and the reaction mixture was heated at 80° C. for 12 h. The solvent was removed in vacuo to afford 3-chloro-5-hydrazineylpyridazine (crude). The crude product was used in the next step without further purification.

LCMS: Not recorded.

¹H NMR: Not recorded.

Step 3. AcOH (0.1 mL) was added to a stirred solution of 3-chloro-5-hydrazineylpyridazine (200 mg, 1.39 mmol) and (E)-3-((dimethylamino)methylene) piperidine-2,4-dione (Intermediate 1, 278 mg, 1.66 mmol) in EtOH (20 mL) the reaction mixture was heated at 80° C. for 12 h. The solvent was removed in vacuo and the residue purified by prep HPLC (Method 1) to afford 1-(6-chloropyridazin-4-yl)-1,5,6,7-tetrahydro-4H-pyrazolo[4,3-c]pyridin-4-one (60 mg, 17%). Data in table 2.

### Intermediate 11, 1-(5-bromopyridin-3-yl)-1,5,6,7-tetrahydro-4H-pyrazolo[4,3-c]pyridin-4-one

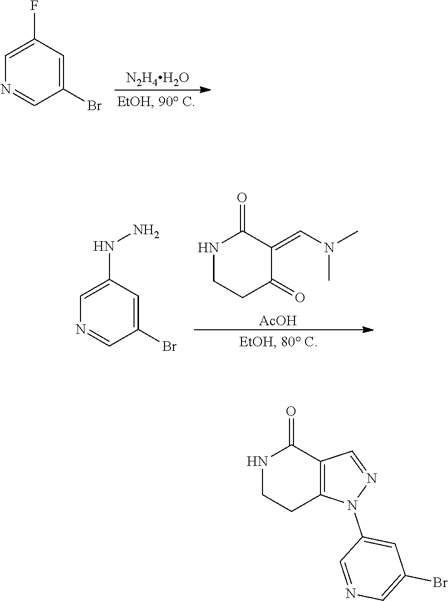

The title compound (200 mg, 98%) was prepared in two steps from 3-bromo-5-fluoropyridine (700 mg, 4.0 mmol) and hydrazine hydrate (1.92 g, 60 mmol) in EtOH (10 mL) heated at 90° C. for 12 h; and (E)-3-((dimethylamino)methylene)piperidine-2,4-dione (Intermediate 1, 402 mg, 2.39 mmol) and acetic acid (0.01 mL) in EtOH (20 mL) heated at 80° C. for 15 h using the methods of Intermediate 10, steps 2 and 3. After completion of step 2, the title compound was isolated as a yellow solid by filtration of the precipitated solid which was washed with EtOH (2×10 mL) and dried in vacuo. Data in table 2.

### Intermediate 40, 1-(5-fluoropyridin-3-yl)-1,5,6,7-tetrahydro-4H-pyrazolo[4,3-c]pyridin-4-one

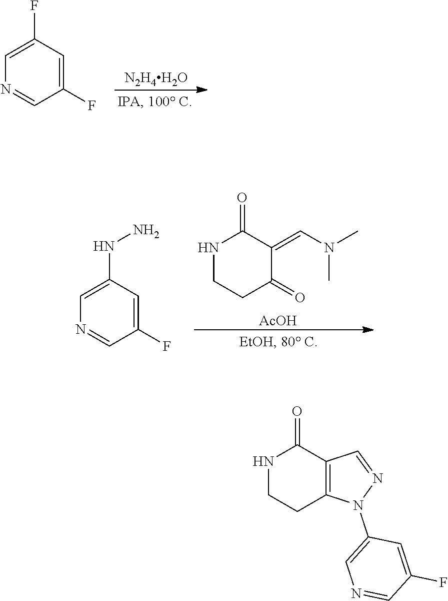

The title compound (370 mg, 37%) was prepared in two steps from 3,5-difluoropyridine (500 mg, 4.34 mmol and hydrazine hydrate (3.29 g, 65.17 mmol) in IPA (50 mL) heated at 100° C. for 12 h; and (E)-3-((dimethylamino)methylene)piperidine-2,4-dione (Intermediate 1, 1.37 g, 8.18 mmol) and acetic acid (0.05 mL) in EtOH (50 mL) heated at 80° C. for 16 h using the methods of Intermediate 10, steps 2 and 3. After completion of step 2, the title compound was isolated as a yellow solid by removal of the solvent in vacuo. The residue was partitioned between EtOAc (200 mL) and sat. NaHCO₃ solution (150 mL). The organic layer was separated, washed with brine solution (2×100 mL), dried (Na₂SO₄) and the solvent removed in vacuo. The residue was purified by gradient flash chromatography eluting with 0-10% MeOH in DCM. Data in table 2.

### Intermediate 12, 1-(4-bromopyridin-2-yl)-1,5,6,7-tetrahydro-4H-pyrazolo[4,3-c]pyridin-4-one

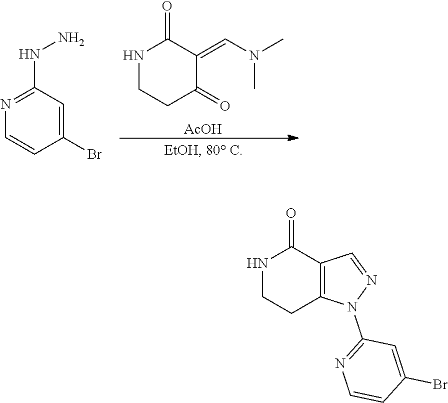 [SMILES: "AcOH" ; NNc1cc(Br)ccn1 ; CN(C)/C=C1\C(=O)CCNC1=O ; O=C1NCCc2c1cnn2-c1cc(Br)ccn1]

The title compound (410 mg, 78%) was prepared from 4-bromo-2-hydrazineylpyridine (300 mg, 1.78 mmol) and (E)-3-((dimethylamino)methylene) piperidine-2,4-dione (Intermediate 1, 332 mg, 1.78 mmol) in EtOH (5 mL) and AcOH (0.01 mL) heated at 100° C. for 10 h using the method of Intermediate 10, step 3. The title compound was isolated as yellow solid by trituration with Et₂O. Data in table 2.

### Typical Procedure 11, Exemplified by the Preparation of Intermediate 13, 1-(2-bromopyridin-4-yl)-1,5,6,7-tetrahydro-4H-pyrazolo[4,3-c]pyridin-4-one

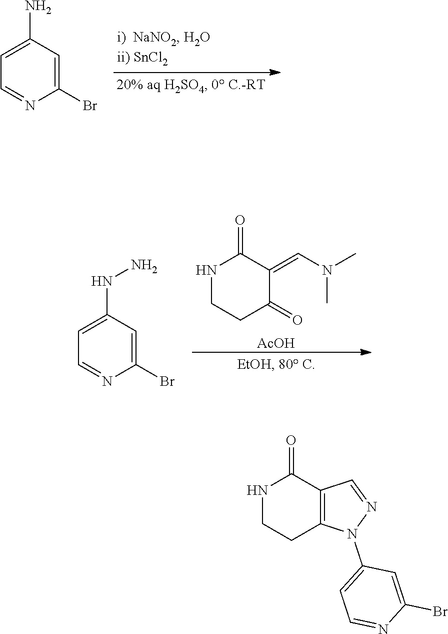

Step 1. NaNO₂ (4.7 g, 69.4 mmol) in water (30 mL) was added to a stirred solution of 2-bromopyridin-4-amine (10 g, 57.8 mmol) in 20% aqueous H₂SO₄ solution (300 mL) at 0° C. and stirred for 1 h, followed by the addition of SnCl₂ (21.8 g, 116 mmol) in 20% aqueous H₂SO₄ solution (100 mL) at 0° C. The reaction mixture was stirred at RT for 1 h. The reaction mixture was basified with ammonium hydroxide solution (200 mL) up to pH-8 and the reaction mixture was filtered through Celite. The filtrate was partitioned between water (200 mL) and 10% MeOH in DCM (400 mL). The organic layer was separated, washed with brine (50 mL), dried (Na₂SO₄) and the solvent removed in vacuo to afford 2-bromo-4-hydrazineylpyridine as a brown solid (7.0 g, 64%).

LCMS (Method 3): m/z 188.0 (ES+), at 0.37 min.

¹H NMR: (400 MHz, DMSO-d₆) δ: 7.95 (s, 1H), 7.75 (d, J=6.0 Hz, 1H), 6.81 (s, 1H), 6.59 (d, J=3.6 Hz, 1H), 4.32 (s, 2H).

Step 2. A catalytic amount of acetic acid (0.2 mL) was added to a stirred solution of 2-bromo-4-hydrazineylpyridine (7.0 g, 37.2 mmol) and (E)-3-((dimethylamino)methylene)piperidine-2,4-dione (Intermediate 1, 4.3 g, 26.1 mmol) in EtOH (150 mL) and the reaction mixture was heated at 80° C. for 15 h. Precipitated solid was filtered, washed with EtOH (2×50 mL) and pet-ether (2×50 mL) to afford 1-(2-bromopyridin-4-yl)-1,5,6,7-tetrahydro-4H-pyrazolo[4,3-c]pyridin-4-one as a yellow solid (3.5 g, 32%). Data in table 2.

### Intermediate 37, 1-(5-bromopyridin-3-yl)-5-(2,4-dimethoxybenzyl)-3-methyl-1,5,6,7-tetrahydro-4H-pyrazolo[4,3-c]pyridin-4-one

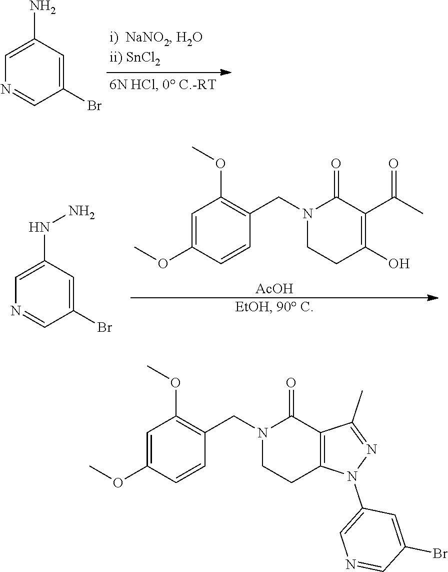

The title compound (400 mg, 38%) was prepared in two steps from 5-bromopyridin-3-amine (1 g, 5.78 mmol) in 6N HCl (10 mL) and NaNO₂ (590 mg, 8.67 mmol) in water (20 mL) stirred for 15 min at 0° C., followed by the addition of SnCl₂ (2.1 g, 11.56 mmol) in 6N HCl (20 mL) and stirred at RT for 3 h; and 3-acetyl-1-(2,4-dimethoxybenzyl)-4-hydroxy-5,6-dihydropyridin-2(1H)-one (Intermediate 35, 700 mg, 2.29 mmol) and acetic acid (14 mg, 0.229 mmol) in EtOH (20 mL) heated at 90° C. for 15 h using the methods of Intermediate 13. After completion of step 2, the title compound was isolated as a yellow solid by removal of the solvent in vacuo. The residue was purified by gradient flash column chromatography eluting with 0-100% EtOAc in pet-ether Data in table 2.

### Typical Procedure 12, Exemplified by the Preparation of Intermediate 14, tert-butyl 1-(2-bromopyridin-4-yl)-4-oxo-1,4,6,7-tetrahydro-5H-pyrazolo[4,3-c]pyridine-5-carboxylate

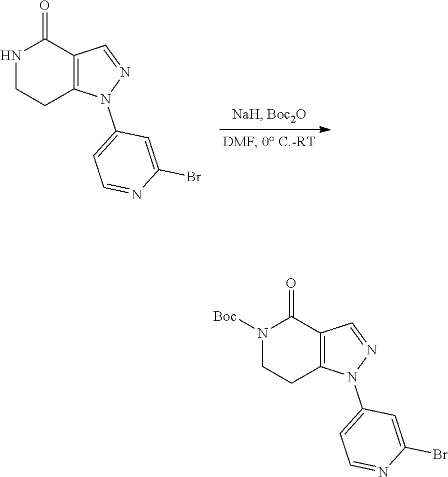 [SMILES: O=C1NCCc2c1cnn2-c1ccnc(Br)c1 ; CC(C)(C)OC(=O)N1CCc2c(cnn2-c2ccnc(Br)c2)C1=O]

NaH (368 mg, 15.3 mmol) and Boc anhydride (3.34 g, 15.3 mmol) were added to a stirred solution of 1-(2-bromopyridin-4-yl)-1,5,6,7-tetrahydro-4H-pyrazolo[4,3-c]pyridin-4-one (Intermediate 13, 3 g, 10.2 mmol) in DMF (20 mL) at 0° C. and the reaction mixture was stirred at RT for 12 h. The reaction mixture was partitioned between ice-cold water (500 mL) and EtOAc (250 mL). The organic layer was separated, dried (Na₂SO₄) and the solvent removed in vacuo. The residue was purified by gradient flash column chromatography eluting with 0-80% EtOAc in hexane to afford tert-butyl 1-(2-bromopyridin-4-yl)-4-oxo-1,4,6,7-tetrahydro-5H-pyrazolo[4,3-c]pyridine-5-carboxylate as a white solid (2.6 g. 64%). Data in table 2.

### Typical Procedure 13, Exemplified by the Preparation of Intermediate 21, tert-butyl 1-(2-bromopyridin-4-yl)-4-oxo-1,4,6,7-tetrahydro-5H-pyrrolo[3,2-c]pyridine-5-carboxylate

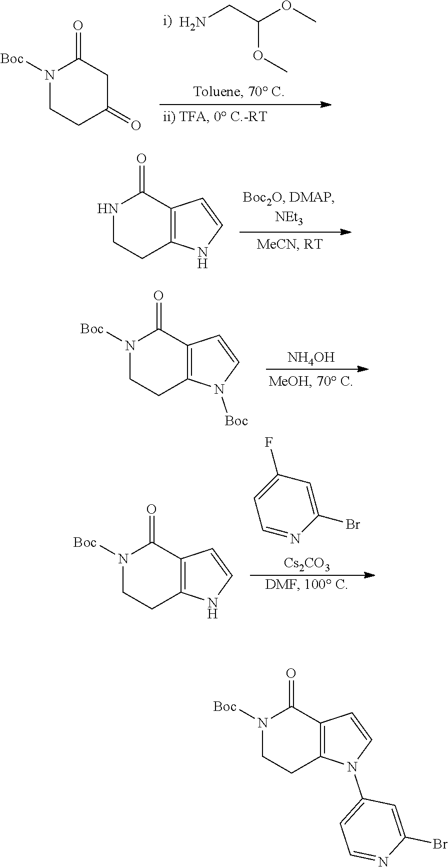

Step 1. Molecular sieves (3 g) were added to a stirred solution of tert-butyl 2,4-dioxopiperidine-1-carboxylate (3 g, 14.1 mmol) in toluene (50 mL) followed by the addition of 2,2-dimethoxyethan-1-amine (1.53 mL, 14.1 mmol). The reaction mixture was heated at 70° C. for 1 h. The reaction mixture was diluted with water (50 mL) and extracted with EtOAc (3×50 mL). The combined organic layers were dried (Na₂SO₄) and the solvent removed in vacuo. The residue was dissolved in DCM (30 mL) and cooled to 0° C. TFA (30 mL) was added and the reaction mixture was stirred at RT for 2 h. The solvent was removed in vacuo. The residue was purified by gradient flash column chromatography eluting with 0-4% MeOH in DCM to afford 1,5,6,7-tetrahydro-4H-pyrrolo[3,2-c]pyridin-4-one as an off-white solid.

LCMS (Method 7): m/z 137.2 (ES+), at 0.53 min.

Step 2. NEt₃ (11.2 mL, 79.3 mmol) and DMAP (0.242 g, 1.98 mmol) were added to a stirred solution of 1,5,6,7-tetrahydro-4H-pyrrolo[3,2-c]pyridin-4-one (2.4 g, 13.23 mmol) in MeCN (50 mL) followed by the addition of Boc anhydride (15.9 mL, 69.4 mmol). The reaction mixture was stirred at RT for 16 h. The reaction mixture was diluted with water (50 mL) and extracted with EtOAc (3×50 mL). The combined organic layers were dried (Na₂SO₄) and the solvent removed in vacuo. The residue was purified by flash column chromatography eluting with 0-25% EtOAc in pet-ether to afford di-tert-butyl 4-oxo-6,7-dihydro-1H-pyrrolo[3,2-c]pyridine-1,5(4H)-dicarboxylate as a yellow solid (2.91 g, 65%).

LCMS (Method 7): m/z 281.2 (ES+), at 2.56 min.

¹H NMR: (400 MHz, DMSO-d₆) δ: 7.25 (d, J=4.8 Hz, 1H), 6.50 (d, J=4.4 Hz, 1H), 3.95 (t, J=8.8 Hz, 2H), 3.25 (t, J=26.8 Hz, 2H), 1.54 (s, 9H), 1.29 (s, 9H).

Step 3. Ammonium hydroxide solution (20 mL) was added to a stirred solution of di-tert-butyl 4-oxo-6,7-dihydro-1H-pyrrolo[3,2-c]pyridine-1,5(4H)-dicarboxylate (2.9 g, 8.63 mmol) in MeOH (30 mL) and the reaction mixture was heated at 70° C. for 8 h. The solvent was removed in vacuo. The residue was purified by gradient flash column chromatography eluting with 0-40% EtOAc in pet-ether to afford tert-butyl 4-oxo-1,4,6,7-tetrahydro-5H-pyrrolo[3,2-c]pyridine-5-carboxylate as a white solid (0.8 g, 39%).

LCMS (Method 7): m/z 235.2 (ES+), at 1.66 min.

¹H NMR: (400 MHz, DMSO-d₆) δ: 11.36 (s, 1H), 6.74 (d, J=1.2 Hz, 1H), 6.32 (d, J=2.8 Hz, 1H), 3.91 (t, J=6.4 Hz, 2H), 2.80 (t, J=6.0 Hz, 2H), 1.45 (s, 9H).

Step 4. Cs₂CO₃ (1.1 g, 3.409 mmol) was added to a stirred solution of 2-bromo-4-fluoropyridine (0.2 g, 1.13 mmol) and tert-butyl 4-oxo-1,4,6,7-tetrahydro-5H-pyrrolo[3,2-c]pyridine-5-carboxylate (0.268 g, 1.13 mmol) in DMF (10 mL) and the reaction mixture was heated at 100° C. for 2 h. The reaction mixture was diluted with water (50 mL) and extracted with EtOAc (2×50 mL). The combined organic layers were dried (Na₂SO₄) and the solvent removed in vacuo. The residue was purified by gradient flash column chromatography eluting with 0-40% EtOAc in pet-ether to afford tert-butyl 1-(2-bromopyridin-4-yl)-4-oxo-1,4,6,7-tetrahydro-5H-pyrrolo[3,2-c]pyridine-5-carboxylate as a white solid (0.38 g, 89%). Data in table 2.

### Typical Procedure 14, Exemplified by the Preparation of Intermediate 23, tert-butyl 1-(6-chloropyrimidin-4-yl)-4-oxo-1,4,6,7-tetrahydro-5H-pyrazolo[4,3-c]pyridine-5-carboxylate

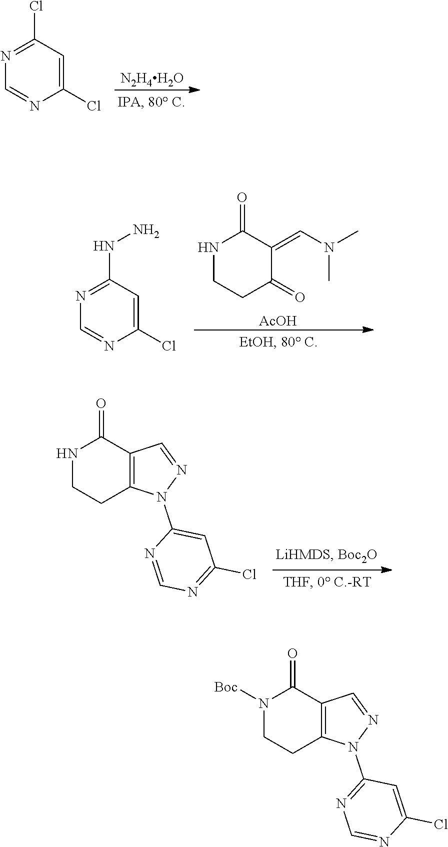

Step 1. Hydrazine hydrate (3 g, 60.39 mmol) was added to a stirred solution of 4,6-dichloropyrimidine (3 g, 20.1 mmol) in IPA (50 mL) and the reaction mixture was heated at 80° C. for 16 h. The solvent was removed in vacuo and the residue azeotroped with toluene to afford 4-chloro-6-hydrazineylpyrimidine as a pale yellow solid (2.5 g, 86%).

LCMS (Method 10): m/z 145.1 (ES+), at 0.86 min.

¹H NMR: (400 MHz, DMSO-d₆) δ: 8.83 (s, 1H), 8.17 (s, 1H), 6.75 (s, 1H), 4.49 (s, 2H).

Step 2. A catalytic amount of acetic acid (1 mL) was added to a stirred solution of 4-chloro-6-hydrazineylpyrimidine (1 g, 6.94 mmol) and (E)-3-((dimethylamino)methylene)piperidine-2,4-dione (Intermediate 1, 1.2 g, 6.94 mmol) in ethanol (30 mL) and the reaction mixture was heated at 80° C. for 16 h. The solvent was removed in vacuo and the residue was partitioned between sat. NaHCO₃ solution (50 mL) and EtOAc (50 mL). The residue was purified by gradient flash column chromatography eluting with 0-100% EtOAc in pet-ether to afford 1-(6-chloropyrimidin-4-yl)-1,5,6,7-tetrahydro-4H-pyrazolo[4,3-c]pyridin-4-one as an off-white solid (300 mg, 17%).

LCMS (Method 3): m/z 250.0 (ES+), at 1.41 min.

¹H NMR: (400 MHz, DMSO-d₆) δ: 9.02 (d, J=0.8 Hz, 1H), 8.18 (s, 1H), 8.06 (d, J=0.8 Hz, 1H), 7.61 (s, 1H), 3.50 (d, J=2.0 Hz, 4H).

Step 3. LiHMDS (1M in THF, 0.6 mL, 0.60 mmol) was added to a stirred solution of 1-(6-chloropyrimidin-4-yl)-1,5,6,7-tetrahydro-4H-pyrazolo[4,3-c]pyridin-4-one (100 mg, 0.40 mmol) in THF (5 mL) at 0° C. and then stirred at RT for 30 min. Boc anhydride (130 mg, 0.60 mmol) was then added and the reaction mixture was stirred at RT for 16 h. The reaction mixture was quenched with ice water (50 mL) and extracted with EtOAc (50 mL). The organic layer was separated, washed with brine solution (50 mL), dried (Na₂SO₄) and the solvent removed in vacuo. The residue was purified by gradient flash column chromatography eluting with 0-50% EtOAc in pet-ether to afford tert-butyl 1-(6-chloropyrimidin-4-yl)-4-oxo-1,4,6,7-tetrahydro-5H-pyrazolo[4,3-c]pyridine-5-carboxylate as a yellow solid (60 mg, 42%). Data in table 2.

### Typical Procedure 15, Exemplified by the Preparation of Intermediate 36, 1-(2-bromopyridin-4-yl)-5-(2,4-dimethoxybenzyl)-3-methyl-1,5,6,7-tetrahydro-4H-pyrazolo[4,3-c]pyridin-4-one

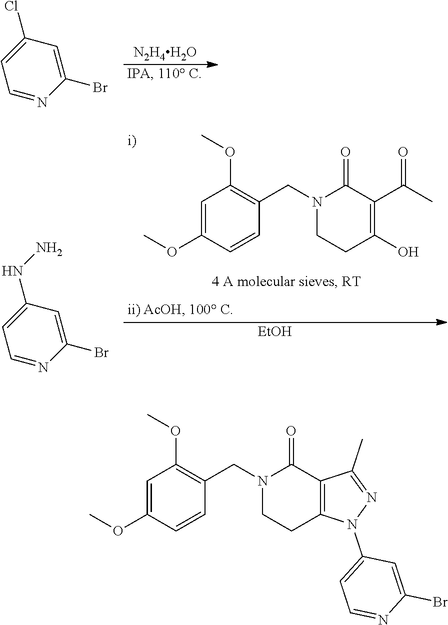

Step 1. Hydrazine hydrate (26 g, 521 mmol) was added to a stirred solution of 2-bromo-4-chloropyridine (20 g, 104 mmol) in IPA (100 mL) and the reaction mixture was heated at 110° C. for 16 h. The solvent was removed in vacuo and the residue was dissolved in EtOAc (500 mL) and washed with water (500 mL). The organic layer was separated, washed with brine solution (500 mL), dried (Na₂SO₄) and the solvent removed in vacuo to afford 2-bromo-4-hydrazineylpyridine as a white solid (15 g, 77%).

LCMS (Method 10): m/z 188.2 (ES+), at 1.06 min.

¹H NMR: (400 MHz, DMSO-d₆) δ: 7.95-7.93 (m, 1H), 7.74 (d, J=7.2 Hz, 1H), 6.80 (d, J=6.4 Hz, 1H), 6.75 (s, 1H), 4.32 (s, 2H).

Step 2. 4 A Molecular sieves were added to a stirred solution of 2-bromo-4-hydrazineylpyridine (5 g, 26.6 mmol) and 3-acetyl-1-(2,4-dimethoxybenzyl)-4-hydroxy-5,6-dihydropyridin-2(1H)-one (Intermediate 35, 9.7 g, 31.9 mmol) in EtOH (100 mL) and the reaction mixture was stirred at RT for 10 h. Acetic acid (1.6 mL, 26.6 mmol) was then added and the reaction mixture was heated at 100° C. for 16 h. The reaction mixture was filtered through celite, washed with EtOAc (2×50 mL) and the solvent removed in vacuo. The residue was dissolved in EtOAc (300 mL) and washed with 10% NaHCO₃ solution (100 mL). The organic layer was separated, dried (Na₂SO₄) and the solvent removed in vacuo. The residue was purified by gradient flash column chromatography eluting with 0-50% EtOAc in pet-ether to afford 1-(2-bromopyridin-4-yl)-5-(2,4-dimethoxybenzyl)-3-methyl-1,5,6,7-tetrahydro-4H-pyrazolo[4,3-c]pyridin-4-one as a white solid (4.1 g, 28%). Data in table 2.

### Intermediate 38, 1-(4-bromopyridin-2-yl)-5-(2,4-dimethoxybenzyl)-3-methyl-1,5,6,7-tetrahydro-4H-pyrazolo[4,3-c]pyridin-4-one

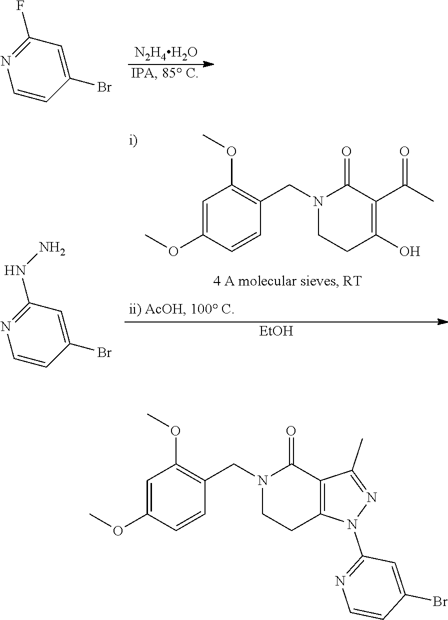

The title compound (700 mg, 25%) was prepared in two steps from 4-bromo-2-fluoropyridine (2 g, 11.4 mmol) and hydrazine hydrate (5.6 g, 114 mmol) in IPA (20 mL) heated at 85° C. for 12 h; and 4 Å Molecular sieves (1 g) and 3-acetyl-1-(2,4-dimethoxybenzyl)-4-hydroxy-5,6-dihydropyridin-2(1H)-one (Intermediate 35, 1.6 g, 5.31 mmol) in EtOH (20 mL) stirred at RT for 12 h, followed by addition of acetic acid (0.1 mL) heated at 90° C. for 12 h using the methods of Intermediate 36. After completion of step 2, the title compound was isolated as a yellow solid by filtration, rinsing with EtOAc (2×50 mL) and removal of the solvent in vacuo. The residue was dissolved in EtOAc (50 mL) and washed with 10% NaHCO₃ solution (50 mL). The organic layer was separated, washed with brine solution (20 mL), dried (Na₂SO₄) and the solvent removed in vacuo. The residue was purified by gradient flash column chromatography eluting with 0-70% EtOAc in pet-ether. Data in table 2.

### Typical Procedure 16, Exemplified by the Preparation of Intermediate 41, tert-butyl 1-(2-bromo-5-methylpyridin-4-yl)-4-oxo-1,4,6,7-tetrahydro-5H-pyrazolo[4,3-c]pyridine-5-carboxylate

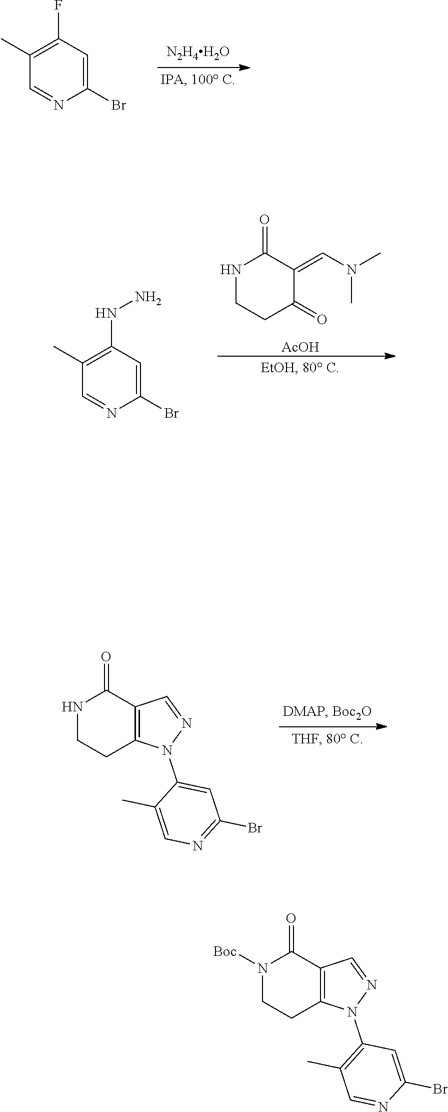

Step 1. Hydrazine hydrate (0.16 mL, 3.16 mmol) was added to a stirred solution of 2-bromo-4-fluoro-5-methylpyridine (200 mg, 1.05 mmol) in IPA (10 mL) at RT and the reaction mixture was heated at 100° C. for 16 h. The solvent was removed in vacuo. The residue was triturated with 10% NaHCO₃ solution (5 mL) and the solids filtered, washed with water (10 mL) and dried in vacuo to afford 2-bromo-4-hydrazineyl-5-methylpyridine as an off-white solid (130 mg, 61%).

LCMS (Method 2): m/z 201.7 (ES+), at 1.07 min. ¹H NMR: (300 MHz, DMSO-d₆) δ: 7.60 (s, 1H), 7.50 (s, 1H), 6.99 (s, 1H), 4.29 (s, 2H), 1.92 (s, 3H).

Step 2. (E)-3-((Dimethylamino)methylene)piperidine-2,4-dione (Intermediate 1, 162 mg, 0.97 mmol) and acetic acid (19 mg, 0.32 mmol) were added to a solution of 2-bromo-4-hydrazineyl-5-methylpyridine (130 mg, 0.64 mmol) in EtOH (10 mL) at RT. The reaction mixture was heated at 80° C. for 16 h. The reaction mixture was partitioned between 10% NaHCO₃ solution (15 mL) and EtOAc (15 mL). The organic layer was separated, dried (Na₂SO₄) and the solvent removed in vacuo to afford 1-(2-bromo-5-methylpyridin-4-yl)-1,5,6,7-tetrahydro-4H-pyrazolo[4,3-c]pyridin-4-one as a yellow gum (100 mg, 51%).

LCMS (Method 10): m/z 307.0 (ES+), at 1.12 min.

¹H NMR: (400 MHz, DMSO-d₆) δ: 8.52 (s, 1H), 8.03 (s, 1H), 7.85 (s, 1H), 7.45 (s, 1H), 3.42-3.41 (m, 2H), 2.89 (t, J=9.2 Hz, 2H), 2.27 (s, 3H).

Step 3. DMAP (7.96 mg, 0.07 mmol) and Boc anhydride (0.09 mL, 0.39 mmol) were added to a solution of 1-(2-bromo-5-methylpyridin-4-yl)-1,5,6,7-tetrahydro-4H-pyrazolo[4,3-c]pyridin-4-one (100 mg, 0.33 mmol) in THF (10 mL) at RT and the reaction mixture was heated at 80° C. for 15 h. The solvent was removed in vacuo. The residue was purified by gradient flash column chromatography eluting with 0-60% EtOAc in pet-ether to afford tert-butyl 1-(2-bromo-5-methylpyridin-4-yl)-4-oxo-1,4,6,7-tetrahydro-5H-pyrazolo[4,3-c]pyridine-5-carboxylate as a white solid (100 mg, 75%). Data in table 2.

### Typical Procedure 17, Exemplified by the Preparation of Intermediate 45, 1-(2-(3-fluoro-5-(trifluoromethyl)benzyl)pyridin-4-yl)-3-iodo-1,5,6,7-tetrahydro-4H-pyrazolo[4,3-c]pyridin-4-one

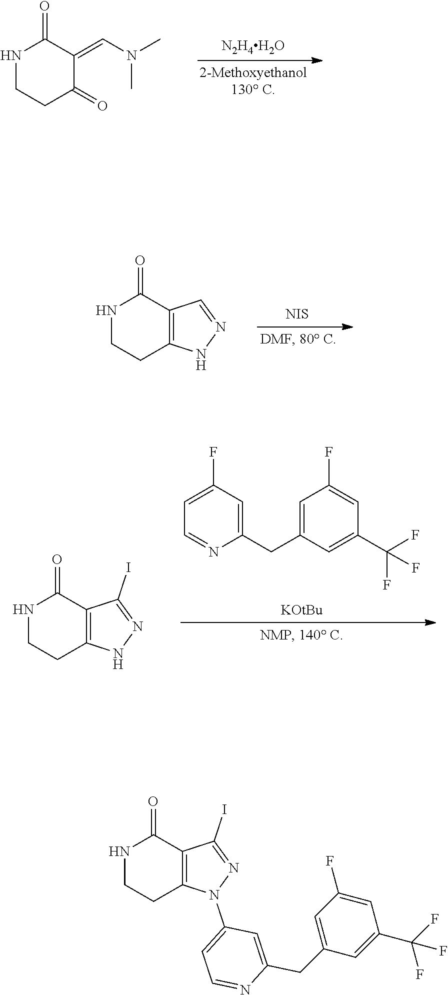

Step 1. Hydrazine hydrate (297 mg, 5.95 mmol) was added to a stirred solution of (E)-3-((dimethylamino)methylene)piperidine-2,4-dione (Intermediate 1, 500 mg, 2.97 mmol) in 2-methoxyethanol (10 mL) and the reaction mixture was heated at 130° C. for 12 h. The solvent was removed in vacuo. The residue was purified by gradient flash column chromatography eluting with 0-15% MeOH in DCM to afford (1,5,6,7-tetrahydro-4H-pyrazolo[4,3-c]pyridin-4-one as a brown solid (300 mg, 73%).

LCMS (Method 6): m/z 138.2 (ES+), at 0.63 min.

¹H NMR: (400 MHz, DMSO-d₆) δ: 7.92 (s, 1H), 7.28 (s, 1H), 3.39-3.35 (m, 2H), 2.81-2.77 (m, 2H). 1 exchangeable proton not observed.

Step 2. NIS (980 mg, 4.37 mmol) was added to a stirred solution of (1,5,6,7-tetrahydro-4H-pyrazolo[4,3-c]pyridin-4-one (300 mg, 2.18 mmol) in DMF (10 mL) at RT and the reaction mixture was heated at 80° C. for 12 h. The solvent was removed in vacuo. The residue was purified by gradient flash column chromatography eluting with 0-15% MeOH in DCM to afford 3-iodo-1,5,6,7-tetrahydro-4H-pyrazolo[4,3-c]pyridin-4-one as an off-white solid (290 mg, 51%).

LCMS (Method 3): m/z 263.9 (ES+), at 0.61 min.

¹H NMR: (400 MHz, DMSO-d₆) δ: 13.41 (s, 1H), 7.30 (s, 1H), 2.84-2.63 (m, 4H).

Step 3. KOt-Bu (246 mg, 2.20 mmol) was added to a stirred solution of 3-iodo-1,5,6,7-tetrahydro-4H-pyrazolo[4,3-c]pyridin-4-one (290 mg, 1.10 mmol) in NMP (10 mL) followed by the addition of 4-fluoro-2-(3-fluoro-5-(trifluoromethyl)benzyl)pyridine (Intermediate 3, 362 mg, 1.32 mmol). The reaction mixture was heated in a microwave at 140° C. for 1 h. The reaction mixture was diluted with water (20 mL) and extracted with EtOAc (2×50 mL). The combined organic layers were washed with brine solution (20 mL), dried (Na₂SO₄) and the solvent removed in vacuo. The residue was purified by gradient flash column chromatography eluting with 0-15% MeOH in DCM to afford 1-(2-(3-fluoro-5-(trifluoromethyl)benzyl)pyridin-4-yl)-3-iodo-1,5,6,7-tetrahydro-4H-pyrazolo[4,3-c]pyridin-4-one as a white solid (150 mg, 26%). Data in table 2.

### Preparation of Substituted Triazole and Pyrazole Intermediates

### Typical Procedure 18, Exemplified by the Preparation of Intermediate 15, 1,5,6,7-tetrahydro-4H-benzo[d][1,2,3]triazol-4-one

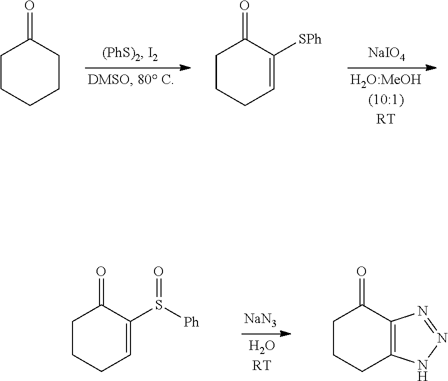

Step 1. Iodine (0.26 g, 1 mmol) was added to a stirred mixture of cyclohexanone (2 g, 20 mmol) and 1,2-diphenyldisulfane (1.7 g, 80 mmol) in DMSO (12 mL) and the reaction mixture was heated at 80° C. for 12 h. The reaction was quenched by the addition of water (100 mL) and the aqueous layer was extracted with EtOAc (3×100 mL). The organic layers were combined, dried (Na₂SO₄) and the solvent removed in vacuo to afford 2-(phenylthio)cyclohex-2-en-1-one as a yellow liquid (4.5 g crude). The crude product was used in the next step without further purification.

MS (Method 1): m/z 205 (ES+).

Step 2. Sodium periodate (9.39 g, 40 mmol) was added to a stirred solution of 2-(phenylthio)cyclohex-2-en-1-one (4.5 g, 22 mmol) in MeOH (1.2 mL) and H₂O (12 mL) and the reaction mixture was stirred at RT for 16 h. The reaction was quenched by the addition of water (100 mL) and the aqueous layer was extracted with EtOAc (3×100 mL). The organic layers were combined, dried (Na₂SO₄) and the solvent removed in vacuo. The residue was purified by gradient flash column chromatography eluting with 30-35% EtOAc in hexane to afford 2-(phenylsulfinyl) cyclohex-2-en-1-one as an orange gum (2.1 g, 43%).

MS (Method 1): m/z 221 (ES+).

Step 3. Sodium azide (324 mg, 4 mmol) was added to a stirred solution of 2-(phenylsulfinyl)cyclohex-2-en-1-one (1 g, 4 mmol) in H₂O (17 mL) and the reaction mixture was stirred at RT for 16 h. The reaction mixture was acidified to pH 2 using 1 N HCl (19 mL) and water (100 mL) was added. The aqueous layer was extracted with EtOAc (3×100 mL). The organic layers were combined, dried (Na₂SO₄) and the solvent removed in vacuo to afford 1,5,6,7-tetrahydro-4H-benzo[d][1,2,3]triazol-4-one as an off white solid (220 mg, 35%). Data in table 2.

### Typical Procedure 19, Exemplified by the Preparation of Intermediate 16, 1,5,6,7-tetrahydro-4H-[1,2,3]triazolo[4,5-c]pyridin-4-one

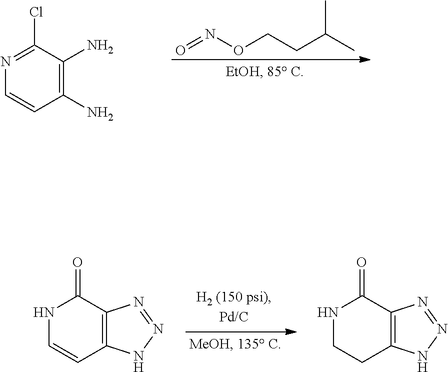

Step 1. iso-Amyl nitrite (1.41 mL, 10.48 mmol) was added to a solution of 2-chloropyridine-3,4-diamine (300 mg, 2.09 mmol) in EtOH (5 mL). The reaction mixture was heated at 85° C. for 16 h. The solvent was removed in vacuo to afford 1,5-dihydro-4H-[1,2,3]triazolo[4,5-c]pyridin-4-one as a brown solid (310 mg crude). The crude product was used in the next step without further purification.

MS (Method 1): m/z 134.9 (ES−)

¹H NMR: (400 MHz, DMSO-d₆) δ: 11.46 (bs, 1H), 7.30-7.21 (m, 1H), 6.65-6.58 (m, 1H). 1 exchangeable proton not observed.

Step 2. 10% Pd/C (50% w/w, 400 mg) was slowly added to a mixture of 1,5-dihydro-4H-[1,2,3]triazolo[4,5-c]pyridin-4-one (300 mg, 2.20 mmol) in MeOH (35 mL) and the reaction mixture was heated at 135° C. in a H₂ atmosphere (150 psi) for 16 h. The reaction mixture was filtered through Celite which was washed with MeOH (25 mL). The solvent was removed in vacuo to afford 1,5,6,7-tetrahydro-4H-[1,2,3]triazolo[4,5-c]pyridin-4-one as an off white solid (200 mg, crude). Data in table 2.

### Typical Procedure 20, Exemplified by the Preparation of Intermediate 43, ethyl 5-(2-((tert-butoxycarbonyl)amino)ethoxy)-1H-pyrazole-4-carboxylate

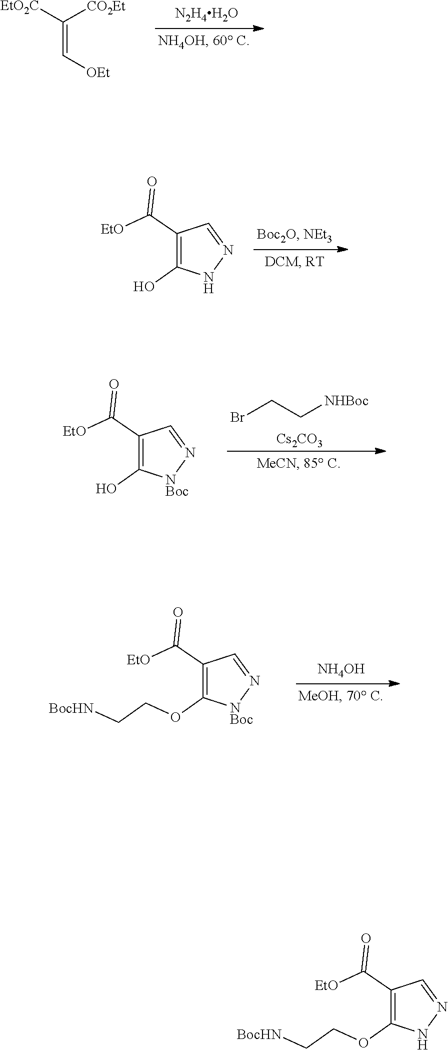

Step 1. Hydrazine hydrate (2.5 g, 48 mmol) was added to a suspension of diethyl 2-(ethoxymethylene)malonate (8.5 g, 40 mmol) in 25% aqueous ammonium hydroxide solution (11 mL, 320 mmol) at RT and the reaction mixture was heated at 60° C. for 4 h. The reaction mixture was diluted with water (200 mL), adjusted pH-7 with 1.5 N HCl (30 mL) and extracted with EtOAc (500 mL). The organic layer was separated, washed with brine solution (100 mL), dried (Na₂SO₄) and the solvent removed in vacuo to afford ethyl 5-hydroxy-1H-pyrazole-4-carboxylate as an off-white solid (3 g, 48%).

LCMS (Method 7): m/z 157.2 (ES+), at 0.43 min.

¹H NMR: (400 MHz, DMSO-d₆) δ: 12.22 (s, 1H), 10.19 (s, 1H), 7.85 (s, 1H), 4.14 (q, J=7.2 Hz, 2H), 1.23 (t, J=6.8 Hz, 3H).

Step 2. NEt₃ (1 g, 10.25 mmol) was added to a stirred solution of ethyl 5-hydroxy-1H-pyrazole-4-carboxylate (1.6 g, 10.3 mmol) in DCM (30 mL) followed by the addition of Boc anhydride (2.23 g, 10.25 mmol) and the reaction mixture was stirred at RT for 4 h. The solvent was removed in vacuo and the residue was triturated with Et₂O (2×30 mL), and dried in vacuo to afford 1-(tert-butyl) 4-ethyl 5-hydroxy-1H-pyrazole-1,4-dicarboxylate as a yellow solid (1.9 g, 73%).

LCMS (Method 7): m/z 201.0 (ES+), at 1.85 min.

¹H NMR: (400 MHz, DMSO-d₆) δ: 11.69 (s, 1H), 8.40 (s, 1H), 4.20 (q, J=6.8 Hz, 2H), 1.56 (s, 9H), 1.26 (t, J=6.8 Hz, 3H).

Step 3. Cs₂CO₃ (127 mg, 0.39 mmol) was added to a stirred solution of 1-(tert-butyl) 4-ethyl 5-hydroxy-1H-pyrazole-1,4-dicarboxylate (100 mg, 0.39 mmol) in MeCN (2 mL) followed by the addition of tert-butyl (2-bromoethyl)carbamate (87 mg, 0.39 mmol) at RT and the reaction mixture was heated at 85° C. for 4 h. The reaction mixture was diluted with water (20 mL) and extracted with EtOAc (40 mL). The organic layer was separated, washed with brine solution (20 mL), dried (Na₂SO₄) and the solvent removed in vacuo. The residue was purified by gradient flash column chromatography eluting with 0-40% EtOAc in pet-ether to afford 1-(tert-butyl) 4-ethyl 5-(2-((tert-butoxycarbonyl)amino)ethoxy)-1H-pyrazole-1,4-dicarboxylate as a white solid (50 mg, 32%).

LCMS (Method 7): m/z 300.1 (ES+), at 2.33 min.

¹H NMR: (400 MHz, CDCl₃) δ: 8.37 (s, 1H), 5.13 (s, 1H), 4.45 (t, J=4.8 Hz, 2H), 4.36-4.31 (m, 2H), 3.59 (d, J=1.2 Hz, 2H), 1.57 (s, 9H), 1.49 (s, 9H), 1.39 (t, J=7.2 Hz, 3H).

Step 4. NH₄OH solution (2 mL) was added to a stirred solution of 1-(tert-butyl) 4-ethyl 5-(2-((tert-butoxycarbonyl)amino)ethoxy)-1H-pyrazole-1,4-dicarboxylate (50 mg, 0.13 mmol) in MeOH (2 mL) and the reaction mixture was heated at 70° C. for 3 h. The reaction mixture was diluted with water (20 mL) and extracted with EtOAc (40 mL). The organic layer was separated, washed with brine solution (20 mL), dried (Na₂SO₄) and the solvent removed in vacuo to afford ethyl 5-(2-((tert-butoxycarbonyl)amino)ethoxy)-1H-pyrazole-4-carboxylate as a white solid (35 mg, 93%). Data in table 2.

### Preparation of Substituted Benzyl Halide Intermediates

### Typical Procedure 21, Exemplified by the Preparation of Intermediate 22, 1-(chloromethyl)-3-cyclopropyl-5-fluorobenzene

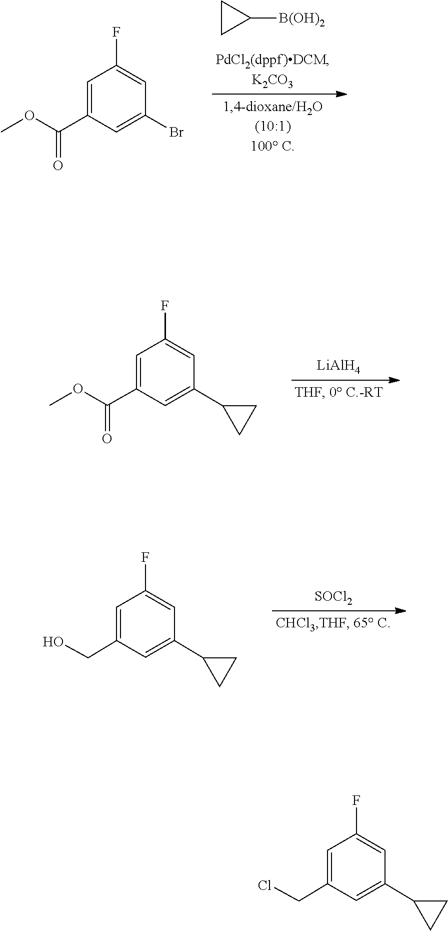

Step 1. K₂CO₃ (1.0 g, 12.87 mmol) was added to a stirred solution of methyl 3-bromo-5-fluorobenzoate (1.0 g, 4.29 mmol) and cyclopropylboronic acid (401 mg, 4.72 mmol) in 1,4-dioxane (10 mL) and water (1 mL). The reaction mixture was degassed with N₂ for 20 min then Pd(dppf)₂Cl₂.DCM complex (350 mg, 0.429 mmol) was added. The reaction mixture was heated at 100° C. for 12 h. The reaction mixture was partitioned between water (200 mL) and EtOAc (80 mL). The organic layer was separated, dried (Na₂SO₄) and the solvent removed in vacuo. The residue was purified by flash column chromatography eluting with 0-50% EtOAc in hexane to afford methyl 3-cyclopropyl-5-fluorobenzoate as a yellow liquid (750 mg, 90%).

GCMS (Method 2): m/z 194.0 (ES+), at 3.43 min.

¹H NMR: (400 MHz, DMSO-d₆) δ: 7.54 (t, J=1.6 Hz, 1H), 7.46-7.45 (m, 1H), 7.24-7.23 (m, 1H), 3.86 (s, 3H), 2.11-2.09 (m, 1H), 1.05-1.04 (m, 2H), 0.79-0.78 (m, 2H).

Step 2. LiAlH₄ (1.0 M in THF, 7.72 mL, 7.72 mmol) was added to a stirred solution of methyl 3-cyclopropyl-5-fluorobenzoate (1 g, 5.15 mmol) in THF (10 mL) at 0° C. and the reaction mixture was stirred at RT for 3 h. The reaction mixture was neutralized with 1.5 N HCl (25 mL) to pH-7. The reaction mixture was partitioned between water (50 mL) and EtOAc (50 mL). The organic layer was separated, dried (Na₂SO₄) and the solvent removed in vacuo to afford (3-cyclopropyl-5-fluorophenyl) methanol as a colourless liquid (810 mg, 94%).

GCMS (Method 1): m/z 166.1 (ES+), at 7.15 min.

¹H NMR: (400 MHz, DMSO-d₆) δ: 6.88-6.87 (m, 2H), 6.76-6.75 (m, 1H), 5.25 (t, J=5.6 Hz, 1H), 4.46-4.45 (m, 2H), 1.92 (d, J=5.6 Hz, 1H), 0.98-0.97 (m, 2H), 0.71-0.70 (m, 2H).

Step 3. Thionyl chloride (1.3 mL, 18.07 mmol) was added to a stirred solution of (3-cyclopropyl-5-fluorophenyl)methanol (300 mg, 1.80 mmol) in chloroform (10 mL) at RT and the reaction mixture was heated at 65° C. for 12 h. The reaction mixture was basified with 10% NaHCO₃ solution (20 mL) to pH-7 and partitioned between water (50 mL) and EtOAc (50 mL). The organic layer was separated, dried (Na₂SO₄) and the solvent removed in vacuo to afford 1-(chloromethyl)-3-cyclopropyl-5-fluorobenzene as a brown liquid (300 mg, crude). The crude product was used in the next step without further purification. Data in table 2.

### Intermediate 31, 1-chloro-5-(chloromethyl)-2-fluoro-3-(trifluoromethyl)benzene

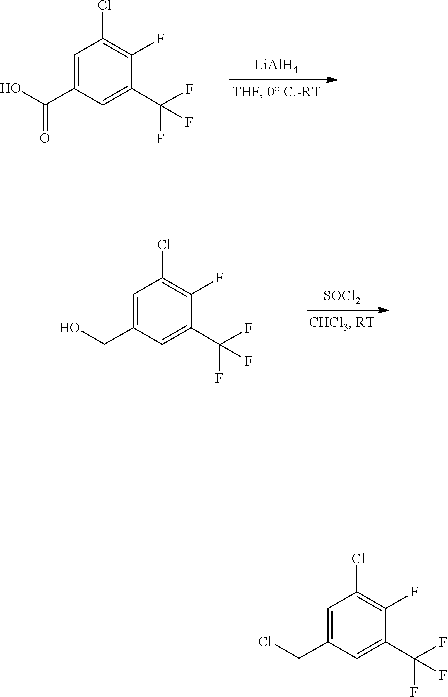 [SMILES: O=C(O)c1cc(Cl)c(F)c(C(F)(F)F)c1 ; "LiAlH4" ; OCc1cc(Cl)c(F)c(C(F)(F)F)c1 ; "SOCl2" ; Fc1c(Cl)cc(CCl)cc1C(F)(F)F]

The title compound (70 mg, crude) was prepared in two steps from 3-chloro-4-fluoro-5-(trifluoromethyl)benzoic acid (0.5 g, 2.06 mmol) and LiAlH₄ (2 M in THF, 1.5 mL, 3.1 mmol) in THF (10 mL) stirred at RT for 1 h; and thionyl chloride (0.03 mL, 0.43 mmol) in chloroform (5 mL) stirred at RT for 1 h using the methods of Intermediate 22, steps 2 and 3. After completion of step 2, the title compound was isolated as an off-white solid by quenching with 10% NaHCO₃ solution (50 mL) and then extraction with DCM (50 mL). The organic layer was separated, dried (Na₂SO₄) and the solvent removed in vacuo. Data in table 2.

### Typical Procedure 22, Exemplified by the Preparation of Intermediate 24, 4-(chloromethyl)-1-(difluoromethoxy)-2-fluorobenzene

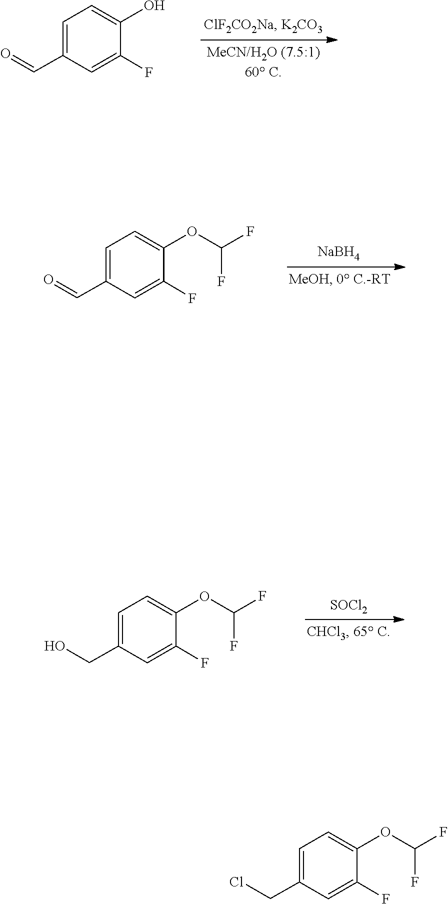

Step 1. K₂CO₃ (2.95 g, 21.42 mmol) was added to a stirred solution of 3-fluoro-4-hydroxybenzaldehyde (1 g, 7.14 mmol) in MeCN:H₂O (15 mL:2 mL) at RT, followed by the addition of sodium 2-chloro-2,2-difluoroacetate (1.2 g, 8.57 mmol) and the reaction mixture was heated at 60° C. for 12 h. The solvent was removed in vacuo and the residue was partitioned between water (20 mL) and EtOAc (50 mL). The organic layer was separated, washed with brine solution (20 mL), dried (Na₂SO₄) and the solvent removed in vacuo. The residue was purified by gradient flash column chromatography eluting with 0-100% EtOAc in pet-ether to afford 4-(difluoromethoxy)-3-fluorobenzaldehyde as a colourless liquid (400 mg, 30%).

GCMS (Method 2): m/z 190.0 (ES+), at 2.19 min.

¹H NMR: (400 MHz, DMSO-d₆) δ: 9.97 (d, J=2.0 Hz, 1H), 7.93-7.85 (m, 2H), 7.64-7.60 (m, 1H), 7.45-7.26 (m, 1H).

Step 2. NaBH₄ (160 mg, 4.21 mmol) was added slowly to a suspension of 4-(difluoromethoxy)-3-fluorobenzaldehyde (400 mg, 2.10 mmol) in MeOH (10 mL) at 0° C. and the reaction mixture was stirred at RT for 3 h. The reaction mixture was quenched with ice water (10 mL), the organic layer was separated and the solvent removed in vacuo. The residue was partitioned between water (30 mL) and EtOAc (50 mL). The organic layer was separated, washed with brine solution (20 mL), dried (Na₂SO₄) and the solvent removed in vacuo. The residue was purified by gradient flash column chromatography eluting with 0-100% EtOAc in pet-ether gradient to afford (4-(difluoromethoxy)-3-fluorophenyl)methanol as a colourless liquid (260 mg, 64%).

GCMS (Method 2): m/z 192.0 (ES+), at 2.63 min.

¹H NMR: (400 MHz, DMSO-d₆) δ: 7.34-7.29 (m, 2H), 7.21-7.03 (m, 2H), 5.37-5.34 (m, 1H), 4.50 (d, J=4.4 Hz, 2H).

Step 3. SOCl₂ (798 mg, 6.77 mmol) was added to a stirred solution of (4-(difluoromethoxy)-3-fluorophenyl)methanol (260 mg, 1.35 mmol) in CHCl₃ (15 mL) and the reaction mixture was heated at 65° C. for 12 h. The reaction mixture was partitioned between sat. NaHCO₃ solution (50 mL) and DCM (50 mL). The organic layer was separated, and the aqueous layer was extracted with DCM (50 mL). The combined organic layers were dried (Na₂SO₄) and the solvent removed in vacuo to afford 4-(chloromethyl)-1-(difluoromethoxy)-2-fluorobenzene as a colourless liquid (210 mg, crude). The crude product was used in the next step without further purification. Data in table 2.

### Typical Procedure 23, Exemplified by the Preparation of Intermediate 25, 5-(bromomethyl)-1,2-difluoro-3-(trifluoromethyl)benzene

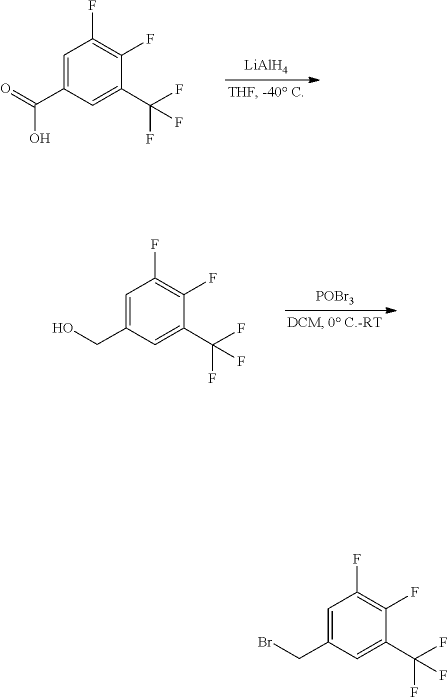 [SMILES: O=C(O)c1cc(F)c(F)c(C(F)(F)F)c1 ; "LiAlH4" ; OCc1cc(F)c(F)c(C(F)(F)F)c1 ; "POBr3" ; Fc1cc(CBr)cc(C(F)(F)F)c1F]

Step 1. LiAlH₄ (2 M in THF, 1.6 mL, 3.32 mmol) was slowly added dropwise to a suspension of 3,4-difluoro-5-(trifluoromethyl)benzoic acid (250 mg, 1.11 mmol) in THF (20 mL) at −40° C. and the reaction mixture was stirred at −40° C. for 3 h. The reaction mixture was quenched with sat. NH₄Cl solution (50 mL) and the aqueous layer was extracted with EtOAc (2×100 mL). The organic layer was separated, washed with brine solution (100 mL), dried (Na₂SO₄) and the solvent removed in vacuo. The residue was purified by gradient flash column chromatography eluting with 0-60% EtOAc in pet-ether to afford (3,4-difluoro-5-(trifluoromethyl)phenyl)methanol as a colourless liquid (130 mg, 56%).

GCMS (Method 1): m/z 212.0 (ES+), at 2.19 min.

¹H NMR: (400 MHz, DMSO-d₆) δ: 7.75-7.71 (m, 1H), 7.56-7.41 (m, 1H), 5.58 (t, J=5.6 Hz, 1H), 4.60 (d, J=5.6 Hz, 2H).

Step 2. POBr₃ (526 mg, 1.81 mmol) was added dropwise to a suspension of (3,4-difluoro-5-(trifluoromethyl)phenyl)methanol (130 mg, 0.613 mmol) in DCM (15 mL) at 0° C. and the reaction mixture was stirred at RT for 12 h. The reaction mixture was diluted with ice cold water (50 mL) and neutralised with sat. NaHCO₃ solution (40 mL) up to pH-7. The organic layer was separated and the aqueous layer extracted with DCM (2×50 mL). The combined organic layers were dried (Na₂SO₄) and the solvent removed in vacuo to afford 5-(bromomethyl)-1,2-difluoro-3-(trifluoromethyl)benzene as a brown gum (140 mg, crude). The crude product was used in the next step without further purification. Data in table 2.

### Typical Procedure 24, Exemplified by the Preparation of Intermediate 26, 1-(chloromethyl)-3-(difluoromethyl)-5-fluorobenzene

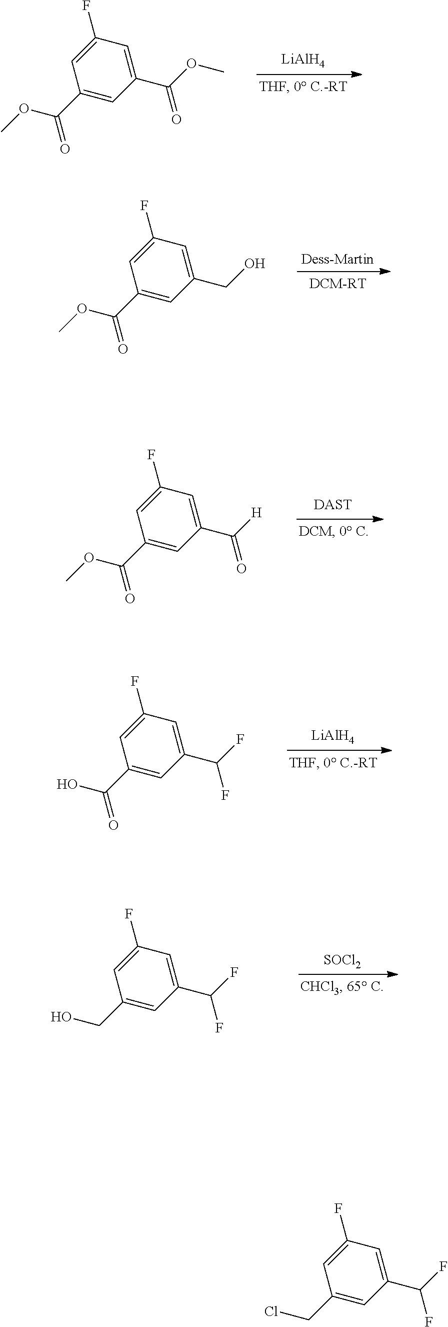

Step 1. LiAlH₄ (1.0 M in THF, 7.0 mL, 7.0 mmol) was added to a stirred solution of dimethyl 5-fluoroisophthalate (3 g, 14.1 mmol) in THF (10 mL), at 0° C. and the reaction mixture was stirred at RT for 3 h. The reaction mixture was neutralized with 1.5 N HCl (50 mL) up to pH-7, and the reaction mixture was partitioned between water (100 mL) and EtOAc (50 mL). The organic layer was separated, dried (Na₂SO₄) and the solvent removed in vacuo to afford methyl 3-fluoro-5-(hydroxymethyl)benzoate as a colourless liquid (1.12 g. 43%).

GCMS (Method 1): m/z 184.0 (ES+), at 7.34 min.

¹H NMR: (400 MHz, DMSO-d₆) δ: 7.79 (s, 1H), 7.55 (d, J=12.8 Hz, 1H), 7.43 (d, J=12.8 Hz, 1H), 5.49 (t, J=7.6 Hz, 1H), 4.58 (d, J=7.6 Hz, 2H), 3.87 (d, J=2.4 Hz, 3H).

Step 2. Dess-Martin periodinane (2.3 g, 5.54 mmol) was added to a solution of methyl 3-fluoro-5-(hydroxymethyl)benzoate (510 mg, 2.77 mmol) in DCM (10 mL) and the reaction mixture was stirred at RT for 2 h. The reaction mixture was filtered and the filtrate was concentrated in vacuo. The residue was purified by gradient flash column chromatography eluting with 0-30% EtOAc in hexane to afford methyl 3-fluoro-5-formylbenzoate as a white solid (410 mg, 81%).

GCMS (Method 1): m/z 182.0 (ES+), at 6.76 min.

¹H NMR: (400 MHz, DMSO-d₆) δ: 10.08 (d, J=2.4 Hz, 1H), 8.33 (d, J=1.6 Hz, 1H), 8.05-8.04 (m, 2H), 3.92 (s, 3H).

Step 3. DAST (0.44 mL, 3.37 mmol) was added to a solution of methyl 3-fluoro-5-formylbenzoate (410 mg, 2.25 mmol) at 0° C. and the reaction mixture was stirred at RT for 2 h. The reaction mixture was neutralized with 10% NaHCO₃ (20 mL) up to pH-7, and reaction mixture was partitioned between water (100 mL) and DCM (50 mL). The organic layer was separated, dried (Na₂SO₄) and the solvent removed in vacuo. The residue was purified by gradient flash column chromatography eluting with 0-30% EtOAc in hexane to afford methyl 3-(difluoromethyl)-5-fluorobenzoate as a colourless liquid (400 mg, 87%).

GCMS (Method 2): m/z 204.0 (ES+), at 2.36 min.

¹H NMR: (400 MHz, DMSO-d₆) δ: 7.99 (s, 1H), 7.89 (d, J=11.2 Hz, 1H), 7.80 (d, J=11.2 Hz, 1H), 7.35-6.98 (m, 1H), 3.91 (s, 3H).

Step 4. LiAlH₄ (2.0 M in THF, 0.45 mL, 0.90 mmol) was added to a solution of methyl 3-(difluoromethyl)-5-fluorobenzoate (390 mg, 1.81 mmol) in THF (10 mL) at 0° C. and the reaction mixture was stirred at RT for 1 h. The reaction mixture was neutralized with 1.5 N HCl (50 mL) up to pH-7 and then partitioned between water (100 mL) and EtOAc (50 mL). The organic layer was separated, dried (Na₂SO₄) and the solvent removed in vacuo to afford (3-(difluoromethyl)-5-fluorophenyl)methanol as a colourless liquid (230 mg, 72%).

GCMS (Method 2): m/z 176.0 (ES+), at 6.36 min.

¹H NMR: (400 MHz, DMSO-d₆) δ: 7.39 (s, 1H), 7.32-7.29 (m, 3H), 5.46 (d, J=6.4 Hz, 1H), 4.57 (t, J=6.4 Hz, 2H).

Step 5. Thionyl chloride (3 mL, 43.2 mmol) was added to a solution of (3-(difluoromethyl)-5-fluorophenyl)methanol (170 mg, 0.96 mmol) in chloroform (10 mL) at RT and the reaction mixture was heated at 65° C. for 12 h. The reaction mixture was neutralized with 10% NaHCO₃ (20 mL) up to pH-7, then partitioned between water (50 mL) and EtOAc (50 mL). The organic layer was separated, dried (Na₂SO₄) and the solvent removed in vacuo to afford 1-(chloromethyl)-3-(difluoromethyl)-5-fluorobenzene as a colourless liquid (170 mg, crude). The crude product was used in the next step without further purification. Data in table 2.

### Typical Procedure 25, Exemplified by the Preparation of Intermediate 27, 1-chloro-5-(chloromethyl)-2,3-difluorobenzene

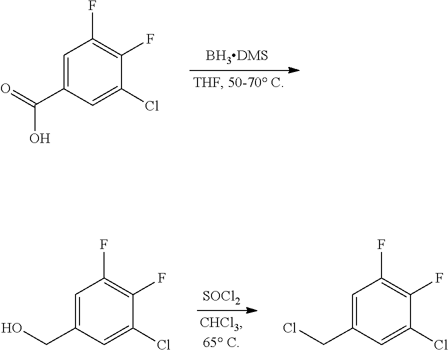

Step 1. Borane dimethyl sulfide complex (0.58 mL, 6.25 mmol) was added to a solution of 3-chloro-4,5-difluorobenzoic acid (400 mg, 2.08 mmol) in THF (6 mL) at 50° C. and the reaction mixture was heated at 70° C. for 2 h. The reaction mixture was cooled to 0° C., quenched with methanol and stirred at RT for 12 h. The solvent was removed in vacuo. The residue was purified by gradient flash column chromatography eluting with 0-30% EtOAc in hexane to afford (3-chloro-4,5-difluorophenyl)methanol as a pink liquid (237 mg, 63%).

Step 2. Thionyl chloride (1.8 mL, 25.2 mmol) was added to a solution of (3-chloro-4,5-difluorophenyl)methanol (100 mg, 0.56 mmol) in chloroform (5 mL) and the reaction mixture was heated at 65° C. for 12 h. The reaction mixture was neutralized with 10% NaHCO₃ solution (20 mL) up to pH-7, and then partitioned between water (50 mL) and DCM (50 mL). The organic layer was separated, dried (Na₂SO₄) and the solvent removed in vacuo to afford 1-chloro-5-(chloromethyl)-2,3-difluorobenzene as a colourless liquid (90 mg, crude). The crude product was used in the next step without further purification. Data in table 2.

### Intermediate 28, 5-(chloromethyl)-1,3-difluoro-2-(trifluoromethyl)benzene

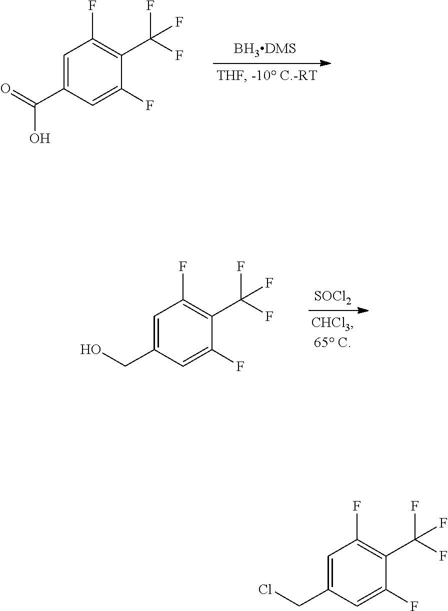

The title compound (128 mg, 25%) was prepared in two steps from 3,5-difluoro-4-(trifluoromethyl)benzoic acid (500 mg, 2.21 mmol) and borane dimethyl sulfide complex (1 M in THF, 11.1 mL, 11.1 mmol) in THF (50 mL) stirred at RT for 12 h; and thionyl chloride (130 μL, 1.76 mmol) in chloroform (10 mL) heated at 70° C. for 12 h using the methods of Intermediate 27. After completion of step 2, the title compound was isolated as a brown oil by neutralization with 10% NaHCO₃ solution (20 mL) up to pH-7, and then partitioning between water (50 mL) and DCM (50 mL). The organic layer was separated, dried (Na₂SO₄) and the solvent removed in vacuo. Data in table 2.

### Intermediate 34, 5-(chloromethyl)-2-(difluoromethoxy)pyridine

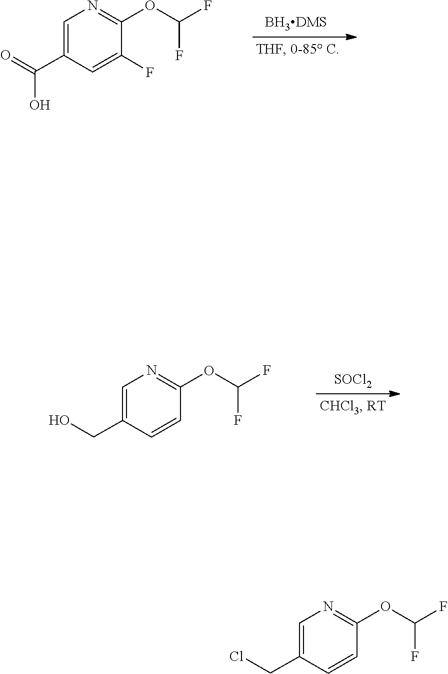

The title compound (140 mg, 54%) was prepared in two steps from 6-(difluoromethoxy)nicotinic acid (250 mg, 1.32 mmol) and borane dimethyl sulfide complex (2 M in THF, 3.3 mL, 6.6 mmol) in THF (2 mL) heated at 85° C. for 16 h; and thionyl chloride (0.2 mL, 2.4 mmol) in chloroform (2 mL) stirred at RT for 3 h using the methods of Intermediate 27. After completion of step 2, the title compound was isolated as a colourless liquid by quenching with ice cold water (50 mL) and extraction with DCM (2×50 mL). The combined organic layers were washed with brine solution (100 mL), dried (Na₂SO₄) and the solvent removed in vacuo. Data in table 2.

### Typical Procedure 26, Exemplified by the Preparation of Intermediate 29, 1-(chloromethyl)-3-fluoro-5-isopropylbenzene

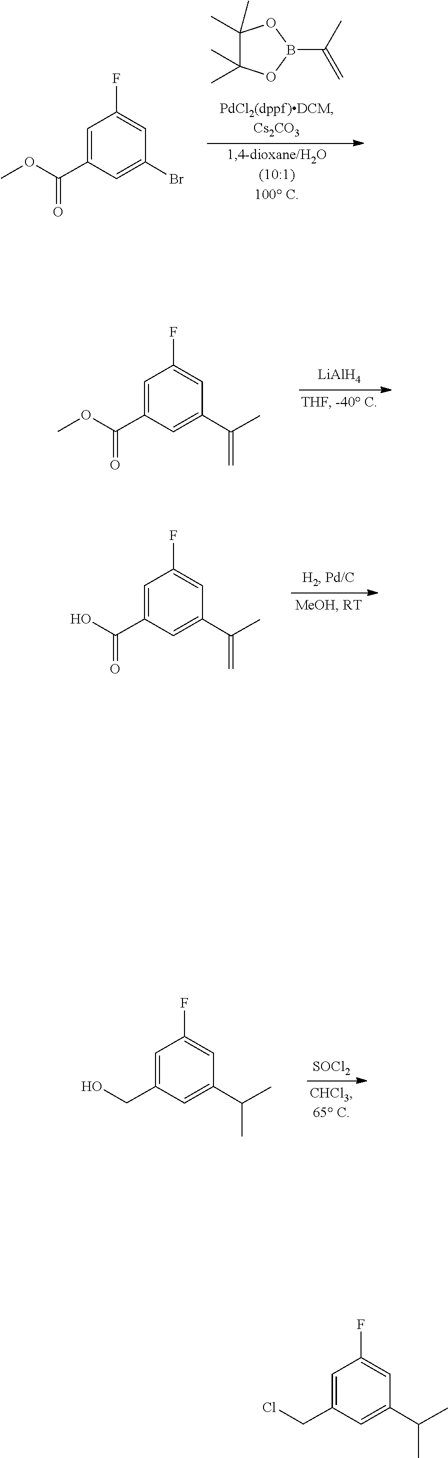

Step 1. In a sealed tube, Cs₂CO₃ (1.39 g, 4.29 mmol), Pd(dppf)Cl₂.DCM (175 mg, 0.214 mmol) and 4,4,5,5-tetramethyl-2-(prop-1-en-2-yl)-1,3,2-dioxaborolane (541 mg, 3.21 mmol) were added to a degassed suspension of methyl 3-bromo-5-fluorobenzoate (500 mg, 2.145 mmol) in 1,4-dioxane:H₂O (10 mL:1 mL) and the reaction mixture was heated at 100° C. for 16 h. The reaction mixture was partitioned between water (100 mL) and EtOAc (100 mL). The organic layer was removed and the aqueous layer was extracted with EtOAc (100 mL). The combined organic layers were washed with brine solution (50 mL), dried (Na₂SO₄) and the solvent removed in vacuo. The residue was purified by gradient flash column chromatography eluting with 0-20% EtOAc in pet-ether to afford methyl 3-fluoro-5-(prop-1-en-2-yl)benzoate as a colourless liquid (380 mg, 91%).

GCMS (Method 1): m/z 194.0 (ES+), at 6.89 min.

¹H NMR: (300 MHz, DMSO-d₆) δ: 7.88 (s, 1H), 7.71-7.61 (m, 2H), 5.60 (s, 1H), 5.27 (s, 1H), 3.89 (s, 3H), 2.14 (s, 3H).

Step 2. LiAlH₄ (1 M in THF, 2.93 mL, 2.93 mmol) was added dropwise to a suspension of methyl 3-fluoro-5-(prop-1-en-2-yl)benzoate (380 mg, 1.95 mmol) in THF (20 mL) at −40° C. and the reaction mixture was stirred at −40° C. for 3 h. The reaction mixture was quenched with sat. NH₄Cl solution (100 mL) and extracted with EtOAc (2×150 mL). The combined organic layers were washed with brine solution (100 mL), dried (Na₂SO₄) and the solvent removed in vacuo. The residue was purified by gradient flash column chromatography eluting with 0-60% EtOAc in pet-ether to afford (3-fluoro-5-(prop-1-en-2-yl)phenyl) methanol as a colourless liquid (250 mg, 77%).

GCMS (Method 1): m/z 166.0 (ES+), at 6.94 min.

¹H NMR: (400 MHz, DMSO-d₆) δ: 7.29 (s, 1H), 7.20-7.16 (m, 1H), 7.07-7.04 (m, 1H), 5.49 (s, 1H), 5.34 (t, J=5.6 Hz, 1H), 5.16 (s, 1H), 4.52 (d, J=6.0 Hz, 2H), 2.10 (s, 3H).

Step 3. Pd/C (100 mg) was added to a solution of (3-fluoro-5-(prop-1-en-2-yl)phenyl)methanol (250 mg, 1.50 mmol) in MeOH (25 mL) and the reaction mixture was stirred under a hydrogen atmosphere at RT for 4 h. The reaction mixture was filtered through a celite pad, washed with MeOH (2×20 mL) and the filtrate was concentrated in vacuo to afford (3-fluoro-5-isopropylphenyl)methanol as a colourless liquid (230 mg, 91%).

¹H NMR: (400 MHz, DMSO-d₆) δ: 7.03 (s, 1H), 6.94-6.91 (m, 2H), 5.28 (t, J=6.0 Hz, 1H), 4.48 (d, J=5.6 Hz, 2H), 2.91-2.88 (m, 1H), 1.20-1.18 (m, 6H).

Step 4. Thionyl chloride (500 μL, 6.84 mmol) was added to a suspension of (3-fluoro-5-isopropylphenyl)methanol (230 mg, 1.36 mmol) in chloroform (10 mL) at 0° C. and the reaction mixture was stirred at RT for 16 h. The reaction mixture was partitioned between 10% NaHCO₃ solution (100 mL) and EtOAc (100 mL). The organic layer was removed and the aqueous layer was extracted with EtOAc (100 mL). The combined organic layers were washed with brine solution (50 mL), dried (Na₂SO₄) and the solvent removed in vacuo to afford 1-(chloromethyl)-3-fluoro-5-isopropylbenzene as a yellow liquid (204 mg, 80%). Data in table 2.

### Typical Procedure 27, Exemplified by the Preparation of Intermediate 30, 1-(chloromethyl)-3-fluoro-5-(1-(trifluoromethyl)cyclopropyl)benzene

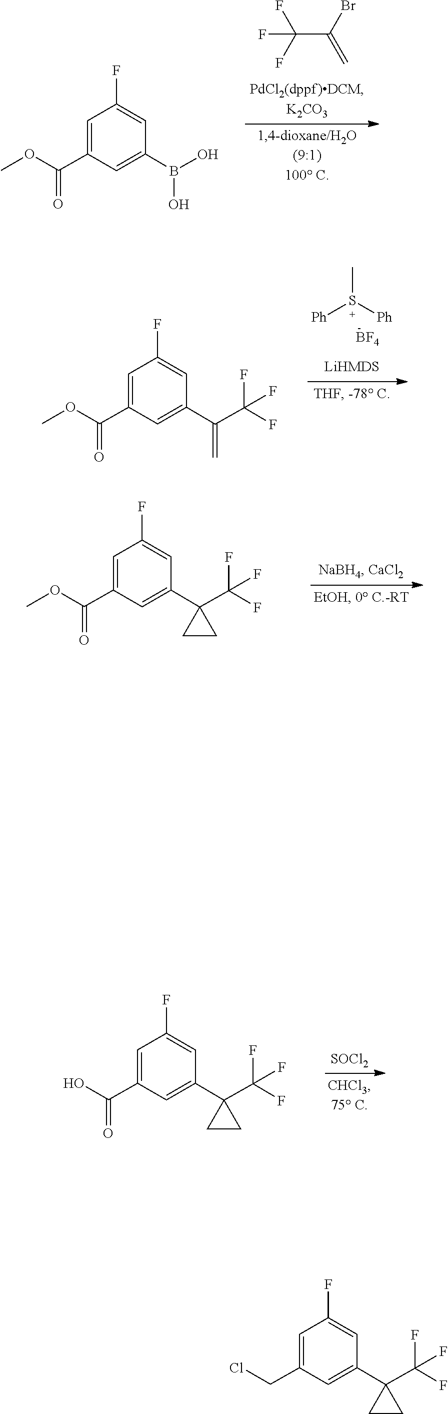

Step 1. K₂CO₃ (2.36 g, 17.14 mmol) was added to a solution of 2-bromo-3,3,3-trifluoroprop-1-ene (1.5 g, 8.57 mmol) and (3-fluoro-5-(methoxycarbonyl)phenyl)boronic acid (1.86 g, 9.43 mmol) in 1,4-dioxane:water (18 mL:2 mL). The reaction mixture was degassed with N₂ for 10 minutes, followed by the addition of Pd(dppf)₂Cl₂.DCM complex (351 mg, 0.43 mmol). The reaction mixture was heated at 100° C. for 16 h. The reaction mixture was filtered through celite pad, washed with DCM (100 mL) and the filtrate was concentrated in vacuo. The residue was purified by gradient flash column chromatography eluting with 0-15% EtOAc in pet-ether to afford methyl 3-fluoro-5-(3,3,3-trifluoroprop-1-en-2-yl)benzoate as a colourless liquid (1.2 g, 57%).

GCMS (Method 2): m/z 248.0 (ES+), at 2.67 min.

¹H NMR: (400 MHz, CDCl₃) δ: 7.96 (s, 1H), 7.79-7.76 (m, 1H), 7.41-7.37 (m, 1H), 6.10-6.09 (m, 1H), 5.91-5.90 (m, 1H), 3.97 (s, 3H).

Step 2. Diphenyl(methyl)sulfonium tetrafluoroborate (1.39 g, 4.84 mmol) and lithium bis(hexamethyldisilazide) solution (1 M in THF, 9.67 mL, 9.67 mmol) were added sequentially to a solution of methyl 3-fluoro-5-(3,3,3-trifluoroprop-1-en-2-yl)benzoate (0.8 g, 3.22 mmol) in THF (50 mL) at −78° C. The reaction mixture was stirred at −78° C. for 1 h. The reaction mixture was quenched with sat. NH₄Cl solution (100 mL) and the aqueous layer extracted with EtOAc (2×100 mL). The combined organic layers were dried (Na₂SO₄) and the solvent removed in vacuo. The residue was purified by gradient flash column chromatography eluting with 0-10% EtOAc in pet-ether to afford methyl 3-fluoro-5-(1-(trifluoromethyl)cyclopropyl)benzoate as a brown liquid (180 mg, 7%).

GCMS (Method 2): m/z 262.0 (ES+), at 3.07 min.

Step 3. NaBH₄ (0.121 g, 3.2 mmol) was added portion wise, followed by the addition of a solution of CaCl₂ (0.711 g, 6.41 mmol) in EtOH (5 mL) drop wise to a solution of methyl 3-fluoro-5-(1-(trifluoromethyl)cyclopropyl)benzoate (0.28 g, 1.06 mmol) in EtOH (5 mL) at 0° C. The reaction mixture was stirred at RT for 20 h. The reaction mixture was quenched with ice cold water (50 mL), and the aqueous layer extracted with EtOAc (2×100 mL). The combined organic layers were dried (Na₂SO₄) and the solvent removed in vacuo to afford (3-fluoro-5-(1-(trifluoromethyl)cyclopropyl)phenyl)methanol as a colourless liquid (130 mg, 52%).

LCMS (Method 3): m/z No ionisation observed (ES+), at 2.13 min.

¹H NMR: (300 MHz, DMSO-d₆) δ: 7.28 (s, 1H), 7.16-7.12 (m, 2H), 5.38 (t, J=8.0 Hz, 1H), 4.52 (d, J=8.0 Hz, 2H), 1.35 (t, J=8.0 Hz, 2H), 1.24-1.15 (m, 2H).

Step 4. Thionyl chloride (1.2 mL, 16.6 mmol) was added dropwise to a solution of (3-fluoro-5-(1-(trifluoromethyl)cyclopropyl)phenyl)methanol (0.13 g, 0.55 mmol) in CHCl₃ (20 mL), at 0° C. and the reaction mixture was heated at 75° C. for 48 h. The solvent was removed in vacuo to afford 1-(chloromethyl)-3-fluoro-5-(1-(trifluoromethyl)cyclopropyl)benzene as a brown liquid (150 mg, crude). The crude product was used in the next step without further purification. Data in table 2.

### Intermediate 32, 5-(chloromethyl)-3-fluoro-2-methoxypyridine

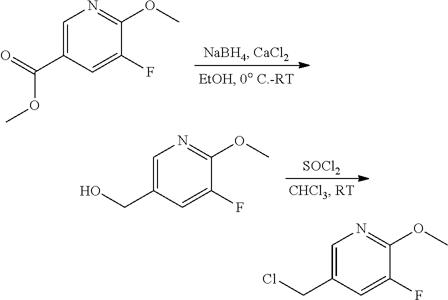

The title compound (294 mg, 62%) was prepared in two steps from methyl 5-fluoro-6-methoxynicotinate (0.5 g, 2.6 mmol) and NaBH₄ (0.15 g, 4.04 mmol) in EtOH (5 mL) and CaCl₂ (0.44 g, 4.04 mmol) in EtOH (5 mL) stirred at RT for 1 h; and thionyl chloride (1.13 g, 9.54 mmol) in chloroform (10 mL) stirred at RT for 1 h using the methods of Intermediate 30, steps 3 and 4. After completion of step 2, the title compound was isolated as a colourless gum by quenching the reaction mixture with 10% NaHCO₃ solution (50 mL) and extraction with DCM (50 mL). The organic layer was separated, dried (Na₂SO₄) and the solvent removed in vacuo. The residue was purified by gradient flash column chromatography eluting with 0-10% EtOAc in pet-ether gradient. Data in table 2.

### Intermediate 33, 5-(chloromethyl)-2-methoxypyridine

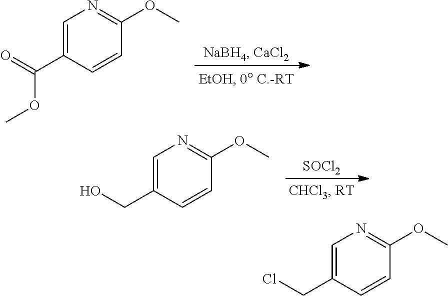

The title compound (180 mg, 75%) was prepared in two steps from methyl 6-methoxynicotinate (0.5 g, 2.99 mmol), NaBH₄ (0.37 g, 8.97 mmol) and CaCl₂ (0.49 g 4.48 mmol) in EtOH (30 mL) stirred at RT for 16 h; and thionyl chloride (0.52 mL, 7.18 mmol) in chloroform (20 mL) stirred at RT for 2 h using the methods of Intermediate 30, steps 3 and 4. After completion of step 2, the title compound was isolated as a colourless liquid by quenching the reaction mixture with 10% NaHCO₃ solution (50 mL) and extraction with DCM (2×50 mL). The combined organic layers washed with brine solution (50 mL), dried (Na₂SO₄) and the solvent removed in vacuo. Data in table 2.

### SYNTHESIS OF EXAMPLES

Typical procedures for the preparation of examples, as exemplified by the preparation of the below examples in Procedures 1-27.

### Procedure 1:

### Example 1, 1-(2-(3-fluoro-5-(trifluoromethyl)benzyl)pyridin-4-yl)-1,5,6,7-tetrahydro-4H-pyrazolo[4,3-c]pyridin-4-one

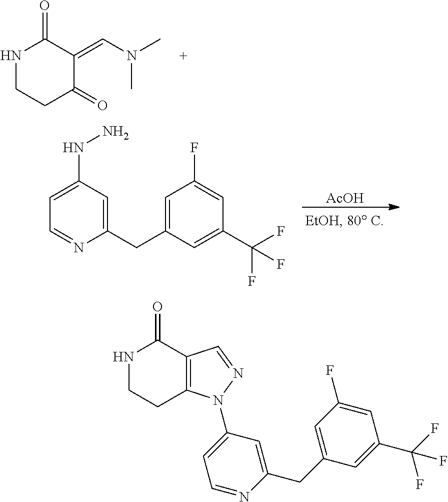

A catalytic amount of acetic acid (1 mL) was added to a solution of 2-(3-fluoro-5-(trifluoromethyl)benzyl)-4-hydrazineylpyridine (Intermediate 6, 5 g, 17.5 mmol) in EtOH (250 mL) at RT, followed by the addition of (E)-3-((dimethylamino)methylene)piperidine-2,4-dione (Intermediate 1, 3.5 g, 21.1 mmol) and the reaction mixture was heated at 80° C. for 16 h. The solvent was removed in vacuo and the residue was partitioned between EtOAc (100 mL) and sat. NaHCO₃ solution (100 mL). The organic layer was separated, dried (Na₂SO₄) and the solvent removed in vacuo. The residue was purified by gradient flash chromatography eluting with 0-10% MeOH in DCM to afford 1-(2-(3-fluoro-5-(trifluoromethyl)benzyl)pyridin-4-yl)-1,5,6,7-tetrahydro-4H-pyrazolo[4,3-c]pyridin-4-one as an off-white solid (2.0 g, 30%). Data in table 3.

### Procedure 2:

### Example 3, 5-methyl-1-(4-(3-(trifluoromethyl)benzyl)pyridin-2-yl)-1,5,6,7-tetrahydro-4H-pyrazolo[4,3-c]pyridin-4-one

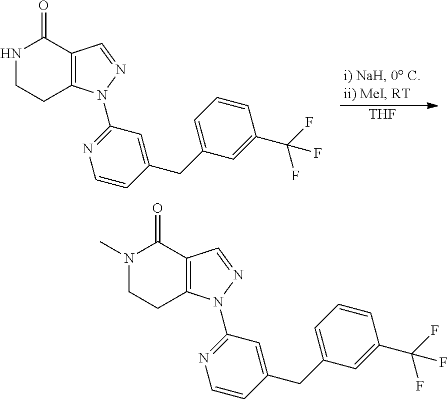

NaH (60% in mineral oil, 21 mg, 0.53 mmol) was added to a solution of 1-(4-(3-(trifluoromethyl)benzyl)pyridin-2-yl)-1,5,6,7-tetrahydro-4H-pyrazolo[4,3-c]pyridin-4-one (Example 2, 100 mg, 0.26 mmol) in THF (4 mL) at 0° C. After stirring for 30 min, Mel (0.03 mL, 0.53 mmol) in THF (1 mL) was slowly added and the reaction mixture stirred at RT for 1 h. The reaction mixture was quenched with water (15 mL) and the aqueous layer was extracted with EtOAc (3×15 mL). The combined organic layers were dried (Na₂SO₄) and the solvent removed in vacuo. The residue was purified by gradient flash column chromatography eluting with 27-29% EtOAc in hexane to afford 5-methyl-1-(4-(3-(trifluoromethyl)benzyl)pyridin-2-yl)-1,5,6,7-tetrahydro-4H-pyrazolo[4,3-c]pyridin-4-one as a colourless gel (43 mg, 43%). Data in table 3.

### Procedure 3:

### Example 6, 1-(4-((6-fluoro-5-methylpyridin-3-yl)methyl)pyridin-2-yl)-1,5,6,7-tetrahydro-4H-pyrazolo[4,3-c]pyridin-4-one

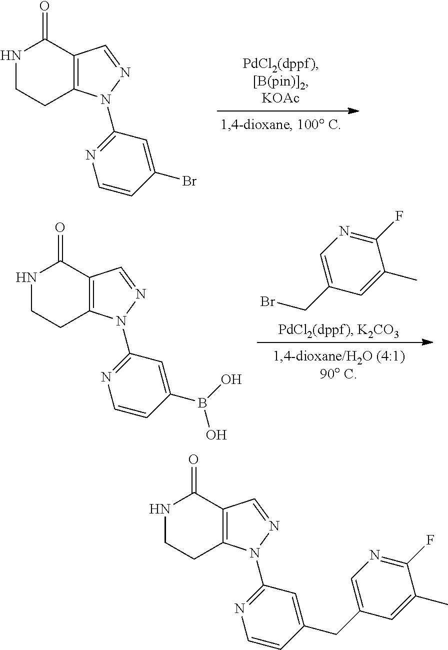

Step 1. bis(Pinacolato)diboron (260 mg, 1.02 mmol) was added to a solution of 1-(4-bromopyridin-2-yl)-1,5,6,7-tetrahydro-4H-pyrazolo[4,3-c]pyridin-4-one (Intermediate 12, 200 mg, 0.68 mmol) in 1,4-dioxane (8 mL), the reaction mixture was degassed under argon for 25 min and then KOAc (201 mg, 2.05 mmol) and PdCl₂(dppf).DCM (28 mg, 0.03 mmol) were added. The reaction mixture was heated at 100° C. for 16 h. The reaction mixture was filtered through Celite and washed with 5% MeOH in DCM (100 mL). The solvent was removed in vacuo and the residue was triturated with MTBE (20 mL) to afford (2-(4-oxo-4,5,6,7-tetrahydro-1H-pyrazolo[4,3-c]pyridin-1-yl)pyridin-4-yl)boronic acid as a brown solid (415 mg, crude). The crude product was used in the next step without further purification.

MS (Method 1): m/z 259.0 (ES+).

Step 2. 5-(Bromomethyl)-2-fluoro-3-methylpyridine (200 mg, 0.98 mmol) and K₂CO₃ (407 mg, 2.95 mmol) were added to a solution of (2-(4-oxo-4,5,6,7-tetrahydro-1H-pyrazolo[4,3-c]pyridin-1-yl)pyridin-4-yl)boronic acid (432 mg, 1.67 mmol) in 1,4-dioxane:H₂O (4:1). After degassing with argon for 25 min, PdCl₂(dppf).DCM (40 mg, 0.04 mmol) was added and the reaction mixture was heated at 90° C. for 1 h. The reaction mixture was quenched by the addition of water (100 mL) and the aqueous layer was extracted with EtOAc (3×50 mL). The organic layers were combined, dried (Na₂SO₄) and the solvent removed in vacuo. The residue was triturated with Et₂O, filtered and washed with 2% MeOH in DCM to afford 1-(4-((6-fluoro-5-methylpyridin-3-yl)methyl)pyridin-2-yl)-1,5,6,7-tetrahydro-4H-pyrazolo[4,3-c]pyridin-4-one as an off white solid (180 mg, 54%). Data in table 3.

### Procedure 4:

### Example 8, 2-(4-(3-(trifluoromethyl)benzyl)pyridin-2-yl)-2,5,6,7-tetrahydro-4H-pyrazolo[4,3-c]pyridin-4-one

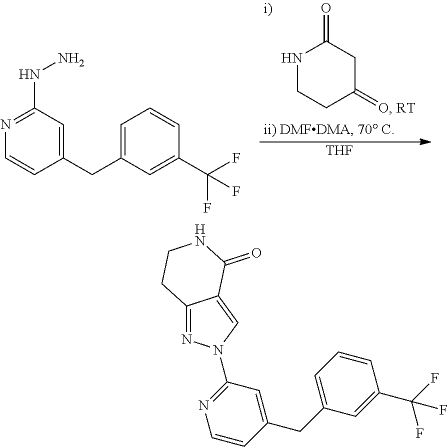

2-Hydrazineyl-4-(3-(trifluoromethyl)benzyl)pyridine (Intermediate 7, 54 mg, 0.2 mmol) in THF (1 mL) was added to a suspension of piperidine-2,4-dione (22 mg, 0.2 mmol) in THF (1 mL) and the reaction mixture stirred at RT for 2 h. DMF-DMA (0.08 mL, 0.6 mmol) was added and the reaction mixture heated at 70° C. for 16 h. The solvent was removed in vacuo and the residue was purified by gradient flash chromatography eluting with 0-10% MeOH in DCM. The residue was further purified by prep HPLC (Method 2-40-70% gradient) to afford 2-(4-(3-(trifluoromethyl)benzyl)pyridin-2-yl)-2,5,6,7-tetrahydro-4H-pyrazolo[4,3-c]pyridin-4-one as a white solid (13 mg, 18%). Data in table 3.

### Procedure 5:

### Example 10, 1-(4-(3-fluoro-5-(trifluoromethyl)benzyl)pyridin-2-yl)-5,6,7,8-tetrahydropyrazolo[4,3-c]azepin-4(1H)-one

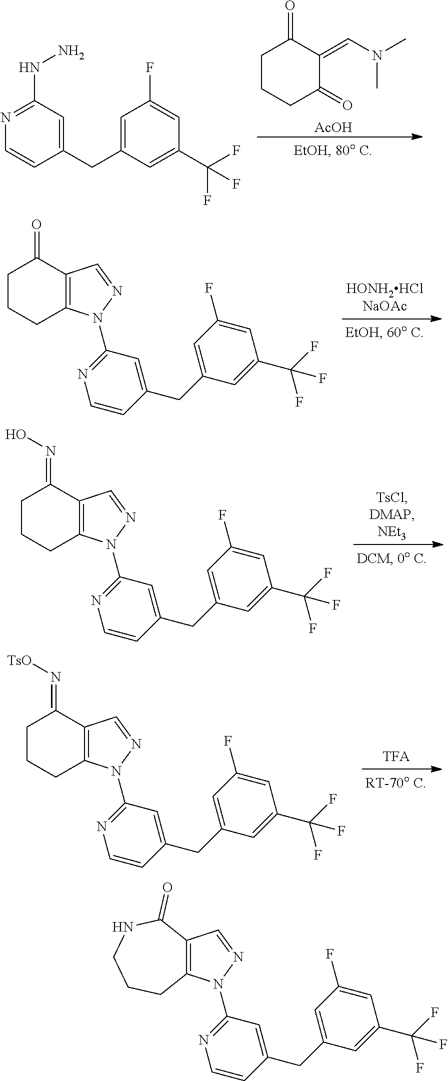

Step 1. 2-((Dimethylamino)methylene)cyclohexane-1,3-dione (Intermediate 2, 3.37 g, 20.2 mmol) and acetic acid (2.7 mL) were added to a suspension of 4-(3-fluoro-5-(trifluoromethyl)benzyl)-2-hydrazineylpyridine (Intermediate 8, 5.82 g, 20.4 mmol) in EtOH (70 mL) and the reaction mixture heated at 80° C. for 3 h. The solvent was removed in vacuo. The residue was purified by gradient flash column chromatography eluting with 12-100% EtOAc in i-hexane and the solvent removed in vacuo to afford 1-(4-(3-fluoro-5-(trifluoromethyl)benzyl)pyridin-2-yl)-1,5,6,7-tetrahydro-4H-indazol-4-one as a dark orange solid (5.55 g, 71% yield).

LCMS (Method 4): m/z 390.4 (ES+), at 1.63 min.

¹H NMR: (400 MHz, DMSO-d₆) δ: 8.46 (dd, J=5.1, 0.7 Hz, 1H), 8.09 (s, 1H), 7.88 (dd, J=1.6, 0.8 Hz, 1H), 7.66 (dt, J=1.6, 0.8 Hz, 1H), 7.63-7.54 (m, 2H), 7.40 (dd, J=5.1, 1.5 Hz, 1H), 4.24 (s, 2H), 3.45-3.36 (m, 2H), 2.49-2.37 (m, 2H), 2.08 (p, J=6.2 Hz, 2H).

Step 2. Hydroxylamine hydrochloride (444 mg, 6.39 mmol) and sodium acetate (524 mg, 6.39 mmol) were added to a suspension of 1-(4-(3-fluoro-5-(trifluoromethyl)benzyl)pyridin-2-yl)-1,5,6,7-tetrahydro-4H-indazol-4-one (500 mg, 1.28 mmol) in EtOH (30 mL) and the reaction mixture was heated at 60° C. for 3 h. The reaction mixture was partitioned between water (20 mL) and EtOAc (20 mL) and the organic layer removed. The aqueous layer was extracted with EtOAc (2×20 mL), the combined organic layers were dried (phase separator) and the solvent removed in vacuo to afford 1-(4-(3-fluoro-5-(trifluoromethyl)benzyl)pyridin-2-yl)-1,5,6,7-tetrahydro-4H-indazol-4-one oxime as a beige semi-solid (497 mg, 96%, mixture of oxime isomers).

LCMS (Method 4): m/z 405.4 (ES+), at 1.59 min.

¹H NMR: (400 MHz, DMSO-d₆) δ: 10.74 (s, 0.8H), 10.68 (s, 0.2H), 8.45-8.36 (m, 1H), 8.30 (s, 1H), 7.88 (dd, J=1.6, 0.8 Hz, 0.9H), 7.82 (d, J=1.6 Hz, 0.1H), 7.65 (s, 1H), 7.63-7.52 (m, 1.8H), 7.51-7.43 (m, 0.2H), 7.34 (dd, J=5.1, 1.5 Hz, 0.7H), 7.31 (d, J=5.3 Hz, 0.3H), 4.22 (d, J=3.2 Hz, 2H), 3.24 (t, J=6.1 Hz, 2H), 2.46-2.38 (m, 2H), 1.96-1.87 (m, 2H).

Step 3. DMAP (10.0 mg, 0.08 mmol) was added to a suspension of 1-(4-(3-fluoro-5-(trifluoromethyl)benzyl)pyridin-2-yl)-1,5,6,7-tetrahydro-4H-indazol-4-one oxime (487 mg, 1.20 mmol) and triethyl amine (0.5 mL, 3.55 mmol) in DCM (20 mL), and the reaction mixture cooled to 0° C. To this was added para-toluenesulfonyl chloride (273 mg, 1.43 mmol) and the reaction mixture stirred for 2 h at 0° C. The residue was partitioned between DCM (10 mL) and water (10 mL) and the aqueous layer removed. The organic layer was washed with water (2×10 mL), dried (phase separator) and the solvent removed in vacuo. The residue was purified by gradient flash column chromatography eluting with 10-60% EtOAc in i-hexane to afford 1-(4-(3-fluoro-5-(trifluoromethyl)benzyl)pyridin-2-yl)-1,5,6,7-tetrahydro-4H-indazol-4-one O-tosyl oxime as a beige semi-solid (428 mg, 64%).

LCMS (Method 4): m/z 599.3 (ES+), at 1.99 min.

¹H NMR: (400 MHz, DMSO-d₆) δ: 8.44 (dd, J=5.1, 0.7 Hz, 0.9H), 8.41 (dd, J=5.1, 0.7 Hz, 0.1H), 8.18 (s, 1H), 7.93-7.82 (m, 3H), 7.69-7.64 (m, 1H), 7.64-7.53 (m, 2H), 7.52-7.46 (m, 2H), 7.40 (dd, J=5.1, 1.5 Hz, 0.9H), 7.35 (dd, J=5.1, 1.5 Hz, 0.1H), 4.25 (s, 1.8H), 4.22 (s, 0.2H), 3.25 (t, J=6.2 Hz, 2H), 2.49-2.44 (m, 2H), 2.42 (s, 3H), 1.96-1.81 (m, 2H).

Step 4. A suspension of 1-(4-(3-fluoro-5-(trifluoromethyl)benzyl)pyridin-2-yl)-1,5,6,7-tetrahydro-4H-indazol-4-one O-tosyl oxime (428 mg, 0.77 mmol) in TFA (20 mL) was stirred at RT for 1 h. The reaction mixture was then heated at 70° C. for 16 h. The residue was partitioned between ice and DCM (10 mL) and the organic layer separated. The aqueous layer was washed with DCM (2×10 mL), the combined organic layers dried (phase separator) and the solvent removed in vacuo. The residue was purified by gradient flash column chromatography eluting with 2-7% MeOH in DCM to afford 1-(4-(3-fluoro-5-(trifluoromethyl)benzyl)pyridin-2-yl)-5,6,7,8-tetrahydropyrazolo[4,3-c]azepin-4(1H)-one as a white solid (218 mg, 70%). Data in table 3.

Procedure 6:

### Example 13, 1-(4-(3-(trifluoromethyl)benzyl)pyridin-2-yl)-6,7-dihydro-1H-pyrazolo[4,3-f][1,4]oxazepin-4(5H)-one

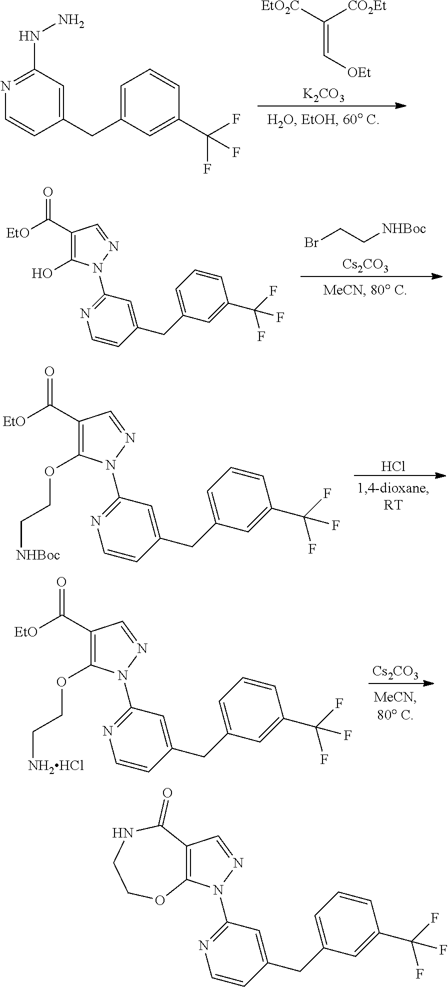

Step 1. K₂CO₃ (5.16 g, 37.45 mmol) was added to a solution 2-hydrazineyl-4-(3-(trifluoromethyl)benzyl)pyridine (Intermediate 7, 4 g, 14.98 mmol) and diethyl 2-(ethoxymethylene)malonate (8.08 g, 37.45 mmol) in EtOH:H₂O (85 mL:15 mL) and the reaction mixture heated at 60° C. for 16 h. The reaction mixture was partitioned between water (100 mL) and EtOAc (100 mL). The organic layer was separated, dried (Na₂SO₄) and the solvent removed in vacuo. The residue was purified by gradient flash chromatography eluting with 0-5% MeOH in DCM to afford ethyl 5-hydroxy-1-(4-(3-(trifluoromethyl)benzyl)pyridin-2-yl)-1H-pyrazole-4-carboxylate as a yellow solid (1.5 g, 25%).

LCMS (Method 3): m/z 392.0 (ES+), at 2.55 min.

¹H NMR: (400 MHz, DMSO-d₆) δ: 8.20 (d, J=4.8 Hz, 2H), 7.63-7.57 (m, 4H), 7.42 (s, 1H), 6.88 (d, J=6.3 Hz, 1H), 4.05-3.96 (m, 4H), 1.17 (t, J=7.1 Hz, 3H). 1 exchangeable proton not observed.

Step 2. K₂CO₃ (0.79 g, 5.75 mmol) was added to a solution of ethyl 5-hydroxy-1-(4-(3-(trifluoromethyl)benzyl)pyridin-2-yl)-1H-pyrazole-4-carboxylate (1.5 g, 3.83 mmol) and tert-butyl (2-bromoethyl)carbamate (1.02 g, 4.59 mmol) in MeCN (30 mL) and the reaction mixture was heated at 80° C. for 16 h. The reaction mixture was partitioned between water (50 mL) and EtOAc (50 mL). The organic layer was separated, dried (Na₂SO₄) and the solvent removed in vacuo to afford ethyl 5-(2-((tert-butoxycarbonyl)amino)ethoxy)-1-(4-(3-(trifluoromethyl)benzyl)pyridin-2-yl)-1H-pyrazole-4-carboxylate as a colourless liquid (500 mg, crude).

LCMS (Method 3): m/z 535.0 (ES+), at 3.03 min.

Step 3. HCl solution (4 M in 1,4-dioxane, 0.4 mL) was added to a solution of ethyl 5-(2-((tert-butoxycarbonyl)amino)ethoxy)-1-(4-(3-(trifluoromethyl)benzyl)pyridin-2-yl)-1H-pyrazole-4-carboxylate (200 mg, 0.374 mmol in 1,4-dioxane (2 mL) at 0° C. and the reaction mixture stirred at RT for 1 h. The solvent was removed in vacuo to afford ethyl 5-(2-aminoethoxy)-1-(4-(3-(trifluoromethyl)benzyl)pyridin-2-yl)-1H-pyrazole-4-carboxylate hydrochloride as a white solid (250 mg, crude).

LCMS (Method 3): m/z 435.3 (ES+), at 1.85 min.

Step 4. Cs₂CO₃ (169 mg, 0.518 mmol) was added to a solution of ethyl 5-(2-aminoethoxy)-1-(4-(3-(trifluoromethyl)benzyl)pyridin-2-yl)-1H-pyrazole-4-carboxylate hydrochloride (150 mg) in MeCN (1.5 mL) and the reaction mixture was heated at 80° C. for 16 h. The reaction mixture was partitioned between water (50 mL) and EtOAc (50 mL). The organic layer was separated, dried (Na₂SO₄) and the solvent removed in vacuo. The residue was purified by prep HPLC (Method 1) to afford 1-(4-(3-(trifluoromethyl)benzyl)pyridin-2-yl)-6,7-dihydro-1H-pyrazolo[4,3-f][1,4]oxazepin-4(5H)-one as an off-white solid (39 mg, 29%). Data in table 3.

### Procedure 7:

### Example 16, 1-(5-(3-fluoro-5-(trifluoromethyl)benzyl)pyridin-3-yl)-1,5,6,7-tetrahydro-4H-pyrazolo[4,3-c]pyridin-4-one

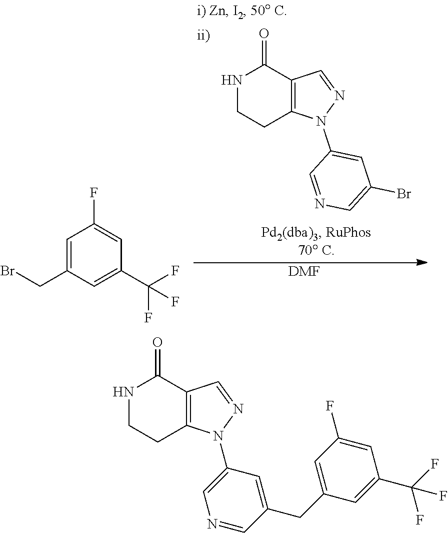

A pinch of iodine was added to a solution of activated zinc (665 mg, 10.2 mmol) in DMF (10 mL) and heated to 50° C. for 5 min followed by the addition of 1-(bromomethyl)-3-fluoro-5-(trifluoromethyl)benzene (526 mg, 2.04 mmol) in DMF (5 mL). The reaction mixture was heated at 50° C. for 1 h and then allowed to cool to RT. The supernatant DMF layer was transferred to a degassed suspension of 1-(5-bromopyridin-3-yl)-1,5,6,7-tetrahydro-4H-pyrazolo[4,3-c]pyridin-4-one (Intermediate 11, 200 mg, 0.68 mmol) and RuPhos (63 mg, 0.13 mmol) in DMF (10 mL) followed by the addition of tris(dibenzylideneacetone)dipalladium(0) (63 mg, 0.06 mmol). The reaction mixture was heated to 70° C. for 16 h and then filtered through Celite and washed with EtOAc (2×50 mL). The filtrate was washed with water (2×50 mL). The organic layer was separated, dried (Na₂SO₄) and the solvent removed in vacuo. The residue was purified by gradient flash chromatography eluting with 0-100% EtOAc in pet-ether to afford 1-(5-(3-fluoro-5-(trifluoromethyl)benzyl)pyridin-3-yl)-1,5,6,7-tetrahydro-4H-pyrazolo[4,3-c]pyridin-4-one as an off-white solid (170 mg, 64%). Data in table 3.

### Procedure 8:

### Example 18, 1-(4-(3-fluoro-5-(trifluoromethyl)phenoxy)pyridin-2-yl)-1,5,6,7-tetrahydro-4H-pyrazolo[4,3-c]pyridin-4-one

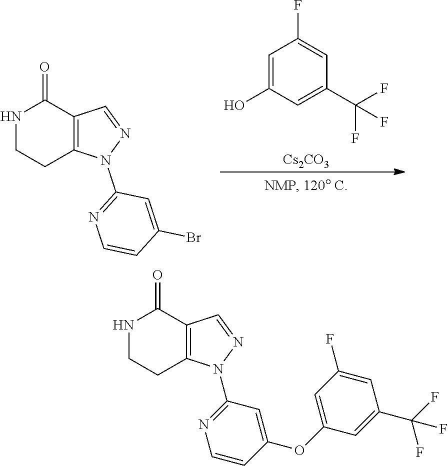

Cs₂CO₃ (1.08 g, 3.33 mmol) was added to a solution of 3-fluoro-5-(trifluoromethyl)phenol (200 mg, 1.11 mmol) in NMP (5 mL), the reaction mixture stirred at RT for 10 min followed by the addition of 1-(4-bromopyridin-2-yl)-1,5,6,7-tetrahydro-4H-pyrazolo[4,3-c]pyridin-4-one (Intermediate 12, 259 mg, 0.88 mmol). The reaction mixture was heated at 120° C. for 16 h. The reaction mixture was poured into water (30 mL) and the precipitate which formed filtered, washed with water (3×20 mL) and dried in vacuo. The residue was purified by prep HPLC (Method 3—100% isocratic gradient) to afford 1-(4-(3-fluoro-5-(trifluoromethyl)phenoxy)pyridin-2-yl)-1,5,6,7-tetrahydro-4H-pyrazolo[4,3-c]pyridin-4-one as a white solid (12 mg, 12%). Data in table 3.

### Procedure 9:

### Example 19, 1-(2-(4-chlorobenzyl)pyridin-4-yl)-1,5,6,7-tetrahydro-4H-pyrazolo[4,3-c]pyridin-4-one

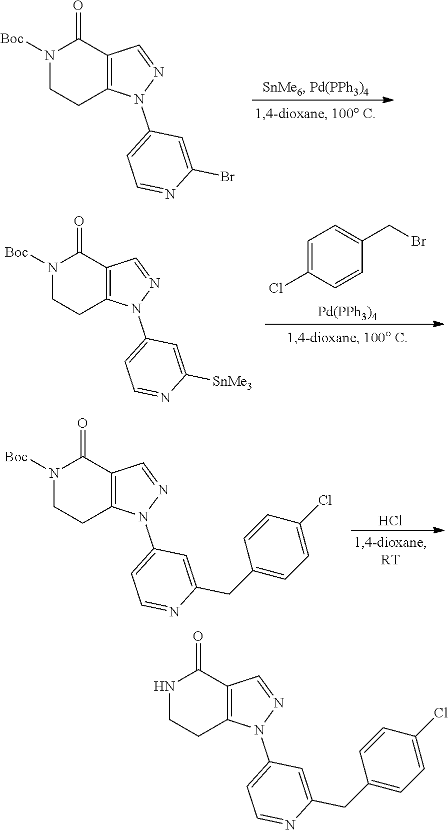

Step 1. Hexamethylditin (1.24 g, 3.81 mmol) was added to a solution of tert-butyl 1-(2-bromopyridin-4-yl)-4-oxo-1,4,6,7-tetrahydro-5H-pyrazolo[4,3-c]pyridine-5-carboxylate (Intermediate 14, 1 g, 2.54 mmol) in 1,4-dioxane (20 mL) and followed by the addition of Pd(PPh₃)₄ (293 mg, 0.25 mmol) and the reaction mixture was heated at 100° C. for 2 h. The solvent was removed in vacuo afford tert-butyl 4-oxo-1-(2-(trimethylstannyl)pyridin-4-yl)-1,4,6,7-tetrahydro-5H-pyrazolo[4,3-c]pyridine-5-carboxylate as a yellow gum (1.5 g, crude).

LCMS (Method 3): m/z 479.1 (ES+), at 1.47 min.

Step 2. 1-(Bromomethyl)-4-chlorobenzene (36 mg, 0.18 mmol) was added to a solution of tert-butyl 4-oxo-1-(2-(trimethylstannyl)pyridin-4-yl)-1,4,6,7-tetrahydro-5H-pyrazolo[4,3-c]pyridine-5-carboxylate (60 mg) in 1,4-dioxane (10 mL) followed by the addition of Pd(PPh₃)₄ (14 mg, 0.01 mmol) under argon, then the reaction mixture was heated at 100° C. for 12 h. The solvent was removed in vacuo and the residue was purified by gradient flash chromatography eluting with 0-50% EtOAc in pet-ether to afford tert-butyl 1-(2-(4-chlorobenzyl)pyridin-4-yl)-4-oxo-1,4,6,7-tetrahydro-5H-pyrazolo[4,3-c]pyridine-5-carboxylate as a colourless liquid (40 mg, 72%).

LCMS (Method 3): m/z 439.2 (ES+), at 2.40 min.

Step 3. HCl solution (4 M in 1,4-dioxane, 5 mL) was added to a solution of tert-butyl 1-(2-(4-chlorobenzyl)pyridin-4-yl)-4-oxo-1,4,6,7-tetrahydro-5H-pyrazolo[4,3-c]pyridine-5-carboxylate (40 mg, 0.09 mmol) in 1,4-dioxane (5 mL) at RT and the reaction mixture was stirred at RT for 2 h. The solvent was removed in vacuo and the residue was purified by prep HPLC (Method 4). The solvent was removed in vacuo and the residue was partitioned between 10% NaHCO₃ solution (5 mL) and EtOAc (10 mL). The organic layer was separated, washed with saturated brine solution (5 mL), dried (Na₂SO₄) and the solvent removed in vacuo to afford 1-(2-(4-chlorobenzyl)pyridin-4-yl)-1,5,6,7-tetrahydro-4H-pyrazolo[4,3-c]pyridin-4-one as a white solid (6 mg, 20%). Data in table 3.

### Procedure 10:

### Example 33, 3-((4-(4-oxo-4,5,6,7-tetrahydro-1H-pyrazolo[4,3-c]pyridin-1-yl)pyridin-2-yl)methyl)-5-(trifluoromethyl)benzonitrile

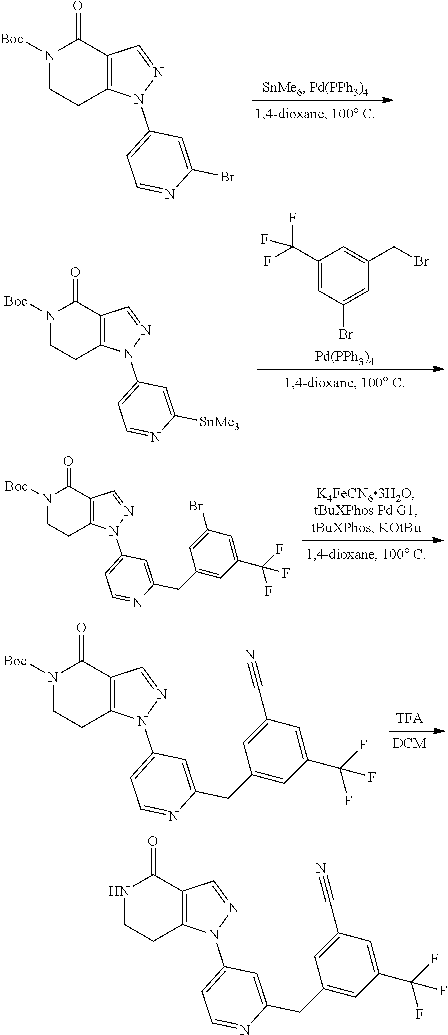

Steps 1 and 2. tert-Butyl 1-(2-(3-bromo-5-(trifluoromethyl)benzyl)pyridin-4-yl)-4-oxo-1,4,6,7-tetrahydro-5H-pyrazolo[4,3-c]pyridine-5-carboxylate (150 mg, 54%) was prepared from tert-butyl 1-(2-bromopyridin-4-yl)-4-oxo-1,4,6,7-tetrahydro-5H-pyrazolo[4,3-c]pyridine-5-carboxylate (Intermediate 14, 200 mg, 0.50 mmol) and 1-bromo-3-(bromomethyl)-5-(trifluoromethyl)benzene (322 mg, 1.01 mmol) using the methods of Procedure 9, steps 1 and 2.

LCMS (Method 2): m/z 450.9 (ES+), at 2.63 min.

¹H NMR: (400 MHz, DMSO-d₆) δ: 8.68 (d, J=5.2 Hz, 1H), 8.22 (s, 1H), 7.92 (s, 1H), 7.84 (s, 1H), 7.78 (s, 1H), 7.74-7.61 (m, 1H), 7.54-7.52 (m, 1H), 4.32 (s, 2H), 4.05-4.01 (m, 2H), 3.32-3.29 (m, 2H), 1.49 (s, 9H).

Step 3. 1 M KOAc in H₂O (8.8 mg, 0.09 mmol) was added to a solution of tert-butyl 1-(2-(3-bromo-5-(trifluoromethyl)benzyl)pyridin-4-yl)-4-oxo-1,4,6,7-tetrahydro-5H-pyrazolo[4,3-c]pyridine-5-carboxylate (100 mg, 0.18 mmol) and K₄FeCN₆.3H₂O (114 mg, 0.27 mmol) in 1,4-dioxane (5 mL) in a sealed tube, and degassed with argon for 15 minutes. t-ButylXPhos (7.6 mg, 0.018 mmol) and t-butylXPhos Pd G1 (12.3 mg, 0.018 mmol) were then added and the reaction mixture degassed with argon for another 5 minutes. The reaction mixture was heated at 100° C. for 3 h. The reaction mixture was partitioned between H₂O (10 mL) and EtOAc (15 mL). The organic layer was separated, dried (Na₂SO₄) and the solvent removed in vacuo. The residue was purified by gradient flash column chromatography eluting with 0-100% EtOAc in pet-ether to afford tert-butyl 1-(2-(3-cyano-5-(trifluoromethyl)benzyl)pyridin-4-yl)-4-oxo-1,4,6,7-tetrahydro-5H-pyrazolo[4,3-c]pyridine-5-carboxylate as a yellow solid (80 mg, 88%).

LCMS (Method 2): m/z 498.0 (ES+), at 2.47 min.

¹H NMR: (400 MHz, DMSO-d₆) δ: 8.67 (d, J=5.6 Hz, 1H), 8.21 (d, J=6.0 Hz, 1H), 8.17 (s, 1H), 7.74 (d, J=2.0 Hz, 1H), 7.65-7.61 (m, 1H), 7.58-7.53 (m, 2H), 4.77 (s, 2H), 4.39-4.31 (m, 2H), 4.02-3.99 (m, 2H), 1.49 (s, 9H).

Step 4. 25% TFA in DCM (1.5 mL) was added to a solution of tert-butyl 1-(2-(3-cyano-5-(trifluoromethyl)benzyl)pyridin-4-yl)-4-oxo-1,4,6,7-tetrahydro-5H-pyrazolo[4,3-c]pyridine-5-carboxylate (80 mg, 0.16 mmol) in DCM (1.5 mL) and the reaction mixture was stirred at RT for 2 h. The solvent was removed in vacuo and the residue was purified by prep HPLC (Method 4). The solvent was removed in vacuo and the residue was partitioned between 10% NaHCO₃ solution (20 mL) and EtOAc (20 mL). The organic layer was separated, dried (Na₂SO₄) and the solvent removed in vacuo to afford 3-((4-(4-oxo-4,5,6,7-tetrahydro-1H-pyrazolo[4,3-c]pyridin-1-yl)pyridin-2-yl)methyl)-5-(trifluoromethyl)benzonitrile as a white solid (12 mg, 15%). Data in table 3.

### Procedure 11:

### Example 35, 1-(2-(3-fluoro-5-(trifluoromethyl)benzoyl)pyridin-4-yl)-1,5,6,7-tetrahydro-4H-pyrazolo[4,3-c]pyridin-4-one

### Example 36, 1-(2-((3-fluoro-5-(trifluoromethyl)phenyl)(hydroxy)methyl)pyridin-4-yl)-1,5,6,7-tetrahydro-4H-pyrazolo[4,3-c]pyridin-4-one

### Example 37, 1-(2-(fluoro(3-fluoro-5-(trifluoromethyl)phenyl)methyl)pyridin-4-yl)-1,5,6,7-tetrahydro-4H-pyrazolo[4,3-c]pyridin-4-one

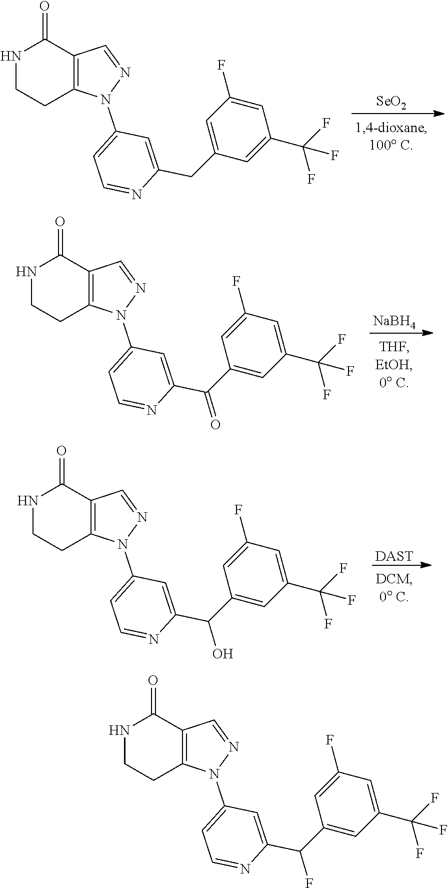

Step 1. Selenium dioxide (113 mg, 1.02 mmol) was added to a solution of 1-(2-(3-fluoro-5-(trifluoromethyl)benzyl)pyridin-4-yl)-1,5,6,7-tetrahydro-4H-pyrazolo[4,3-c]pyridin-4-one (Example 1, 600 mg, 1.53 mmol) in 1,4-dioxane (15 mL) and the reaction mixture was heated at 100° C. for 3 h. The solvent was removed in vacuo and the residue was purified by gradient flash column chromatography eluting with 0-100% EtOAc in pet-ether to afford 1-(2-(3-fluoro-5-(trifluoromethyl)benzoyl)pyridin-4-yl)-1,5,6,7-tetrahydro-4H-pyrazolo[4,3-c]pyridin-4-one as an off-white solid (Example 35, 400 mg, 64%). Data in table 3.

Step 2. Sodium borohydride (28 mg, 0.742 mmol) was added to a solution of 1-(2-(3-fluoro-5-(trifluoromethyl)benzoyl)pyridin-4-yl)-1,5,6,7-tetrahydro-4H-pyrazolo[4,3-c]pyridin-4-one (200 mg, 0.495 mmol) in EtOH:THF (1 mL:10 mL) at 0° C. and the reaction mixture was stirred at RT for 3 h. The reaction mixture was partitioned between EtOAc (20 mL) and water (20 mL). The organic layer was separated, dried (Na₂SO₄) and the solvent removed in vacuo. The residue was purified by gradient flash column chromatography eluting with 0-100% EtOAc in pet-ether to afford 1-(2-((3-fluoro-5-(trifluoromethyl)phenyl)(hydroxy)methyl)pyridin-4-yl)-1,5,6,7-tetrahydro-4H-pyrazolo[4,3-c]pyridin-4-one as a white solid (Example 36, 135 mg, 68%). Data in table 3.

Step 3. DAST (22 mg, 0.13 mmol) was added to a solution of 1-(2-((3-fluoro-5-(trifluoromethyl)phenyl)(hydroxy)methyl)pyridin-4-yl)-1,5,6,7-tetrahydro-4H-pyrazolo[4,3-c]pyridin-4-one (50 mg, 0.12 mmol) in DCM (10 mL) at 0° C. and the reaction mixture was stirred at RT for 16 h. The reaction mixture was partitioned between NaHCO₃ solution (10 mL) and DCM (10 mL). The organic layer was separated, dried (Na₂SO₄) and the solvent removed in vacuo. The residue was purified by gradient flash column chromatography eluting with 0-100% EtOAc in pet-ether to afford 1-(2-(fluoro(3-fluoro-5-(trifluoromethyl)phenyl)methyl)pyridin-4-yl)-1,5,6,7-tetrahydro-4H-pyrazolo[4,3-c]pyridin-4-one as a white solid (Example 37, 13 mg, 26%). Data in table 3.

### Procedure 12:

### Example 38, 1-(2-(difluoro(3-fluoro-5-(trifluoromethyl)phenyl)methyl)pyridin-4-yl)-1,5,6,7-tetrahydro-4H-pyrazolo[4,3-c]pyridin-4-one

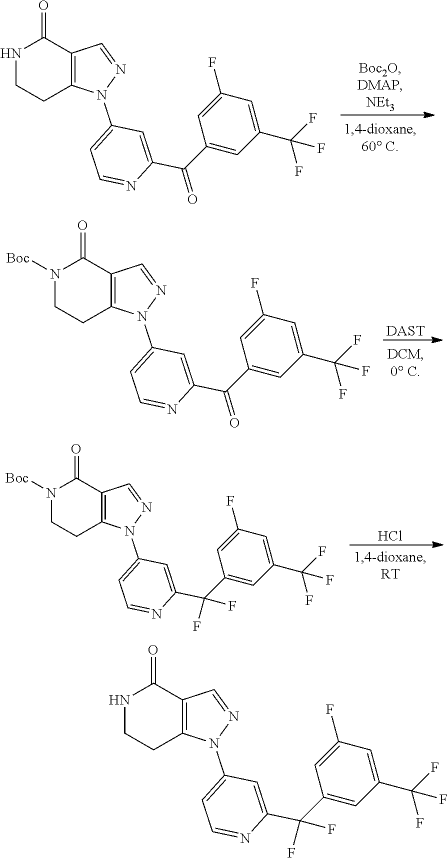

Step 1. NEt₃ (0.2 ml, 1.48 mmol), DMAP (12 mg, 0.09 mmol) and Boc anhydride (128 mg, 0.15 mmol) were added to a solution of 1-(2-(3-fluoro-5-(trifluoromethyl)benzoyl)pyridin-4-yl)-1,5,6,7-tetrahydro-4H-pyrazolo[4,3-c]pyridin-4-one (Example 35, 200 mg, 0.49 mmol) in 1,4-dioxane (10 mL) and the reaction mixture was heated at 60° C. for 16 h. The solvent was removed in vacuo and the residue was purified by gradient flash column chromatography eluting with 0-80% EtOAc in pet-ether to afford tert-butyl 1-(2-(3-fluoro-5-(trifluoromethyl)benzoyl)pyridin-4-yl)-4-oxo-1,4,6,7-tetrahydro-5H-pyrazolo[4,3-c]pyridine-5-carboxylate as an off-white solid (100 mg, 40%).

LCMS (Method 2): m/z 505.0 (ES+), at 2.89 min.

¹H NMR: (400 MHz, DMSO-d₆) δ: 8.94-8.93 (m, 1H), 8.32 (d, J=1.6 Hz, 2H), 8.21-8.21 (m, 2H), 8.10-8.00 (m, 1H), 8.00-7.98 (m, 1H), 4.06-4.03 (m, 2H), 3.42-3.39 (m, 2H), 1.49 (d, J=8.0 Hz, 9H).

Step 2. DAST (70 mg, 0.42 mmol) was added to a solution of tert-butyl 1-(2-(3-fluoro-5-(trifluoromethyl)benzoyl)pyridin-4-yl)-4-oxo-1,4,6,7-tetrahydro-5H-pyrazolo[4,3-c]pyridine-5-carboxylate (100 mg, 0.19 mmol) in DCM (10 mL) at 0° C. and the reaction mixture was stirred at RT for 16 h. The reaction mixture was partitioned between 10% NaHCO₃ solution (10 mL) and EtOAc (10 mL). The organic layer was separated, dried (Na₂SO₄) and the solvent removed in vacuo to afford tert-butyl 1-(2-(difluoro(3-fluoro-5-(trifluoromethyl)phenyl)methyl)pyridin-4-yl)-4-oxo-1,4,6,7-tetrahydro-5H-pyrazolo[4,3-c]pyridine-5-carboxylate as a yellow gum (70 mg, crude).

LCMS (Method 7): m/z 527.0 (ES+), at 2.56 min.

Step 3. tert-Butyl 1-(2-(difluoro(3-fluoro-5-(trifluoromethyl)phenyl)methyl)pyridin-4-yl)-4-oxo-1,4,6,7-tetrahydro-5H-pyrazolo[4,3-c]pyridine-5-carboxylate (70 mg) was dissolved in HCl solution (4 M in 1,4-dioxane, 5 mL) and the reaction mixture stirred at RT for 4 h. The reaction mixture was partitioned between 10% NaHCO₃ solution (20 mL) and EtOAc (20 mL). The organic layer was separated, dried (Na₂SO₄) the solvent removed in vacuo. The residue was purified by prep HPLC (Method 4), The solvent was removed in vacuo and the residue was partitioned between 10% NaHCO₃ solution (10 mL) and EtOAc (10 mL). The organic layer was separated, dried (Na₂SO₄) and the solvent removed in vacuo to afford 1-(2-(difluoro(3-fluoro-5-(trifluoromethyl)phenyl)methyl)pyridin-4-yl)-1,5,6,7-tetrahydro-4H-pyrazolo[4,3-c]pyridin-4-one as a white solid (6 mg, 10%). Data in table 3.

### Procedure 13:

### Example 39, 1-(2-((3-fluoro-5-(trifluoromethyl)phenyl)methyl-d₂)pyridin-4-yl)-1,5,6,7-tetrahydro-4H-pyrazolo[4,3-c]pyridin-4-one

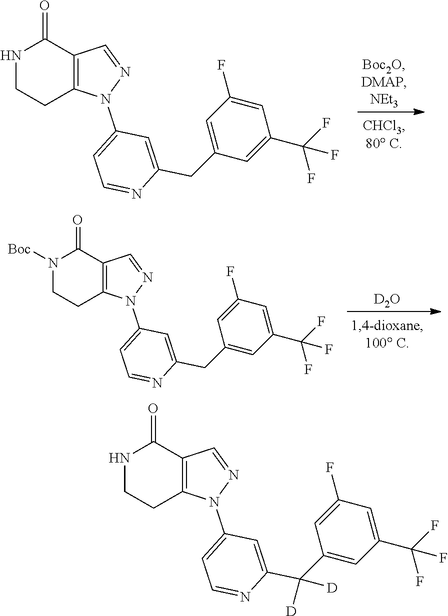

Step 1. NEt₃ (0.155 mg, 1.53 mmol), DMAP (12 mg, 0.102 mmol) and Boc anhydride (134 mg, 0.615 mmol) were added to a solution of 1-(2-(3-fluoro-5-(trifluoromethyl)benzyl)pyridin-4-yl)-1,5,6,7-tetrahydro-4H-pyrazolo[4,3-c]pyridin-4-one (Example 1, 200 mg, 0.512 mmol) in CHCl₃ (10 mL) and the reaction mixture was heated at 80° C. for 16 h. The solvent was removed in vacuo and the residue was purified by gradient flash column chromatography eluting with 0-50% EtOAc in pet-ether to afford tert-butyl 1-(2-(3-fluoro-5-(trifluoromethyl)benzyl)pyridin-4-yl)-4-oxo-1,4,6,7-tetrahydro-5H-pyrazolo[4,3-c]pyridine-5-carboxylate as an off-white solid (120 mg, 47%).

LCMS (Method 7): m/z 491.0 (ES+), at 2.46 min.

¹H NMR: (400 MHz, DMSO-d₆) δ: 8.67 (d, J=7.2 Hz, 1H), 8.21 (s, 1H), 7.71 (s, 1H), 7.61 (s, 1H), 7.56-7.51 (m, 3H), 4.32 (s, 2H), 3.99 (t, J=8.0 Hz, 2H), 3.20 (t, J=6.8 Hz, 2H), 1.48 (s, 9H).

Step 2. D₂O (5 mL) was added to a solution of tert-butyl 1-(2-(3-fluoro-5-(trifluoromethyl)benzyl)pyridin-4-yl)-4-oxo-1,4,6,7-tetrahydro-5H-pyrazolo[4,3-c]pyridine-5-carboxylate (100 mg, 0.20 mmol) in 1,4-dioxane (5 mL) and the reaction mixture was heated at 100° C. for 24 h. After 24 h, the solvent was removed in vacuo, the residue dissolved in D₂O (10 mL) and the reaction mixture heated at 100° C. for 24 h. This process was repeated for another 3 times. (Reaction carried out for 5 days). The solvent was removed in vacuo and the residue was purified by gradient flash column chromatography eluting with 0-10% MeOH in DCM to afford 1-(2-((3-fluoro-5-(trifluoromethyl)phenyl)methyl-d₂)pyridin-4-yl)-1,5,6,7-tetrahydro-4H-pyrazolo[4,3-c]pyridin-4-one as a white solid (20 mg, 24%). Data in table 3.

### Procedure 14:

### Example 41, 1-(4-(3-(trifluoromethyl)benzyl)pyridin-2-yl)-5,6,7,8-tetrahydro-[1,2,3]triazolo[4,5-c]azepin-4(1H)-one

### Example 45, 2-(4-(3-(trifluoromethyl)benzyl)pyridin-2-yl)-5,6,7,8-tetrahydro-[1,2,3]triazolo[4,5-c]azepin-4(2H)-one

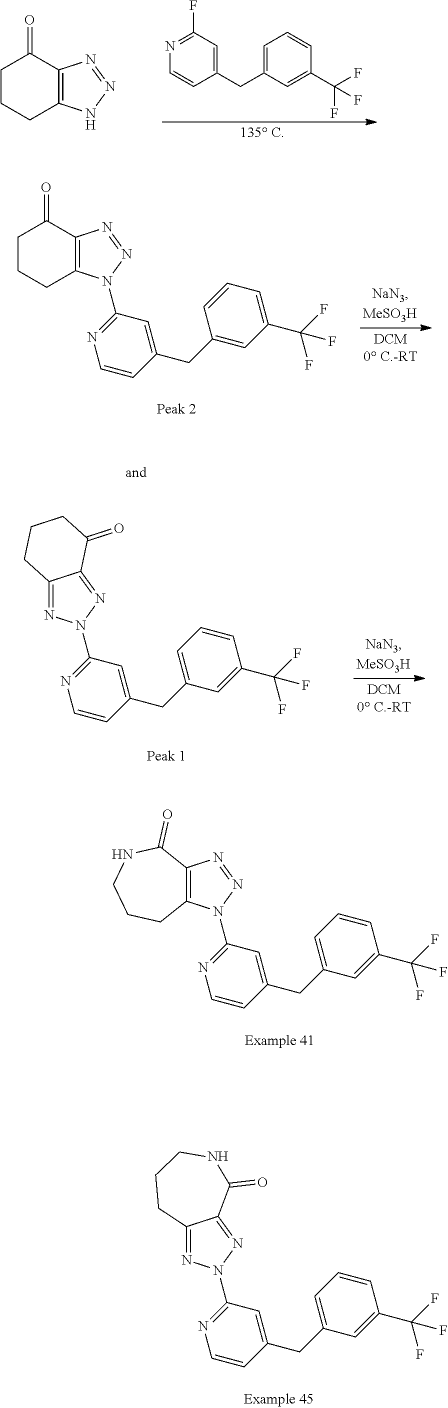 [SMILES: O=C1CCCc2[nH]nnc21 ; Fc1cc(Cc2cccc(C(F)(F)F)c2)ccn1 ; O=C1CCCc2c1nnn2-c1cc(Cc2cccc(C(F)(F)F)c2)ccn1 ; O=C1CCCc2nn(-c3cc(Cc4cccc(C(F)(F)F)c4)ccn3)nc21 ; O=C1NCCCc2c1nnn2-c1cc(Cc2cccc(C(F)(F)F)c2)ccn1 ; O=C1NCCCc2nn(-c3cc(Cc4cccc(C(F)(F)F)c4)ccn3)nc21]

Step 1. 2-Fluoro-4-(3-(trifluoromethyl)benzyl)pyridine (Intermediate 4, 465 mg, 1.82 mmol) was added to 1,5,6,7-tetrahydro-4H-benzo[d][1,2,3]triazol-4-one (Intermediate 15, 250 mg, 1.82 mmol) and the reaction mixture heated at 135° C. for 16 h. The reaction mixture was dissolved in 5% MeOH/DCM (10 mL) and the solvent removed in vacuo. The residue was purified by gradient flash column chromatography eluting with 35-40% EtOAc in hexane to afford 2-(4-(3-(trifluoromethyl)benzyl)pyridin-2-yl)-2,5,6,7-tetrahydro-4H-benzo[d][1,2,3]triazol-4-one as a white solid (Peak 1, 24 mg, 4%) and 1-(4-(3-(trifluoromethyl)benzyl)pyridin-2-yl)-1,5,6,7-tetrahydro-4H-benzo[d][1,2,3]triazol-4-one as a white solid (Peak 2, 30 mg, 4%).

### Peak 1:

MS (Method 1): m/z 373 (ES+).

¹H NMR: (400 MHz, DMSO-d₆) δ: 8.54 (d, J=5.0 Hz, 1H), 8.05 (s, 1H), 7.79 (s, 1H), 7.68 (d, J=7.1 Hz, 1H), 7.63-7.54 (m, 2H), 7.52 (d, J=4.9 Hz, 1H), 4.26 (s, 2H), 3.01 (t, J=6.1 Hz, 2H), 2.66 (t, J=6.2 Hz, 2H), 2.23-2.12 (m, 2H).

### Peak 2:

MS (Method 1): m/z 373 (ES+).

¹H NMR: (400 MHz, DMSO-d₆) δ: 8.51 (d, J=5.1 Hz, 1H), 8.03 (s, 1H), 7.76 (s, 1H), 7.66 (d, J=7.2 Hz, 1H), 7.63-7.56 (m, 2H), 7.49 (d, J=4.8 Hz, 1H), 4.27 (s, 2H), 3.32 (t, J=6.1 Hz, 2H), 2.57 (t, J=6.3 Hz, 2H), 2.15-2.09 (m, 2H).

Step 2 (Peak 2). NaN₃ (62 mg, 0.96 mmol) was slowly added to a solution of 1-(4-(3-(trifluoromethyl)benzyl)pyridin-2-yl)-1,5,6,7-tetrahydro-4H-benzo[d][1,2,3]triazol-4-one (Step 1, Peak 2, 120 mg, 0.32 mmol) in DCM:MeSO₃H (6 mL, 2:1), at 0° C. and the reaction mixture was stirred at RT for 2 h. The reaction mixture was quenched with 2 N aq NaOH solution and the aqueous layer was extracted with EtOAc (3×30 mL). The organic layers were combined, dried (Na₂SO₄) and the solvent removed in vacuo. The residue was triturated with MTBE to afford 1-(4-(3-(trifluoromethyl)benzyl)pyridin-2-yl)-5,6,7,8-tetrahydro-[1,2,3]triazolo[4,5-c]azepin-4(1H)-one) as a white solid (Example 41, 90 mg, 72%). Data in table 3.

Step 2 (Peak 1). NaN₃ (104 mg, 1.61 mmol) was slowly added to a solution of 2-(4-(3-(trifluoromethyl)benzyl)pyridin-2-yl)-2,5,6,7-tetrahydro-4H-benzo[d][1,2,3]triazol-4-one (Step 1, Peak 1, 200 mg, 0.53 mmol) in DCM:MeSO₃H (9 mL, 2:1) at 0° C. and the reaction mixture was stirred at RT for 2 h. The reaction mixture was quenched with 2 N aq NaOH solution and the aqueous layer was extracted with EtOAc (3×30 mL). The organic layers were combined, dried (Na₂SO₄) and the solvent removed in vacuo. The residue was triturated with MTBE to afford 2-(4-(3-(trifluoromethyl)benzyl)pyridin-2-yl)-5,6,7,8-tetrahydro-[1,2,3]triazolo[4,5-c]azepin-4(2H)-one as a white solid (Example 45, 75 mg, 36%). Data in table 3.

### Procedure 15:

### Example 42, 1-(4-(3-(trifluoromethyl)benzyl)pyridin-2-yl)-1,5,6,7-tetrahydro-4H-[1,2,3]triazolo[4,5-c]pyridin-4-one

### Example 43, 2-(4-(3-(trifluoromethyl)benzyl)pyridin-2-yl)-2,5,6,7-tetrahydro-4H-[1,2,3]triazolo[4,5-c]pyridin-4-one

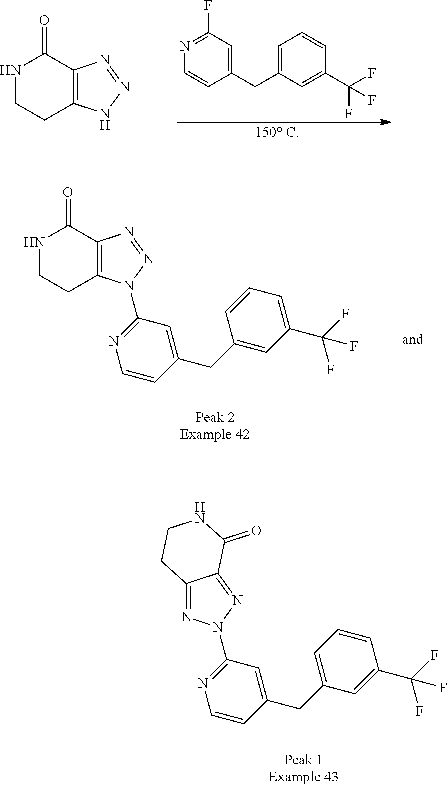 [SMILES: O=C1NCCc2[nH]nnc21 ; Fc1cc(Cc2cccc(C(F)(F)F)c2)ccn1 ; O=C1NCCc2c1nnn2-c1cc(Cc2cccc(C(F)(F)F)c2)ccn1 ; O=C1NCCc2nn(-c3cc(Cc4cccc(C(F)(F)F)c4)ccn3)nc21]

2-Fluoro-4-(3-(trifluoromethyl)benzyl)pyridine (Intermediate 4, 350 mg, 1.36 mmol) was added to 1,5,6,7-tetrahydro-4H-[1,2,3]triazolo[4,5-c]pyridin-4-one (Intermediate 16, 190 mg, 1.36 mmol) and the reaction mixture heated at 150° C. for 16 h. The reaction mixture was triturated with hexane (10 mL). The residue was purified by prep-HPLC (Method 5—35-50% gradient) to afford 1-(4-(3-(trifluoromethyl)benzyl)pyridin-2-yl)-1,5,6,7-tetrahydro-4H-[1,2,3]triazolo[4,5-c]pyridin-4-one as a white solid (Example 42, Peak 2, 14 mg, 3%) and 2-(4-(3-(trifluoromethyl)benzyl)pyridin-2-yl)-2,5,6,7-tetrahydro-4H-[1,2,3]triazolo[4,5-c]pyridin-4-one as a white solid (Example 43, Peak 1, 42 mg, 8%). Data in table 3.

### Procedure 16:

### Example 46, 1-(4-(3-fluoro-5-(trifluoromethyl)benzyl)pyridin-2-yl)-5,6,7,8-tetrahydro-[1,2,3]triazolo[4,5-c]azepin-4(1H)-one

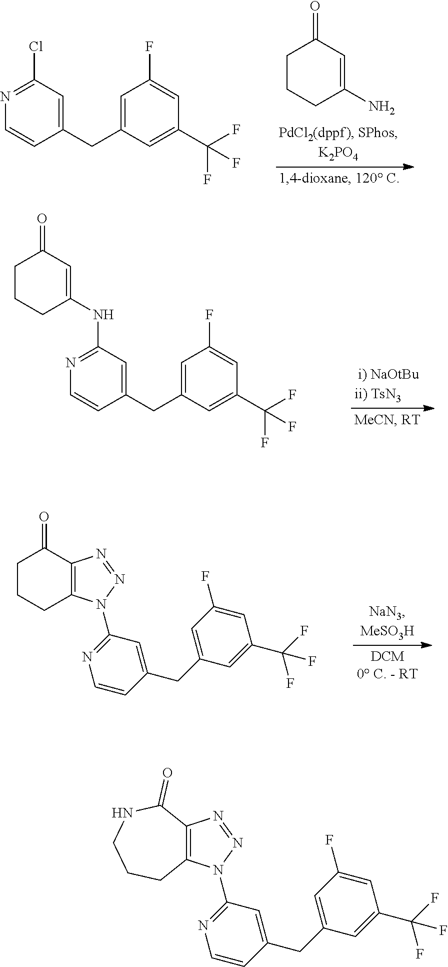 [SMILES: Fc1cc(Cc2ccnc(Cl)c2)cc(C(F)(F)F)c1 ; NC1=CC(=O)CCC1 ; O=C1C=C(Nc2cc(Cc3cc(F)cc(C(F)(F)F)c3)ccn2)CCC1 ; O=C1CCCc2c1nnn2-c1cc(Cc2cc(F)cc(C(F)(F)F)c2)ccn1 ; O=C1NCCCc2c1nnn2-c1cc(Cc2cc(F)cc(C(F)(F)F)c2)ccn1]

Step 1. 2-Chloro-4-(3-fluoro-5-(trifluoromethyl)benzyl)pyridine (Intermediate 18, 2.61 g, 9.00 mmol) was added to a solution of 3-aminocyclohex-2-en-1-one (1.00 g, 9.00 mmol) in 1,4-dioxane (10 mL). The reaction mixture was degassed with argon for 10 min and PdCl₂(dppf).DCM (367 mg, 0.45 mmol), SPhos (369 mg, 0.90 mmol) and K₃PO₄ (5.73 g, 27.0 mmol) were sequentially added to the reaction mixture. The reaction mixture was heated at 120° C. for 16 h and then filtered through Celite and washed with EtOAc (300 mL). The solvent was removed in vacuo. The residue was purified by gradient flash column chromatography eluting with 3-5% MeOH in DCM to afford 3-((4-(3-fluoro-5-(trifluoromethyl)benzyl)pyridin-2-yl)amino)cyclohex-2-en-1-one as a brown solid (1.50 g, 43%).

LCMS (Method 9): m/z 365.0 (ES+), at 2.33 min.

Step 2. A solution of 3-((4-(3-fluoro-5-(trifluoromethyl)benzyl)pyridin-2-yl)amino)cyclohex-2-en-1-one (1.5 g, 4.11 mmol) in MeCN (45 mL) was added dropwise to a suspension of sodium tert-butoxide (600 mg, 6.20 mmol) in MeCN (45 mL). After 30 min stirring at RT, a solution of tosyl azide (1.05 g, 6.50 mmol) in MeCN (10 mL) was added dropwise. The reaction mixture was stirred for 2 min at RT and water (60 mL) was added. The aqueous layer was extracted with EtOAc (3×50 mL) and the combined organic layers were dried (Na₂SO₄) and the solvent removed in vacuo. The residue was triturated with Et₂O (30 mL) to afford 1-(4-(3-fluoro-5-(trifluoromethyl)benzyl)pyridin-2-yl)-1,5,6,7-tetrahydro-4H-benzo[d][1,2,3]triazol-4-one as an off white solid (1.0 g, 63%).

LCMS (Method 9): m/z 391.0 (ES+), at 2.33 min.

¹H NMR: (400 MHz, DMSO-d₆) δ: 8.55 (d, J=5.0 Hz, 1H), 8.10 (t, J=4.8 Hz, 1H), 7.75 (s, 1H), 7.65-7.60 (m, 1H), 7.59-7.54 (m, 2H), 4.28 (s, 2H), 2.60-2.51 (m, 4H), 2.20-2.10 (m, 2H).

Step 3. 1-(4-(3-fluoro-5-(trifluoromethyl)benzyl)pyridin-2-yl)-5,6,7,8-tetrahydro-[1,2,3]triazolo[4,5-c]azepin-4(1H)-one (35 mg, 32%) was prepared from 1-(4-(3-fluoro-5-(trifluoromethyl)benzyl)pyridin-2-yl)-1,5,6,7-tetrahydro-4H-benzo[d][1,2,3]triazol-4-one (100 mg, 0.26 mmol) and NaN₃ (50 mg, 0.78 mmol) using the methods of procedure 14, step 2. The residue was purified by gradient flash column chromatography eluting with 20-30% EtOAc in hexane. Data in table 3.

### Procedure 17:

### Example 47, 1-(4-(3-fluoro-5-(trifluoromethyl)benzyl)pyridin-2-yl)-1,7-dihydropyrazolo[4,3-d][1,2]oxazin-4(5H)-one

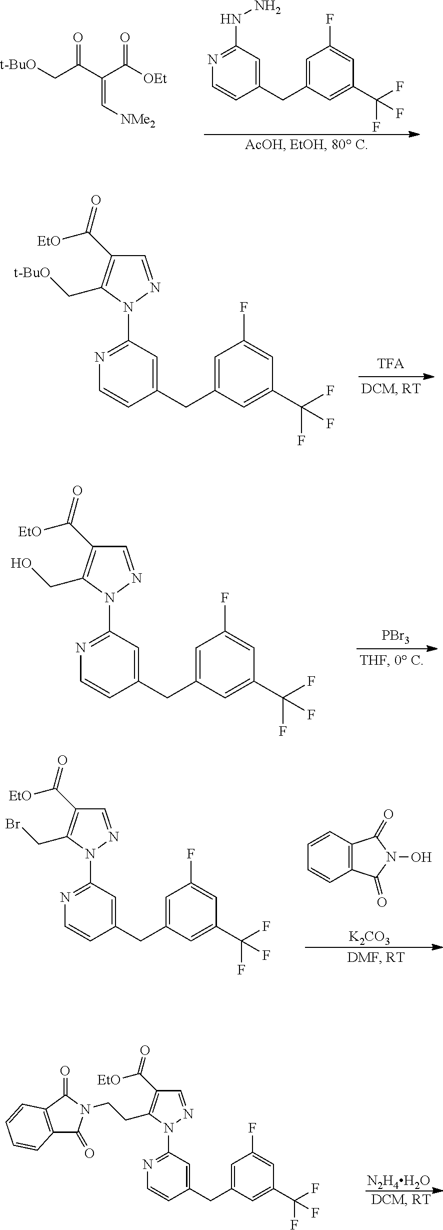

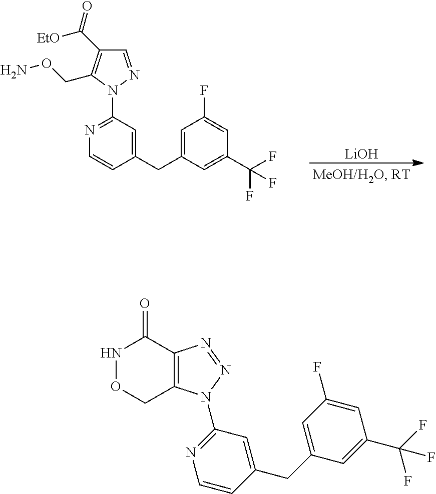 [SMILES: CCOC(=O)c1cnn(-c2cc(Cc3cc(F)cc(C(F)(F)F)c3)ccn2)c1CON ; "LiOH" ; O=C1NOCc2c1nnn2-c1cc(Cc2cc(F)cc(C(F)(F)F)c2)ccn1]

Step 1. A solution of ethyl (Z)-4-(tert-butoxy)-2-((dimethylamino)methylene)-3-oxobutanoate (Intermediate 19, 500 mg, 1.94 mmol) and 4-(3-fluoro-5-(trifluoromethyl)benzyl)-2-hydrazineylpyridine (Intermediate 8, 554 mg, 1.94 mmol) in EtOH (10 mL) and acetic acid (0.5 mL) was heated at 80° C. for 16 h. The reaction mixture was concentrated in vacuo and the residue was purified by gradient flash column chromatography eluting with 15-16% EtOAc in hexane to afford ethyl 5-(tert-butoxymethyl)-1-(4-(3-fluoro-5-(trifluoromethyl)benzyl)pyridin-2-yl)-1H-pyrazole-4-carboxylate as a light yellow solid (1.30 g, 71%).

LCMS (Method 9): m/z 478.4 (ES+), at 2.54 min.

Step 2. TFA (10 mL) was added to a solution of ethyl 5-(tert-butoxymethyl)-1-(4-(3-fluoro-5-(trifluoromethyl)benzyl)pyridin-2-yl)-1H-pyrazole-4-carboxylate (500 mg, 1.04 mmol) in DCM (10 mL) at 0° C. and the reaction mixture was stirred at RT for 24 h. The reaction mixture was concentrated in vacuo to afford ethyl 1-(4-(3-fluoro-5-(trifluoromethyl)benzyl)pyridin-2-yl)-5-(hydroxymethyl)-1H-pyrazole-4-carboxylate as a brown semi-solid (400 mg, crude). The crude product was used in the next step without further purification.

LCMS (Method 9): m/z 424.0 (ES+), at 2.39 min.

Step 3. Tribromophosphane (0.27 mL, 2.83 mmol) was added to a solution of ethyl 1-(4-(3-fluoro-5-(trifluoromethyl)benzyl)pyridin-2-yl)-5-(hydroxymethyl)-1H-pyrazole-4-carboxylate (400 mg, 0.945 mmol) in THF (15 mL) and the reaction mixture was stirred at 0° C. for 3 h. The reaction was quenched with sat. NaHCO₃ solution (5 mL) and the aqueous layer was extracted with EtOAc (3×50 mL). The organic layer was washed with brine, dried (Na₂SO₄) and the solvent removed in vacuo to afford ethyl 5-(bromomethyl)-1-(4-(3-fluoro-5-(trifluoromethyl)benzyl)pyridin-2-yl)-1H-pyrazole-4-carboxylate as a liquid (450 mg, crude). The crude product was used in the next step without further purification.

Step 4. Ethyl 5-(bromomethyl)-1-(4-(3-fluoro-5-(trifluoromethyl)benzyl)pyridin-2-yl)-1H-pyrazole-4-carboxylate (450 mg, 0.93 mmol) was added to a solution of 2-hydroxy-2,3-dihydro-1H-isoindole-1,3-dione (302 mg, 1.85 mmol) and K₂CO₃ (389 mg, 2.78 mmol) in DMF (10 mL) and the reaction mixture was stirred at RT for 16 h. The reaction was quenched with water (10 mL) and the aqueous layer extracted with EtOAc (3×30 mL). The combined organic layers were concentrated in vacuo. The residue was purified by gradient flash column chromatography eluting with 30-35% EtOAc in hexane to afford ethyl 5-(((1,3-dioxoisoindolin-2-yl)oxy)methyl)-1-(4-(3-fluoro-5-(trifluoromethyl)benzyl)pyridin-2-yl)-1H-pyrazole-4-carboxylate as a solid (180 mg, 34%).

LCMS (Method 9): m/z 569.0 (ES+), at 2.44 min.

Step 5. Hydrazine hydrate (220 mg, 4.40 mmol) was added to a solution of ethyl 5-(((1,3-dioxoisoindolin-2-yl)oxy)methyl)-1-(4-(3-fluoro-5-(trifluoromethyl)benzyl)pyridin-2-yl)-1H-pyrazole-4-carboxylate (500 mg, 0.88 mmol) in DCM (10 mL) and the reaction mixture stirred at RT for 3 h. The reaction was filtered and the filtrate was concentrated in vacuo to afford ethyl 5-((aminooxy)methyl)-1-(4-(3-fluoro-5-(trifluoromethyl)benzyl)pyridin-2-yl)-1H-pyrazole-4-carboxylate as semi-solid (280 mg, crude). The crude product was used in the next step without further purification.

LCMS (Method 9): m/z 437.4 (ES+), at 2.27 min.

Step 6. LiOH.H₂O (77 mg, 1.83 mmol) and water (0.2 mL) were added to a solution of ethyl 5-((aminooxy)methyl)-1-(4-(3-fluoro-5-(trifluoromethyl)benzyl)pyridin-2-yl)-1H-pyrazole-4-carboxylate (250 mg, 0.61 mmol) in MeOH (5 mL) and the reaction mixture was stirred at RT for 16 h. The solvent was removed in vacuo and the residue was dissolved in EtOAc (10 mL). This solution was washed with brine (5 mL), dried (Na₂SO₄) and the solvent removed in vacuo. The residue was purified by gradient flash column chromatography eluting with 30-35% EtOAc in hexane to afford 1-(4-(3-fluoro-5-(trifluoromethyl)benzyl)pyridin-2-yl)-1,7-dihydropyrazolo[4,3-d][1,2]oxazin-4(5H)-one as a white solid (25 mg, 10%). Data in table 3.

### Procedure 18:

### Example 48, 1-(2-(3-fluoro-5-(trifluoromethyl)benzyl)pyridin-4-yl)-5,6-dihydropyrrolo[3,4-c]pyrazol-4(1H)-one

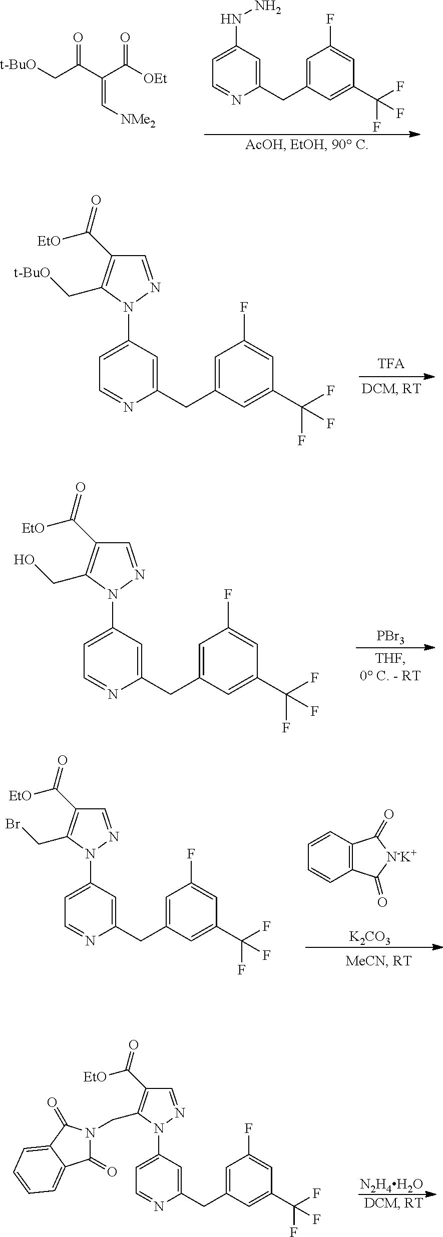

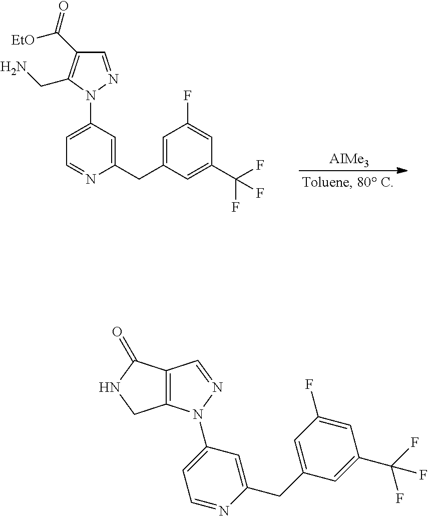 [SMILES: CCOC(=O)c1cnn(-c2ccnc(Cc3cc(F)cc(C(F)(F)F)c3)c2)c1CN ; O=C1NCc2c1cnn2-c1ccnc(Cc2cc(F)cc(C(F)(F)F)c2)c1 ; "AIMe3"]

Steps 1, 2 and 3. Ethyl 5-(bromomethyl)-1-(2-(3-fluoro-5-(trifluoromethyl)benzyl)pyridin-4-yl)-1H-pyrazole-4-carboxylate (165 mg, crude) was prepared from ethyl (Z)-4-(tert-butoxy)-2-((dimethylamino)methylene)-3-oxobutanoate (Intermediate 19, 433 mg, 0.56 mmol) and 2-(3-fluoro-5-(trifluoromethyl)benzyl)-4-hydrazineylpyridine (Intermediate 6, 160 mg, 0.56 mmol) using the methods of Procedure 17, steps 1, 2 and 3. The crude product was used in the next step without further purification.

LCMS (Method 9): m/z 486.2 (ES+), at 2.46 min.

Step 4. Potassium 1,3-dioxo-2,3-dihydro-1H-isoindol-2-ide (63 mg, 0.34 mmol) was added to a solution of ethyl 5-(bromomethyl)-1-(2-(3-fluoro-5-(trifluoromethyl)benzyl)pyridin-4-yl)-1H-pyrazole-4-carboxylate (160 mg, 0.34 mmol) in MeCN (4 mL) and the reaction mixture was stirred at RT for 16 h. The reaction was quenched with water (10 mL) and extracted with EtOAc (3×30 mL). The combined organic layers were concentrated in vacuo to afford ethyl 5-((1,3-dioxoisoindolin-2-yl)methyl)-1-(2-(3-fluoro-5-(trifluoromethyl)benzyl)pyridin-4-yl)-1H-pyrazole-4-carboxylate as a white semi-solid (241 mg, crude). The crude product was used in the next step without further purification.

LCMS (Method 9): m/z 552.9 (ES+), at 2.46 min.

Step 5. Ethyl 5-(aminomethyl)-1-(2-(3-fluoro-5-(trifluoromethyl)benzyl)pyridin-4-yl)-1H-pyrazole-4-carboxylate (110 mg, crude) was prepared from ethyl 5-((1,3-dioxoisoindolin-2-yl)methyl)-1-(2-(3-fluoro-5-(trifluoromethyl)benzyl)pyridin-4-yl)-1H-pyrazole-4-carboxylate (241 mg, 0.44 mmol) and hydrazine hydrate (0.06 mL, 1.31 mmol) according to the methods of Procedure 17, step 5. The crude product was used in the next step without further purification.

LCMS (Method 9): m/z 423.0 (ES+), at 2.24 min.

Step 6. Trimethylaluminium (0.18 mL, 0.36 mmol) was added to a solution of ethyl 5-(aminomethyl)-1-(2-(3-fluoro-5-(trifluoromethyl)benzyl)pyridin-4-yl)-1H-pyrazole-4-carboxylate 100 mg, 0.24 mmol) in toluene (4 mL) at 0° C. and the reaction mixture was heated at 80° C. for 2 h. The reaction mixture was quenched with water (2 mL), filtered through a pad of Celite and washed with DCM (2×20 mL). The organic layers were combined, dried (Na₂SO₄) and the solvent removed in vacuo. The residue was washed with Et₂O (10 mL) and dried in vacuo to afford 1-(2-(3-fluoro-5-(trifluoromethyl)benzyl)pyridin-4-yl)-5,6-dihydropyrrolo[3,4-c]pyrazol-4(1H)-one as a white solid (38 mg, 43%). Data in table 3.

### Procedure 19:

### Example 49, 1-(2-((3-fluoro-5-(trifluoromethyl)phenyl)methyl-d)pyridin-4-yl)-1,5,6,7-tetrahydro-4H-pyrazolo[4,3-c]pyridin-4-one

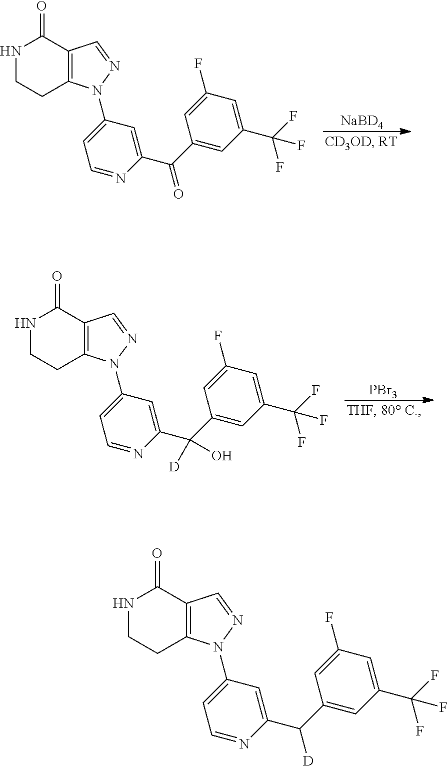

Step 1. NaBD₄ (16 mg, 0.37 mmol) was added to a solution of 1-(2-(3-fluoro-5-(trifluoromethyl)benzoyl)pyridin-4-yl)-1,5,6,7-tetrahydro-4H-pyrazolo[4,3-c]pyridin-4-one (Example 35, 100 mg, 0.24 mmol) in CD₃OD (10 mL) and the reaction mixture was stirred at RT for 1 h. The reaction mixture was partitioned between sat. NH₄Cl solution (20 mL) and EtOAc (20 mL). The organic layer was separated, dried (Na₂SO₄) and the solvent removed in vacuo. The residue was triturated with pet-ether (5 mL) and dried in vacuo to afford 1-(2-((3-fluoro-5-(trifluoromethyl)phenyl)(hydroxy)methyl-d)pyridin-4-yl)-1,5,6,7-tetrahydro-4H-pyrazolo[4,3-c]pyridin-4-one as a yellow solid (80 mg, 79%).

LCMS (Method 7): m/z 408.2 (ES+), at 1.80 min.

¹H NMR: (400 MHz, DMSO-d₆) δ: 8.64 (d, J=5.6 Hz, 1H), 8.07 (s, 1H), 7.93 (d, J=2.0 Hz, 1H), 7.71 (s, 1H), 7.63-7.51 (m, 4H), 6.63 (s, 1H), 3.46-3.43 (m, 2H), 3.33-3.27 (m, 2H).

Step 2. PBr₃ (322 mg, 1.19 mmol) was added dropwise to a solution of 1-(2-((3-fluoro-5-(trifluoromethyl)phenyl)(hydroxy)methyl-d)pyridin-4-yl)-1,5,6,7-tetrahydro-4H-pyrazolo[4,3-c]pyridin-4-one (80 mg, 0.19 mmol) in THF (10 mL) and the reaction mixture was heated at 80° C. for 4 h. The reaction mixture was partitioned between 10% NaHCO₃ solution (20 mL) and EtOAc (20 mL). The organic layer was separated, dried (Na₂SO₄) and the solvent removed in vacuo. The residue was purified by prep HPLC (Method 4). The solvent was removed in vacuo and the residue was partitioned between 10% NaHCO₃ solution (10 mL) and EtOAc (10 mL). Organic layer was separated, dried (Na₂SO₄) and the solvent removed in vacuo to afford 1-(2-((3-fluoro-5-(trifluoromethyl)phenyl)methyl-d)pyridin-4-yl)-1,5,6,7-tetrahydro-4H-pyrazolo[4,3-c]pyridin-4-one as a white solid (8 mg, 10%). Data in table 3.

### Procedure 20:

### Example 105, 1-(2-(3-fluoro-5-(trifluoromethyl)benzyl)pyridin-4-yl)-5-(2-hydroxyethyl)-1,5,6,7-tetrahydro-4H-pyrazolo[4,3-c]pyridin-4-one

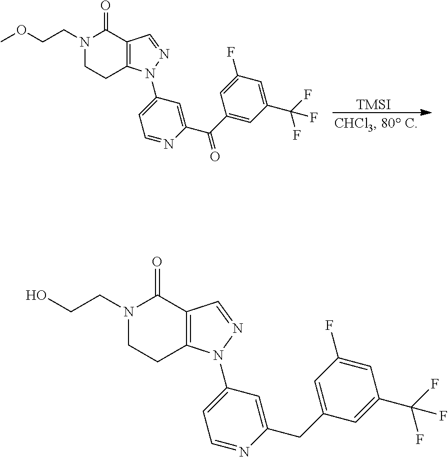 [SMILES: "TMSI" ; COCCN1CCc2c(cnn2-c2ccnc(C(=O)c3cc(F)cc(C(F)(F)F)c3)c2)C1=O ; O=C1c2cnn(-c3ccnc(Cc4cc(F)cc(C(F)(F)F)c4)c3)c2CCN1CCO]

Iodotrimethylsilane (223 mg, 1.12 mmol) was added to a solution of 1-(2-(3-fluoro-5-(trifluoromethyl)benzyl)pyridin-4-yl)-5-(2-methoxyethyl)-1,5,6,7-tetrahydro-4H-pyrazolo[4,3-c]pyridin-4-one (Example 104, 100 mg, 0.223 mmol) in chloroform (10 mL) at RT and the reaction mixture was heated at 80° C. for 4 h. The reaction mixture was partitioned between water (20 mL) and DCM (20 mL). The aqueous layer was extracted with DCM (20 mL) and the combined organic layers were washed with water (10 mL) and brine solution (10 mL), dried over anhydrous (Na₂SO₄) and the solvent removed in vacuo. The residue was purified by gradient flash column chromatography eluting with 0-10% MeOH in DCM to afford 1-(2-(3-fluoro-5-(trifluoromethyl)benzyl)pyridin-4-yl)-5-(2-hydroxyethyl)-1,5,6,7-tetrahydro-4H-pyrazolo[4,3-c]pyridin-4-one as a white gum (24 mg, 24%). Data in table 3.

### Procedure 21:

### Example 107, 1-(4-(3-fluoro-5-(trifluoromethyl)benzyl)pyridin-2-yl)-5-(2-hydroxyethyl)-1,5,6,7-tetrahydro-4H-pyrazolo[4,3-c]pyridin-4-one

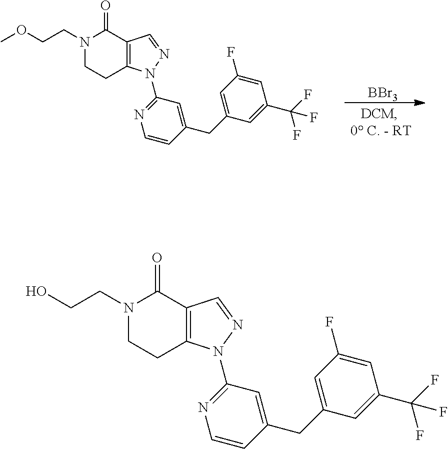 [SMILES: COCCN1CCc2c(cnn2-c2cc(Cc3cc(F)cc(C(F)(F)F)c3)ccn2)C1=O ; "BBr3" ; O=C1c2cnn(-c3cc(Cc4cc(F)cc(C(F)(F)F)c4)ccn3)c2CCN1CCO]

A solution of BBr₃ (1.0 M in DCM, 0.75 mL, 0.75 mmol) was added dropwise to a solution of 1-(4-(3-fluoro-5-(trifluoromethyl)benzyl)pyridin-2-yl)-5-(2-methoxyethyl)-1,5,6,7-tetrahydro-4H-pyrazolo[4,3-c]pyridin-4-one (Example 106, 65 mg, 0.15 mmol) in DCM (2 mL) at 0° C. The reaction mixture was stirred at RT for 4 h. The reaction mixture was diluted with water and the aqueous layer was extracted with DCM (2×20 mL). The combined organic layers were dried (Na₂SO₄) and the solvent removed in vacuo. The residue was purified by prep HPLC (Method 6—gradient 40-75%) to afford 1-(4-(3-fluoro-5-(trifluoromethyl)benzyl)pyridin-2-yl)-5-(2-hydroxyethyl)-1,5,6,7-tetrahydro-4H-pyrazolo[4,3-c]pyridin-4-one as white solid (6 mg, 8%).

### Procedure 22:

### Example 109, 1-(3-(3-fluoro-5-(trifluoromethyl)benzyl)phenyl)-1,5,6,7-tetrahydro-4H-pyrazolo[4,3-c]pyridin-4-one

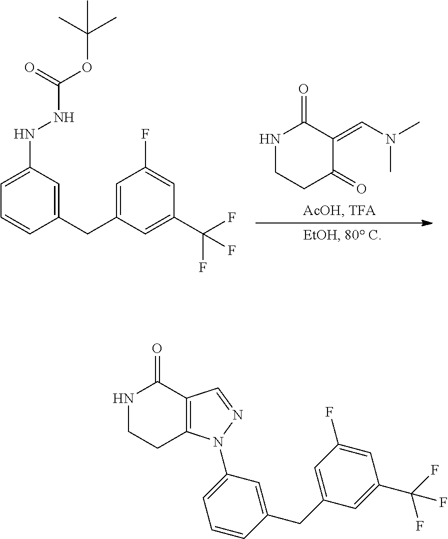 [SMILES: CC(C)(C)OC(=O)NNc1cccc(Cc2cc(F)cc(C(F)(F)F)c2)c1 ; CN(C)/C=C1\C(=O)CCNC1=O ; O=C1NCCc2c1cnn2-c1cccc(Cc2cc(F)cc(C(F)(F)F)c2)c1]

tert-Butyl 2-(3-(3-fluoro-5-(trifluoromethyl)benzyl)phenyl)hydrazine-1-carboxylate (181 mg, 0.47 mmol) and (E)-3-((dimethylamino)methylene)piperidine-2,4-dione (Intermediate 1, 95 mg, 0.57 mmol) were dissolved in ethanol (5 mL) and acetic acid (0.03 mL, 0.57 mmol) was added. The reaction mixture was heated at 80° C. for 3 hours. TFA (0.06 mL) was added and the reaction mixture heated at 80° C. for 2 hours. Further TFA (0.74 mL) was added and the reaction mixture heated at 80° C. overnight. The solvent was removed in vacuo and the residue partitioned between sat. NaHCO₃ soln (5 mL) and EtOAc (5 mL). The organic layer was removed and the aqueous layer extracted with EtOAc (2×5 mL), the combined organic layers dried (phase separator) and the solvent removed in vacuo. The residue was purified by gradient flash chromatography eluting with 2-7% MeOH in DCM. The residue was further purified via prep HPLC (Method 2—40-70% gradient) to give 1-(3-(3-fluoro-5-(trifluoromethyl)benzyl)phenyl)-1,5,6,7-tetrahydro-4H-pyrazolo[4,3-c]pyridin-4-one as a white solid (35 mg, 19%). Data in table 3.

### Procedure 23:

### Example 111, 1-(2-((6-(difluoromethoxy)-5-fluoropyridin-3-yl)methyl)pyridin-4-yl)-3-methyl-1,5,6,7-tetrahydro-4H-pyrazolo[4,3-c]pyridin-4-one

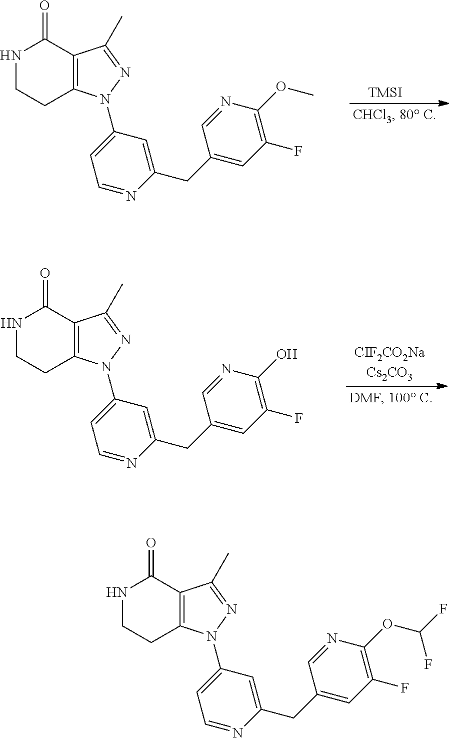 [SMILES: "TMSI" ; COc1ncc(Cc2cc(-n3nc(C)c4c3CCNC4=O)ccn2)cc1F ; Cc1nn(-c2ccnc(Cc3cnc(O)c(F)c3)c2)c2c1C(=O)NCC2 ; Cc1nn(-c2ccnc(Cc3cnc(OC(F)F)c(F)c3)c2)c2c1C(=O)NCC2]

Step 1. 1-(2-((5-fluoro-6-hydroxypyridin-3-yl)methyl)pyridin-4-yl)-3-methyl-1,5,6,7-tetrahydro-4H-pyrazolo[4,3-c]pyridin-4-one (100 mg, 55%) was prepared from 1-(2-((5-fluoro-6-methoxypyridin-3-yl)methyl)pyridin-4-yl)-3-methyl-1,5,6,7-tetrahydro-4H-pyrazolo[4,3-c]pyridin-4-one (Example 93, 0.19 g, 0.52 mmol) using the methods of Procedure 20.

LCMS (Method 11): m/z 354.1 (ES+), at 1.27 min.

¹H NMR: (400 MHz, DMSO-d₆) δ: 11.85 (s, 1H), 8.60 (d, J=7.6 Hz, 1H), 7.58 (s, 1H), 7.46-7.44 (m, 3H), 7.22 (s, 1H), 3.92 (s, 2H), 3.47-3.40 (m, 2H), 3.15 (t, J=8.8 Hz, 2H), 2.40 (s, 3H).

Step 2. Cs₂CO₃ (138 mg, 0.42 mmol) was added to a solution of 1-(2-((5-fluoro-6-hydroxypyridin-3-yl)methyl)pyridin-4-yl)-3-methyl-1,5,6,7-tetrahydro-4H-pyrazolo[4,3-c]pyridin-4-one (100 mg, 0.28 mmol) in DMF (10 mL), followed by the addition of sodium 2-chloro-2,2-difluoroacetate (44 mg, 0.282 mmol) and the reaction mixture was heated at 100° C. for 3 h. The reaction mixture was partitioned between water (50 mL) and 10% MeOH in DCM (50 mL). The organic layer was separated, washed with brine solution (50 mL), dried (Na₂SO₄) and the solvent removed in vacuo. The residue was purified by prep HPLC (Method 4). The residue was partitioned between 10% NaHCO₃ solution (25 mL) and EtOAc (25 mL). The organic layer was separated, dried (Na₂SO₄) and the solvent removed in vacuo to afford 1-(2-((6-(difluoromethoxy)-5-fluoropyridin-3-yl)methyl)pyridin-4-yl)-3-methyl-1,5,6,7-tetrahydro-4H-pyrazolo[4,3-c]pyridin-4-one as a white solid (10 mg, 9%). Data in table 3.

### Procedure 24:

### Example 112, 2-(2-(3-fluoro-5-(trifluoromethyl)benzyl)pyridin-4-yl)-6,7-dihydro-2H-pyrazolo[4,3-f][1,4]oxazepin-4(5H)-one

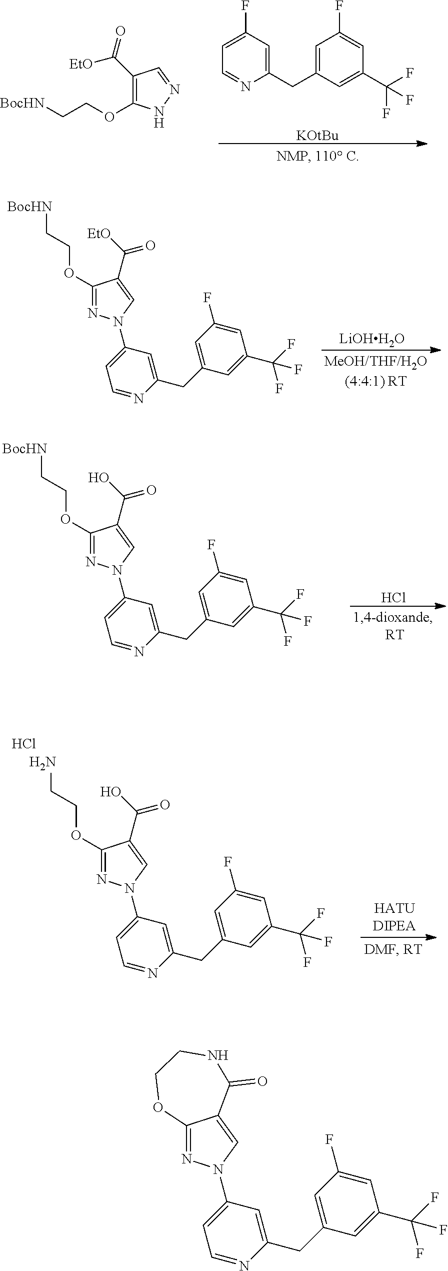

Step 1. Potassium t-butoxide (150 mg, 1.336 mmol) was added to a solution of ethyl 5-(2-((tert-butoxycarbonyl)amino)ethoxy)-1H-pyrazole-4-carboxylate (Intermediate 43, 200 mg, 0.67 mmol) and 4-fluoro-2-(3-fluoro-5-(trifluoromethyl)benzyl)pyridine (Intermediate 3, 183 mg, 0.67 mmol) in NMP (10 mL) at RT and the reaction mixture was heated at 110° C. for 16 h. The solvent was removed in vacuo. The residue was purified by gradient flash column chromatography eluting with 0-40% EtOAc in pet-ether to afford ethyl 3-(2-((tert-butoxycarbonyl)amino)ethoxy)-1-(2-(3-fluoro-5-(trifluoromethyl)benzyl)pyridin-4-yl)-1H-pyrazole-4-carboxylate as an off-white solid (200 mg, 54%).

LCMS (Method 3): m/z 553.1 (ES+), at 2.69 min.

¹H NMR: (400 MHz, DMSO-d₆) δ: 9.11 (s, 1H), 8.56 (d, J=5.6 Hz, 1H), 7.96 (s, 1H), 7.76-7.74 (m, 1H), 7.58-7.52 (m, 3H), 6.98 (t, J=5.6 Hz, 1H), 4.32-4.21 (m, 6H), 3.38-3.36 (m, 2H), 1.37 (s, 9H), 1.29 (t, J=6.8 Hz, 3H).

Step 2. LiOH.H₂O (27 mg, 0.63 mmol) was added to a solution of ethyl 3-(2-((tert-butoxycarbonyl)amino)ethoxy)-1-(2-(3-fluoro-5-(trifluoromethyl)benzyl)pyridin-4-yl)-1H-pyrazole-4-carboxylate (120 mg, 0.217 mmol) in MeOH:THF:H₂O (2 mL:2 mL:0.5 mL) and the reaction mixture was stirred at RT for 6 h. The reaction mixture was diluted with water (20 mL), neutralised with 10% citric acid solution (10 mL) to pH-7 and extracted with EtOAc (2×30 mL). The combined organic layers were washed with brine solution (30 mL), dried (Na₂SO₄) and the solvent removed in vacuo to afford 3-(2-((tert-butoxycarbonyl)amino)ethoxy)-1-(2-(3-fluoro-5-(trifluoromethyl)benzyl)pyridin-4-yl)-1H-pyrazole-4-carboxylic acid as a yellow solid (110 mg, 95%).

LCMS (Method 3): m/z 525.1 (ES+), at 2.15 min.

¹H NMR: (400 MHz, DMSO-d₆) δ: 12.38 (s, 1H), 9.06 (s, 1H), 8.54 (d, J=7.6 Hz, 1H), 7.93 (s, 1H), 7.93-7.71 (m, 1H), 7.58-7.51 (m, 3H), 7.00 (t, J=5.2 Hz, 1H), 4.29-4.26 (m, 4H), 2.44-2.39 (m, 2H), 1.37 (s, 9H).

Step 3. HCl solution (4 M in 1,4-dioxane, 2.2 mL) was added to a suspension of 3-(2-((tert-butoxycarbonyl)amino)ethoxy)-1-(2-(3-fluoro-5-(trifluoromethyl)benzyl)pyridin-4-yl)-1H-pyrazole-4-carboxylic acid (110 mg, 0.21 mmol) in 1,4-dioxane (2 mL) and the reaction mixture was stirred at RT for 3 h. The solvent was removed in vacuo to afford 3-(2-aminoethoxy)-1-(2-(3-fluoro-5-(trifluoromethyl)benzyl)pyridin-4-yl)-1H-pyrazole-4-carboxylic acid hydrochloride as a yellow solid (80 mg, 83%).

LCMS (Method 3): m/z 425.1 (ES+), at 1.16 min.

Step 4. DIPEA (48 mg, 0.38 mmol) and HATU (143 mg, 0.38 mmol) were added to a solution of 3-(2-aminoethoxy)-1-(2-(3-fluoro-5-(trifluoromethyl)benzyl)pyridin-4-yl)-1H-pyrazole-4-carboxylic acid hydrochloride (80 mg, 0.17 mmol) in DMF (1.6 mL) and the reaction mixture was stirred at RT for 8 h. The reaction mixture was diluted with water (50 mL) and extracted with EtOAc (2×30 mL). The combined organic layers were washed with brine solution (50 mL), dried (Na₂SO₄) and the solvent removed in vacuo. The residue was purified by prep HPLC (Method 4). The residue was partitioned between 10% NaHCO₃ solution (20 mL) and EtOAc (20 mL). The organic layer was separated, dried (Na₂SO₄) and the solvent removed in vacuo to afford 2-(2-(3-fluoro-5-(trifluoromethyl)benzyl)pyridin-4-yl)-6,7-dihydro-2H-pyrazolo[4,3-f][1,4]oxazepin-4(5H)-one as a white solid (30 mg, 42%). Data in table 3.

### Procedure 25:

### Example 116, 1-(2-(3-fluoro-5-(trifluoromethyl)benzyl)pyridin-4-yl)-3-(trifluoromethyl)-1,5,6,7-tetrahydro-4H-pyrazolo[4,3-c]pyridin-4-one

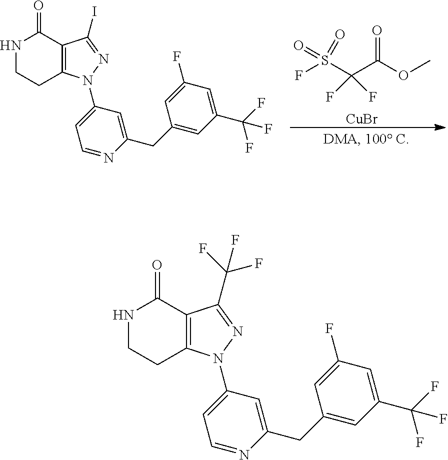

CuBr (8.3 mg, 0.058 mmol) was added to a solution of 1-(2-(3-fluoro-5-(trifluoromethyl)benzyl)pyridin-4-yl)-3-iodo-1,5,6,7-tetrahydro-4H-pyrazolo[4,3-c]pyridin-4-one (150 mg, 0.290 mmol) in DMA (8 mL) followed by the addition of methyl 2,2-difluoro-2-(fluorosulfonyl)acetate (334 mg, 1.74 mmol). The reaction mixture was heated in a microwave at 100° C. for 1 h. The reaction mixture was partitioned between water (10 mL) and EtOAc (20 mL). The organic layer was separated, dried (Na₂SO₄) and the solvent removed in vacuo. The residue was purified by prep HPLC (Method 4). The residue was partitioned between 10% NaHCO₃ solution (5 mL) and EtOAc (10 mL). The organic layer was separated, dried (Na₂SO₄) and the solvent removed in vacuo to afford 1-(2-(3-fluoro-5-(trifluoromethyl)benzyl)pyridin-4-yl)-3-(trifluoromethyl)-1,5,6,7-tetrahydro-4H-pyrazolo[4,3-c]pyridin-4-one as a white solid (14 mg, 10%). Data in table 3.

### Procedure 26:

### Example 117, 1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one

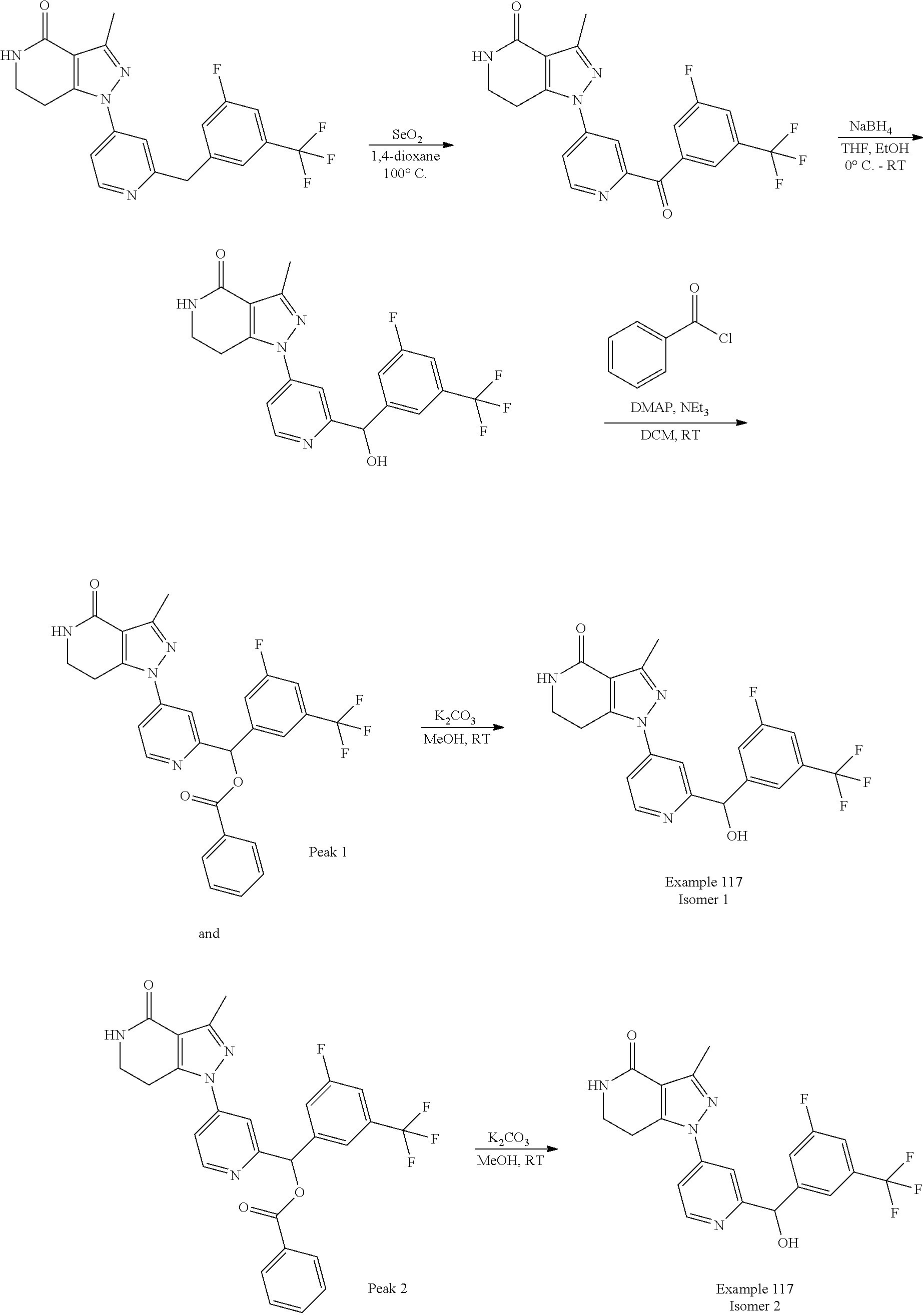

Steps 1 and 2. 1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one (300 mg, 58%) was prepared from 1-(2-(3-fluoro-5-(trifluoromethyl)benzyl)pyridin-4-yl)-3-methyl-1,5,6,7-tetrahydro-4H-pyrazolo[4,3-c]pyridin-4-one (Example 75, 500 mg, 1.23 mmol) using the methods of Procedure 11, steps 1 and 2.

LCMS (Method 3): m/z 421.1 (ES+), at 1.80 min.

¹H NMR: (400 MHz, DMSO-d₆) δ: 8.60 (d, J=5.2 Hz, 1H), 7.90 (d, J=2.4 Hz, 1H), 7.70 (s, 1H), 7.65-7.62 (m, 2H), 7.49-7.48 (m, 1H), 7.43 (s, 1H), 6.65 (d, J=4.4 Hz, 1H), 5.94 (d, J=4.4 Hz, 1H), 3.43-3.41 (m, 2H), 3.19-3.15 (m, 2H), 2.42 (s, 3H).

Step 3. NEt₃ (217 mg, 2.14 mmol) and DMAP (17.4 mg, 0.143 mmol) were added to a solution of 1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one (300 mg, 0.714 mmol) in DCM (10 mL) followed by the addition of benzoyl chloride (110 mg, 0.785 mmol) and the reaction mixture was stirred at RT for 15 h. The solvent was removed in vacuo. The residue was purified by gradient flash column chromatography eluting with 0-60% EtOAc in pet-ether. The residue was subjected to chiral SFC (Method 1—isocratic run 20% co-solvent) to afford (3-fluoro-5-(trifluoromethyl)phenyl)(4-(3-methyl-4-oxo-4,5,6,7-tetrahydro-1H-pyrazolo[4,3-c]pyridin-1-yl)pyridin-2-yl)methyl benzoate as an off-white solid (Peak 1, 100 mg, 26%) and (3-fluoro-5-(trifluoromethyl)phenyl)(4-(3-methyl-4-oxo-4,5,6,7-tetrahydro-1H-pyrazolo[4,3-c]pyridin-1-yl)pyridin-2-yl)methyl benzoate as an off-white solid (Peak 2, 110 mg, 29%).

### Peak 1:

LCMS (Method 3): m/z 525.1 (ES+), at 2.33 min.

Chiral SFC purity analysis (Method 3—isocratic run 20% co-solvent): 4.25 min, 100%.

### Peak 2:

LCMS (Method 3): m/z 525.1 (ES+), at 2.32 min.

Chiral SFC purity analysis (Method 3—isocratic run 20% co-solvent): 4.76 min, 89%.

Step 4 (Peak 1). K₂CO₃ (79 mg, 0.572 mmol) was added to a solution of (3-fluoro-5-(trifluoromethyl)phenyl)(4-(3-methyl-4-oxo-4,5,6,7-tetrahydro-1H-pyrazolo[4,3-c]pyridin-1-yl)pyridin-2-yl)methyl benzoate (Peak 1, 100 mg, 0.191 mmol) in MeOH (1 mL) and the reaction mixture was stirred at RT for 2 h. The reaction mixture was partitioned between DCM (10 mL) and H₂O (10 mL). The organic layer was separated, dried (Na₂SO₄) and the solvent removed in vacuo. The residue was purified by chiral SFC (Method 5—isocratic run 20% co-solvent) to afford 1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one (Isomer 1) as an off-white solid (28 mg, 35%). Data in table 3.

Step 4 (Peak 2). K₂CO₃ (87 mg, 0.629 mmol) was added to a solution of (3-fluoro-5-(trifluoromethyl)phenyl)(4-(3-methyl-4-oxo-4,5,6,7-tetrahydro-1H-pyrazolo[4,3-c]pyridin-1-yl)pyridin-2-yl)methyl benzoate (Peak 2, 110 mg, 0.210 mmol) in MeOH (1 mL) and the reaction mixture was stirred at RT for 2 h. The reaction mixture was partitioned between DCM (10 mL) and H₂O (10 mL). The organic layer was separated, dried (Na₂SO₄) and the solvent removed in vacuo. The residue was purified by chiral SFC (Method 5—isocratic run 20% co-solvent) to afford 1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one (Isomer 2) as an off-white solid (41 mg, 46%). Data in table 3.

### Procedure 27:

### Example 118, 1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one

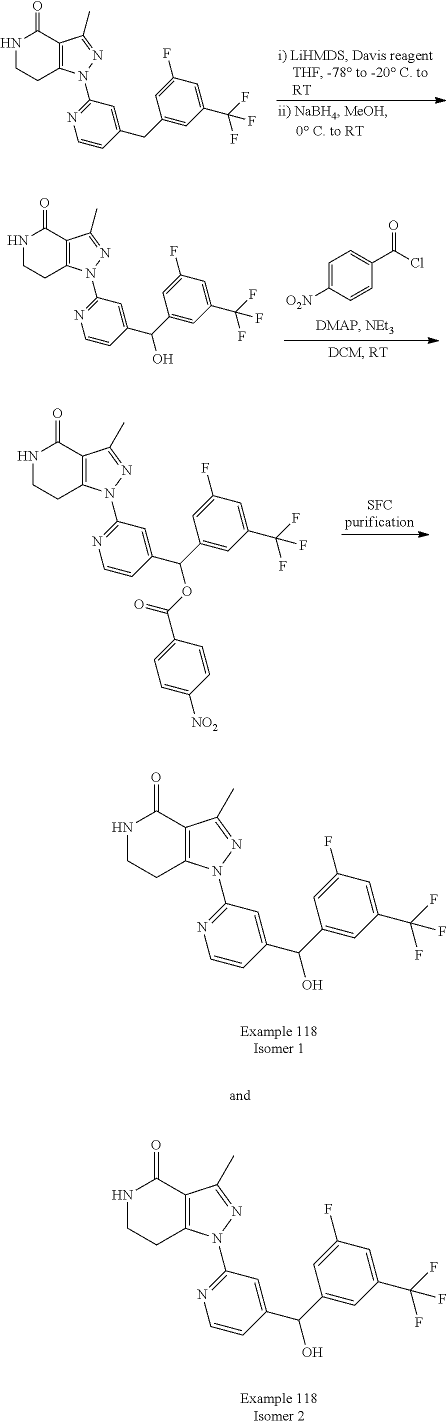 [SMILES: Cc1nn(-c2cc(Cc3cc(F)cc(C(F)(F)F)c3)ccn2)c2c1C(=O)NCC2 ; Cc1nn(-c2cc(C(O)c3cc(F)cc(C(F)(F)F)c3)ccn2)c2c1C(=O)NCC2 ; O=C(Cl)c1ccc([N+](=O)[O-])cc1 ; Cc1nn(-c2cc(C(OC(=O)c3ccc([N+](=O)[O-])cc3)c3cc(F)cc(C(F)(F)F)c3)ccn2)c2c1C(=O)NCC2 ; Cc1nn(-c2cc(C(O)c3cc(F)cc(C(F)(F)F)c3)ccn2)c2c1C(=O)NCC2 ; Cc1nn(-c2cc(C(O)c3cc(F)cc(C(F)(F)F)c3)ccn2)c2c1C(=O)NCC2]

Step 1. LiHMDS solution (1 M in THF, 3.71 mL, 3.71 mmol) was added to a solution of 1-(4-(3-fluoro-5-(trifluoromethyl)benzyl)pyridin-2-yl)-3-methyl-1,5,6,7-tetrahydro-4H-pyrazolo[4,3-c]pyridin-4-one (Example 95, 500 mg, 1.23 mmol) in THF (5 mL) at −78° C. and stirred for 10 minutes, followed by the addition of Davis reagent (1 g, 3.71 mmol) at −20° C. The reaction mixture was stirred at RT for 16 h. The reaction mixture was quenched with sat. NH₄Cl solution (40 mL) and the aqueous layer extracted with EtOAc (2×50 mL). The combined organic layers were dried (Na₂SO₄) and the solvent removed in vacuo. The residue was purified by gradient flash column chromatography eluting with 0-100% EtOAc in pet-ether. After removal of the solvent in vacuo the residue was dissolved MeOH (5 mL) and sodium borohydride (52 mg, 0.69 mmol) was added at 0° C. The reaction mixture was stirred at RT for 3 h. The reaction mixture was partitioned between EtOAc (20 mL) and water (20 mL). The organic layer was separated, dried (Na₂SO₄) and the solvent removed in vacuo. The residue was purified by gradient flash column chromatography eluting with 0-80% EtOAc in pet-ether to afford 1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one as an off-white solid (250 mg, 48%).

LCMS (Method 6): m/z 421.1 (ES+), at 2.12 min.

¹H NMR: (400 MHz, CDCl₃) δ: 8.42 (s, 1H), 8.05 (d, J=2.4 Hz, 1H), 7.51 (s, 1H), 7.37-7.30 (m, 2H), 7.29-7.26 (m, 1H), 6.86-6.70 (m, 1H), 3.70-3.63 (m, 4H), 2.54 (s, 3H). 2 exchangeable protons not observed.

Step 2. 4-Nitrobenzoyl chloride (105 mg, 0.571 mmol) and NEt₃ (0.2 mL, 1.71 mmol) were added to a solution of 1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one (240 mg, 0.57 mmol) in DCM (5 mL) and the reaction mixture was stirred at RT for 3 h. The solvent was removed in vacuo and the residue was purified by prep HPLC (Method 1). The residue obtained was dissolved in EtOAC (50 mL) and washed with water (50 mL). The organic layer was separated, dried (Na₂SO₄) and the solvent removed in vacuo. The residue was subjected to chiral SFC (Method 7—isocratic run 20% co-solvent). During evaporation of chiral SFC fractions, the 4-nitro benzoyl ester was hydrolysed therefore both elutions were further purified by gradient flash column chromatography eluting with 0-80% EtOAc in pet-ether to afford 1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one (Isomer 1) as an off-white solid (25 mg, 11%) and 1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one (Isomer 2) as an off-white solid (27 mg, 11%). Data in table 3.

Further examples prepared by the above procedures are detailed in Table 3.

TABLE 2
Intermediates

| Intermediate | Name | Structure | Data |
|---|---|---|---|
| 1 | (E)-3- ((dimethylamino) methylene)piperidine- 2,4-dione | 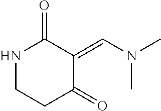 | LCMS (Method 2): m/z 169.2 (ES+), at 0.57 min & 0.73 min. ¹H NMR: (400 MHz, DMSO-d₆) δ: 7.83 (s, 1H), 7.15 (s, 1H), 3.29 (s, 3H), 3.17-3.15 (m, 2H), 3.10 (s, 3H), 2.30 (t, J = 6.4 Hz, 2H). |
| 2 | 2- ((dimethylamino) methylene)cyclohexane- 1,3-dione | 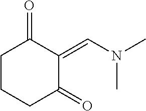 | LCMS (Method 4): m/z 168.2 (ES+), at 0.24 min. ¹H NMR: (400 MHz, DMSO-d₆) δ: 7.99 (s, 1H), 3.39 (s, 3H), 2.90 (s, 3H), 2.53-2.50 (m, 4H), 1.81 (t, J = 6.2 Hz, 2H). |
| 3 | 4-fluoro-2- (3-fluoro-5- (trifluoromethyl) benzyl)pyridine | 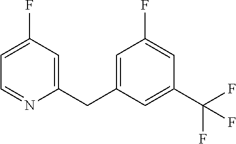 [SMILES: Fc1cc(Cc2cc(F)ccn2)cc(C(F)(F)F)c1] | LCMS (Method 5): m/z 274.1 (ES+), at 6.26 min. ¹H NMR: (400 MHz, DMSO-d₆) δ: 8.54 (dd, J = 9.1, 5.7 Hz, 1H), 7.60-7.45 (m, 3H), 7.36 (dd, J = 10.2, 2.5 Hz, 1H), 7.20 (ddd, J = 8.6, 5.7, 2.5 Hz, 1H), 4.24 (s, 2H). |
| 4 | 2-fluoro-4-(3- (trifluoromethyl) benzyl)pyridine | 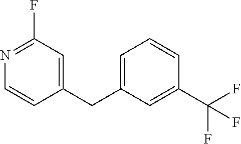 [SMILES: Fc1cc(Cc2cccc(C(F)(F)F)c2)ccn1] | LCMS (Method 4): m/z 256.2 (ES+), at 1.52 min. ¹H NMR: (400 MHz, Methanol-d₄) δ: 7.99 (dt, J = 5.2, 0.6 Hz, 1H), 7.52-7.35 (m, 4H), 7.06 (dddt, J = 5.2, 2.0, 1.3, 0.6 Hz, 1H), 6.83 (tq, J = 1.4, 0.7 Hz, 1H), 4.05 (s, 2H). |
| 5 | 2-fluoro-4- (3-fluoro-5- (trifluoromethyl) benzyl)pyridine | 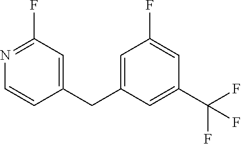 [SMILES: Fc1cc(Cc2ccnc(F)c2)cc(C(F)(F)F)c1] | LCMS (Method 3): m/z 274.0 (ES+), at 2.54 min. ¹H NMR: (400 MHz, DMSO-d₆) δ: 8.17 (d, J = 4.4 Hz, 1H), 7.65-7.51 (m, 3H), 7.29 (d, J = 1.2 Hz, 1H), 7.16 (s, 1H), 4.17 (d, J = 3.6 Hz, 2H). |
| 6 | 2-(3-fluoro-5- (trifluoromethyl) benzyl)-4- hydrazineylpyridine | 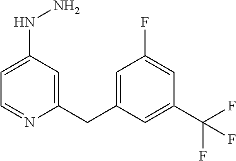 [SMILES: NNc1ccnc(Cc2cc(F)cc(C(F)(F)F)c2)c1] | LCMS (Method 3): m/z 286.1 (ES+), at 1.19 min. ¹H NMR: (400 MHz, DMSO-d₆) δ: 7.95 (d, J = 6.0 Hz, 1H), 7.56-7.21 (m, 4H), 6.59 (d, J = 1.6 Hz, 1H), 6.51-6.49 (m, 1H), 4.15 (s, 2H), 3.99 (s, 2H). |
| 7 | 2-hydrazineyl-4-(3- (trifluoromethyl) benzyl)pyridine | 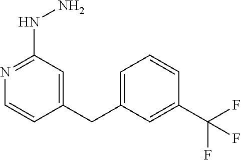 [SMILES: NNc1cc(Cc2cccc(C(F)(F)F)c2)ccn1] | LCMS (Method 4): m/z 268.2 (ES+), at 1.24 min. ¹H NMR: (400 MHz, DMSO-d₆) δ: 7.88 (dd, J = 5.2, 0.7 Hz, 1H), 7.69-7.43 (m, 4H), 7.36 (s, 1H), 6.57 (s, 1H), 6.43 (dd, J = 5.2, 1.6 Hz, 1H), 4.10 (brs, 2H), 3.93 (s, 2H). |
| 8 | 4-(3-fluoro-5- (trifluoromethyl) benzyl)-2- hydrazineylpyridine | 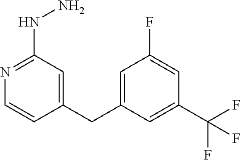 [SMILES: NNc1cc(Cc2cc(F)cc(C(F)(F)F)c2)ccn1] | LCMS (Method 3): m/z 286.2 (ES+), at 1.37 min. ¹H NMR: (400 MHz, DMSO-d₆) δ: 7.88 (d, J = 5.2 Hz, 1H), 7.53-7.44 (m, 3H), 7.36 (s, 1H), 6.57 (s, 1H), 6.45-6.44 (m, 1H), 4.07 (s, 2H), 3.95 (s, 2H). |
| 9 | 5-(3-fluoro-5- (trifluoromethyl) benzyl)-3- hydrazineylpyridazine | 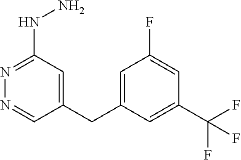 [SMILES: NNc1cc(Cc2cc(F)cc(C(F)(F)F)c2)cnn1] | LCMS (Method 3): m/z 287.1 (ES+), at 1.37 min. ¹H NMR: Not recorded. |
| 10 | 1-(6-chloropyridazin- 4-yl)-1,5,6,7- tetrahydro-4H- pyrazolo[4,3-c]pyridin- 4-one | 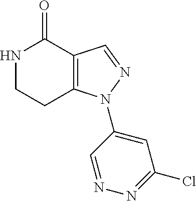 [SMILES: O=C1NCCc2c1cnn2-c1cnnc(Cl)c1] | LCMS (Method 3): m/z 250.0 (ES+), at 1.02 min. ¹H NMR: (400 MHz, DMSO-d₆) δ: 9.66 (d, J = 1.6 Hz, 1H), 8.20 (d, J = 1.4 Hz, 1H), 8.13 (s, 1H), 7.61 (s, 1H), 3.45-3.44 (m, 2H), 3.31- 3.30 (m, 2H). |
| 11 | 1-(5-bromopyridin- 3-yl)-1,5,6,7- tetrahydro-4H- pyrazolo[4,3-c]pyridin- 4-one | 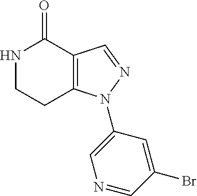 [SMILES: O=C1NCCc2c1cnn2-c1cncc(Br)c1] | LCMS (Method 3): m/z 293.1 (ES+), at 1.26 min. ¹H NMR: (400 MHz, DMSO-d₆) δ: 8.89-8.81 (m, 2H), 8.38-8.37 (m, 1H), 8.06 (s, 1H), 7.49 (s, 1H), 3.45-3.41 (m, 2H), 3.16-3.12 (m, 2H). |
| 12 | 1-(4-bromopyridin- 2-yl)-1,5,6,7- tetrahydro-4H- pyrazolo[4,3-c]pyridin- 4-one | 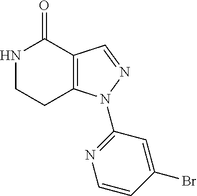 [SMILES: O=C1NCCc2c1cnn2-c1cc(Br)ccn1] | MS (Method 1): m/z 293, 295 (ES+). ¹H NMR: (400 MHz, DMSO-d₆) δ: 8.41 (d, J = 5.3 Hz, 1H), 8.12 (s, 1H), 8.03 (s, 1H), 7.69 (d, J = 4.0 Hz, 1H), 7.49 (bs, 1H), 3.62-3.50 (m, 4H). |
| 13 | 1-(2-bromopyridin- 4-yl)-1,5,6,7- tetrahydro-4H- pyrazolo[4,3-c]pyridin- 4-one | 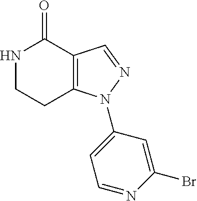 [SMILES: O=C1NCCc2c1cnn2-c1ccnc(Br)c1] | LCMS (Method 3): m/z 293.1 (ES+), at 1.24 min. ¹H NMR: (400 MHz, DMSO-d₆) δ: 8.54 (d, J = 7.6 Hz, 1H), 8.09 (s, 1H), 7.90 (d, J = 2.0 Hz, 1H), 7.76- 7.75 (m, 1H), 7.54 (s, 1H), 3.43 (d, J = 8.8 Hz, 2H), 3.23 (t, J = 8.8 Hz, 2H). |
| 14 | tert-butyl 1-(2- bromopyridin- 4-yl)-4-oxo- 1,4,6,7-tetrahydro- 5H-pyrazolo[4,3-c] pyridine-5-carboxylate | 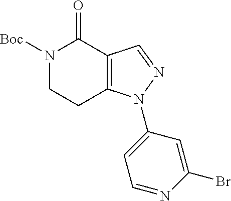 [SMILES: CC(C)(C)OC(=O)N1CCc2c(cnn2-c2ccnc(Br)c2)C1=O] | LCMS (Method 3): m/z 393.1 (ES+), at 2.11 min. ¹H NMR: (400 MHz, DMSO-d₆) δ: 8.57 (d, J = 5.6 Hz, 1H), 8.25 (s, 1H), 7.91 (d, J = 1.6 Hz, 1H), 7.76- 7.75 (m, 1H), 4.01 (t, J = 6.0 Hz, 2H), 3.36-3.34 (m, 2H), 1.49 (s, 9H). |
| 15 | 1,5,6,7- tetrahydro-4H- benzo[d][1,2,3] triazol-4-one | 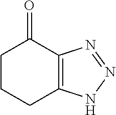 | MS (Method 1): m/z 138 (ES+). ¹H NMR: (400 MHz, DMSO-d₆) δ: 2.91 (t, J = 6.0 Hz, 2H), 2.56-2.43 (m, 2H), 2.13-2.02 (m, 2H). 1 exchangeable proton not observed. |
| 16 | 1,5,6,7-tetrahydro- 4H-[1,2,3]triazolo[4,5- c]pyridin-4-one | 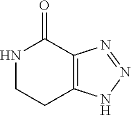 | MS (Method 1): m/z 139.0 (ES+). ¹H NMR: Not recorded. |
| 17 | (E)-3- ((dimethylamino) methylene)-6- methylpiperidine- 2,4-dione | 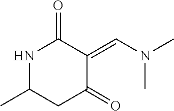 | MS (Method 1): m/z 183.0 (ES+). ¹H NMR: Not recorded. |
| 18 | 2-chloro-4- (3-fluoro-5- (trifluoromethyl) benzyl)pyridine | 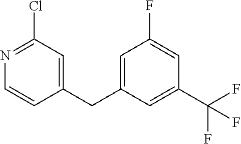 [SMILES: Fc1cc(Cc2ccnc(Cl)c2)cc(C(F)(F)F)c1] | LCMS (Method 9): m/z 290.2 (ES+), at 2.30 min. ¹H NMR: Not recorded. |
| 19 | ethyl (Z)-4- (tert-butoxy)-2- ((dimethylamino) methylene)-3- oxobutanoate | 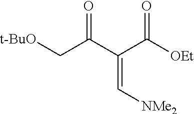 [SMILES: CCOC(=O)/C(=C\N(C)C)C(=O)COC(C)(C)C] | LCMS (Method 9): m/z 258.2 (ES+), at 1.57 min. ¹H NMR: Not recorded. |
| 20 | (E)-3- ((dimethylamino) methylene)-5- methylpiperidine- 2,4-dione | 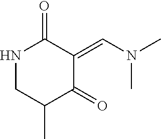 | LCMS: Not recorded. ¹H NMR: (400 MHz, DMSO-d₆) δ: 7.81 (s, 1H), 7.15 (s, 1H), 3.32 (s, 3H), 3.28-3.17 (m, 1H), 3.03 (s, 3 H), 2.89-2.88 (m, 1H), 2.32-2.28 (m, 1H), 0.97 (d, J = 6.9 Hz, 3H). |
| 21 | tert-butyl 1-(2- bromopyridin-4- yl)-4-oxo- 1,4,6,7-tetrahydro- 5H-pyrrolo[3,2-c] pyridine-5-carboxylate | 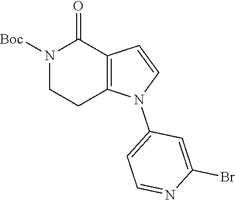 [SMILES: CC(C)(C)OC(=O)N1CCc2c(ccn2-c2ccnc(Br)c2)C1=O] | LCMS (Method 7): m/z 336.0 (ES+), at 2.14 min. ¹H NMR: (400 MHz, DMSO-d₆) δ: 8.52 (d, J = 7.6 Hz, 1H), 7.87 (s, 1H), 7.62 (t, J = 2.8 Hz, 1H), 7.38 (d, J = 4.0 Hz, 1H), 6.64 (d, J = 4.4 Hz, 1H), 3.94 (t, J = 8.0 Hz, 2H), 3.09 (t, J = 8.0 Hz, 2H), 1.48 (s, 9H). |
| 22 | 1-(chloromethyl)- 3-cyclopropyl-5- fluorobenzene | 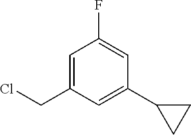 [SMILES: Fc1cc(CCl)cc(C2CC2)c1] | GCMS (Method 1): m/z 184.0 (ES+), at 7.02 min. ¹H NMR: Not recorded. |
| 23 | tert-butyl 1-(6- chloropyrimidin-4-yl)-4- oxo-1,4,6,7-tetrahydro- 5H-pyrazolo[4,3-c] pyridine-5-carboxylate | 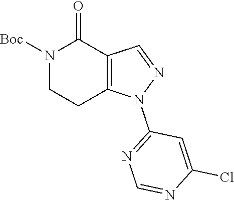 [SMILES: CC(C)(C)OC(=O)N1CCc2c(cnn2-c2cc(Cl)ncn2)C1=O] | LCMS (Method 3): m/z 348.0 (ES+), at 2.25 min. ¹H NMR: (400 MHz, DMSO-d₆) δ: 9.05 (d, J = 1.2 Hz, 1H), 8.32 (s, 1H), 8.08 (d, J = 0.8 Hz, 1H), 4.06- 4.02 (m, 2H), 3.59 (t, J = 6.4 Hz, 2H), 1.49 (s, 9H). |
| 24 | 4-(chloromethyl)-1- (difluoromethoxy)-2- fluorobenzene | 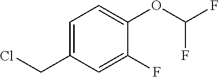 [SMILES: Fc1cc(CCl)ccc1OC(F)F] | GCMS (Method 2): m/z 210.0 (ES+), at 2.46 min. ¹H NMR: Not recorded. |
| 25 | 5-(bromomethyl)- 1,2-difluoro-3- (trifluoromethyl) benzene | 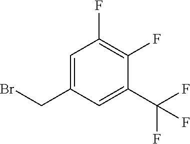 [SMILES: Fc1cc(CBr)cc(C(F)(F)F)c1F] | GCMS: Not recorded. ¹H NMR: Not recorded. TLC: R_{f} = 0.7 (25% EtOAc in pet- ether) |
| 26 | 1-(chloromethyl)-3- (difluoromethyl)-5- fluorobenzene | 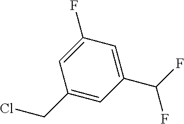 [SMILES: Fc1cc(CCl)cc(C(F)F)c1] | GCMS (Method 2): m/z 193.9 (ES+), at 2.25 min. ¹H NMR: Not recorded. |
| 27 | 1-chloro-5- (chloromethyl)-2,3- difluorobenzene | 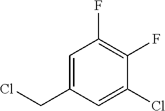 [SMILES: Fc1cc(CCl)cc(Cl)c1F] | GCMS (Method 2): m/z 197.0 (ES+), at 2.51 min. ¹H NMR: (400 MHz, DMSO-d₆) δ: 7.63-7.58 (m, 2H), 4.76 (s, 2H). |
| 28 | 5-(chloromethyl)- 1,3-difluoro-2- (trifluoromethyl) benzene | 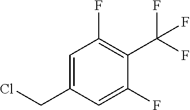 [SMILES: Fc1cc(CCl)cc(F)c1C(F)(F)F] | GCMS (Method 2): m/z 230.0 (ES+), at 2.04 min. ¹H NMR: (400 MHz, CDCl₃) δ: 7.10 (s, 1H), 7.08 (s, 1H), 4.57 (s, 2H). |
| 29 | 1-(chloromethyl)- 3-fluoro-5- isopropylbenzene | 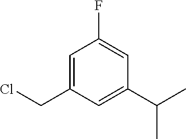 | GCMS (Method 2): m/z 186.0 (ES+), at 2.77 min. ¹H NMR: (400 MHz, DMSO-d₆) δ: 7.18 (s, 1H), 7.11-7.07 (m, 2H), 4.75 (s, 2H), 2.96-2.89 (m, 1H), 1.20 (d, J = 6.9 Hz, 6H). |
| 30 | 1-(chloromethyl)- 3-fluoro-5-(1- (trifluoromethyl) cyclopropyl)benzene | 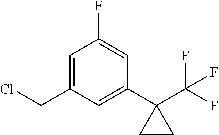 [SMILES: Fc1cc(CCl)cc(C2(C(F)(F)F)CC2)c1] | LCMS (Method 2): m/z No ionisation observed (ES+), at 2.80 min. ¹H NMR: (400 MHz, CDCl₃) δ: 7.28 (s, 1H), 7.16-7.11 (m, 2H), 4.58 (S,2H), 1.40 (t, J = 6.8 Hz, 2H), 1.07 (t, J = 4.8 Hz, 2H). |
| 31 | 1-chloro-5- (chloromethyl)- 2-fluoro-3- (trifluoromethyl) benzene | 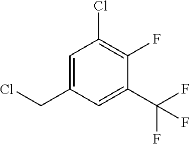 [SMILES: Fc1c(Cl)cc(CCl)cc1C(F)(F)F] | LCMS (Method 7): m/z No ionisation observed (ES+), at 2.15 min. ¹H NMR: Not recorded. |
| 32 | 5-(chloromethyl)- 3-fluoro-2- methoxypyridine | 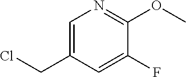 | LCMS (Method 7): m/z 176.1 (ES+), at 1.77 min. ¹H NMR: (400 MHz, DMSO-d₆) δ: 8.08 (d, J = 2.0 Hz, 1H), 7.82-7.77 (m, 1H), 4.78 (s, 2H), 3.95 (s, 3H). |
| 33 | 5-(chloromethyl)- 2-methoxypyridine | 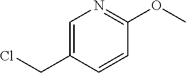 | LCMS (Method 2): m/z 157.7 (ES+), at 2.04 min. ¹H NMR: (400 MHz, DMSO-d₆) δ: 8.24 (s, 1H), 7.80 (d, J = 3.6 Hz, 1H), 6.85 (d, J = 11.6 Hz, 1H), 4.77 (s, 2H), 3.86 (s, 3H). |
| 34 | 5-(chloromethyl)-2- (difluoromethoxy)pyridine | 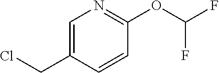 [SMILES: FC(F)Oc1ccc(CCl)cn1] | LCMS (Method 10): m/z 194.1 (ES+), at 2.09 min. ¹H NMR: (400 MHz, DMSO-d₆) δ: 8.35 (d, J = 2.4 Hz, 1H), 8.03-8.00 (m, 1H), 7.90-7.54 (m, 1H), 7.13 (d, J = 8.4 Hz, 1H), 4.83 (s, 2H). |
| 35 | 3-acetyl-1-(2,4- dimethoxybenzyl)- 4-hydroxy-5,6- dihydropyridin- 2(1H)-one | 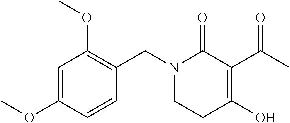 [SMILES: COc1ccc(CN2CCC(O)=C(C(C)=O)C2=O)c(OC)c1] | LCMS (Method 3): m/z 306.0 (ES+), at 2.17 min. ¹H NMR: (400 MHz, DMSO-d₆) δ: 7.12 (d, J = 8.0 Hz, 1H), 6.59 (d, J = 2.4 Hz, 1H), 6.52-6.50 (m, 1H), 4.53 (s, 2H), 3.80 (s, 3H), 3.76 (s, 3H), 3.42 (t, J = 6.8 Hz, 2H), 3.31 (s, 2H), 2.44 (s, 3H). 1 exchangeable proton not observed. |
| 36 | 1-(2-bromopyridin- 4-yl)-5-(2,4- dimethoxybenzyl)- 3-methyl-1,5,6,7- tetrahydro-4H- pyrazolo[4,3-c]pyridin- 4-one | 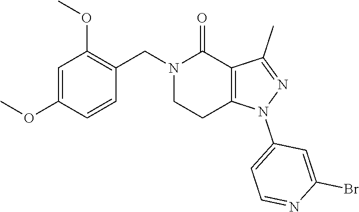 [SMILES: COc1ccc(CN2CCc3c(c(C)nn3-c3ccnc(Br)c3)C2=O)c(OC)c1] | LCMS (Method 7): m/z 457.1 (ES+), at 1.96 min. ¹H NMR: (400 MHz, DMSO-d₆) δ: 8.49 (d, J = 7.2 Hz, 1H), 7.84 (d, J = 2.0 Hz, 1H), 7.70-7.68 (m, 1H), 7.11 (d, J = 11.6 Hz, 1H), 6.59 (d, J = 2.4 Hz, 1H), 6.51-6.48 (m, 1H), 4.51 (s, 2H), 3.81 (s, 3H), 3.75 (s, 3H), 3.54 (t, J = 9.2 Hz, 2H), 3.26 (t, J = 8.4 Hz, 2H), 2.43 (s, 3H). |
| 37 | 1-(5-bromopyridin- 3-yl)-5-(2,4- dimethoxybenzyl)- 3-methyl-1,5,6,7- tetrahydro-4H-pyrazolo [4,3-c]pyridin-4- one | 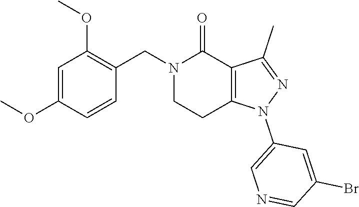 [SMILES: COc1ccc(CN2CCc3c(c(C)nn3-c3cncc(Br)c3)C2=O)c(OC)c1] | LCMS (Method 6): m/z 457.1 (ES+), at 1.92 min. ¹H NMR: (400 MHz, DMSO-d₆) δ: 8.84 (d, J = 2.0 Hz, 1H), 8.76 (d, J = 1.6 Hz, 1H), 8.31-8.30 (m, 1H), 7.12-7.10 (m, 1H), 6.59 (d, J = 2.4 Hz, 1H), 6.51-6.49 (m, 1H), 4.15 (s, 2H), 3.81-3.75 (m, 6H), 3.55- 3.51 (m, 2H), 3.18-3.15 (m, 2H), 2.43 (s, 3H). |
| 38 | 1-(4-bromopyridin- 2-yl)-5-(2,4- dimethoxybenzyl)- 3-methyl-1,5,6,7- tetrahydro-4H- pyrazolo[4,3-c]pyridin- 4-one | 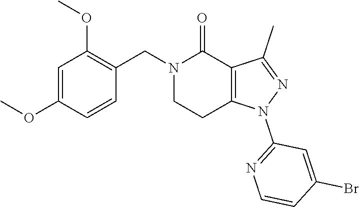 [SMILES: COc1ccc(CN2CCc3c(c(C)nn3-c3cc(Br)ccn3)C2=O)c(OC)c1] | LCMS (Method 3): m/z 459.0 (ES+), at 2.60 min. ¹H NMR: (400 MHz, DMSO-d₆) δ: 8.37 (d, J = 5.6 Hz, 1H), 8.07 (d, J = 1.2 Hz, 1H), 7.65-7.63 (m, 1H), 7.10 (d, J = 8.4 Hz, 1H), 6.58 (d, J = 2.0 Hz, 1H), 6.51-6.48 (m, 1H), 4.51 (s, 2H), 3.81 (s, 3H), 3.75 (s, 3H), 3.56 (t, J = 13.6 Hz, 2H), 3.44 (t, J = 13.6 Hz, 2H), 2.44 (s, 3H). |
| 39 | 4-(3-(difluoromethyl)- 5-fluorobenzyl)-2- hydrazineylpyridine | 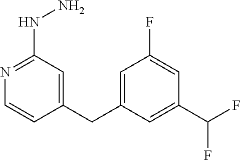 [SMILES: NNc1cc(Cc2cc(F)cc(C(F)F)c2)ccn1] | LCMS (Method 2): m/z 267.9 (ES+), at 3.00 min. ¹H NMR: Not recorded. |
| 40 | 1-(5-fluoropyridin- 3-yl)-1,5,6,7- tetrahydro-4H- pyrazolo[4,3-c]pyridin- 4-one | 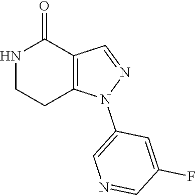 [SMILES: O=C1NCCc2c1cnn2-c1cncc(F)c1] | LCMS (Method 10): m/z 233.2 (ES+), at 1.20 min. ¹H NMR: (400 MHz, DMSO-d₆) δ: 8.79 (s, 1H), 8.72-8.71 (m, 1H), 8.13-8.10 (m, 1H), 8.08 (s, 1H), 7.50 (s, 1H), 3.46-3.42 (m, 2H), 3.15 (t, J = 6.4 Hz, 2H). |
| 41 | tert-butyl 1-(2-bromo- 5-methylpyridin-4- yl)-4-oxo-1,4,6,7- tetrahydro-5H- pyrazolo[4,3-c] pyridine-5-carboxylate | 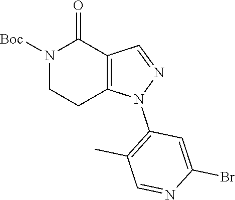 [SMILES: Cc1cnc(Br)cc1-n1ncc2c1CCN(C(=O)OC(C)(C)C)C2=O] | LCMS (Method 3): m/z 407.0 (ES+), at 2.00 min. ¹H NMR: (400 MHz, DMSO-d₆) δ: 8.54 (s, 1H), 8.19 (s, 1H), 7.88 (s, 1H), 4.00 (t, J = 2.8 Hz, 2H), 3.00 (t, J = 6.0 Hz, 2H), 2.19 (s, 3H), 1.48 (s, 9H). |
| 42 | tert-butyl 2-(3- (3-fluoro-5- (trifluoromethyl)benzyl) phenyl)hydrazine- 1-carboxylate | 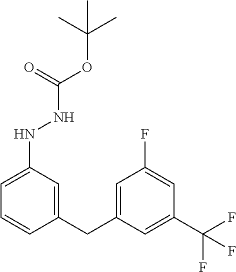 [SMILES: CC(C)(C)OC(=O)NNc1cccc(Cc2cc(F)cc(C(F)(F)F)c2)c1] | LCMS (Method 1): m/z 407.0 (ES−), at 5.56 min. ¹H NMR: (400 MHz, DMSO-d₆) δ: 8.71 (s, 1H), 7.59-7.29 (m, 2H), 7.08 (t, J = 7.7 Hz, 1H), 6.62 (d, J = 7.4 Hz, 1H), 6.58-6.43 (m, 2H), 5.03 (s, 1H), 4.06 (s, 1H), 3.97 (s, 2H), 1.38 (d, J = 2.4 Hz, 9H). |
| 43 | ethyl 5-(2-((tert- butoxycarbonyl) amino)ethoxy)-1H- pyrazole-4- carboxylate | 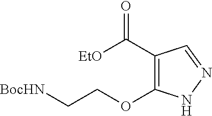 [SMILES: CCOC(=O)c1cn[nH]c1OCCNC(=O)OC(C)(C)C] | LCMS (Method 7): m/z 298.0 (ES−), at 1.84 min. ¹H NMR: (400 MHz, CDCl₃) δ: 7.93 (s, 1H), 5.33 (s, 1H), 4.39- 4.29 (m, 4H), 3.58 (t, J = 4.0 Hz, 2H), 1.57 (s, 9H), 1.37 (t, J = 7.2 Hz, 3H). 1 exchangeable proton not observed. |
| 44 | tert-butyl 3-(2- methoxyacetyl)-2,4- dioxopiperidine- 1-carboxylate | 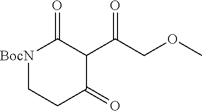 [SMILES: COCC(=O)C1C(=O)CCN(C(=O)OC(C)(C)C)C1=O] | LCMS (Method 3): m/z 284.1 (ES−), at 1.79 min. ¹H NMR: (400 MHz, DMSO-d₆) δ: 4.63 (s, 3H), 3.76 (s, 3H), 2.79- 2.73 (m, 4H), 1.47 (s, 9H). |
| 45 | 1-(2-(3-fluoro-5- (trifluoromethyl)benzyl) pyridin-4-yl)-3- iodo-1,5,6,7- tetrahydro-4H- pyrazolo[4,3- c]pyridin-4-one | 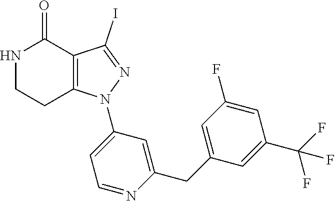 [SMILES: O=C1NCCc2c1c(I)nn2-c1ccnc(Cc2cc(F)cc(C(F)(F)F)c2)c1] | LCMS (Method 10): m/z 517.0 (ES+), at 1.88 min. ¹H NMR: (400 MHz, DMSO-d₆) δ: 8.65 (d, J = 7.2 Hz, 1H), 7.71 (s, 1H), 7.62 (s, 1H), 7.56-7.48 (m, 4H), 4.32 (s, 2H), 3.40-3.26 (m, 2H), 3.21-3.16 (m, 2H). |

TABLE 3
Examples

| Ex. No. | Name | Intermediate/procedure | ¹H NMR | LCMS |
|---|---|---|---|---|
| 1 | 1-(2-(3-fluoro-5-(trifluoromethyl)benzyl)pyridin- | Intermediates 1 and 6 | (400 MHz, DMSO-d₆) δ: 8.65 (d, J = | m/z 391.1 |
| | 4-yl)-1,5,6,7-tetrahydro-4H-pyrazolo[4,3- | Procedure 1 | 5.2 Hz, 1H), 8.06 (s, 1H), 7.72 (d, J = | (M + H)+ |
| | c]pyridin-4-one | | 2.1 Hz, 1H), 7.63 (s, 1H), 7.57-7.52 | (ES+), at |
| | | | (m, 4H), 4.33 (s, 2H), 3.43 (td, J = | 1.89 min, |
| | | | 6.7, 2.5 Hz, 2H), 3.20 (t, J = 6.7 Hz, | 95% |
| | | | 2H). | (Method 3) |
| 2 | 1-(4-(3-(trifluoromethyl)benzyl)pyridin-2-yl)- | Intermediates 1 and 7 | (400 MHz, DMSO-d₆) δ: 8.42 (dd, J = | m/z 373.1 |
| | 1,5,6,7-tetrahydro-4H-pyrazolo[4,3-c]pyridin-4- | Procedure 1 | 5.1, 0.7 Hz, 1H), 7.99 (s, 1H), 7.85 | (M + H)+ |
| | one | | (dd, J = 1.6, 0.8 Hz, 1H), 7.75 (s, 1H), | (ES+), at |
| | | | 7.71-7.52 (m, 3H), 7.46 (s, 1H), 7.35 | 4.42 min, |
| | | | (dd, J = 5.1, 1.5 Hz, 1H), 4.23 (s, 2H), | 100% |
| | | | 3.49-3.35 (m, 4H). | (Method 1) |
| 3 | 5-methyl-1-(4-(3-(trifluoromethyl)benzyl)pyridin- | Example 2 | (400 MHz, DMSO-d₆) δ: 8.41 (d, J = | m/z 387.2 |
| | 2-yl)-1,5,6,7-tetrahydro-4H-pyrazolo[4,3- | Procedure 2 | 5.2 Hz, 1H), 7.98 (s, 1H), 7.85 (s, | (M + H)+ |
| | c]pyridin-4-one | | 1H), 7.74 (s, 1H), 7.70-7.50 (m, 3H), | (ES+), at |
| | | | 7.33 (d, J = 5.1 Hz, 1H), 4.22 (s, 2H), | 4.53 min, |
| | | | 3.59 (t, J = 6.9 Hz, 2H), 3.49 (t, J = | 97% |
| | | | 6.9 Hz, 2H), 2.93 (s, 3H). | (Method 1) |
| 4 | 1-(4-(3-fluoro-5-(trifluoromethyl)benzyl)pyridin- | Intermediates 1 and 8 | (400 MHz, DMSO-d₆) δ: 8.42 (d, J = | m/z 391.1 |
| | 2-yl)-1,5,6,7-tetrahydro-4H-pyrazolo[4,3- | Procedure 1 | 5.2 Hz, 1H), 7.99 (s, 1H), 7.88 (s, | (M + H)+ |
| | c]pyridin-4-one | | 1H), 7.65 (s, 1H), 7.61-7.55 (m, 2H), | (ES+), at |
| | | | 7.45 (s, 1H), 7.37-7.35 (m, 1H), 4.23 | 2.16 min, |
| | | | (s, 2H), 3.43-3.39 (m, 4H). | 99% |
| | | | | (Method 3) |
| 5 | 1-(4-(3-fluoro-5-(trifluoromethyl)benzyl)pyridin- | Example 4 | (400 MHz, DMSO-d₆) δ: 8.43 (d, J = | m/z 405.2 |
| | 2-yl)-5-methyl-1,5,6,7-tetrahydro-4H- | Procedure 2 | 5.2 Hz, 1H), 7.99 (s, 1H), 7.90 (s, | (M + H)+ |
| | pyrazolo[4,3-c]pyridin-4-one | | 1H), 7.60 (t, J = 11.8 Hz, 3H), 7.37 | (ES+), at |
| | | | (d, J = 5.1 Hz, 1H), 4.24 (s, 2H), 3.62 | 2.40 min, |
| | | | (t, J = 7.3 Hz, 2H), 3.33 (s, 2H), 2.94 | 99% |
| | | | (s, 3H). | (Method 3) |
| 6 | 1-(4-((6-fluoro-5-methylpyridin-3- | Intermediate 12 and | (400 MHz, DMSO-d₆) δ: | m/z 338.2 |
| | yl)methyl)pyridin-2-yl)-1,5,6,7-tetrahydro-4H- | CAS: 1260812-39-2 | 8.41 (d, J = 5.1 Hz, 1H), 8.05 (s, 1H), | (M + H)+ |
| | pyrazolo[4,3-c]pyridin-4-one | Procedure 3 | 7.98 (s, 1H), 7.83 (s, 1H), 7.80-7.71 | (ES+), at |
| | | | (m, 1H), 7.45 (s, 1H), 7.36-7.26 (m, | 4.83 min, |
| | | | 1H), 4.10 (s, 2H), 3.51-3.35 (m, 4H), | 95% |
| | | | 2.20 (s, 3H). | (Method 5) |
| 7 | 1-(4-((6-fluoro-5-methylpyridin-3- | Example 6 | (400 MHz, DMSO-d₆) δ: 8.41 (d, J = | m/z 352.3 |
| | yl)methyl)pyridin-2-yl)-5-methyl-1,5,6,7- | Procedure 2 | 5.1 Hz, 1H), 8.05 (d, J = 2.2 Hz, 1H), | (M + H)+ |
| | tetrahydro-4H-pyrazolo[4,3-c]pyridin-4-one | | 7.98 (s, 1H), 7.83 (s, 1H), 7.81-7.73 | (ES+), at |
| | | | (m, 1H), 7.31 (dd, J = 5.1, 1.6 Hz, | 5.18 min, |
| | | | 1H), 4.10 (s, 2H), 3.60 (t, J = 6.9 Hz, | 100% |
| | | | 2H), 3.49 (t, J = 6.9 Hz, 2H), 2.93 (s, | (Method 5) |
| | | | 3H), 2.20 (s, 3H). | |
| 8 | 2-(4-(3-(trifluoromethyl)benzyl)pyridin-2-yl)- | Intermediate 7 and | (400 MHz, DMSO-d₆) δ: 8.74 (d, J = | m/z 373.2 |
| | 2,5,6,7-tetrahydro-4H-pyrazolo[4,3-c]pyridin-4- | CAS: 50607-30-2 | 0.8 Hz, 1H), 8.48-8.36 (m, 1H), 7.86 | (M + H)+ |
| | one | Procedure 4 | (d, J = 1.2 Hz, 1H), 7.75 (s, 1H), | (ES+), at |
| | | | 7.72-7.52 (m, 4H), 7.37-7.27 (m, 1H), | 4.15 min, |
| | | | 4.22 (s, 2H), 3.45 (td, J = 6.6, 2.6 Hz, | 100% |
| | | | 2H), 2.89 (t, J = 6.6 Hz, 2H). | (Method 1) |
| 9 | 2-(2-(3-fluoro-5-(trifluoromethyl)benzyl)pyridin- | Intermediate 6 and | (400 MHz, DMSO-d₆) δ: 9.12 (s, 1H), | m/z 391.1 |
| | 4-yl)-2,5,6,7-tetrahydro-4H-pyrazolo[4,3- | CAS: 50607-30-2 | 8.57 (d, J = 5.6 Hz, 1H), 8.00 (d, J = | (M + H)+ |
| | c]pyridin-4-one | Procedure 4 | 2.0 Hz, 1H), 7.79 (t, J = 2.0 Hz, 1H), | (ES+), at |
| | | | 7.69 (s, 1H), 7.60 (s, 1H), 7.54 (t, J = | 1.91 min, |
| | | | 2.4 Hz, 2H), 4.27 (s, 2H), 3.46 (t, J = | 99% |
| | | | 2.8 Hz, 2H), 2.90 (t, J = 6.4 Hz, 2H). | (Method 2) |
| 10 | 1-(4-(3-fluoro-5-(trifluoromethyl)benzyl)pyridin- | Intermediates 2 and 8 | (400 MHz, DMSO-d₆) δ: 8.44 (dd, J = | m/z 405.2 |
| | 2-yl)-5,6,7,8-tetrahydropyrazolo[4,3-c]azepin- | Procedure 5 | 5.1, 0.7 Hz, 1H), 8.01 (s, 1H), 7.86- | (M + H)+ |
| | 4(1H)-one | | 7.77 (m, 2H), 7.66 (d, J = 1.6 Hz, | (ES+), at |
| | | | 1H), 7.58 (dd, J = 13.2, 9.5 Hz, 2H), | 4.24 min, |
| | | | 7.39 (dd, J = 5.1, 1.5 Hz, 1H), 4.23 | 99% |
| | | | (s, 2H), 3.28-3.20 (m, 2H), 1.96 (dd, | (Method 1) |
| | | | J = 15.1, 6.2 Hz, 2H). 2 protons | |
| | | | obscured by solvent peak. | |
| 11 | 1-(4-(3-(trifluoromethyl)benzyl)pyridin-2-yl)- | Intermediates 2 and 7 | (400 MHz, DMSO-d₆) δ: 8.55-8.34 | m/z 387.2 |
| | 5,6,7,8-tetrahydropyrazolo[4,3-c]azepin-4(1H)- | Procedure 5 | (m, 1H), 8.00 (s, 1H), 7.83 (d, J = 4.8 | (M + H)+ |
| | one | | Hz, 1H), 7.80-7.74 (m, 2H), 7.61 | (ES+), at |
| | | | (ddd, J = 21.1, 14.1, 7.5 Hz, 3H), | 4.14 min, |
| | | | 7.37 (dd, J = 5.1, 1.5 Hz, 1H), 4.22 | 100% |
| | | | (s, 2H), 3.26-3.13 (m, 2H), 2.04-1.84 | (Method 1) |
| | | | (m, 2H). 2 protons obscured by | |
| | | | solvent peak. | |
| 12 | 1-(2-(3-fluoro-5-(trifluoromethyl)benzyl)pyridin- | Intermediates 2 and 6 | (400 MHz, DMSO-d₆) δ: 8.65 (d, J = | m/z 405.2 |
| | 4-yl)-5,6,7,8-tetrahydropyrazolo[4,3-c]azepin- | Procedure 5 | 5.6 Hz, 1H), 8.05 (s, 1H), 7.83 (s, | (M + H)+ |
| | 4(1H)-one | | 1H), 7.71 (d, J = 1.6 Hz, 1H), 7.61 (s, | (ES+), at |
| | | | 1H), 7.56-7.52 (m, 3H), 4.31 (s, 2H), | 1.94 min, |
| | | | 3.25-3.24 (m, 2H), 3.09 (t, J = 6.4 Hz, | 95% |
| | | | 2H), 1.98-1.93 (m, 2H). | (Method 3) |
| 13 | 1-(4-(3-(trifluoromethyl)benzyl)pyridin-2-yl)-6,7- | Intermediate 7, | (400 MHz, DMSO-d₆) δ: 9.27 (s, 1H), | m/z 389.0 |
| | dihydro-1H-pyrazolo[4,3-f][1,4]oxazepin-4(5H)- | CAS: 87-13-8 and | 8.34 (d, J = 5.2 Hz, 1H), 7.85 (s, 1H), | (M + H)+ |
| | one | 39684-80-5 | 7.77 (s, 1H), 7.72 (s, 1H), 7.63-7.57 | (ES+), at |
| | | Procedure 6 | (m, 3H), 7.24 (d, J = 5.2 Hz, 1H), | 2.37 min, |
| | | | 4.41 (t, J = 3.2 Hz, 2H), 4.20 (s, 2H), | 99% |
| | | | 3.72 (t, 2H). | (Method 3) |
| 14 | 1-(4-(3-fluoro-5-(trifluoromethyl)benzyl)pyridin- | Intermediate 8, | (400 MHz, DMSO-D₆) δ: 9.27 (s, 1H), | m/z 407.1 |
| | 2-yl)-6,7-dihydro-1H-pyrazolo[4,3- | CAS: 87-13-8 and | 8.36 (d, J = 5.2 Hz, 1H), 7.87 (s, 1H), | (M + H)+ |
| | f][1,4]oxazepin-4(5H)-one | 39684-80-5 | 7.82 (s, 1H), 7.63 (s, 1H), 7.58 (d, J = | (ES+), at |
| | | Procedure 6 | 11.2 Hz, 2H), 7.28 (t, J = 1.2 Hz, 1H), | 2.46 min, |
| | | | 4.42 (t, J = 3.6 Hz, 2H), 4.22 (s, 2H), | 99% |
| | | | 3.75-3.70 (m, 2H). | (Method 3) |
| 15 | 1-(5-(3-fluoro-5- | Intermediates 1 and 9 | (400 MHz, DMSO-d₆) δ: 9.28 (d, J = | m/z 391.3 |
| | (trifluoromethyl)benzyl)pyridazin-3-yl)-1,5,6,7- | Procedure 1 | 2.0 Hz, 1H), 8.15 (d, J = 2.0 Hz, 1H), | (M − H)− |
| | tetrahydro-4H-pyrazolo[4,3-c]pyridin-4-one | | 8.10 (s, 1H), 7.72 (s, 1H), 7.66 (d, J = | (ES−), at |
| | | | 10.0 Hz, 1H), 7.59 (d, J = 8.8 Hz, | 1.94 min, |
| | | | 1H), 7.53 (s, 1H), 4.28 (s, 2H), 3.45 | 100% |
| | | | (t, J = 6.8 Hz, 4H). | (Method 3) |
| 16 | 1-(5-(3-fluoro-5-(trifluoromethyl)benzyl)pyridin- | Intermediate 11 and | (400 MHz, DMSO-d₆) δ: 8.73 (d, J = | m/z 391.2 |
| | 3-yl)-1,5,6,7-tetrahydro-4H-pyrazolo[4,3- | CAS: 239087-09-3 | 2.4 Hz, 1H), 8.68 (d, J = 2.0 Hz, 1H), | (M + H)+ |
| | c]pyridin-4-one | Procedure 7 | 8.05-8.04 (m, 1H), 8.02 (s, 1H), 7.68 | (ES+), at |
| | | | (s, 1H), 7.63-7.61 (m, 1H), 7.56-7.54 | 1.91 min, |
| | | | (m, 1H), 7.45 (s, 1H), 4.22 (s, 2H), | 98% |
| | | | 3.43-3.39 (m, 2H), 3.10-3.07 (m, 2H). | (Method 3) |
| 17 | 1-(6-(3-fluoro-5- | Intermediate 10 and | (400 MHz, DMSO-d₆) δ: 9.52 (d, J = | m/z 392.2 |
| | (trifluoromethyl)benzyl)pyridazin-4-yl)-1,5,6,7- | CAS: 239087-09-3 | 2.4 Hz, 1H), 8.15 (s, 1H), 7.99 (d, J = | (M + H)+ |
| | tetrahydro-4H-pyrazolo[4,3-c]pyridin-4-one | Procedure 7 | 2.4 Hz, 1H), 7.69 (s, 1H), 7.62-7.57 | (ES+), at |
| | | | (m, 3H), 4.54 (s, 2H), 3.45-3.43 (m, | 1.93 min, |
| | | | 2H), 3.30-3.25 (m, 2H). | 99% |
| | | | | (Method 3) |
| 18 | 1-(4-(3-fluoro-5- | Intermediate 12 and | (400 MHz, DMSO-d₆) δ: 8.45 (d, J = | m/z 338.2 |
| | (trifluoromethyl)phenoxy)pyridin-2-yl)-1,5,6,7- | CAS: 172333-87-8 | 5.7 Hz, 1H), 7.98 (s, 1H), 7.72-7.66 | (M + H)+ |
| | tetrahydro-4H-pyrazolo[4,3-c]pyridin-4-one | Procedure 8 | (m, 2H), 7.61 (s, 1H), 7.48 (bs, 1H), | (ES+), at |
| | | | 7.42 (d, J = 2.2 Hz, 1H), 7.11 (d, J = | 5.93 min, |
| | | | 5.6 2.2 Hz, 1H), 3.50-3.40 (m, 4H). | 100% |
| | | | | (Method 5) |
| 19 | 1-(2-(4-chlorobenzyl)pyridin-4-yl)-1,5,6,7- | Intermediate 14 and | (400 MHz, DMSO-d₆) δ: 8.63 (d, J = | m/z 339.1 |
| | tetrahydro-4H-pyrazolo[4,3-c]pyridin-4-one | CAS: 622-95-7 | 5.2 Hz, 1H), 8.04 (s, 1H), 7.62 (d, J = | (M + H)+ |
| | | Procedure 9 | 2.0 Hz, 1H), 7.51-7.49 (m, 2H), 7.37 | (ES+), at |
| | | | (s, 4H), 4.39 (s, 2H), 3.45-3.41 (m, | 1.57 min, |
| | | | 2H), 3.20-3.17 (m, 2H). | 98% |
| | | | | (Method 3) |
| 20 | 1-(2-(3-chloro-5-fluorobenzyl)pyridin-4-yl)- | Intermediate 14 and | (400 MHz, DMSO-d₆) δ: 8.65 (d, J = | m/z 356.9 |
| | 1,5,6,7-tetrahydro-4H-pyrazolo[4,3-c]pyridin-4- | CAS: 493024-39-8 | 5.2 Hz, 1H), 8.06 (s, 1H), 7.69 (d, J = | (M + H)+ |
| | one | Procedure 9 | 2.0 Hz, 1H), 7.53-7.53 (m, 2H), 7.31- | (ES+), at |
| | | | 7.30 (m, 2H), 7.22 (d, J = 9.6 Hz, | 1.74 min, |
| | | | 1H), 4.22 (s, 2H), 3.44-3.43 (m, 2H), | 100% |
| | | | 3.20 (t, J = 6.8 Hz, 2H). | (Method 3) |
| 21 | 1-(2-(3,5-difluorobenzyl)pyridin-4-yl)-1,5,6,7- | Intermediate 14 and | (400 MHz, DMSO-d₆) δ: 8.65 (d, J = | m/z 340.9 |
| | tetrahydro-4H-pyrazolo[4,3-c]pyridin-4-one | CAS: 141776-91-2 | 5.2 Hz, 1H), 8.05 (s, 1H), 7.67 (d, J = | (M + H)+ |
| | | Procedure 9 | 2.0 Hz, 1H), 7.53-7.53 (m, 2H), 7.10- | (ES+), at |
| | | | 7.10 (m, 3H), 4.22 (s, 2H), 3.45 (d, | 1.58 min, |
| | | | J = 2.4 Hz, 2H), 3.20 (t, J = 6.8 Hz, | 100% |
| | | | 2H). | (Method 3) |
| 22 | 1-(2-(3-(trifluoromethyl)benzyl)pyridin-4-yl)- | Intermediate 14 and | (400 MHz, DMSO-d₆) δ: 8.64 (d, J = | m/z 373.1 |
| | 1,5,6,7-tetrahydro-4H-pyrazolo[4,3-c]pyridin-4- | CAS: 402-23-3 | 7.2 Hz, 1H), 8.05 (s, 1H), 7.74 (s, | (M + H)+ |
| | one | Procedure 9 | 1H), 7.69-7.55 (m, 3H), 7.52-7.50 (m, | (ES+), at |
| | | | 3H), 4.31 (s, 2H), 3.42-3.40 (m, 2H), | 1.75 min, |
| | | | 3.21-3.16 (m, 2H). | 99% |
| | | | | (Method 3) |
| 23 | 1-(2-(4-fluoro-3-(trifluoromethyl)benzyl)pyridin- | Intermediate 14 and | (400 MHz, DMSO-d₆) δ: 8.64 (d, J = | m/z 391.0 |
| | 4-yl)-1,5,6,7-tetrahydro-4H-pyrazolo[4,3- | CAS: 184970-26-1 | 5.2 Hz, 1H), 8.05 (s, 1H), 7.79 (d, J = | (M + H)+ |
| | c]pyridin-4-one | Procedure 9 | 7.2 Hz, 1H), 7.74-7.72 (m, 2H), 7.52- | (ES+), at |
| | | | 7.52 (m, 3H), 4.28 (s, 2H), 3.45-3.44 | 1.88 min, |
| | | | (m, 2H), 3.19 (t, J = 6.8 Hz, 2H). | 99% |
| | | | | (Method 2) |
| 24 | 1-(2-(3,4,5-trifluorobenzyl)pyridin-4-yl)-1,5,6,7- | Intermediate 14 and | (400 MHz, DMSO-d₆) δ: 8.65 (d, J = | m/z 359.1 |
| | tetrahydro-4H-pyrazolo[4,3-c]pyridin-4-one | CAS: 220141-72-0 | 5.6 Hz, 1H), 8.06 (s, 1H), 7.67 (d, J = | (M + H)+ |
| | | Procedure 9 | 2.0 Hz, 1H), 7.54-7.53 (m, 2H), 7.35- | (ES+), at |
| | | | 7.34 (m, 2H), 4.19 (s, 2H), 3.46-3.45 | 1.69 min, |
| | | | (m, 2H), 3.20 (t, J = 6.8 Hz, 2H). | 100% |
| | | | | (Method 3) |
| 25 | 1-(2-(3-chloro-4-fluorobenzyl)pyridin-4-yl)- | Intermediate 14 and | (400 MHz, DMSO-d₆) δ: 8.64 (d, J = | m/z 357.1 |
| | 1,5,6,7-tetrahydro-4H-pyrazolo[4,3-c]pyridin-4- | CAS: 192702-01-5 | 5.6 Hz, 1H), 8.05 (s, 1H), 7.66 (d, J = | (M + H)+ |
| | one | Procedure 9 | 1.6 Hz, 1H), 7.58 (d, J = 7.6 Hz, 1H), | (ES+), at |
| | | | 7.5-7.51 (m, 2H), 7.36-7.36 (m, 2H), | 1.68 min, |
| | | | 4.19 (s, 2H), 3.45-3.44 (m, 2H), 3.19 | 100% |
| | | | (t, J = 6.4 Hz, 2H). | (Method 3) |
| 26 | 1-(2-(3-fluoro-5-methylbenzyl)pyridin-4-yl)- | Intermediate 14 and | (400 MHz, CDCl₃) δ: 8.71 (d, J = 5.2 | m/z 337.1 |
| | 1,5,6,7-tetrahydro-4H-pyrazolo[4,3-c]pyridin-4- | CAS: 212268-39-8 | Hz, 1H), 8.14 (s, 1H), 7.37-7.35 (m, | (M + H)+ |
| | one | Procedure 9 | 2H), 6.92 (s, 1H), 6.80 (d, J = 18.4 | (ES+), at |
| | | | Hz, 2H), 5.58 (s, 1H), 4.21 (s, 2H), | 1.69 min, |
| | | | 3.67-3.63 (m, 2H), 3.15-3.12 (m, 2H), | 100% |
| | | | 2.35 (s, 3H). | (Method 2) |
| 27 | 1-(2-(3-chloro-5-(trifluoromethyl)benzyl)pyridin- | Intermediate 14 and | (400 MHz, DMSO-d₆) δ: 8.65 (d, J = | m/z 407.0 |
| | 4-yl)-1,5,6,7-tetrahydro-4H-pyrazolo[4,3- | CAS: 886496-91-9 | 5.2 Hz, 1H), 8.06 (s, 1H), 7.78 (s, | (M + H)+ |
| | c]pyridin-4-one | Procedure 9 | 1H), 7.73 (t, J = 6.0 Hz, 3H), 7.54- | (ES+), at |
| | | | 7.50 (m, 2H), 4.32 (s, 2H), 3.46-3.27 | 2.03 min, |
| | | | (m, 2H), 3.20 (t, J = 6.8 Hz, 2H). | 100% |
| | | | | (Method 2) |
| 28 | 1-(2-(3-methyl-5-(trifluoromethyl)benzyl)pyridin- | Intermediate 14 and | (400 MHz, DMSO-d₆) δ: 8.67 (d, J = | m/z 387.1 |
| | 4-yl)-1,5,6,7-tetrahydro-4H-pyrazolo[4,3- | CAS: 116070-36-1 | 5.6 Hz, 1H), 8.07 (s, 1H), 7.72 (d, J = | (M + H)+ |
| | c]pyridin-4-one | Procedure 9 | 2.0 Hz, 1H), 7.57-7.51 (m, 3H), 7.48 | (ES+), at |
| | | | (s, 1H), 7.42 (s, 1H), 4.28 (s, 2H), | 1.94 min, |
| | | | 3.45-3.41 (m, 2H), 3.19 (t, J = 6.4 Hz, | 98% |
| | | | 2H), 2.37 (s, 3H). | (Method 2) |
| 29 | 1-(2-(2-fluoro-3-(trifluoromethyl)benzyl)pyridin- | Intermediate 14 and | (400 MHz, DMSO-d₆) δ: 8.63 (d, J = | m/z 391.1 |
| | 4-yl)-1,5,6,7-tetrahydro-4H-pyrazolo[4,3- | CAS: 184970-25-0 | 5.6 Hz, 1H), 8.06 (s, 1H), 7.76-7.69 | (M + H)+ |
| | c]pyridin-4-one | Procedure 9 | (m, 1H), 7.66 (d, J = 2.0 Hz, 2H), | (ES+), at |
| | | | 7.54-7.50 (m, 2H), 7.39 (t, J = 7.6 Hz, | 1.87 min, |
| | | | 1H), 4.34 (s, 2H), 3.46-3.42 (m, 2H), | 99% |
| | | | 3.19 (t, J = 6.4 Hz, 2H). | (Method 2) |
| 30 | 1-(2-(2-fluoro-5-(trifluoromethyl)benzyl)pyridin- | Intermediate 14 and | (400 MHz, DMSO-d₆) δ: 8.62 (d, J = | m/z 391.0 |
| | 4-yl)-1,5,6,7-tetrahydro-4H-pyrazolo[4,3- | CAS: 220239-69-0 | 5.6 Hz, 1H), 8.08 (d, J = 17.6 Hz, | (M + H)+ |
| | c]pyridin-4-one | Procedure 9 | 1H), 7.87 (d, J = 5.6 Hz, 1H), 7.72 (d, | (ES+), at |
| | | | J = 3.6 Hz, 1H), 7.66 (s, 1H), 7.52 (d, | 1.90 min, |
| | | | J = 6.0 Hz, 2H), 7.47-7.42 (m, 1H), | 94% |
| | | | 4.34 (s, 2H), 3.45-3.42 (m, 2H), 3.20- | (Method 2) |
| | | | 3.17 (m, 2H). | |
| 31 | 1-(2-(3,5-dichlorobenzyl)pyridin-4-yl)-1,5,6,7- | Intermediate 14 and | (400 MHz, DMSO-d₆) δ: 8.65 (d, J = | m/z 372.9 |
| | tetrahydro-4H-pyrazolo[4,3-c]pyridin-4-one | CAS: 7778-01-0 | 5.6 Hz, 1H), 8.06 (s, 1H), 7.70 (d, J = | (M + H)+ |
| | | Procedure 9 | 1.6 Hz, 1H), 7.53-7.51 (m, 2H), 7.48- | (ES+), at |
| | | | 7.44 (m, 3H), 4.21 (s, 2H), 3.45-3.38 | 1.94 min, |
| | | | (m, 2H), 3.22-3.17 (m, 2H). | 100% |
| | | | | (Method 2) |
| 32 | 1-(2-(4-(trifluoromethyl)benzyl)pyridin-4-yl)- | Intermediate 14 and | (400 MHz, DMSO-d₆) δ: 8.64 (d, J = | m/z 373.2 |
| | 1,5,6,7-tetrahydro-4H-pyrazolo[4,3-c]pyridin-4- | CAS: 402-49-3 | 5.2 Hz, 1H), 8.05 (s, 1H), 7.68 (t, J = | (M + H)+ |
| | one | Procedure 9 | 2.4 Hz, 3H), 7.57 (d, J = 8.0 Hz, 2H), | (ES+), at |
| | | | 7.52-7.52 (m, 2H), 4.30 (s, 2H), 3.45- | 1.81 min, |
| | | | 3.44 (m, 2H), 3.19 (t, J = 6.8 Hz, 2H). | 99% |
| | | | | (Method 3) |
| 33 | 3-((4-(4-oxo-4,5,6,7-tetrahydro-1H- | Intermediate 14 and | (400 MHz, DMSO-d₆) δ: 8.65 (d, J = | m/z 397.9 |
| | pyrazolo[4,3-c]pyridin-1-yl)pyridin-2-yl)methyl)- | CAS: 954123-46-7 | 5.6 Hz, 1H), 8.22 (s, 1H), 8.17 (s, | (M + H)+ |
| | 5-(trifluoromethyl)benzonitrile | Procedure 10 | 1H), 8.12 (s, 1H), 8.07 (s, 1H), 7.73 | (ES+), at |
| | | | (d, J = 1.6 Hz, 1H), 7.55-7.51 (m, | 2.64 min, |
| | | | 2H), 4.38 (s, 2H), 3.46-3.42 (m, 2H), | 99% |
| | | | 3.20 (t, J = 6.8 Hz, 2H). | (Method 6) |
| 34 | 1-(2-(3,5-bis(trifluoromethyl)benzyl)pyridin-4- | Intermediate 14 and | (400 MHz, DMSO-d₆) δ: 8.65 (d, J = | m/z 441.0 |
| | yl)-1,5,6,7-tetrahydro-4H-pyrazolo[4,3- | CAS: 32247-96-4 | 5.6 Hz, 1H), 8.11 (s, 2H), 8.06 (s, | (M + H)+ |
| | c]pyridin-4-one | Procedure 9 | 1H), 7.98 (s, 1H), 7.77-7.76 (m, 1H), | (ES+), at |
| | | | 7.54-7.54 (m, 2H), 4.43 (s, 2H), 3.45- | 1.20 min, |
| | | | 3.44 (m, 2H), 3.20 (t, J = 6.8 Hz, 2H). | 98% |
| | | | | (Method 2) |
| 35 | 1-(2-(3-fluoro-5- | Example 1 | (400 MHz, DMSO-d₆) δ: 8.91 (d, J = | m/z 405.0 |
| | (trifluoromethyl)benzoyl)pyridin-4-yl)-1,5,6,7- | Procedure 11 | 5.6 Hz, 1H), 8.32 (d, J = 2.0 Hz, 1H), | (M + H)+ |
| | tetrahydro-4H-pyrazolo[4,3-c]pyridin-4-one | | 8.21-8.18 (m, 2H), 8.15 (s, 1H), 8.09 | (ES+), at |
| | | | (d, J = 8.4 Hz, 1H), 8.01-7.99 (m, | 2.05 min, |
| | | | 1H), 7.57 (s, 1H), 3.48-3.46 (m, 2H), | 98% |
| | | | 3.30 (t, J = 6.4 Hz, 2H). | (Method 2) |
| 36 | 1-(2-((3-fluoro-5- | Example 1 | (400 MHz, DMSO-d₆) δ: 8.64 (d, J = | m/z 407.0 |
| | (trifluoromethyl)phenyl)(hydroxy)methyl)pyridin- | Procedure 11 | 5.6 Hz, 1H), 8.07 (s, 1H), 7.93 (d, J = | (M + H)+ |
| | 4-yl)-1,5,6,7-tetrahydro-4H-pyrazolo[4,3- | | 2.0 Hz, 1H), 7.70 (s, 1H), 7.63-7.52 | (ES+), at |
| | c]pyridin-4-one | | (m, 4H), 6.66 (d, J = 4.0 Hz, 1H), | 1.76 min, |
| | | | 5.96 (d, J = 2.8 Hz, 1H), 3.45-3.43 | 98% |
| | | | (m, 2H), 3.22-3.21 (m, 2H). | (Method 2) |
| 36 | 1-(2-((3-fluoro-5- | Example 36 | (400 MHz, DMSO-d₆) δ: 8.64 (d, J = | m/z 406.9 |
| Isomer 1 | (trifluoromethyl)phenyl)(hydroxy)methyl)pyridin- | Chiral SFC method 1 | 5.2 Hz, 1H), 8.07 (s, 1H), 7.93 (d, J = | (M + H)+ |
| | 4-yl)-1,5,6,7-tetrahydro-4H-pyrazolo[4,3- | (Isocratic run 20% co- | 2.0 Hz, 1H), 7.70 (s, 1H), 7.55-7.53 | (ES+), at |
| | c]pyridin-4-one | solvent) | (m, 4H), 6.66 (d, J = 4.8 Hz, 1H), | 2.09 min, |
| | (Single enantiomer of unknown absolute | Chiral SFC purity analysis: | 5.96 (d, J = 4.8 Hz, 1H), 3.45-3.36 | 99% |
| | stereochemistry) | 4.75 min, 100% | (m, 2H), 3.22-3.21 (m, 2H). | (Method 2) |
| | | (Method 3 - isocratic run | | |
| | | 20% co-solvent) | | |
| 36 | 1-(2-((3-fluoro-5- | Example 36 | (400 MHz, DMSO-d₆) δ: 8.64 (d, J = | m/z 406.9 |
| Isomer 2 | (trifluoromethyl)phenyl)(hydroxy)methyl)pyridin- | Chiral SFC method 1 | 5.2 Hz, 1H), 8.07 (s, 1H), 7.93 (d, J = | (M + H)+ |
| | 4-yl)-1,5,6,7-tetrahydro-4H-pyrazolo[4,3- | (Isocratic run 20% co- | 2.0 Hz, 1H), 7.70 (s, 1H), 7.63-7.52 | (ES+), at |
| | c]pyridin-4-one | solvent) | (m, 4H), 6.66 (d, J = 4.4 Hz, 1H), | 2.09 min, |
| | (Single enantiomer of unknown absolute | Chiral SFC purity analysis: | 5.96 (d, J = 4.4 Hz, 1H), 3.46-3.43 | 99% |
| | stereochemistry) | 5.60 min, 99% (Method 3 - | (m, 2H), 3.24-3.21 (m, 2H). | (Method 2) |
| | | isocratic run 20% co- | | |
| | | solvent) | | |
| 37 | 1-(2-(fluoro(3-fluoro-5- | Example 1 | (400 MHz, DMSO-d₆) δ: 8.73 (d, J = | m/z 409.2 |
| | (trifluoromethyl)phenyl)methyl)pyridin-4-yl)- | Procedure 11 | 5.2 Hz, 1H), 8.10 (s, 1H), 7.97 (d, J = | (M + H)+ |
| | 1,5,6,7-tetrahydro-4H-pyrazolo[4,3-c]pyridin-4- | | 2.0 Hz, 1H), 7.77-7.67 (m, 4H), 7.54 | (ES+), at |
| | one | | (s, 1H), 6.96 (d, J = 45.2 Hz, 1H), | 2.21 min, |
| | | | 3.47-3.43 (m, 2H), 3.33-3.30 (m, 2H). | 97% |
| | | | | (Method 7) |
| 38 | 1-(2-(difluoro(3-fluoro-5- | Example 35 | (400 MHz, DMSO-d₆) δ: 8.81 (d, J = | m/z 427.1 |
| | (trifluoromethyl)phenyl)methyl)pyridin-4-yl)- | Procedure 12 | 5.2 Hz, 1H), 8.17 (d, J = 2.0 Hz, 1H), | (M + H)+ |
| | 1,5,6,7-tetrahydro-4H-pyrazolo[4,3-c]pyridin-4- | | 8.14 (s, 1H), 7.97-7.91 (m, 2H), 7.86- | (ES+), at |
| | one | | 7.83 (m, 2H), 7.56 (s, 1H), 3.48-3.44 | 2.22 min, |
| | | | (m, 2H), 3.30-3.27 (m, 2H). | 93% |
| | | | | (Method 3) |
| 39 | 1-(2-((3-fluoro-5-(trifluoromethyl)phenyl)methyl- | Example 1 | (400 MHz, DMSO-d₆) δ: 8.67 (d, J = | m/z 393.0 |
| | d₂)pyridin-4-yl)-1,5,6,7-tetrahydro-4H- | Procedure 13 | 7.6 Hz, 1H), 8.06 (s, 1H), 7.84 (s, | (M + H)+ |
| | pyrazolo[4,3-c]pyridin-4-one | | 1H), 7.63 (s, 1H), 7.55-7.50 (m, 4H), | (ES+), at |
| | | | 3.46-3.42 (m, 2H), 3.22-3.17 (m, 2H). | 1.94 min, |
| | | | | 100% |
| | | | | (Method 7) |
| 40 | 1-(2-(3-fluoro-5-(trifluoromethyl)benzyl)pyridin- | Intermediates 6 and 17 | (400 MHz, DMSO-d₆) δ: 8.64 (d, J = | m/z 405.2 |
| | 4-yl)-6-methyl-1,5,6,7-tetrahydro-4H- | Procedure 1 | 5.4 Hz, 1H), 8.04 (s, 1H), 7.69 (d, J = | (M + H)+ |
| | pyrazolo[4,3-c]pyridin-4-one | | 1.8 Hz, 1H), 7.62 (s, 1H), 7.57-7.48 | (ES+), at |
| | | | (m, 4H), 4.32 (s, 2H), 3.77 (dt, J = | 5.51 min, |
| | | | 11.4, 5.9 Hz, 1H), 3.21 (dd, J = 16.4, | 98% |
| | | | 5 Hz, 1H), 2.97 (dd, J = 16.5, 10.6 | (Method 5) |
| | | | Hz, 1H), 1.23 (d, J = 6.4 Hz, 3H). | |
| 40 | 1-(2-(3-fluoro-5-(trifluoromethyl)benzyl)pyridin- | Example 40 | (400 MHz, DMSO-d₆) δ: 8.65 (dd, J = | m/z 405.2 |
| Isomer 1 | 4-yl)-6-methyl-1,5,6,7-tetrahydro-4H- | Chiral SFC method 2 | 5.5, 0.6 Hz, 1H), 8.05 (s, 1H), 7.70 | (M + H)+ |
| | pyrazolo[4,3-c]pyridin-4-one | (Isocratic run 30% co- | (dd, J = 2.2, 0.7 Hz, 1H), 7.63 (td, J = | (ES+), at |
| | (Single enantiomer of unknown absolute | solvent) | 1.5, 0.8 Hz, 1H), 7.60-7.37 (m, 4H), | 3.97 min, |
| | stereochemistry) | Chiral SFC purity analysis: | 4.33 (s, 2H), 3.86-3.71 (m, 1H), 3.20 | 100% |
| | | 1.94 min, 99% (Method 4 - | (dd, J = 16.4, 5.1 Hz, 1H), 2.98 (dd, | (Method 1) |
| | | gradient run 3-50% co- | J = 16.5, 10.6 Hz, 1H), 1.24 (d, J = 6.4 | |
| | | solvent) | Hz, 3H). | |
| 40 | 1-(2-(3-fluoro-5-(trifluoromethyl)benzyl)pyridin- | Example 40 | (400 MHz, DMSO-d₆) δ: 8.65 (dd, J = | m/z 405.1 |
| Isomer 2 | 4-yl)-6-methyl-1,5,6,7-tetrahydro-4H- | Chiral SFC method 2 | 5.5, 0.6 Hz, 1H), 8.05 (s, 1H), 7.70 | (M + H)+ |
| | pyrazolo[4,3-c]pyridin-4-one | (Isocratic run 30% co- | (dd, J = 2.1, 0.7 Hz, 1H), 7.63 (tt, J = | (ES+), at |
| | (Single enantiomer of unknown absolute | solvent) | 1.6, 0.7 Hz, 1H), 7.60-7.38 (m, 4H), | 3.97 min, |
| | stereochemistry) | Chiral SFC purity analysis: | 4.33 (s, 2H), 3.84-3.71 (m, 1H), 3.20 | 100% |
| | | 2.02 min, 99% (Method 4 - | (dd, J = 16.4, 5.1 Hz, 1H), 2.98 (dd, | (Method 1) |
| | | gradient run 3-50% co- | J = 16.5, 10.6 Hz, 1H), 1.24 (d, J = 6.5 | |
| | | solvent) | Hz, 3H). | |
| 41 | 1-(4-(3-(trifluoromethyl)benzyl)pyridin-2-yl)- | Intermediates 4 and 15 | (400 MHz, DMSO-d₆) δ: 8.54 (d, J = | m/z 388.2 |
| | 5,6,7,8-tetrahydro-[1,2,3]triazolo[4,5-c]azepin- | Procedure 14 | 5.0 Hz, 1H), 8.09 (t, J = 4.9 Hz, 1H), | (M + H)+ |
| | 4(1H)-one | | 7.93 (s, 1H), 7.78 (s, 1H), 7.67 (d, J = | (ES+), at |
| | | | 7.3 Hz, 1H), 7.64-7.55 (m, 2H), 7.52 | 5.47 min, |
| | | | (d, J = 4.7 Hz, 1H), 4.26 (s, 2H), | 99% |
| | | | 3.26-3.19 (m, 4H), 2.00-1.94 (m, 2H). | (Method 8) |
| 42 | 1-(4-(3-(trifluoromethyl)benzyl)pyridin-2-yl)- | Intermediates 4 and 16 | (400 MHz, DMSO-d₆) δ: 8.53 (d, J = | m/z 374.0 |
| | 1,5,6,7-tetrahydro-4H-[1,2,3]triazolo[4,5- | Procedure 15 | 4.9 Hz, 1H), 8.07 (bs, 1H), 7.82-7.72 | (M + H)+ |
| | c]pyridin-4-one | | (m, 2H), 7.72-7.65 (m, 1H), 7.63-7.53 | (ES+), at |
| | | | (m, 2H), 7.50 (d, J = 4.8 Hz, 1H), | 5.65 min, |
| | | | 4.27 (s, 2H), 3.51-3.45 (m, 2H), 3.43- | 100% |
| | | | 3.37 (m, 2H). | (Method 5) |
| 43 | 2-(4-(3-(trifluoromethyl)benzyl)pyridin-2-yl)- | Intermediates 4 and 16 | (400 MHz, DMSO-d₆) δ: 8.50 (d, J = | m/z 374.1 |
| | 2,5,6,7-tetrahydro-4H-[1,2,3]triazolo[4,5- | Procedure 15 | 4.9 Hz, 1H), 8.12 (bs, 1H), 7.98 (s, 1H), | (M + H)+ |
| | c]pyridin-4-one | | 7.76 (s, 1H), 7.70-7.63 (m, 1H), 7.62- | (ES+), at |
| | | | 7.55 (m, 2H), 7.45 (d, J = 4.5 Hz, | 5.44 min, |
| | | | 1H), 4.25 (s, 2H), 3.60-3.50 (m, 2H), | 100% |
| | | | 3.10-3.00 (m, 2H). | (Method 5) |
| 44 | 5-methyl-2-(4-(3-(trifluoromethyl)benzyl)pyridin- | Example 43 | (400 MHz, DMSO-d₆) δ: 8.49 (d, J = | m/z 388.1 |
| | 2-yl)-2,5,6,7-tetrahydro-4H-[1,2,3]triazolo[4,5- | Procedure 2 | 5.0 Hz, 1H), 7.99 (s, 1H), 7.77 (s, | (M + H)+ |
| | c]pyridin-4-one | | 1H), 7.66 (d, J = 7.3 Hz, 1H), 7.59 | (ES+), at |
| | | | (dt, J = 15.1, 7.6 Hz, 2H), 7.49-7.40 | 5.62 min, |
| | | | (m, 1H), 4.26 (s, 2H), 3.73 (t, J = 6.8 | 99% |
| | | | Hz, 2H), 3.13 (t, J = 6.8 Hz, 2H), 3.03 | (Method 5) |
| | | | (s, 3H). | |
| 45 | 2-(4-(3-(trifluoromethyl)benzyl)pyridin-2-yl)- | Intermediates 4 and 15 | (400 MHz, DMSO-d₆) δ: 8.49 (d, J = | m/z 388.2 |
| | 5,6,7,8-tetrahydro-[1,2,3]triazolo[4,5-c]azepin- | Procedure 14 | 4.9 Hz, 1H), 8.29 (t, J = 4.8 Hz, 1H), | (M + H)+ |
| | 4(2H)-one | | 7.97 (s, 1H), 7.75 (s, 1H), 7.65 (d, J = | (ES+), at |
| | | | 7.2 Hz, 1H), 7.62-7.55 (m, 2H), 7.45 | 5.74 min, |
| | | | (d, J = 4.8 Hz, 1H), 4.26 (s, 2H), 3.26 | 99% |
| | | | (t, J = 4.4 Hz, 2H), 3.06 (t, J = 6.7 Hz, | (Method 8) |
| | | | 2H), 2.05-1.98 (m, 2H). | |
| 46 | 1-(4-(3-fluoro-5-(trifluoromethyl)benzyl)pyridin- | Intermediate 18 and | (400 MHz, DMSO-d₆) δ: 8.55 (d, J = | m/z 406.1 |
| | 2-yl)-5,6,7,8-tetrahydro-[1,2,3]triazolo[4,5- | CAS: 5220-49-5 | 5.0 Hz, 1H), 8.09 (t, J = 4.8 Hz, 1H), | (M + H)+ |
| | c]azepin-4(1H)-one | Procedure 16 | 7.97 (s, 1H), 7.69 (s, 1H), 7.66-7.61 | (ES+), at |
| | | | (m, 1H), 7.59-7.54 (m, 2H), 4.27 (s, | 5.59 min, |
| | | | 2H), 3.28-3.20 (m, 4H), 2.02-1.91 (m, | 96% |
| | | | 2H). | (Method 5) |
| 47 | 1-(4-(3-fluoro-5-(trifluoromethyl)benzyl)pyridin- | Intermediates 8 and 19 | (400 MHz, DMSO-d₆) δ: 10.68 (s, | m/z 393.0 |
| | 2-yl)-1,7-dihydropyrazolo[4,3-c/][1,2]oxazin- | Procedure 17 | 1H), 8.41 (d, J = 5.08 Hz, 1H), 8.19 | (M + H)+ |
| | 4(5H)-one | | (s, 1H), 7.93 (s, 1H), 7.66 (s, 1H), | (ES+), at |
| | | | 7.63-7.54 (m, 2H), 7.41-7.36 (m, 1H), | 6.51 min, |
| | | | 5.52 (s, 2H), 4.25 (s, 2H). | 99% |
| | | | | (Method 8) |
| 48 | 1-(2-(3-fluoro-5-(trifluoromethyl)benzyl)pyridin- | Intermediates 6 and 19 | (400 MHz, DMSO-d₆) δ: 8.61 (d, J = | m/z 377.0 |
| | 4-yl)-5,6-dihydropyrrolo[3,4-c]pyrazol-4(1H)- | Procedure 18 | 5.6 Hz, 1H), 8.30 (s, 1H), 8.03 (s, | (M + H)+ |
| | one | | 1H), 7.71 (d, J = 2.0 Hz, 1H), 7.61 (s, | (ES+), at |
| | | | 1H), 7.56-7.49 (m, 3H), 4.79 (s, 2H), | 5.35 min, |
| | | | 4.31 (s, 2H). | 96% |
| | | | | (Method 5) |
| 49 | 1-(2-((3-fluoro-5-(trifluoromethyl)phenyl)methyl- | Example 35 | (400 MHz, DMSO-d₆) δ: 8.66 (d, J = | m/z 392.0 |
| | d)pyridin-4-yl)-1,5,6,7-tetrahydro-4H- | Procedure 19 | 5.6 Hz, 1H), 8.07 (d, J = 4.8 Hz, 1H), | (M + H)+ |
| | pyrazolo[4,3-c]pyridin-4-one | | 7.73 (d, J = 1.6 Hz, 1H), 7.63 (s, 1H), | (ES+), at |
| | | | 7.57-7.53 (m, 3H), 7.50 (s, 1H), 4.32 | 1.91 min, |
| | | | (d, J = 8.4 Hz, 1H), 3.52-3.43 (m, | 96% |
| | | | 2H), 3.22-3.18 (m, 2H). | (Method 7) |
| 50 | 1-(2-(3-fluoro-5-(trifluoromethyl)benzyl)pyridin- | Intermediates 6 and 20 | (400 MHz, DMSO-d₆) δ: 8.66 (d, J = | m/z 405.2 |
| | 4-yl)-7-methyl-1,5,6,7-tetrahydro-4H- | Procedure 1 | 5.5 Hz, 1H), 8.03 (s, 1H), 7.74 (d, J = | (M + H)+ |
| | pyrazolo[4,3-c]pyridin-4-one | | 2.1 Hz, 1H), 7.65-7.43 (m, 5H), 4.33 | (ES+), at |
| | | | (s, 2H), 3.66 (dd, J = 12.6, 4.7 Hz, | 5.22 min, |
| | | | 1H), 3.63-3.53 (m, 1H), 3.17 (ddd, J = | 97% |
| | | | 12.5, 4.5, 2.1 Hz, 1H), 1.11 (d, J = | (Method 8) |
| | | | 6.8 Hz, 3H). | |
| 51 | 1-(2-(3-fluoro-5-(trifluoromethyl)benzyl)pyridin- | Intermediate 21 and | (400 MHz, DMSO-d₆) δ: 8.61 (d, J = | m/z 389.9 |
| | 4-yl)-1,5,6,7-tetrahydro-4H-pyrrolo[3,2- | CAS: 239087-09-3 | 5.2 Hz, 1H), 7.63 (s, 1H), 7.58 (d, J = | (M + H)+ |
| | c]pyridin-4-one | Procedures 7 and 10, step | 1.6 Hz, 1H), 7.55-7.53 (m, 2H), 7.40 | (ES+), at |
| | | 4 | (d, J = 2.0 Hz, 1H), 7.23 (d, J = 3.2 | 2.11 min, |
| | | | Hz, 1H), 7.20 (s, 1H), 6.52 (d, J = 3.2 | 97% |
| | | | Hz, 1H), 4.28 (s, 2H), 3.38 (t, J = 2.4 | (Method 2) |
| | | | Hz, 2H), 2.94 (t, J = 6.8 Hz, 2H). | |
| 52 | 1-(2-(3-fluoro-5- | Intermediate 13 and | (400 MHz, DMSO-d₆) δ: 8.33 (d, J = | m/z 393.2 |
| | (trifluoromethyl)phenoxy)pyridin-4-yl)-1,5,6,7- | CAS: 172333-87-8 | 5.6 Hz, 1H), 8.09 (s, 1H), 7.63-7.56 | (M + H)+ |
| | tetrahydro-4H-pyrazolo[4,3-c]pyridin-4-one | Procedure 8 | (m, 2H), 7.55-7.50 (m, 3H), 7.38 (d, | (ES+), at |
| | | | J = 1.6 Hz, 1H), 3.44 (t, J = 6.8 Hz, | 5.64 min, |
| | | | 2H), 2.24 (t, J = 6.7 Hz, 2H). | 96% |
| | | | | (Method 5) |
| 53 | 1-(2-(3-cyclopropyl-5-fluorobenzyl)pyridin-4-yl)- | Intermediates 14 and 22 | (400 MHz, DMSO-d₆) δ: 8.64 (d, J = | m/z 363.1 |
| | 1,5,6,7-tetrahydro-4H-pyrazolo[4,3-c]pyridin-4- | Procedure 9 | 5.6 Hz, 1H), 8.05 (s, 1H), 7.62 (d, J = | (M + H)+ |
| | one | | 1.6 Hz, 1H), 7.51-7.50 (m, 2H), 6.9- | (ES+), at |
| | | | 6.93 (m, 2H), 6.73-6.72 (m, 1H), 4.14 | 1.88 min, |
| | | | (s, 2H), 3.45-3.44 (m, 2H), 3.18 (t, J = | 100% |
| | | | 6.8 Hz, 2H), 1.95-1.94 (m, 1H), 0.98- | (Method 2) |
| | | | 0.97 (m, 2H), 0.69 (d, J = 4.4 Hz, | |
| | | | 2H). | |
| 54 | 1-(2-(3-fluoro-4-(trifluoromethyl)benzyl)pyridin- | Intermediate 14 and | (400 MHz, CDCl₃) δ: 8.73 (d, J = 5.6 | m/z 391.2 |
| | 4-yl)-1,5,6,7-tetrahydro-4H-pyrazolo[4,3- | CAS: 213203-65-7 | Hz, 1H), 8.16 (s, 1H), 7.60-7.54 (m, | (M + H)+ |
| | c]pyridin-4-one | Procedure 9 | 1H), 7.52 (d, J = 1.6 Hz, 1H), 7.37 (d, | (ES+), at |
| | | | J = 3.6 Hz, 1H), 7.24-7.17 (m, 2H), | 1.97 min, |
| | | | 5.56 (s, 1H), 4.32 (s, 2H), 3.70-3.66 | 97% |
| | | | (m, 2H), 3.21-3.18 (m, 2H). | (Method 3) |
| 55 | 1-(2-(4-methoxy-3- | Intermediate 14 and | (400 MHz, CDCl₃) δ: 8.71 (d, J = 5.6 | m/z 403.2 |
| | (trifluoromethyl)benzyl)pyridin-4-yl)-1,5,6,7- | CAS: 261951-89-7 | Hz, 1H), 8.14 (s, 1H), 7.52 (d, J = 1.6 | (M + H)+ |
| | tetrahydro-4H-pyrazolo[4,3-c]pyridin-4-one | Procedure 9 | Hz, 1H), 7.48 (d, J = 8.8 Hz, 1H), | (ES+), at |
| | | | 7.37 (d, J = 5.6 Hz, 1H), 7.34 (s, 1H), | 1.71 min, |
| | | | 7.00 (d, J = 8.4 Hz, 1H), 5.52 (s, 1H), | 99% |
| | | | 4.25 (s, 2H), 3.92 (s, 3H), 3.67-3.66 | (Method 3) |
| | | | (m, 2H), 3.13 (t, J = 6.8 Hz, 2H). | |
| 56 | 1-(6-(3-fluoro-5- | Intermediate 23 and | (400 MHz, DMSO-d₆) δ: 9.04 (d, J = | m/z 392.1 |
| | (trifluoromethyl)benzyl)pyrimidin-4-yl)-1,5,6,7- | CAS: 239087-09-3 | 1.2 Hz, 1H), 8.12 (s, 1H), 8.01 (d, J = | (M + H)+ |
| | tetrahydro-4H-pyrazolo[4,3-c]pyridin-4-one | Procedure 9 | 0.8 Hz, 1H), 7.66 (s, 1H), 7.61-7.56 | (ES+), at |
| | | | (m, 3H), 4.36 (s, 2H), 3.52-3.42 (m, | 2.01 min, |
| | | | 4H). | 95% |
| | | | | (Method 3) |
| 57 | 1-(2-(3-methoxy-5- | Intermediate 14 and | (400 MHz, DMSO-d₆) δ: 8.65 (d, J = | m/z 402.9 |
| | (trifluoromethyl)benzyl)pyridin-4-yl)-1,5,6,7- | CAS: 916421-00-6 | 5.6 Hz, 1H), 8.05 (s, 1H), 7.68 (d, J = | (M + H)+ |
| | tetrahydro-4H-pyrazolo[4,3-c]pyridin-4-one | Procedure 9 | 2.0 Hz, 1H), 7.52-7.52 (m, 2H), 7.30 | (ES+), at |
| | | | (s, 1H), 7.24 (s, 1H), 7.10 (s, 1H), | 2.07 min, |
| | | | 4.26 (s, 2H), 3.82 (s, 3H), 3.45-3.44 | 99% |
| | | | (m, 2H), 3.19 (t, J = 6.8 Hz, 2H). | (Method 2) |
| 58 | 1-(2-(4-chloro-3,5-difluorobenzyl)pyridin-4-yl)- | Intermediate 14 and | (400 MHz, DMSO-δ₆) δ: 8.65 (d, J = | m/z 375.0 |
| | 1,5,6,7-tetrahydro-4H-pyrazolo[4,3-c]pyridin-4- | CAS: 1400991-56-1 | 5.6 Hz, 1H), 8.06 (s, 1H), 7.69 (d, J = | (M + H)+ |
| | one | Procedure 9 | 1.6 Hz, 1H), 7.54-7.52 (m, 2H), 7.34- | (ES+), at |
| | | | 7.32 (m, 2H), 4.23 (s, 2H), 3.44-3.42 | 1.89 min, |
| | | | (m, 2H), 3.22-3.19 (m, 2H). | 98% |
| | | | | (Method 7) |
| 59 | 1-(2-(benzo[b]thiophen-5-ylmethyl)pyridin-4-yl)- | Intermediate 14 and | (400 MHz, DMSO-d₆) δ: 8.63 (t, J = | m/z 361.1 |
| | 1,5,6,7-tetrahydro-4H-pyrazolo[4,3-c]pyridin-4- | CAS: 10133-22-9 | 5.2 Hz, 1H), 8.03 (s, 1H), 7.93 (d, J = | (M + H)+ |
| | one | Procedure 9 | 8.4 Hz, 1H), 7.84 (d, J = 1.2 Hz, 1H), | (ES+), at |
| | | | 7.74 (d, J = 5.2 Hz, 1H), 7.63 (d, J = | 1.91 min, |
| | | | 2.0 Hz, 1H), 7.50-7.48 (m, 2H), 7.43- | 99% |
| | | | 7.42 (m, 1H), 7.37-7.35 (m, 1H), 4.31 | (Method 6) |
| | | | (s, 2H), 3.43-3.40 (m, 2H), 3.17 (t, J = | |
| | | | 6.8 Hz, 2H). | |
| 60 | 1-(2-(3-methoxy-4- | Intermediate 14 and | (400 MHz, DMSO-d₆) δ: 8.65 (d, J = | m/z 403.1 |
| | (trifluoromethyl)benzyl)pyridin-4-yl)-1,5,6,7- | CAS: 853367-87-0 | 5.6 Hz, 1H), 8.05 (s, 1H), 7.67 (d, J = | (M + H)+ |
| | tetrahydro-4H-pyrazolo[4,3-c]pyridin-4-one | Procedure 9 | 1.6 Hz, 1H), 7.55-7.51 (m, 3H), 7.29 | (ES+), at |
| | | | (s, 1H), 7.03 (d, J = 8.4 Hz, 1H), 4.26 | 2.01 min, |
| | | | (s, 2H), 3.88 (s, 3H), 3.44-3.41 (m, | 100% |
| | | | 2H), 3.19 (t, J = 6.8 Hz, 2H). | (Method 6) |
| 61 | 1-(2-(3-fluoro-5-(pentafluoro-λ⁶- | Intermediate 14 and | (400 MHz, DMSO-d₆) δ: 8.66 (d, J = | m/z 449.1 |
| | sulfaneyl)benzyl)pyridin-4-yl)-1,5,6,7- | CAS: 1240257-20-8 | 5.6 Hz, 1H), 8.06 (s, 1H), 7.84 (s, | (M + H)+ |
| | tetrahydro-4H-pyrazolo[4,3-c]pyridin-4-one | Procedure 9 | 1H), 7.81-7.77 (m, 1H), 7.73 (d, J = | (ES+), at |
| | | | 2.0 Hz, 1H), 7.58 (d, J = 9.2 Hz, 1H), | 2.13 min, |
| | | | 7.55-7.52 (m, 2H), 4.35 (s, 2H), 3.45- | 100% |
| | | | 3.41 (m, 2H), 3.20 (t, J = 6.8 Hz, 2H). | (Method 6) |
| 62 | 1-(2-(3-(difluoromethoxy)-5- | Intermediate 14 and | (400 MHz, DMSO-d₆) δ: 8.65 (d, J = | m/z 389.1 |
| | fluorobenzyl)pyridin-4-yl)-1,5,6,7-tetrahydro- | CAS: 1017779-39-3 | 5.2 Hz, 1H), 8.06 (s, 1H), 7.67 (d, J = | (M + H)+ |
| | 4H-pyrazolo[4,3-c]pyridin-4-one | Procedure 9 | 2.0 Hz, 1H), 7.53-7.52 (m, 2H), 7.47- | (ES+), at |
| | | | 7.28 (m, 1H), 7.12-7.08 (m, 1H), 7.05 | 1.93 min, |
| | | | (s, 1H), 7.01-6.98 (m, 1H), 4.22 (s, | 100% |
| | | | 2H), 3.45-3.41 (m, 2H), 3.19 (t, J = | (Method 6) |
| | | | 6.4 Hz, 2H). | |
| 63 | 1-(2-(4-(difluoromethoxy)-3- | Intermediates 14 and 24 | (400 MHz, DMSO-d₆) δ: 8.64 (d, J = | m/z 389.1 |
| | fluorobenzyl)pyridin-4-yl)-1,5,6,7-tetrahydro- | Procedures 9, step 1 and | 5.6 Hz, 1H), 8.05 (s, 1H), 7.66 (d, J = | (M + H)+ |
| | 4H-pyrazolo[4,3-c]pyridin-4-one | 2, and 10, step 4 | 2.0 Hz, 1H), 7.52-7.50 (m, 2H), 7.41- | (ES+), at |
| | | | 7.38 (m, 1H), 7.33-7.29 (m, 1H), | 1.67 min, |
| | | | 7.22-7.02 (m, 2H), 4.20 (s, 2H), 3.45- | 97% |
| | | | 3.41 (m, 2H), 3.21-3.17 (m, 2H). | (Method 3) |
| 64 | 1-(2-(3,4-difluoro-5- | Intermediates 14 and 25 | (400 MHz, DMSO-d₆) δ: 8.65 (d, J = | m/z 409.2 |
| | (trifluoromethyl)benzyl)pyridin-4-yl)-1,5,6,7- | Procedures 9, step 1 and | 5.6 Hz, 1H), 8.06 (s, 1H), 7.86-7.81 | (M + H)+ |
| | tetrahydro-4H-pyrazolo[4,3-c]pyridin-4-one | 2, and 10, step 4 | (m, 1H), 7.71-7.70 (m, 1H), 7.66-7.65 | (ES+), at |
| | | | (m, 1H), 7.54-7.51 (m, 2H), 4.29 (s, | 2.03 min, |
| | | | 2H), 3.46-3.42 (m, 2H), 3.24-3.18 (m, | 99% |
| | | | 2H). | (Method 2) |
| 65 | 1-(2-(3-(difluoromethyl)-5-fluorobenzyl)pyridin- | Intermediates 14 and 26 | (400 MHz, DMSO-d₆) δ: 8.65 (d, J = | m/z 373.1 |
| | 4-yl)-1,5,6,7-tetrahydro-4H-pyrazolo[4,3- | Procedures 9, step 1 and | 5.6 Hz, 1H), 8.06 (s, 1H), 7.69 (d, J = | (M + H)+ |
| | c]pyridin-4-one | 2, and 10, step 4 | 2.0 Hz, 1H), 7.53-7.51 (m, 2H), 7.41 | (ES+), at |
| | | | (d, J = 12.4 Hz, 2H), 7.29 (d, J = 8.8 | 1.47 min, |
| | | | Hz, 1H), 7.03 (t, J = 55.6 Hz, 1H), | 99% |
| | | | 4.28 (s, 2H), 3.45-3.41 (m, 2H), 3.19 | (Method 7) |
| | | | (t, J = 6.8 Hz, 2H). | |
| 66 | 1-(2-(3-chloro-4,5-difluorobenzyl)pyridin-4-yl)- | Intermediates 14 and 27 | (400 MHz, DMSO-d₆) δ: 8.65 (d, J = | m/z 375.1 |
| | 1,5,6,7-tetrahydro-4H-pyrazolo[4,3-c]pyridin-4- | Procedures 9, step 1 and | 5.6 Hz, 1H), 8.06 (s, 1H), 7.68 (d, J = | (M + H)+ |
| | one | 2, and 10, step 4 | 2.0 Hz, 1H), 7.54-7.54 (m, 4H), 4.20 | (ES+), at |
| | | | (s, 2H), 3.46-3.42 (m, 2H), 3.20 (t, J = | 1.59 min, |
| | | | 6.8 Hz, 2H). | 98% |
| | | | | (Method 7) |
| 67 | 1-(2-(quinolin-3-ylmethyl)pyridin-4-yl)-1,5,6,7- | Intermediate 14 and | (400 MHz, DMSO-d₆) δ: 8.93 (d, J = | m/z 356.2 |
| | tetrahydro-4H-pyrazolo[4,3-c]pyridin-4-one | CAS: 120277-70-5 | 2.4 Hz, 1H), 8.65 (d, J = 5.6 Hz, 1H), | (M + H)+ |
| | | Procedures 9, step 1 and | 8.27 (d, J = 1.6 Hz, 1H), 8.04 (s, 1H), | (ES+), at |
| | | 2, and 10, step 4 | 7.99 (s, 1H), 7.94 (d, J = 0.8 Hz, 1H), | 0.97 min, |
| | | | 7.75-7.70 (m, 2H), 7.61-7.57 (m, 1H), | 99% |
| | | | 7.53-7.51 (m, 2H), 4.43 (s, 2H), 3.43- | (Method 3) |
| | | | 3.41 (m, 2H), 3.20 (t, J = 6.4 Hz, 2H). | |
| 68 | 1-(2-(4-chloro-3-fluoro-5- | Intermediate 14 and | (400 MHz, DMSO-d₆) δ: 8.65 (d, J = | m/z 424.9 |
| | (trifluoromethyl)benzyl)pyridin-4-yl)-1,5,6,7- | CAS: 1431329-80-4 | 5.6 Hz, 1H), 8.06 (s, 1H), 7.80-7.72 | (M + H)+ |
| | tetrahydro-4H-pyrazolo[4,3-c]pyridin-4-one | Procedures 9, step 1 and | (m, 3H), 7.55-7.51 (m, 2H), 4.33 (s, | (ES+), at |
| | | 2, and 10, step 4 | 2H), 3.46-3.42 (m, 2H), 3.20 (t, J = | 2.26 min, |
| | | | 6.4 Hz, 2H). | 94% |
| | | | | (Method 2) |
| 69 | 1-(2-(benzo[b]thiophen-2-ylmethyl)pyridin-4-yl)- | Intermediate 14 and | (400 MHz, DMSO-d₆) δ: 8.68 (d, J = | m/z 361.1 |
| | 1,5,6,7-tetrahydro-4H-pyrazolo[4,3-c]pyridin-4- | CAS: 2076-88-2 | 5.2 Hz, 1H), 8.05 (s, 1H), 7.88-7.86 | (M + H)+ |
| | one | Procedures 9, step 1 and | (m, 1H), 7.78-7.74 (m, 2H), 7.56-7.50 | (ES+), at |
| | | 2, and 10, step 4 | (m, 2H), 7.35-7.28 (m, 3H), 4.50 (s, | 1.71 min, |
| | | | 2H), 3.45-3.41 (m, 2H), 3.20 (t, J = | 97% |
| | | | 6.8 Hz, 2H). | (Method 7) |
| 70 | 1-(2-(3,5-difluoro-4- | Intermediates 14 and 28 | (400 MHz, DMSO-d₆) δ: 8.66 (d, J = | m/z 409.2 |
| | (trifluoromethyl)benzyl)pyridin-4-yl)-1,5,6,7- | Procedure 9 | 5.6 Hz, 1H), 8.07 (s, 1H), 7.73 (d, J = | (M + H)+ |
| | tetrahydro-4H-pyrazolo[4,3-c]pyridin-4-one | | 1.6 Hz, 1H), 7.56-7.52 (m, 2H), 7.40- | (ES+), at |
| | | | 7.38 (m, 2H), 4.31 (s, 2H), 3.46-3.42 | 1.71 min, |
| | | | (m, 2H), 3.21 (t, J = 6.4 Hz, 2H). | 98% |
| | | | | (Method 7) |
| 71 | 1-(2-(3-fluoro-5-isopropylbenzyl)pyridin-4-yl)- | Intermediates 14 and 29 | (400 MHz, DMSO-d₆) δ: 8.64 (d, J = | m/z 365.0 |
| | 1,5,6,7-tetrahydro-4H-pyrazolo[4,3-c]pyridin-4- | Procedure 9 | 5.2 Hz, 1H), 8.05 (s, 1H), 7.63-7.51 | (M + H)+ |
| | one | | (m, 1H), 7.50-7.50 (m, 2H), 7.09 (s, | (ES+), at |
| | | | 1H), 6.97-6.92 (m, 2H), 4.17 (s, 2H), | 2.11 min, |
| | | | 3.44-3.40 (m, 2H), 3.18 (t, J = 6.8 Hz, | 100% |
| | | | 2H), 2.90-2.86 (m, 1H), 1.18 (d, J = | (Method 2) |
| | | | 6.9 Hz, 6H). | |
| 72 | 1-(2-((5-fluoro-6-methoxypyridin-3- | Intermediates 14 and 32 | (400 MHz, DMSO-d₆) δ: 8.72 (d, J = | m/z 354.0 |
| | yl)methyl)pyridin-4-yl)-1,5,6,7-tetrahydro-4H- | Procedures 9, step 1 and | 5.6 Hz, 1H), 8.16 (s, 1H), 7.92 (d, J = | (M + H)+ |
| | pyrazolo[4,3-c]pyridin-4-one | 2, and 10, step 4 | 2.0 Hz, 1H), 7.49 (s, 1H), 7.38-733 | (ES+), at |
| | | | (m, 2H), 5.59 (s, 1H), 4.22 (s, 2H), | 1.48 min, |
| | | | 4.03 (s, 3H), 3.70-3.66 (m, 2H), 3.19 | 99% |
| | | | (t, J = 6.8 Hz, 2H). | (Method 7) |
| 73 | 1-(2-(3-chloro-4-fluoro-5- | Intermediates 14 and 31 | (400 MHz, DMSO-d₆) δ: 8.65 (d, J = | m/z 425.1 |
| | (trifluoromethyl)benzyl)pyridin-4-yl)-1,5,6,7- | Procedures 9, step 1 and | 5.6 Hz, 1H), 8.07 (s, 1H), 7.98-7.96 | (M + H)+ |
| | tetrahydro-4H-pyrazolo[4,3-c]pyridin-4-one | 2, and 10, step 4 | (m, 1H), 7.82-7.80 (m, 1H), 7.72 (d, | (ES+), at |
| | | | J = 1.6 Hz, 1H), 7.54-7.51 (m, 2H), | 1.77 min, |
| | | | 4.30 (s, 2H), 3.44-3.42 (m, 2H), 3.20 | 99% |
| | | | (t, J = 6.8 Hz, 2H). | (Method 7) |
| 74 | 1-(2-(3-fluoro-5-(1- | Intermediates 14 and 30 | (400 MHz, DMSO-d₆) δ: : 8.65 (t, J = | m/z 431.2 |
| | (trifluoromethyl)cyclopropyl)benzyl)pyridin-4- | Procedures 9, step 1 and | 5.2 Hz, 1H), 8.05 (s, 1H), 7.74-7.66 | (M + H)+ |
| | yl)-1,5,6,7-tetrahydro-4H-pyrazolo[4,3- | 2, and 10, step 4 | (m, 1H), 7.53-7.51 (m, 2H), 7.35 (s, | (ES+), at |
| | c]pyridin-4-one | | 1H), 7.21-7.14 (m, 2H), 4.23 (s, 2H), | 1.90 min, |
| | | | 3.45-3.41 (m, 2H), 3.17 (t, J = 6.4 Hz, | 96% |
| | | | 2H), 1.38-1.33 (m, 2H), 0.90-0.85 (m, | (Method 2) |
| | | | 2H). | |
| 75 | 1-(2-(3-fluoro-5-(trifluoromethyl)benzyl)pyridin- | Intermediate 36 and | (400 MHz, DMSO-d₆) δ: 8.61 (d, J = | m/z 404.9 |
| | 4-yl)-3-methyl-1,5,6,7-tetrahydro-4H- | CAS: 239087-09-3 | 5.6 Hz, 1H), 7.70 (d, J = 2.0 Hz, 1H), | (M + H)+ |
| | pyrazolo[4,3-c]pyridin-4-one | Procedures 9, step 1 and | 7.62 (s, 1H), 7.56-7.53 (m, 2H), 7.48- | (ES+), at |
| | | 2, and 10, step 4 | 7.46 (m, 1H), 7.43 (s, 1H), 4.29 (s, | 2.13 min, |
| | | | 2H), 3.42-3.38 (m, 2H), 3.18-3.14 (m, | 98% |
| | | | 2H), 2.41 (s, 3H). | (Method 2) |
| 76 | 1-(2-((6-fluoro-5-methylpyridin-3- | Intermediate 36 and | (400 MHz, DMSO-d₆) δ: 8.60 (d, J = | m/z 352.2 |
| | yl)methyl)pyridin-4-yl)-3-methyl-1,5,6,7- | CAS: 1260812-39-2 | 5.2 Hz, 1H), 8.03 (s, 1H), 7.78 (d, J = | (M + H)+ |
| | tetrahydro-4H-pyrazolo[4,3-c]pyridin-4-one | Procedures 9, step 1 and | 9.6 Hz, 1H), 7.64 (d, J = 2.0 Hz, 1H), | (ES+), at |
| | | 2, and 10, step 4 | 7.47-7.43 (m, 2H), 4.17 (s, 2H), 3.43- | 1.11 min, |
| | | | 3.39 (m, 2H), 3.16 (t, J = 6.8 Hz, 2H), | 99% |
| | | | 2.41 (s, 3H), 2.21 (s, 3H). | (Method 7) |
| 77 | 3-methyl-1-(2-(3,4,5-trifluorobenzyl)pyridin-4- | Intermediate 36 and | (400 MHz, DMSO-d₆) δ: 8.61 (d, J = | m/z 373.2 |
| | yl)-1,5,6,7-tetrahydro-4H-pyrazolo[4,3- | CAS: 220141-72-0 | 5.2 Hz, 1H), 7.64 (d, J = 2.0 Hz, 1H), | (M + H)+ |
| | c]pyridin-4-one | Procedures 9, step 1 and | 7.48-7.46 (m, 2H), 7.33-7.29 (m, 2H), | (ES+), at |
| | | 2, and 10, step 4 | 4.18 (s, 2H), 3.41-3.39 (m, 2H), 3.18- | 1.80 min, |
| | | | 3.15 (m, 2H), 2.41 (s, 3H). | 100% |
| | | | | (Method 7) |
| 78 | 3-methyl-1-(2-(3-(trifluoromethyl)benzyl)pyridin- | Intermediate 36 and | (400 MHz, DMSO-d₆) δ: 8.61 (d, J = | m/z 387.2 |
| | 4-yl)-1,5,6,7-tetrahydro-4H-pyrazolo[4,3- | CAS: 402-23-3 | 5.6 Hz, 1H), 7.74 (s, 1H), 7.66 (t, J = | (M + H)+ |
| | c]pyridin-4-one | Procedures 9, step 1 and | 1.6 Hz, 2H), 7.60-7.53 (m, 2H), 7.47- | (ES+), at |
| | | 2, and 10, step 4 | 7.42 (m, 2H), 4.29 (s, 2H), 3.40-3.38 | 1.48 min, |
| | | | (m, 2H), 3.15 (t, J = 6.4 Hz, 2H), 2.40 | 100% |
| | | | (s, 3H). | (Method 2) |
| 79 | 1-(2-(3-chloro-5-fluorobenzyl)pyridin-4-yl)-3- | Intermediate 36 and | (400 MHz, DMSO-d₆) δ: 8.61 (d, J = | m/z 371.1 |
| | methyl-1,5,6,7-tetrahydro-4H-pyrazolo[4,3- | CAS: 493024-39-8 | 5.6 Hz, 1H), 7.66 (d, J = 1.6 Hz, 1H), | (M + H)+ |
| | c]pyridin-4-one | Procedures 9, step 1 and | 7.48-7.43 (m, 2H), 7.30-7.20 (m, 3H), | (ES+), at |
| | | 2, and 10, step 4 | 4.20 (s, 2H), 3.41-3.39 (m, 2H), 3.17 | 1.99 min, |
| | | | (t, J = 6.8 Hz, 2H), 2.41 (s, 3H). | 100% |
| | | | | (Method 7) |
| 80 | 1-(2-(3-chloro-4-fluorobenzyl)pyridin-4-yl)-3- | Intermediate 36 and | (400 MHz, DMSO-d₆) δ: 8.60 (d, J = | m/z 370.9 |
| | methyl-1,5,6,7-tetrahydro-4H-pyrazolo[4,3- | CAS: 192702-01-5 | 5.6 Hz, 1H), 7.64 (d, J = 1.6 Hz, 1H), | (M + H)+ |
| | c]pyridin-4-one | Procedures 9, step 1 and | 7.58 (d, J = 7.6 Hz, 1H), 7.46-7.44 | (ES+), at |
| | | 2, and 10, step 4 | (m, 2H), 7.35 (d, J = 8.4 Hz, 2H), | 1.58 min, |
| | | | 4.17 (s, 2H), 3.43-3.39 (m, 2H), 3.15 | 97% |
| | | | (t, J = 6.8 Hz, 2H), 2.40 (s, 3H). | (Method 2) |
| 81 | 1-(2-(4-(difluoromethoxy)-3- | Intermediate 36 and 24 | (400 MHz, DMSO-d₆) δ: 8.60 (d, J = | m/z 402.9 |
| | fluorobenzyl)pyridin-4-yl)-3-methyl-1,5,6,7- | Procedures 9, step 1 and | 7.2 Hz, 1H), 7.63 (d, J = 2.4 Hz, 1H), | (M + H)+ |
| | tetrahydro-4H-pyrazolo[4,3-c]pyridin-4-one | 2, and 10, step 4 | 7.47-6.95 (m, 6H), 4.18 (s, 2H), 3.41- | (ES+), at |
| | | | 3.38 (m, 2H), 3.17-3.12 (m, 2H), 2.27 | 2.05 min, |
| | | | (s, 3H). | 98% |
| | | | | (Method 2) |
| 82 | 1-(5-(3-fluoro-5-(trifluoromethyl)benzyl)pyridin- | Intermediate 37 and | (400 MHz, DMSO-d₆) δ: 8.68-8.64 | m/z 405.2 |
| | 3-yl)-3-methyl-1,5,6,7-tetrahydro-4H- | CAS: 239087-09-3 | (m, 2H), 8.02 (s, 1H), 7.68-7.53 (m, | (M + H)+ |
| | pyrazolo[4,3-c]pyridin-4-one | Procedures 3 and 10, step | 3H), 7.35 (s, 1H), 4.21 (s, 2H), 3.39- | (ES+), at |
| | | 4 | 3.37 (m, 2H), 3.06-3.02 (m, 2H), 2.28 | 1.98 min, |
| | | | (s, 3H). | 99% |
| | | | | (Method 6) |
| 83 | 1-(4-((6-fluoro-5-methylpyridin-3- | Intermediate 38 and | (400 MHz, DMSO-d₆) δ: 8.38 (d, J = | m/z 352.2 |
| | yl)methyl)pyridin-2-yl)-3-methyl-1,5,6,7- | CAS: 1260812-39-2 | 5.2 Hz, 1H), 8.06 (s, 1H), 7.78 (t, J = | (M + H)+ |
| | tetrahydro-4H-pyrazolo[4,3-c]pyridin-4-one | Procedures 3 and 10, step | 16.0 Hz, 2H), 7.38 (s, 1H), 7.27 (d, | (ES+), at |
| | | 4 | J = 4.0 Hz, 1H), 4.09 (s, 2H), 3.74-3.41 | 1.64 min, |
| | | | (m, 4H), 2.41 (s, 3H), 2.21 (s, 3H). | 98% |
| | | | | (Method 2) |
| 84 | 1-(2-(3-chloro-4,5-difluorobenzyl)pyridin-4-yl)- | Intermediates 36 and 27 | (400 MHz, DMSO-d₆) δ: 8.61 (d, J = | m/z 389.2 |
| | 3-methyl-1,5,6,7-tetrahydro-4H-pyrazolo[4,3- | Procedures 9, step 1 and | 5.6 Hz, 1H), 7.66 (d, J = 2.0 Hz, 1H), | (M + H)+ |
| | c]pyridin-4-one | 2, and 10, step 4 | 7.48-7.42 (m, 4H), 4.18 (s, 2H), 3.43- | (ES+), at |
| | | | 3.41 (m, 2H), 3.18-3.15 (m, 2H), 2.50 | 1.52 min, |
| | | | (s, 3H). | 100% |
| | | | | (Method 2) |
| 85 | 3-fluoro-5-((4-(3-methyl-4-oxo-4,5,6,7- | Intermediate 36 and | (400 MHz, DMSO-d₆) δ: 8.61 (d, J = | m/z 362.2 |
| | tetrahydro-1H-pyrazolo[4,3-c]pyridin-1- | CAS: 853368-35-1 | 5.2 Hz, 1H), 7.73-7.67 (m, 3H), 7.61 | (M + H)+ |
| | yl)pyridin-2-yl)methyl)benzonitrile | Procedures 9, step 1 and | (d, J = 10.0 Hz, 1H), 7.49-7.43 (m, | (ES+), at |
| | | 2, and 10, step 4 | 2H), 4.27 (s, 2H), 3.41 (t, J = 1.6 Hz, | 1.34 min, |
| | | | 2H), 3.17 (t, J = 6.8 Hz, 2H), 2.41 (s, | 99% |
| | | | 3H). | (Method 7) |
| 86 | 1-(2-(3,4-difluoro-5- | Intermediates 36 and 25 | (400 MHz, DMSO-d₆) δ: 8.61 (d, J = | m/z 423.0 |
| | (trifluoromethyl)benzyl)pyridin-4-yl)-3-methyl- | Procedures 9, step 1 and | 5.6 Hz, 1H), 7.85-7.80 (m, 1H), 7.69- | (M + H)+ |
| | 1,5,6,7-tetrahydro-4H-pyrazolo[4,3-c]pyridin-4- | 2, and 10, step 4 | 7.64 (m, 2H), 7.48-7.43 (m, 2H), 4.27 | (ES+), at |
| | one | | (s, 2H), 3.42-3.34 (m, 2H), 3.14-3.14 | 1.90 min, |
| | | | (m, 2H), 2.41 (s, 3H). | 100% |
| | | | | (Method 3) |
| 87 | 1-(2-(4-chloro-3-fluoro-5- | Intermediate 36 and | (400 MHz, DMSO-d₆) δ: 8.62-8.61 | m/z 439.0 |
| | (trifluoromethyl)benzyl)pyridin-4-yl)-3-methyl- | CAS: 1431329-80-4 | (m, 1H), 7.79-7.77 (m, 2H), 7.70 (d, | (M + H)+ |
| | 1,5,6,7-tetrahydro-4H-pyrazolo[4,3-c]pyridin-4- | Procedures 9, step 1 and | J = 1.6 Hz, 1H), 7.48 (dd, J = 2.4, 5.4 | (ES+), at |
| | one | 2, and 10, step 4 | Hz, 1H), 7.42 (s, 1H), 4.31 (s, 2H), | 2.10 min, |
| | | | 3.41 (td, J = 6.8, 2.4 Hz, 2H), 3.16 (t, | 100% |
| | | | J = 6.8 Hz, 2H), 2.41 (s, 3H). | (Method 7) |
| 88 | 1-(2-(3-chloro-4-fluoro-5- | Intermediates 36 and 31 | (400 MHz, DMSO-d₆) δ: 8.68 (d, J = | m/z 439.1 |
| | (trifluoromethyl)benzyl)pyridin-4-yl)-3-methyl- | Procedures 9, step 1 and | 5.6 Hz, 1H), 7.54-7.30 (m, 4H), 5.39 | (M + H)+ |
| | 1,5,6,7-tetrahydro-4H-pyrazolo[4,3-c]pyridin-4- | 2, and 10, step 4 | (s, 1H), 4.25 (s, 2H), 3.66-3.63 (m, | (ES+), at |
| | one | | 2H), 3.16 (t, J = 6.4 Hz, 2H), 2.59 (s, | 1.96 min, |
| | | | 3H). | 95% |
| | | | | (Method 2) |
| 89 | 1-(2-(3-(difluoromethoxy)-5- | Intermediate 36 and | (400 MHz, DMSO-d₆) δ: 8.61 (d, J = | m/z 403.2 |
| | fluorobenzyl)pyridin-4-yl)-3-methyl-1,5,6,7- | CAS: 1017779-39-3 | 5.6 Hz, 1H), 7.65 (s, 1H), 7.64-7.46 | (M + H)+ |
| | tetrahydro-4H-pyrazolo[4,3-c]pyridin-4-one | Procedures 9, step 1 and | (m, 1H), 7.42 (s, 1H), 7.27 (t, J = 72.4 | (ES+), at |
| | | 2, and 10, step 4 | Hz, 1H), 7.10-7.08 (m, 1H), 7.10-7.01 | 1.55 min, |
| | | | (m, 1H), 6.99 (m, 1 H), 4.20 (s, 2H), | 98% |
| | | | 3.42-3.38 (m, 2H), 3.15 (t, J = 6.4 Hz, | (Method 7) |
| | | | 2H), 2.41 (s, 3H). | |
| 90 | 1-(2-(4-chloro-3,5-difluorobenzyl)pyridin-4-yl)- | Intermediate 36 and | (400 MHz, DMSO-d₆) δ: 8.61 (d, J = | m/z 389.0 |
| | 3-methyl-1,5,6,7-tetrahydro-4H-pyrazolo[4,3- | CAS: 1400991-56-1 | 5.6 Hz, 1H), 7.66 (d, J = 2.0 Hz, 1H), | (M + H)+ |
| | c]pyridin-4-one | Procedures 9, step 1 and | 7.49-7.43 (m, 2H), 7.34-7.31 (m, 2H), | (ES+), at |
| | | 2, and 10, step 4 | 4.22 (s, 2H), 3.43-3.39 (m, 2H), 3.17 | 1.92 min, |
| | | | (t, J = 6.0 Hz, 2H), 2.41 (s, 3H). | 98% |
| | | | | (Method 7) |
| 91 | 1-(2-(3-(difluoromethyl)-5-fluorobenzyl)pyridin- | Intermediates 36 and 26 | (400 MHz, DMSO-d₆) δ: 8.61 (d, J = | m/z 387.1 |
| | 4-yl)-3-methyl-1,5,6,7-tetrahydro-4H- | Procedures 9, step 1 and | 5.6 Hz, 1H), 7.67 (s, 1H), 7.48-7.46 | (M + H)+ |
| | pyrazolo[4,3-c]pyridin-4-one | 2, and 10, step 4 | (m, 1H), 7.42-7.38 (m, 3H), 7.30-7.28 | (ES+), at |
| | | | (m, 1H), 7.03 (t, J = 55.6 Hz, 1H), | 1.92 min, |
| | | | 4.27 (s, 2H), 3.43-3.39 (m, 2H), 3.15 | 96% |
| | | | (t, J = 6.8 Hz, 2H), 2.41 (s, 3H). | (Method 3) |
| 92 | 1-(2-((6-methoxypyridin-3-yl)methyl)pyridin-4- | Intermediates 36 and 33 | (400 MHz, DMSO-d₆) δ: 8.59 (d, J = | m/z 350.2 |
| | yl)-3-methyl-1,5,6,7-tetrahydro-4H- | Procedures 9, step 1 and | 5.6 Hz, 1H), 8.15 (d, J = 2.0 Hz, 1H), | (M + H)+ |
| | pyrazolo[4,3-c]pyridin-4-one | 2, and 10, step 4 | 7.67-7.59 (m, 2H), 7.45-7.42 (m, 2H), | (ES+), at |
| | | | 6.77-6.75 (m, 1H), 4.11 (s, 2H), 3.81 | 0.97 min, |
| | | | (s, 3H), 3.42-3.35 (m, 2H), 3.15 (t, J = | 100% |
| | | | 6.8 Hz, 2H), 2.40 (s, 3H). | (Method 7) |
| 93 | 1-(2-((5-fluoro-6-methoxypyridin-3- | Intermediates 36 and 32 | (400 MHz, DMSO-d₆) δ: 8.59 (d, J = | m/z 368.1 |
| | yl)methyl)pyridin-4-yl)-3-methyl-1,5,6,7- | Procedures 9, step 1 and | 5.2 Hz, 1H), 7.98 (d, J = 2.0 Hz, 1H), | (M + H)+ |
| | tetrahydro-4H-pyrazolo[4,3-c]pyridin-4-one | 2, and 10, step 4 | 7.67-7.62 (m, 2H), 7.46-7.42 (m, 2H), | (ES+), at |
| | | | 4.15 (s, 2H), 3.91 (s, 3H), 3.43-3.39 | 1.30 min, |
| | | | (m, 2H), 3.16 (t, J = 6.8 Hz, 2H), 2.33 | 98% |
| | | | (s, 3H). | (Method 7) |
| 94 | 1-(2-((6-(difluoromethoxy)pyridin-3- | Intermediates 36 and 34 | (400 MHz, DMSO-d₆) δ: 8.59 (d, J = | m/z 385.9 |
| | yl)methyl)pyridin-4-yl)-3-methyl-1,5,6,7- | Procedures 9, step 1 and | 5.6 Hz, 1H), 8.26 (d, J = 2.0 Hz, 1H), | (M + H)+ |
| | tetrahydro-4H-pyrazolo[4,3-c]pyridin-4-one | 2, and 10, step 4 | 7.89-7.86 (m, 1H), 7.66 (t, J = 8.4 Hz, | (ES+), at |
| | | | 2H), 7.49-7.42 (m, 2H), 7.04 (d, J = | 1.90 min, |
| | | | 8.4 Hz, 1H), 4.20 (s, 2H), 3.43-3.39 | 97% |
| | | | (m, 2H), 3.16 (t, J = 6.8 Hz, 2H), 2.41 | (Method 2) |
| | | | (s, 3H). | |
| 95 | 1-(4-(3-fluoro-5-(trifluoromethyl)benzyl)pyridin- | Intermediates 8 and 35 | (400 MHz, DMSO-d₆) δ: 8.39 (d, J = | m/z 405.1 |
| | 2-yl)-3-methyl-1,5,6,7-tetrahydro-4H- | Procedures 1 and 10, step | 5.2 Hz, 1H), 7.88 (s, 1H), 7.65 (s, | (M + H)+ |
| | pyrazolo[4,3-c]pyridin-4-one | 4 | 1H), 7.58-7.56 (m, 2H), 7.38 (s, 1H), | (ES+), at |
| | | | 7.33-7.31 (m, 1H), 4.23 (s, 2H), 3.42- | 2.32 min, |
| | | | 3.33 (m, 4H), 2.34 (s, 3H). | 96% |
| | | | | (Method 7) |
| 96 | 3-fluoro-5-((4-(4-oxo-4,5,6,7-tetrahydro-1H- | Intermediate 14 and | (400 MHz, DMSO-d₆) δ: 8.65 (d, J = | m/z 348.2 |
| | pyrazolo[4,3-c]pyridin-1-yl)pyridin-2- | CAS: 216755-57-6 | 5.6 Hz, 1H), 8.06 (s, 1H), 7.74-7.70 | (M + H)+ |
| | yl)methyl)benzonitrile | Procedure 10 | (m, 3H), 7.63-7.61 (m, 1H), 7.60-7.51 | (ES+), at |
| | | | (m, 2H), 4.28 (s, 2H), 3.46-3.42 (m, | 1.60 min, |
| | | | 2H), 3.22-3.12 (m, 2H). | 100% |
| | | | | (Method 6) |
| 97 | 1-(2-(3-fluoro-5-(trifluoromethyl)benzyl)pyridin- | Intermediates 3 and 15 | (400 MHz, DMSO-d₆) δ: 8.77 (d, J = | m/z 406.1 |
| | 4-yl)-5,6,7,8-tetrahydro-[1,2,3]triazolo[4,5- | Procedure 14 | 5.4 Hz, 1H), 8.11 (t, J = 5.3 Hz, 1H), | (M + H)+ |
| | c]azepin-4(1H)-one | | 7.82 (d, J = 2.0 Hz, 1H), 7.72-7.59 | (ES+), at |
| | | | (m, 2H), 7.59-7.42 (m, 2H), 4.36 (s, | 5.18 min, |
| | | | 2H), 3.35-3.19 (m, 2H), 3.04 (t, J = | 98% |
| | | | 6.5 Hz, 2H), 1.98 (dd, J = 9.4, 5.6 Hz, | (Method 5) |
| | | | 2H). | |
| 98 | 2-(4-(3-fluoro-5-(trifluoromethyl)benzyl)pyridin- | Intermediates 5 and 16 | (400 MHz, DMSO-d₆) δ: 8.51 (d, J = | m/z 392.1 |
| | 2-yl)-2,5,6,7-tetrahydro-4H-[1,2,3]triazolo[4,5- | Procedure 15 | 5.0 Hz, 1H), 8.13 (s, 1H), 8.03 (d, J = | (M + H)+ |
| | c]pyridin-4-one | | 1.3 Hz, 1H), 7.67 (s, 1H), 7.62 (dd, | (ES+), at |
| | | | J = 9.8, 2.4 Hz, 1H), 7.56 (dd, J = 8.9, | 5.52 min, |
| | | | 2.0 Hz, 1H), 7.48 (dd, J = 5.0, 1.5 Hz, | 99% |
| | | | 1H), 4.27 (s, 2H), 3.53 (td, J = 6.7, | (Method 5) |
| | | | 2.8 Hz, 2H), 3.05 (t, J = 6.7 Hz, 2H). | |
| 99 | 1-(4-(3-fluoro-5-(trifluoromethyl)benzyl)pyridin- | Intermediates 5 and 16 | (400 MHz, DMSO-d₆) δ: 8.53 (d, J = | m/z 392.1 |
| | 2-yl)-1,5,6,7-tetrahydro-4H-[1,2,3]triazolo[4,5- | Procedure 15 | 5.1 Hz, 1H), 8.13 (s, 1H), 7.78 (s, | (M + H)+ |
| | c]pyridin-4-one | | 1H), 7.70 (s, 1H), 7.69-7.62 (m, 1H), | (ES+), at |
| | | | 7.62-7.50 (m, 2H), 4.28 (s, 2H), 3.48 | 5.71 min, |
| | | | (td, J = 7.0, 5.9, 2.1 Hz, 2H), 3.39 | 99% |
| | | | (dd, J = 7.2, 5.5 Hz, 2H). | (Method 5) |
| 100 | 2-(2-(3-fluoro-5-(trifluoromethyl)benzyl)pyridin- | Intermediates 3 and 16 | (400 MHz, DMSO-d₆) δ: 8.76 (d, J = | m/z 392.1 |
| | 4-yl)-2,5,6,7-tetrahydro-4H-[1,2,3]triazolo[4,5- | Procedure 15 | 5.4 Hz, 1H), 7.86 (d, J = 2.0 Hz, 1H), | (M + H)+ |
| | c]pyridin-4-one | | 7.82 (s, 1H), 7.64 (dd, J = 5.5, 2.0 | (ES+), at |
| | | | Hz, 2H), 7.55 (t, J = 8.4 Hz, 2H), 4.36 | 5.24 min, |
| | | | (s, 2H), 3.49 (td, J = 6.8, 2.6 Hz, 2H), | 98% |
| | | | 3.24 (t, J = 6.8 Hz, 2H). | (Method 5) |
| 101 | 1-(2-(3-fluoro-5-(trifluoromethyl)benzyl)pyridin- | Intermediates 3 and 16 | (400 MHz, DMSO-d₆) δ: 8.68 (d, J = | m/z 392.1 |
| | 4-yl)-1,5,6,7-tetrahydro-4H-[1,2,3]triazolo[4,5- | Procedure 15 | 5.5 Hz, 1H), 8.21 (d, J = 3.0 Hz, 1H), | (M + H)+ |
| | c]pyridin-4-one | | 8.07 (d, J = 2.1 Hz, 1H), 7.86 (dd, J = | (ES+), at |
| | | | 5.5, 2.1 Hz, 1H), 7.63 (s, 1H), 7.61- | 5.64 min, |
| | | | 7.49 (m, 2H), 4.35 (s, 2H), 3.54 (td, | 96% |
| | | | J = 6.7, 2.8 Hz, 2H), 3.07 (t, J = 6.7 Hz, | (Method 5) |
| | | | 2H). | |
| 102 | 1-(4-(3-(difluoromethyl)-5-fluorobenzyl)pyridin- | Intermediates 1 and 39 | (400 MHz, DMSO-d₆) δ: 8.43 (d, J = | m/z 373.1 |
| | 2-yl)-1,5,6,7-tetrahydro-4H-pyrazolo[4,3- | Procedure 1 | 5.2 Hz, 1H), 7.99 (s, 1H), 7.86 (s, | (M + H)+ |
| | c]pyridin-4-one | | 1H), 7.46-7.31 (m, 5H), 7.03 (t, J = | (ES+), at |
| | | | 55.6 Hz, 1H), 4.20 (s, 2H), 3.44- | 1.91 min, |
| | | | 3.44 (m, 4H). | 100% |
| | | | | (Method 3) |
| 103 | 1-(5-(3-fluoro-5- | Intermediate 40 and | (400 MHz, DMSO-d₆) δ: 8.76 (s, 1H), | m/z 393.1 |
| | (trifluoromethyl)phenoxy)pyridin-3-yl)-1,5,6,7- | CAS: 172333-87-8 | 8.58 (d, J = 2.4 Hz, 1H), 8.04 (s, 1H), | (M + H)+ |
| | tetrahydro-4H-pyrazolo[4,3-c]pyridin-4-one | Procedure 8 (2 | 7.89 (d, J = 2.4 Hz, 1H), 7.58-7.56 | (ES+), at |
| | | equivalents KOtBu | (m, 1H), 7.51-7.47 (m, 3H), 3.44-3.40 | 1.73 min, |
| | | substituted for 3 | (m, 2H), 3.12 (t, J = 6.4 Hz, 2H). | 98% |
| | | equivalents of Cs₂CO₃) | | (Method 7) |
| 104 | 1-(2-(3-fluoro-5-(trifluoromethyl)benzyl)pyridin- | Intermediate 13 and | (400 MHz, DMSO-d₆) δ: 8.65 (d, J = | m/z 449.0 |
| | 4-yl)-5-(2-methoxyethyl)-1,5,6,7-tetrahydro-4H- | CAS: 6482-24-2 and | 5.2 Hz, 1H), 8.06 (s, 1H), 7.72 (d, J = | (M + H)+ |
| | pyrazolo[4,3-c]pyridin-4-one | 239087-09-3 | 1.6 Hz, 1H), 7.63 (s, 1H), 7.55-7.52 | (ES+), at |
| | | Procedures 2 (DMF | (m, 3H), 4.32 (s, 2H), 3.68-3.65 (m, | 2.23 min, |
| | | substituted for THF) and | 2H), 3.61-3.58 (m, 2H), 3.51-3.49 (m, | 100% |
| | | 9, step 1 and 2 | 2H), 3.29-3.25 (m, 5H). | (Method 2) |
| 105 | 1-(2-(3-fluoro-5-(trifluoromethyl)benzyl)pyridin- | Example 104 | (400 MHz, DMSO-d₆) δ: 8.65 (d, J = | m/z 435.1 |
| | 4-yl)-5-(2-hydroxyethyl)-1,5,6,7-tetrahydro-4H- | Procedure 20 | 5.6 Hz, 1H), 8.06 (s, 1H), 7.72 (d, J = | (M + H)+ |
| | pyrazolo[4,3-c]pyridin-4-one | | 1.6 Hz, 1H), 7.63-7.75 (m, 4H), 4.77- | (ES+), at |
| | | | 4.74 (m, 1H), 4.33 (s, 2H), 3.71-3.68 | 1.76 min, |
| | | | (m, 2H), 3.56-3.48 (m, 4H), 3.29-3.26 | 99% |
| | | | (m, 2H). | (Method 3) |
| 106 | 1-(4-(3-fluoro-5-(trifluoromethyl)benzyl)pyridin- | Intermediate 12 and | (400 MHz, DMSO-d₆) δ: 8.42 (d, J = | m/z 449.3 |
| | 2-yl)-5-(2-methoxyethyl)-1,5,6,7-tetrahydro-4H- | CAS: 6482-24-2 and | 5.1 Hz, 1H), 7.99 (s, 1H), 7.89 (s, | (M + H)+ |
| | pyrazolo[4,3-c]pyridin-4-one | 239087-09-3 | 1H), 7.65 (s, 1H), 7.58 (dd, J = 14.0, | (ES+), at |
| | | Procedures 2 (DMF | 9.3 Hz, 2H), 7.36 (d, J = 5.2 Hz, 1H), | 6.31 min, |
| | | substituted for THF) and | 4.23 (s, 2H), 3.65 (t, J = 6.9 Hz, 2H), | 100% |
| | | 9, step 1 (additionally with | 3.57 (t, J = 5.6 Hz, 2H), 3.47 (dt, J = | (Method 5) |
| | | 3 equivalents of LiCl) and | 16.8, 6.2 Hz, 4H), 3.26 (s, 3H). | |
| | | 2 (additionally with 0.1 | | |
| | | equivalents of CuI) | | |
| 107 | 1-(4-(3-fluoro-5-(trifluoromethyl)benzyl)pyridin- | Example 106 | (400 MHz, DMSO-d₆) δ: 8.42 (d, J = | m/z 435.2 |
| | 2-yl)-5-(2-hydroxyethyl)-1,5,6,7-tetrahydro-4H- | Procedure 21 | 5.1 Hz, 1H), 7.99 (s, 1H), 7.89 (s, | (M + H)+ |
| | pyrazolo[4,3-c]pyridin-4-one | | 1H), 7.65 (s, 1H), 7.58 (dd, J = 14.1, | (ES+), at |
| | | | 9.4 Hz, 2H), 7.46-7.25 (m, 1H), 4.72 | 5.94 min, |
| | | | (t, J = 5.4 Hz, 1H), 4.23 (s, 2H), 3.68 | 100% |
| | | | (t, J = 6.9 Hz, 2H), 3.55 (q, J = 5.8 | (Method 5) |
| | | | Hz, 2H), 3.51-3.39 (m, 4H). | |
| 108 | 1-(2-(3-fluoro-5-(trifluoromethyl)benzyl)-5- | Intermediate 41 and | (400 MHz, DMSO-d₆) δ: 8.60 (s, 1H), | m/z 405.1 |
| | methylpyridin-4-yl)-1,5,6,7-tetrahydro-4H- | CAS: 239087-09-3 | 8.01 (s, 1H), 7.59 (s, 1H), 7.54-7.52 | (M + H)+ |
| | pyrazolo[4,3-c]pyridin-4-one | Procedures 9, step 1 and | (m, 3H), 7.45 (s, 1H), 4.26 (s, 2H), | (ES+), at |
| | | 2, and 10, step 4 | 3.43-3.39 (m, 2H), 2.84 (t, J = 6.8 Hz, | 1.93 min, |
| | | | 2H), 2.18 (s, 3H). | 100% |
| | | | | (Method 10) |
| 109 | 1-(3-(3-fluoro-5-(trifluoromethyl)benzyl)phenyl)- | Intermediates 1 and 42 | (400 MHz, DMSO-d₆) δ: 7.95 (s, 1H), | m/z 390.0 |
| | 1,5,6,7-tetrahydro-4H-pyrazolo[4,3-c]pyridin-4- | Procedure 22 | 7.69-7.34 (m, 8H), 4.17 (s, 2H), 3.40 | (M + H)+ |
| | one | | (td, J = 6.8, 2.5 Hz, 2H), 3.04 (t, J = | (ES+), at |
| | | | 6.8 Hz, 2H). | 4.33 min, |
| | | | | 95% |
| | | | | (Method 1) |
| 110 | 1-(2-((6-(difluoromethoxy)-5-fluoropyridin-3- | Intermediates 14 and 32 | (400 MHz, DMSO-d₆) δ: 8.64 (d, J = | m/z 390.0 |
| | yl)methyl)pyridin-4-yl)-1,5,6,7-tetrahydro-4H- | Procedures 9, step 1 and | 5.2 Hz, 1H), 8.10 (d, J = 2.0 Hz, 1H), | (M + H)+ |
| | pyrazolo[4,3-c]pyridin-4-one | 2,and 23 | 8.06 (s, 1H), 7.93 (t, J = 10.8 Hz, 1H), | (ES+), at |
| | | | 7.70 (t, J = 3.6 Hz, 2H), 7.54-7.53 (m, | 1.52 min, |
| | | | 2H), 4.25 (s, 2H), 3.45 (t, J = 2.4 Hz, | 96% |
| | | | 2H), 3.20 (t, J = 6.8 Hz, 2H). | (Method 3) |
| 111 | 1-(2-((6-(difluoromethoxy)-5-fluoropyridin-3- | Example 93 | (400 MHz, DMSO-d₆) δ: 8.60 (d, J = | m/z 404.1 |
| | yl)methyl)pyridin-4-yl)-3-methyl-1,5,6,7- | Procedure 23 | 5.6 Hz, 1H), 8.10 (s, 1H), 7.94 (d, J = | (M + H)+ |
| | tetrahydro-4H-pyrazolo[4,3-c]pyridin-4-one | | 1.6 Hz, 1H), 7.70-7.67 (m, 2H), 7.52 | (ES+), at |
| | | | (s, 1H), 7.47 (t, J = 3.2 Hz, 1H), 4.23 | 1.58 min, |
| | | | (s, 2H), 3.42-3.41 (m, 2H), 3.17 (t, J = | 98% |
| | | | 6.8 Hz, 2H), 2.41 (s, 3H). | (Method 3) |
| 112 | 2-(2-(3-fluoro-5-(trifluoromethyl)benzyl)pyridin- | Intermediates 3 and 43 | (400 MHz, DMSO-d₆) δ: 9.04 (s, 1H), | m/z 407.1 |
| | 4-yl)-6,7-dihydro-2H-pyrazolo[4,3- | Procedure 24 | 8.54 (d, J = 5.6 Hz, 1H), 8.06 (t, J = | (M + H)+ |
| | f][1,4]oxazepin-4(5H)-one | | 4.0 Hz, 1H), 7.95 (d, J = 2.0 Hz, 1H), | (ES+), at |
| | | | 7.75-7.73 (m, 1H), 7.60 (s, 1H), 7.56- | 1.75 min, |
| | | | 7.52 (m, 2H), 4.42-4.40 (m, 2H), 4.25 | 99% |
| | | | (s, 2H), 3.49-3.46 (m, 2H). | (Method 3) |
| 113 | 1-(4-(3-fluoro-5-(trifluoromethyl)benzyl)pyridin- | Intermediates 8 and 44 | (400 MHz, DMSO-d₆) δ: 8.42 (d, J = | m/z 420.9 |
| | 2-yl)-3-(hydroxymethyl)-1,5,6,7-tetrahydro-4H- | Procedures 1 and 20 | 5.2 Hz, 1H), 7.89 (s, 1H), 7.83-7.81 | (M + H)+ |
| | pyrazolo[4,3-c]pyridin-4-one | | (m, 1H), 7.65 (s, 1H), 7.57-7.55 (m, | (ES+), at |
| | | | 2H), 7.35 (d, J = 5.2 Hz, 1H), 5.76 (t, | 2.57 min, |
| | | | J = 6.0 Hz, 1H), 4.65 (d, J = 6.4 Hz, | 99% |
| | | | 2H), 4.24 (s, 2H), 3.46-3.45 (m, 4H). | (Method 2) |
| 114 | 1-(2-(3-fluoro-5-(trifluoromethyl)benzyl)pyridin- | Intermediates 6 and 44 | (400 MHz, DMSO-d₆) δ: 8.65 (d, J = | m/z 420.9 |
| | 4-yl)-3-(hydroxymethyl)-1,5,6,7-tetrahydro-4H- | Procedures 1 and 20 | 5.6 Hz, 1H), 7.82 (s, 1H), 7.71 (d, J = | (M + H)+ |
| | pyrazolo[4,3-c]pyridin-4-one | | 2.0 Hz, 1H), 7.62 (s, 1H), 7.56-7.50 | (ES+), at |
| | | | (m, 3H), 5.69 (t, J = 6.4 Hz, 1H), 4.65 | 2.06 min, |
| | | | (d, J = 6.4 Hz, 2H), 4.32 (s, 2H), 3.47 | 97% |
| | | | (t, J = 6.8 Hz, 2H), 3.20 (t, J = 6.8 Hz, | (Method 2) |
| | | | 2H). | |
| 115 | 1-(2-(3-fluoro-5-(trifluoromethyl)benzyl)pyridin- | Intermediate 45 | (400 MHz, DMSO-d₆) δ: 8.73 (d, J = | m/z 416.0 |
| | 4-yl)-4-oxo-4,5,6,7-tetrahydro-1H-pyrazolo[4,3- | Procedure 10, step 3 | 5.2 Hz, 1H), 7.94 (s, 1H), 7.81 (s, | (M + H)+ |
| | c]pyridine-3-carbonitrile | | 1H), 7.63-7.55 (m, 4H), 4.35 (s, 2H), | (ES+), at |
| | | | 3.47 (d, J = 6.4 Hz, 2H), 3.25-3.22 | 2.23 min, |
| | | | (m, 2H). | 98% |
| | | | | (Method 3) |
| 116 | 1-(2-(3-fluoro-5-(trifluoromethyl)benzyl)pyridin- | Intermediate 45 | (400 MHz, DMSO-d₆) δ: 8.71 (d, J = | m/z 459.0 |
| | 4-yl)-3-(trifluoromethyl)-1,5,6,7-tetrahydro-4H- | Procedure 25 | 5.6 Hz, 1H), 7.78 (d, J = 8.4 Hz, 2H), | (M + H)+ |
| | pyrazolo[4,3-c]pyridin-4-one | | 7.63-7.53 (m, 4H), 4.35 (s, 2H), 3.45- | (ES+), at |
| | | | 3.33 (m, 2H), 3.20 (t, J = 6.8 Hz, 2H). | 2.38 min, |
| | | | | 98% |
| | | | | (Method 12) |
| 117 | 1-(2-((3-fluoro-5- | Example 75 | (400 MHz, DMSO-d₆) δ: 8.60 (d, J = | m/z 421.0 |
| Isomer 1 | (trifluoromethyl)phenyl)(hydroxy)methyl)pyridin- | Procedure 26 | 5.2 Hz, 1H), 7.90 (d, J = 2.4 Hz, 1H), | (M + H)+ |
| | 4-yl)-3-methyl-1,5,6,7-tetrahydro-4H- | Chiral SFC purity analysis: | 7.70 (s, 1H), 7.62-7.56 (m, 2H), 7.49- | (ES+), at |
| | pyrazolo[4,3-c]pyridin-4-one | 4.91 min, 99% (Method 6 - | 7.47 (m, 1H), 7.44 (s, 1H), 6.66 (s, | 1.68 min, |
| | (Single enantiomer of unknown absolute | isocratic run 20% co- | 1H), 5.94 (s, 1H), 3.43-3.40 (m, 2H), | 99% |
| | stereochemistry) | solvent) | 3.20-3.15 (m, 2H), 2.42 (s, 3H). | (Method 3) |
| 117 | 1-(2-((3-fluoro-5- | Example 75 | (400 MHz, DMSO-d₆) δ: 8.60 (d, J = | m/z 421.0 |
| Isomer 2 | (trifluoromethyl)phenyl)(hydroxy)methyl)pyridin- | Procedure 26 | 5.2 Hz, 1H), 7.90 (d, J = 2.4 Hz, 1H), | (M + H)+ |
| | 4-yl)-3-methyl-1,5,6,7-tetrahydro-4H- | Chiral SFC purity analysis: | 7.70 (s, 1H), 7.62-7.56 (m, 2H), 7.49- | (ES+), at |
| | pyrazolo[4,3-c]pyridin-4-one | 5.54 min, 99% (Method 6 - | 7.47 (m, 1H), 7.44 (s, 1H), 6.66 (s, | 1.68 min, |
| | (Single enantiomer of unknown absolute | isocratic run 20% co- | 1H), 5.94 (s, 1H), 3.43-3.40 (m, 2H), | 100% |
| | stereochemistry) | solvent) | 3.20-3.15 (m, 2H), 2.42 (s, 3H). | (Method 3) |
| 118 | 1-(4-((3-fluoro-5- | Example 95 | (400 MHz, DMSO-d₆) δ: 8.41 (t, J = | m/z 420.9 |
| Isomer 1 | (trifluoromethyl)phenyl)(hydroxy)methyl)pyridin- | Procedure 27 | 5.2 Hz, 1H), 8.02 (s, 1H), 7.72 (s, | (M + H)+ |
| | 2-yl)-3-methyl-1,5,6,7-tetrahydro-4H- | Chiral SFC purity analysis: | 1H), 7.66-7.59 (m, 2H), 7.42-7.37 (m, | (ES+), at |
| | pyrazolo[4,3-c]pyridin-4-one | Could not be determined - | 2H), 6.63 (d, J = 4.4 Hz, 1H), 6.02 (d, | 2.46 min, |
| | (Single enantiomer of unknown absolute | isomers not separable | J = 4.0 Hz, 1H), 3.41-3.33 (m, 4H), | 98% |
| | stereochemistry) | under available methods | 2.42 (s, 3H). | (Method 2) |
| 118 | 1-(4-((3-fluoro-5- | Example 95 | (400 MHz, DMSO-d₆) δ: 8.41 (t, J = | m/z 420.9 |
| Isomer 2 | (trifluoromethyl)phenyl)(hydroxy)methyl)pyridin- | Procedure 27 | 5.2 Hz, 1H), 8.02 (s, 1H), 7.72 (s, | (M + H)+ |
| | 2-yl)-3-methyl-1,5,6,7-tetrahydro-4H- | Chiral SFC purity analysis: | 1H), 7.66-7.59 (m, 2H), 7.42-7.37 (m, | (ES+), at |
| | pyrazolo[4,3-c]pyridin-4-one | Could not be determined - | 2H), 6.63 (d, J = 4.4 Hz, 1H), 6.02 (d, | 2.46 min, |
| | (Single enantiomer of unknown absolute | isomers not separable | J = 4.0 Hz, 1H), 3.41-3.33 (m, 4H), | 99% |
| | stereochemistry) | under available methods | 2.42 (s, 3H). | (Method 2) |

### Biological Activity

GPR52 Agonist Functional cAMP Assay

HEKf suspension cells were infected for 24 h with 0.1% v/v human GPR52 expressing BacMam virus, a modified baculovirus designed for mammalian gene expression. Following BacMam infection, cells were pelleted by centrifugation (335 g, 5 min), resuspended in cell freezing medium (Sigma) and frozen at −150° C. until required. On experiment day, 25 nL GPR52 compound dilutions, prepared in DMSO, were stamped onto proxiplates (PerkinElmer) by a LabCyte ECHO acoustic dispenser. Frozen cells were thawed and resuspended in assay stimulation buffer (Cisbio) containing 0.5 mM 3-iso-butyl-1-methylxanthine (IBMX, Sigma) to achieve a density of 2000 cells per well. 10 μl cells were added to assay plates using a Multidrop Combi Reagent Dispenser (ThermoFisher) before centrifugation (335 g, 1 min). Cells were incubated with compounds at 37° C. for 30 min prior to addition of cAMP detection reagents (HiRange cAMP kit, Cisbio) which were prepared according to the manufacturer's instructions. Plates were shaken for 1 h at room temperature before reading on a PHERAstar FS plate reader (BMG Labtech) using standard HTRF settings. HTRF ratios were obtained by dividing the acceptor emissions (665 nm) by the donor emissions (620 nm) and multiplying by 10,000. Data were normalised to DMSO (0%) and maximal 3-(2-(3-chloro-5-fluorobenzyl)benzo[b]thiophen-7-yl)-N-(2-methoxyethyl)benzamide (compound 7m in J. Med. Chem., 2014, 57, 5226) responses (100%) and fit to a 4-parameter logistical fit to generate agonist pEC₅₀s and maximal responses which are presented in Table 4 below.

TABLE 4
GPR52 pEC₅₀ data

| Ex. No. | pEC₅₀ average | Eₘₐₓ (%) |
|---|---|---|
| 1 | 7.5 | 97 |
| 2 | 7.2 | 86 |
| 3 | 7.3 | 104 |
| 4 | 8.0 | 95 |
| 5 | 7.6 | 96 |
| 6 | 6.3 | 82 |
| 7 | 6.7 | 83 |
| 8 | 6.5 | 75 |
| 9 | 6.3 | 89 |
| 10 | 8.1 | 90 |
| 11 | 7.2 | 86 |
| 12 | 7.3 | 92 |
| 13 | 7.6 | 89 |
| 14 | 8.0 | 103 |
| 15 | 6.9 | 95 |
| 16 | 7.0 | 95 |
| 17 | 5.9 | 96 |
| 18 | 7.1 | 82 |
| 19 | 6.2 | 73 |
| 20 | 6.8 | 101 |
| 21 | 5.9 | 100 |
| 22 | 6.7 | 91 |
| 23 | 6.8 | 90 |
| 24 | 6.5 | 95 |
| 25 | 6.8 | 89 |
| 26 | 7.1 | 95 |
| 27 | 7.1 | 90 |
| 28 | 7.0 | 88 |
| 29 | 5.6 | 57 |
| 30 | 6.1 | 77 |
| 31 | 6.9 | 95 |
| 32 | 6.4 | 61 |
| 33 | 5.5 | 58 |
| 34 | 6.0 | 54 |
| 35 | 5.5 | 40 |
| 36 | 6.3 | 92 |
| 36 | 5.8 | 72 |
| Isomer 1 | | |
| 36 | 6.6 | 94 |
| Isomer 2 | | |
| 37 | 6.9 | 93 |
| 38 | 6.2 | 55 |
| 39 | 7.4 | 90 |
| 40 | 7.0 | 93 |
| 40 | 7.5 | 95 |
| Isomer 1 | | |
| 40 | 6.9 | 83 |
| Isomer 2 | | |
| 41 | 6.1 | 90 |
| 42 | 6.1 | 90 |
| 43 | 6.6 | 86 |
| 44 | 5.7 | 74 |
| 45 | 6.2 | 75 |
| 46 | 6.7 | 90 |
| 47 | 7.1 | 81 |
| 48 | 6.6 | 83 |
| 49 | 7.4 | 98 |
| 50 | 7.4 | 95 |
| 51 | 6.9 | 95 |
| 52 | 5.8 | 69 |
| 53 | 8.2 | 100 |
| 54 | 6.4 | 63 |
| 55 | 6.4 | 69 |
| 56 | 6.9 | 98 |
| 57 | 6.1 | 44 |
| 58 | 7.0 | 79 |
| 59 | 7.1 | 79 |
| 60 | 6.4 | 83 |
| 61 | 7.3 | 99 |
| 62 | 7.1 | 94 |
| 63 | 6.9 | 70 |
| 64 | 7.7 | 94 |
| 65 | 7.7 | 94 |
| 66 | 7.6 | 100 |
| 67 | 5.8 | 53 |
| 68 | 7.4 | 91 |
| 69 | 6.9 | 60 |
| 70 | 6.5 | 74 |
| 71 | 7.7 | 91 |
| 72 | 5.9 | 83 |
| 73 | 7.3 | 88 |
| 74 | 6.3 | 53 |
| 75 | 8.4 | 98 |
| 76 | 7.1 | 94 |
| 77 | 7.8 | 95 |
| 78 | 7.7 | 94 |
| 79 | 7.9 | 99 |
| 80 | 8.0 | 97 |
| 81 | 8.0 | 70 |
| 82 | 8.0 | 101 |
| 83 | 7.6 | 93 |
| 84 | 8.6 | 95 |
| 85 | 7.2 | 96 |
| 86 | 8.5 | 102 |
| 87 | 8.2 | 95 |
| 88 | 8.3 | 100 |
| 89 | 8.2 | 92 |
| 90 | 7.7 | 87 |
| 91 | 8.5 | 97 |
| 92 | 6.2 | 74 |
| 93 | 7.0 | 83 |
| 94 | 6.4 | 78 |
| 95 | 8.2 | 98 |
| 96 | 6.1 | 94 |
| 97 | 5.6 | 90 |
| 98 | 6.8 | 92 |
| 99 | 6.9 | 95 |
| 100 | 6.2 | 92 |
| 101 | 6.0 | 61 |
| 102 | 8.0 | 98 |
| 103 | 6.0 | 68 |
| 104 | 7.0 | 94 |
| 105 | 7.2 | 97 |
| 106 | 7.5 | 86 |
| 107 | 7.9 | 99 |
| 108 | 7.1 | 80 |
| 109 | 7.9 | 81 |
| 110 | 6.3 | 74 |
| 111 | 7.2 | 87 |
| 112 | 6.4 | 77 |
| 113 | 7.8 | 95 |
| 114 | 7.4 | 93 |
| 115 | 7.6 | 91 |
| 116 | 8.3 | 95 |
| 117 | 6.6 | 83 |
| Isomer 1 | | |
| 117 | 7.6 | 103 |
| Isomer 2 | | |
| 118 | 6.9 | 94 |
| Isomer 1 | | |
| 118 | 8.1 | 101 |
| Isomer 2 | | |

### Pharmacokinetic Profiling

The pharmacokinetic profiles of Example 1 were assessed in male Sprague-Dawley rats via intravenous (IV) and oral (per os, PO) routes of delivery. Pharmacokinetic data (mean values±standard deviation) for Example 1 of the invention are detailed in Table 5.

Methods: For pharmacokinetic analysis, groups of three male Sprague-Dawley rats, ranging in weight between 200 and 230 g, were administered a single dose of Example 1 via IV or PO route, using the doses, dose volumes and vehicles specified in Table 5. Following dosing, blood samples were taken at several time points (pre-dose, 2 min, 5 min, 15 min, 30 min, 1 h, 3 h, 6 h, 12 h and 24 h for IV administration and pre-dose, 5 min, 15 min, 30 min, 1 hr, 2 h, 4 h, 8 h, 12 h and 24 h for PO administration) via serial tail vein bleeds, and centrifuged to separate plasma for analysis by LC-MS/MS. WinNonlin v8.2 statistics software (Pharsight Corporation, California, USA) was used to generate pharmacokinetic parameters using non-compartmental analysis.

### Brain Penetration

Plasma and brain exposure were evaluated to assess the brain penetration of Example 1, following IV administration. Unbound brain-to-plasma ratio (K_{p,uu}) was calculated, as detailed in Table 5, following experimental determination of binding in rat plasma and brain homogenate.

Methods: For brain penetration assessment, male Sprague-Dawley rats (n=3) were administered a single 1 mg/kg dose (formulated in 10% DMAC+10% Solutol HS15+80% saline) via the IV route. After 10 min post-dose, animals were sacrificed and brains extracted, homogenised with 2 volumes (w/v) of 50 mM sodium phosphate buffer (pH 7.4), and analysed by LC-MS/MS. Blood samples were removed at the same time point via tail vein bleed, centrifuged and the plasma analysed by LC-MS/MS.

To permit calculation of unbound brain-to-plasma ratio (K_{p,uu}), test compound binding in rat plasma and brain homogenate was performed, using Rapid Equilibrium Dialysis (RED). Test compound prepared in DMSO (1 μM final, 0.2% DMSO) was added to (i) undiluted male Sprague Dawley rat plasma and (ii) rat brain tissue homogenised with 2 volumes (w/v) of sodium phosphate buffer (pH 7.4), and dialysed against phosphate buffer for 5 h at 37° C. After incubation, the contents of each plasma/brain and buffer compartment were removed and mixed with equal volumes of control dialysed buffer or plasma/brain to maintain matrix similarity for analysis. Proteins were then precipitated by the addition of acetonitrile containing an analytical internal standard (allowing ratio of test compound versus internal standard to be derived), centrifuged and the supernatant removed for analysis by LC-MS/MS. Fraction unbound (Fᵤ) in plasma and brain was calculated using the following formula, then used to correct total plasma and brain concentrations to derive the K_{p,uu}:

Fraction bound=(Total plasma or brain ratio)−(Total buffer ratio)/Total plasma or brain ratio

Fraction unbound (Fᵤ,brain or plasma)=1−Fraction bound

For correction of dilution in brain binding assay:

Undiluted Fᵤ,brain=(1/dilution factor)/((1/Fv diluted))−1)+(1/dilution factor)

Where dilution factor=4

TABLE 5
Pharmacokinetic profiles and brain penetration of Example 1

| | Rat IV pharmacokinetics (n = 3) | | | |
|---|---|---|---|---|
| | Dose | Dose volume | | Clearance |
| | (mg/kg) | (mL/kg) | Dosing vehicle | (mL/min/kg) |
| Example 1 | 1 | 5 | 10% DMAC + 10% Solutol | 16.4 ± 1.0 |
| | | | HS15 + 80% saline | |

| | Rat PO pharmacokinetics (n = 3) | | | |
|---|---|---|---|---|
| | Dose | Dose volume | | Bioavailability |
| | (mg/kg) | (mL/kg) | Dosing vehicle | (%) |
| Example 1 | 3 | 5 | 10% DMAC + 10% Solutol | 40.4 ± 6.0 |
| | | | HS15 + 80% water | |

| | Rat IV brain penetration, 10 min (n = 3) | | | |
|---|---|---|---|---|
| | Dose | Dose volume | | |
| | (mg/kg) | (mL/kg) | Dosing vehicle | Kp, uu |
| Example 1 | 1 | 5 | 10% DMAC + 10% Solutol | 0.35 ± 0.03 |
| | | | HS15 + saline | |

### Amphetamine-Induced Locomotor Activity in Rat

The rat amphetamine-induced locomotor hyperactivity test is used to model the enhanced mesolimbic dopaminergic activity thought to contribute to psychosis in humans. The effects of Example 1 and FTBMT (J. Pharmacol. Exp. Ther., 2017, 363, 253) were evaluated in amphetamine-induced hyperlocomotion in the rat, using risperidone as a positive control. Male Sprague-Dawley rats (200-250 g) were housed in groups with a 12 h light/dark cycle (lights on at 07.00), at an ambient temperature of 21±2° C. and with standard pelleted diet and water ad libitum. Testing was carried out in the light phase. On the day of the experiment, animals were habituated to the locomotor cages for a 60-minute period.

Subsequently, they were dosed with vehicle, Example 1 (1, 3 and 10 mg/kg) or FTBMT (10 mg/kg) by the oral route or with risperidone by the subcutaneous route and returned to the appropriate locomotor cage. Example 1 was formulated in a vehicle of 10% DMAC, 10% solutol (Kolliphor HS15) and 80% water (v/v/v), FTBMT was formulated in 0.5% HPMC/0.1% Tween 80 in de-ionised water and risperidone was dissolved in acidified saline. Sixty minutes later, animals were dosed with vehicle (saline) or D-amphetamine by the subcutaneous route. Locomotor activity was assessed for a two hour period after D-amphetamine treatment. Data are back-transformed means, adjusted for differences between treatment groups in activity during the 30 minutes prior to treatment with test compound or vehicle (n=10-12). Analysis was by general linear model with treatment, cohort and rack as factors. SEMs were calculated from the residuals of the statistical model. Example 1 was compared to D-amphetamine by Williams' test. As shown in FIG. 1, treatment with Example 1 caused a reduction of the D-amphetamine-induced hyperlocomotor response which reached statistical significance at 10 mg/kg, but not 1 or 3 mg/kg. FTBMT showed a trend towards a reduced D-amphetamine-induced hyperlocomotor response but statistical significance was not reached.

### Subchronic PCP-Induced Reversal Learning Deficit in Rat

Subchronic treatment with PCP in the rat results in long-lasting neurobiological alterations and dysfunction in a range of behaviours relevant to neuropsychiatric disorders. Deficits in cognitive flexibility following subchronic PCP treatment have been demonstrated using the reversal learning paradigm (Neuropharmacology, 2017, 142, 41). The ability of Example 1 to attenuate the subchronic PCP induced cognitive deficit was assessed in female Lister Hooded rats in a manner comparable to the D, receptor agonist SKF-38393.

Adult female Lister Hooded rats were housed in groups of 5 under standard laboratory conditions under a 12 h light: dark cycle, lights on at 07.00 and food restricted to 90% of free-feeding bodyweight. Testing was carried out in the light phase. Rats underwent operant training firstly to press the left/right lever for a food reward in the presence or absence of a visual cue, and then to respond to the opposite contingency. The reversal learning task was introduced as follows. On the day before each reversal task session, a full 30-minute operant training session was conducted in order to ensure stable responding. In the initial task, animals were first exposed to a five-minute period during which the contingency (cue position relative to active lever) was the same as for the operant training session. This was followed by a 2-minute time-out period, which was signalled by the house-light being turned off. In the subsequent 5-minute period (reversal task), the contingency was reversed. Responses made on the correct and incorrect levers were recorded. Animals undertook several of these reversal learning task sessions before the beginning of the drug studies in order to ensure that they attained a stable level of performance in both phases of the task. In general, 4-6 sessions were required before this was achieved and in total, animals required about 6 weeks of training to reach this point. At this stage, training ceased and rats were treated with PCP (2 mg/kg, IP or vehicle 0.9% saline, IP) for 7 days followed by at least 7-days washout period.

On the day of testing, rats were randomly assigned to six treatment groups (n=8-10 per group) to receive an acute treatment with Example 1 (3, 10 and 30 mg/kg, PO), SKF-38393 (6 mg/kg, IP) or vehicle. Example 1 was dissolved in 10% DMAC, 10% Solutol (kolliphor H515) and 80% water and administered in a volume of 5 ml/kg via the oral route, 2 hours prior to testing. SKF-38393 was dissolved in 0.9% NaCl and administered IP in a volume of 1 ml/kg, 60 min prior to testing. In the initial phase, the reward contingency was the same as the previous day and in the reversal phase the reward contingency was reversed. Animals underwent 1 reversal session following the first phase (initial phase): i.e. initial phase 5 mins, time out of 2 mins, reversal phase 1 for 5 mins, end of experiment. Rats were randomized into a crossover design using Microsoft Excel 2010 random number generator whereby each rat within a cage receives a different treatment both within and between experiments.

Data are presented as mean total number of lever presses (+SEM) and percent correct response (+SEM). The percent correct response data were used to determine whether there was a significant effect of drugs on response accuracy; these data were Arc-Sin transformed prior to analysis. Statistical significance was assumed when P<0.05 and was determined as follows: one-way ANOVA (IBM SPSS Statistics 22) was performed in order to detect main effect of drug treatment in the initial and reversal tasks. Where a significant effect was detected, a post-hoc LSD t-test was performed in order to compare treatment groups versus the appropriate control in the reversal phase of the test.

Subchronic PCP treatment produced a significant (P<0.001) deficit in reversal learning, measured by a significant and selective reduction in percent correct responding in the reversal stage of the test with initial phase performance unaffected. Treatment with Example 1 at 10 and 30 mg/kg (but not 3 mg/kg), and the positive control compound, SKF-38393, significantly (P<0.05−P<0.001) reversed the deficit compared to the vehicle-treated subchronic PCP group (FIG. 2). There were no significant effects of Example 1 or SKF-38393 on the total number of lever presses (FIG. 3), indicating that any pro-cognitive effects were produced without a reduction in motivation or motor control.

### BRIEF DESCRIPTION OF THE FIGURES

FIG. 1: The effect of acute treatment with Example 1 (1, 3 and 10 mg/kg, PO) and risperidone (0.6 mg/kg, SC) on D-amphetamine-induced hyperlocomotor activity. Significant differences vs D-amphetamine are represented as *p<0.05, **p<0.01, ***p<0.001.

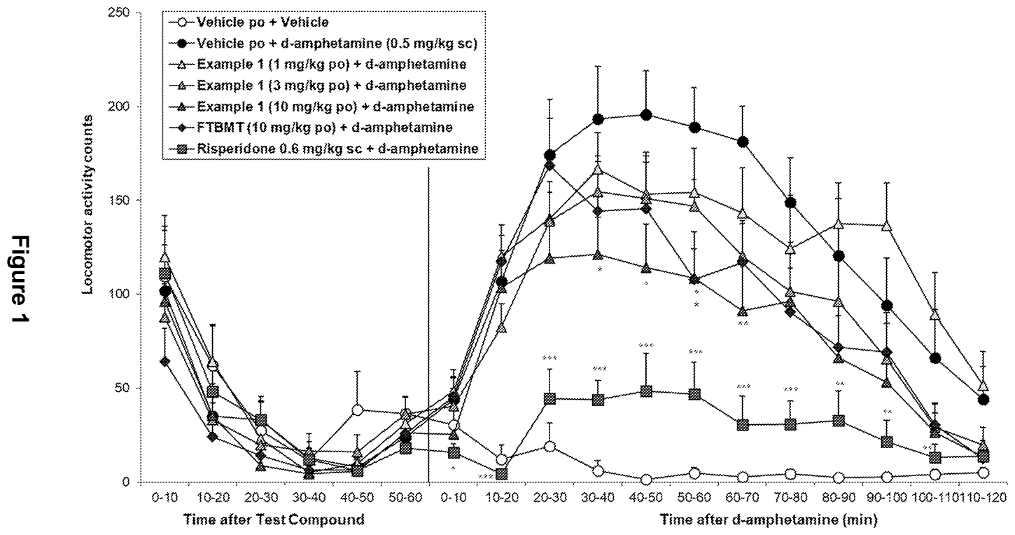

FIG. 2: The effect of acute treatment with Example 1 (3, 10 and 30 mg/kg, PO, 2 h prior to testing) and SKF38393 (6 mg/kg, IP, 60 min prior to testing) in subchronic PCP (scPCP) treated rats (2 mg/kg, IP twice daily for seven days, followed by at least a 7-day washout period) on performance in the reversal learning task. Data are shown as mean±s.e.m. % correct responding (n=8-10) and were analysed by ANOVA and post-hoc Student's t-test. Significant reduction in percent correct responding in the reversal phase compared with the vehicle group **P<0.01, ***P<0.001. Improvement in responding compared to scPCP alone in the reversal phase; #P<0.05−###P<0.001.

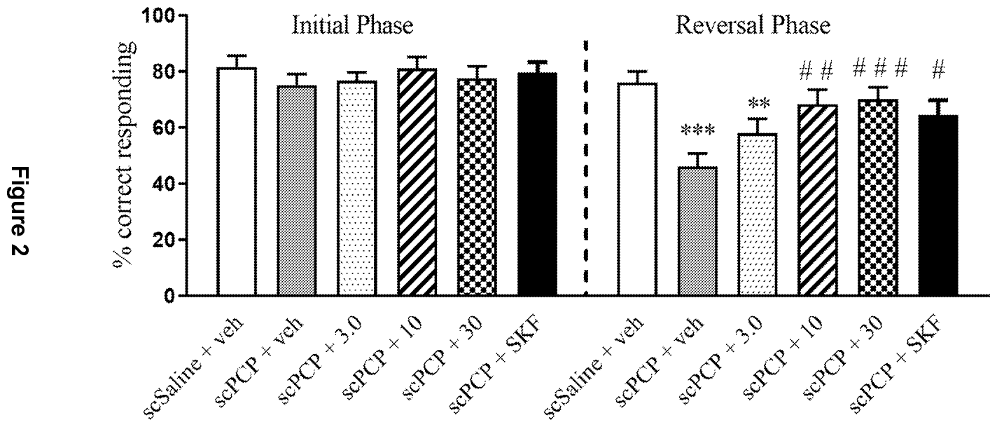

FIG. 3: The effect of acute treatment with Example 1 (3, 10 and 30 mg/kg, PO, 2 h prior to testing) and SKF38393 (6 mg/kg, IP, 60 min prior to testing) in subchronic PCP (scPCP) treated rats (2 mg/kg, IP) twice daily for seven days, followed by at least a 7-day washout period) on performance in the reversal learning task. Data are shown as the mean total number of lever presses, correct responses are shown as clear bars and incorrect responses are shown as shaded bars (n=8-10).

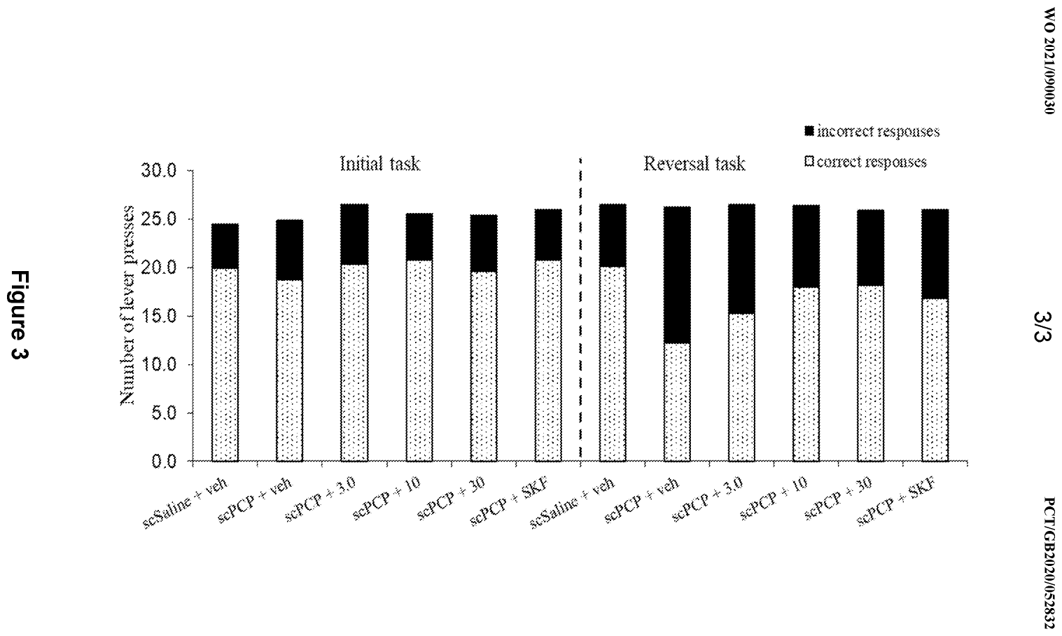

1. A compound of Formula (1): 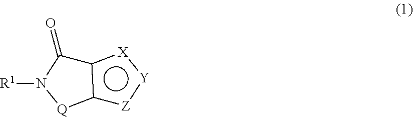
or a salt thereof, wherein;
X is N or CR²
Y is N, NR³ or CR²;
Z is N or NR³;
wherein one but not both of Y and Z is NR³;
Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵— and —OCR⁴R⁵CR⁶R⁷—;
R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring;
R² is H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof;
R³ is a group -V-L-W;
R⁴, R⁵, R⁶, R⁷, R⁸ and R⁹ are independently selected from H and C₁₋₃ alkyl;
wherein V is a 6-membered optionally substituted aryl or heteroaryl ring substituted with L at the meta-position relative to the position of attachment of R³ to the remainder of the molecule;
L is selected from CH₂, CHD, CD₂, CHF, CF₂, C═O, CHOH, O and NH;
and W is an optionally substituted monocyclic or polycyclic ring system.

2. The compound according to claim 1 wherein R³ is a group of the formula: 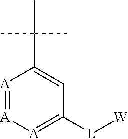
wherein, each A is independently N or CR¹⁰;
L is selected from CH₂, CHD, CD₂, CHF, CF₂, C═O, CHOH, O and NH;
R¹⁰ is selected from H, halo and C₁₋₃ alkyl optionally substituted with 1 to 6 fluorine atoms;
and W is either:
(i) a 6-membered optionally substituted aryl or heteroaryl ring;
or
(ii) a 9-10-membered optionally substituted heterobicyclic ring system, where one or more of the rings is aromatic.

3. The compound according to claim 1 or claim 2, wherein W is a 6-membered optionally substituted aryl or heteroaryl ring.

4. A compound according to claim 1 of Formula (1b): 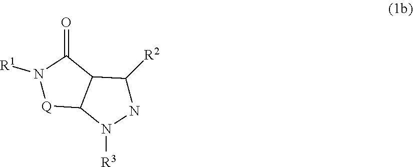
or a salt thereof, wherein;
Q is selected from —CR⁴R⁵—, —CR⁴R⁵CR⁶R⁷—, —CR⁴R⁵CR⁶R⁷CR⁸R⁹—, —CR⁴R⁵CR⁶R⁷O—, —OCR⁴R⁵— and —OCR⁴R⁵CR⁶R⁷—;
R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring;
R² is H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof;
R⁴, R⁵, R⁶, R¹, R⁸ and R⁹ are independently selected from H and C₁₋₃ alkyl;
R³ is a group of the formula: 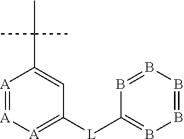
wherein, each A is independently N or CR¹⁰;
L is selected from CH₂, CHD, CD₂, CHF, CF₂, C═O, CHOH, O and NH;
each B is independently N, CR¹¹, CR¹², CR¹³, CR¹⁵ or CR¹⁶—R¹⁰ is selected from H, halo and C₁₋₃ alkyl optionally substituted with 1 to 6 fluorine atoms;
R¹¹, R¹², R¹³, R¹⁵ and R¹⁶ are independently selected from H, CN, SF₅, halo, a C1.6 saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃.

5. The compound according to claim 1, which is a compound of formula (2a), (2b), (2c), (2d), (2e), (2f) or (2g): 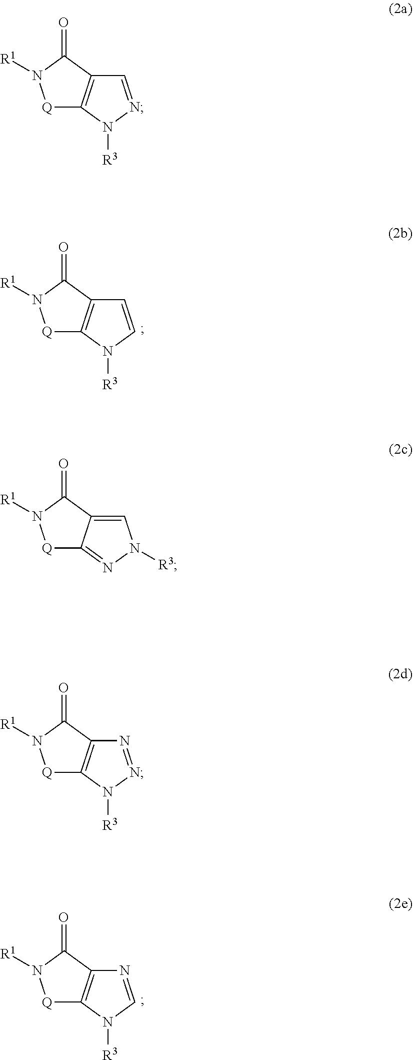 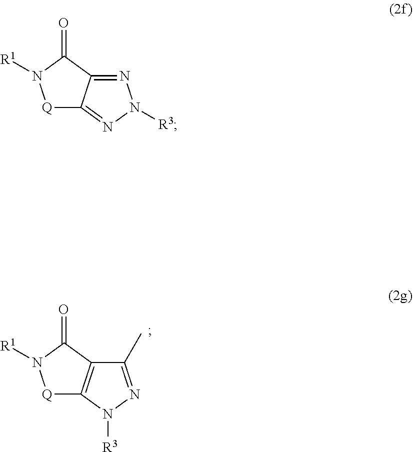
or a salt thereof.

6. The compound according to claim 1, which is a compound of formula (11a): 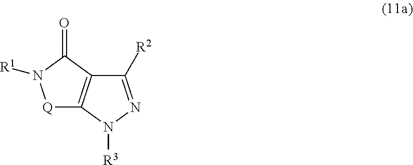
or a salt thereof.

7. The compound according to any one of claims 1 to 6 wherein R³ is a group of the formula: 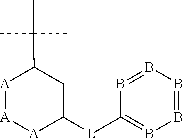
wherein, each A is independently N or CR¹⁰;
L is selected from CH₂, CHD, CD₂, CHF, CF₂, C═O, CHOH, O and NH;
each B is independently N, CR¹¹, CR¹², CR¹³, CR¹⁵ or CR¹⁶;
R¹⁰ is selected from H, halo and C₁₋₃ alkyl optionally substituted with 1 to 6 fluorine atoms;
R¹¹, R¹², R¹³, R¹⁵ and R¹⁶ are independently selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃.

8. The compound according to claim 7, wherein R³ is selected from the group consisting of:

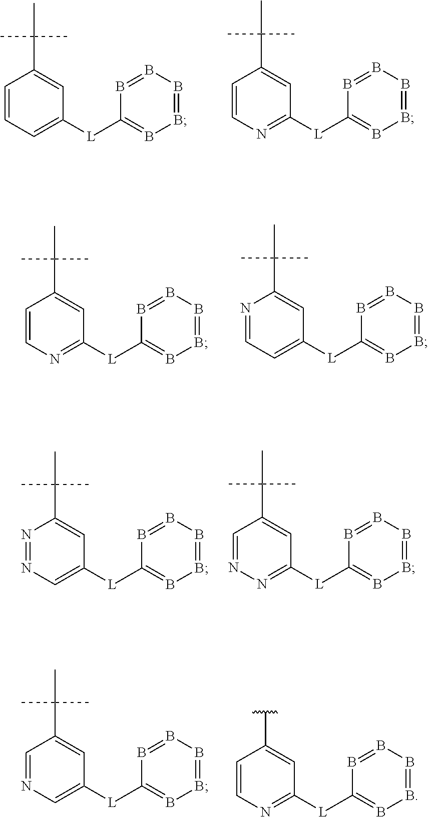

9. The compound according to any one of claims 1 to 8, wherein L is CH₂.

10. The compound according to any one of claims 7 to 9, wherein the group: 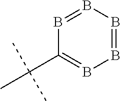
is selected from the group consisting of:

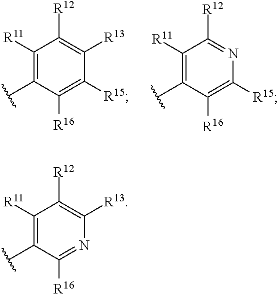

11. The compound according to any one of claims 1 to 10, wherein Q is selected from the group consisting of —CH₂CH₂—; —CH₂CH₂CH₂—; —CH₂—; —OCH₂—; —CH₂CH₂O—, —CH(CH₃)CH₂— and —CH₂CH(CH₃)—.

12. The compound according to claim 11, wherein Q is —CH₂CH₂—.

13. The compound according to claim 1 which is a compound of formula (13a), (13b), (13c), (13d), (13e), (13f), (13g), (13h), (13i) or (13j): 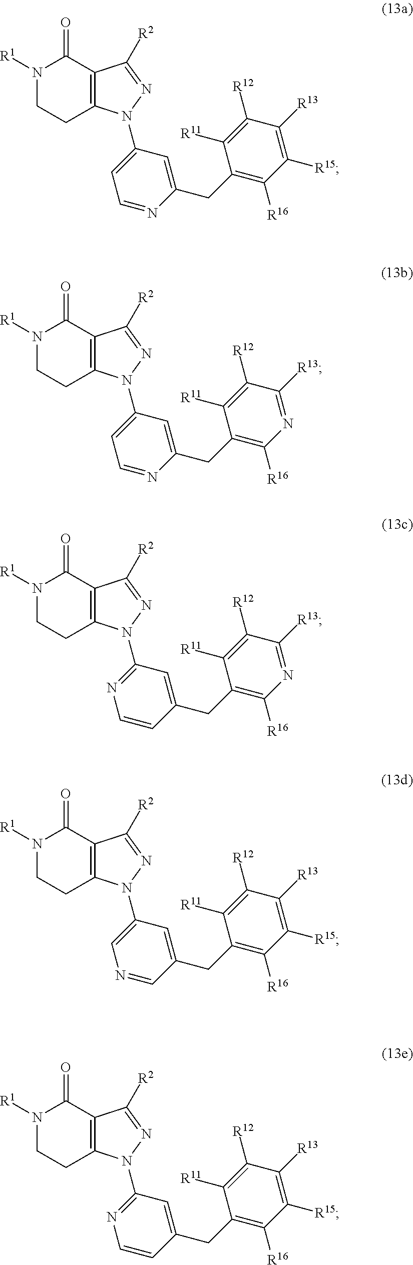 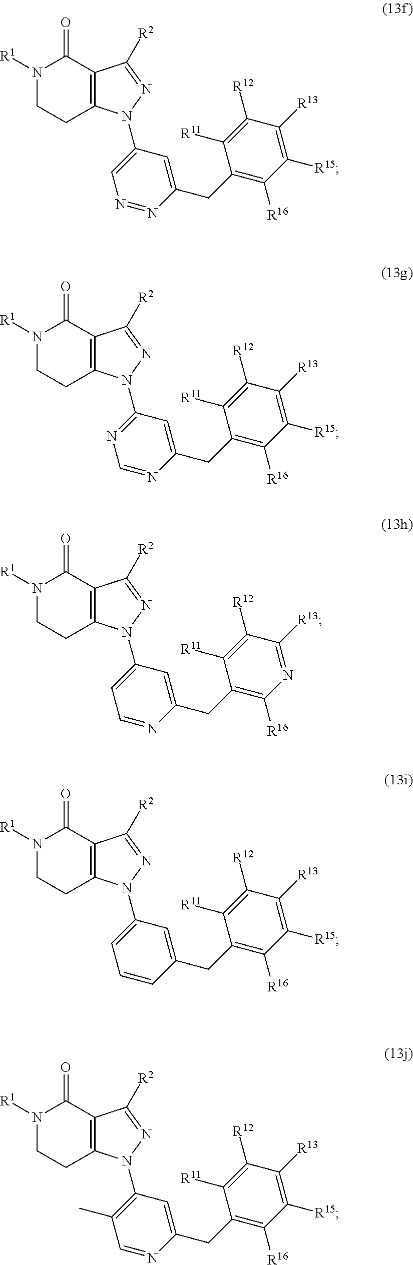
or a salt thereof, wherein;
R¹ is H, C₁₋₆ alkyl optionally substituted with 1 to 6 fluorine atoms, C₃₋₆ cycloalkyl optionally substituted with 1 to 6 fluorine atoms, a group —C(R¹⁴)₂C(R¹⁸)₂OR¹⁷ or a group —C(R¹⁴)₂C(R¹⁹)₂OH, wherein each R¹⁴ is independently H, F or C₁₋₃ alkyl optionally substituted with 1 to 6 fluorine atoms, R¹⁷ is C₁₋₃ alkyl optionally substituted with 1 to 6 fluorine atoms, each R¹⁸ is independently H, F or C₁₋₃ alkyl optionally substituted with 1 to 6 fluorine atoms and each R¹⁹ is independently H or C₁₋₃ alkyl optionally substituted with 1 to 6 fluorine atoms, and wherein R¹⁷ and one R¹⁴ may be joined to form an oxolane or oxetane ring;
R² is H, halo, CN, C₁₋₆ alkyl optionally substituted with OH or 1 to 6 fluorine atoms, C₁₋₆ alkoxy optionally substituted with OH or 1 to 6 fluorine atoms or C₃₋₆ cycloalkyl optionally substituted with OH or 1 to 6 fluorine atoms, wherein one atom of the C₁₋₆ alkyl, C₁₋₆ alkoxy or C₃₋₆ cycloalkyl group may be optionally replaced by a heteroatom selected from O, N, S and oxidised forms thereof;
R¹¹, R¹², R¹³, R¹⁵ and R¹⁶ are independently selected from H, CN, SF₅, halo, a C₁₋₆ saturated hydrocarbon group optionally substituted with 1 to 6 fluorine atoms wherein one atom of the C₁₋₆ saturated hydrocarbon group is optionally replaced by a heteroatom selected from O, N, S and oxidised forms thereof, OC₁₋₆ alkyl optionally substituted with 1 to 6 fluorine atoms, SO₂C₁₋₆ alkyl optionally substituted with 1 to 6 fluorine atoms and C₃₋₆ cycloalkyl optionally substituted with 1 to 6 fluorine atoms or optionally substituted with CF₃.

14. The compound according to claim 16 which is a compound of formula (6a): 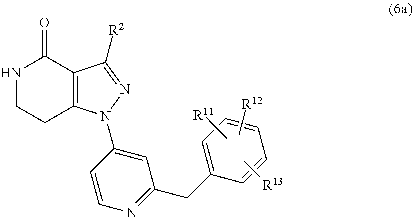
or a salt thereof.

15. The compound according to any one of claims 1 to 14, wherein R¹ is H, methyl, —CH₂CH₂OCH₃ or —CH₂CH₂OH.

16. The compound according to any one of claims 1 to 4 or 13 to 15 wherein R² is selected from H, methyl, F, Cl, CN, CF₃ and CH₂OH.

17. The compound according to claim 16 wherein R² is H or methyl.

18. The compound according to any one of claims 4 to 17, wherein R¹¹, R¹², R¹³, R¹⁵ and R¹⁶ are independently selected from H, CN, F, Cl, methyl, isopropyl, cyclopropyl, CF₃, CF₂H, OCF₂H, OMe and

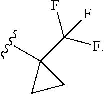

19. The compound according to claim 18, wherein R¹¹, R¹², R¹³, R¹⁵ and R¹⁶ are independently selected from H, F and CF₃.

20. The compound according to claim 1 which is selected from the group consisting of:
1-(2-(3-fluoro-5-(trifluoromethyl)benzyl)pyridin-4-yl)-1,5,6,7-tetrahydro-4H-pyrazolo[4,3-c]pyridin-4-one;
1-(4-(3-(trifluoromethyl)benzyl)pyridin-2-yl)-1,5,6,7-tetrahydro-4H-pyrazolo[4,3-c]pyridin-4-one;
5-methyl-1-(4-(3-(trifluoromethyl)benzyl)pyridin-2-yl)-1,5,6,7-tetrahydro-4H-pyrazolo[4,3-c]pyridin-4-one;
1-(4-(3-fluoro-5-(trifluoromethyl)benzyl)pyridin-2-yl)-1,5,6,7-tetrahydro-4H-pyrazolo[4,3-c]pyridin-4-one;
1-(4-(3-fluoro-5-(trifluoromethyl)benzyl)pyridin-2-yl)-5-methyl-1,5,6,7-tetrahydro-4H-pyrazolo[4,3-c]pyridin-4-one;
1-(4-((6-fluoro-5-methylpyridin-3-yl)methyl)pyridin-2-yl)-1,5,6,7-tetrahydro-4H-pyrazolo[4,3-c]pyridin-4-one;
1-(4-((6-fluoro-5-methylpyridin-3-yl)methyl)pyridin-2-yl)-5-methyl-1,5,6,7-tetrahydro-4H-pyrazolo[4,3-c]pyridin-4-one;
2-(4-(3-(trifluoromethyl)benzyl)pyridin-2-yl)-2,5,6,7-tetrahydro-4H-pyrazolo[4,3-c]pyridin-4-one;
2-(2-(3-fluoro-5-(trifluoromethyl)benzyl)pyridin-4-yl)-2,5,6,7-tetrahydro-4H-pyrazolo[4,3-c]pyridin-4-one;
1-(4-(3-fluoro-5-(trifluoromethyl)benzyl)pyridin-2-yl)-5,6,7,8-tetrahydropyrazolo[4,3-c]azepin-4(1H)-one;
1-(4-(3-(trifluoromethyl)benzyl)pyridin-2-yl)-5,6,7,8-tetrahydropyrazolo[4,3-c]azepin-4(1H)-one;
1-(2-(3-fluoro-5-(trifluoromethyl)benzyl)pyridin-4-yl)-5,6,7,8-tetrahydropyrazolo[4,3-c]azepin-4(1H)-one;
1-(4-(3-(trifluoromethyl)benzyl)pyridin-2-yl)-6,7-dihydro-1H-pyrazolo[4,3-f][1,4]oxazepin-4(5H)-one;
1-(4-(3-fluoro-5-(trifluoromethyl)benzyl)pyridin-2-yl)-6,7-dihydro-1H-pyrazolo[4,3-f][1,4]oxazepin-4(5H)-one;
1-(5-(3-fluoro-5-(trifluoromethyl)benzyl)pyridazin-3-yl)-1,5,6,7-tetrahydro-4H-pyrazolo[4,3-c]pyridin-4-one;
1-(5-(3-fluoro-5-(trifluoromethyl)benzyl)pyridin-3-yl)-1,5,6,7-tetrahydro-4H-pyrazolo[4,3-c]pyridin-4-one;
1-(6-(3-fluoro-5-(trifluoromethyl)benzyl)pyridazin-4-yl)-1,5,6,7-tetrahydro-4H-pyrazolo[4,3-c]pyridin-4-one;
1-{4-[3-fluoro-5-(trifluoromethyl)phenoxy]pyridin-2-yl}-1,5,6,7-tetrahydro-4H-pyrazolo[4,3-c]pyridin-4-one;
1-(2-(4-chlorobenzyl)pyridin-4-yl)-1,5,6,7-tetrahydro-4H-pyrazolo[4,3-c]pyridin-4-one;
1-(2-(3-chloro-5-fluorobenzyl)pyridin-4-yl)-1,5,6,7-tetrahydro-4H-pyrazolo[4,3-c]pyridin-4-one;
1-(2-(3,5-difluorobenzyl)pyridin-4-yl)-1,5,6,7-tetrahydro-4H-pyrazolo[4,3-c]pyridin-4-one;
1-(2-(3-(trifluoromethyl)benzyl)pyridin-4-yl)-1,5,6,7-tetrahydro-4H-pyrazolo[4,3-c]pyridin-4-one;
1-(2-(4-fluoro-3-(trifluoromethyl)benzyl)pyridin-4-yl)-1,5,6,7-tetrahydro-4H-pyrazolo[4,3-c]pyridin-4-one;
1-(2-(3,4,5-trifluorobenzyl)pyridin-4-yl)-1,5,6,7-tetrahydro-4H-pyrazolo[4,3-c]pyridin-4-one;
1-(2-(3-chloro-4-fluorobenzyl)pyridin-4-yl)-1,5,6,7-tetrahydro-4H-pyrazolo[4,3-c]pyridin-4-one;
1-(2-(3-fluoro-5-methylbenzyl)pyridin-4-yl)-1,5,6,7-tetrahydro-4H-pyrazolo[4,3-c]pyridin-4-one;
1-(2-(3-chloro-5-(trifluoromethyl)benzyl)pyridin-4-yl)-1,5,6,7-tetrahydro-4H-pyrazolo[4,3-c]pyridin-4-one;
1-(2-(3-methyl-5-(trifluoromethyl)benzyl)pyridin-4-yl)-1,5,6,7-tetrahydro-4H-pyrazolo[4,3-c]pyridin-4-one;
1-(2-(2-fluoro-3-(trifluoromethyl)benzyl)pyridin-4-yl)-1,5,6,7-tetrahydro-4H-pyrazolo[4,3-c]pyridin-4-one;
1-(2-(2-fluoro-5-(trifluoromethyl)benzyl)pyridin-4-yl)-1,5,6,7-tetrahydro-4H-pyrazolo[4,3-c]pyridin-4-one;
1-(2-(3,5-dichlorobenzyl)pyridin-4-yl)-1,5,6,7-tetrahydro-4H-pyrazolo[4,3-c]pyridin-4-one;
1-(2-(4-(trifluoromethyl)benzyl)pyridin-4-yl)-1,5,6,7-tetrahydro-4H-pyrazolo[4,3-c]pyridin-4-one;
3-((4-(4-oxo-4,5,6,7-tetrahydro-1H-pyrazolo[4,3-c]pyridin-1-yl)pyridin-2-yl)methyl)-5-(trifluoromethyl)benzonitrile;
1-(2-(3,5-bis(trifluoromethyl)benzyl)pyridin-4-yl)-1,5,6,7-tetrahydro-4H-pyrazolo[4,3-c]pyridin-4-one;
1-(2-(3-fluoro-5-(trifluoromethyl)benzoyl)pyridin-4-yl)-1,5,6,7-tetrahydro-4H-pyrazolo[4,3-c]pyridin-4-one;
1-(2-((3-fluoro-5-(trifluoromethyl)phenyl)(hydroxy)methyl)pyridin-4-yl)-1,5,6,7-tetrahydro-4H-pyrazolo[4,3-c]pyridin-4-one;
1-(2-(fluoro(3-fluoro-5-(trifluoromethyl)phenyl)methyl)pyridin-4-yl)-1,5,6,7-tetrahydro-4H-pyrazolo[4,3-c]pyridin-4-one;
1-(2-(difluoro(3-fluoro-5-(trifluoromethyl)phenyl)methyl)pyridin-4-yl)-1,5,6,7-tetrahydro-4H-pyrazolo[4,3-c]pyridin-4-one;
1-(2-((3-fluoro-5-(trifluoromethyl)phenyl)methyl-d₂)pyridin-4-yl)-1,5,6,7-tetrahydro-4H-pyrazolo[4,3-]pyridin-4-one;
1-(2-(3-fluoro-5-(trifluoromethyl)benzyl)pyridin-4-yl)-6-methyl-1,5,6,7-tetrahydro-4H-pyrazolo[4,3-c]pyridin-4-one;
1-(4-(3-(trifluoromethyl)benzyl)pyridin-2-yl)-5,6,7,8-tetrahydro-[1,2,3]triazolo[4,5-c]azepin-4(1H)-one;
1-(4-(3-(trifluoromethyl)benzyl)pyridin-2-yl)-1,5,6,7-tetrahydro-4H-[1,2,3]triazolo[4,5-c]pyridin-4-one;
2-(4-(3-(trifluoromethyl)benzyl)pyridin-2-yl)-2,5,6,7-tetrahydro-4H-[1,2,3]triazolo[4,5-c]pyridin-4-one;
5-methyl-2-(4-(3-(trifluoromethyl)benzyl)pyridin-2-yl)-2,5,6,7-tetrahydro-4H-[1,2,3]triazolo[4,5-c]pyridin-4-one;
2-(4-(3-(trifluoromethyl)benzyl)pyridin-2-yl)-5,6,7,8-tetrahydro-[1,2,3]triazolo[4,5-c]azepin-4(2H)-one;
1-(4-(3-fluoro-5-(trifluoromethyl)benzyl)pyridin-2-yl)-5,6,7,8-tetrahydro-[1,2,3]triazolo[4,5-c]azepin-4(1H)-one;
1-(4-(3-fluoro-5-(trifluoromethyl)benzyl)pyridin-2-yl)-1,7-dihydropyrazolo[4,3-d][1,2]oxazin-4(5H)-one;
1-(2-(3-fluoro-5-(trifluoromethyl)benzyl)pyridin-4-yl)-5,6-dihydropyrrolo[3,4-c]pyrazol-4(1H)-one;
1-(2-((3-fluoro-5-(trifluoromethyl)phenyl)methyl-d)pyridin-4-yl)-1,5,6,7-tetrahydro-4H-pyrazolo[4,3-c]pyridin-4-one;
1-(2-(3-fluoro-5-(trifluoromethyl)benzyl)pyridin-4-yl)-7-methyl-1,5,6,7-tetrahydro-4H-pyrazolo[4,3-c]pyridin-4-one;
1-(2-(3-fluoro-5-(trifluoromethyl)benzyl)pyridin-4-yl)-1,5,6,7-tetrahydro-4H-pyrrolo[3,2-c]pyridin-4-one;
1-(2-(3-fluoro-5-(trifluoromethyl)phenoxy)pyridin-4-yl)-1,5,6,7-tetrahydro-4H-pyrazolo[4,3-c]pyridin-4-one;
1-(2-(3-cyclopropyl-5-fluorobenzyl)pyridin-4-yl)-1,5,6,7-tetrahydro-4H-pyrazolo[4,3-c]pyridin-4-one;
1-(2-(3-fluoro-4-(trifluoromethyl)benzyl)pyridin-4-yl)-1,5,6,7-tetrahydro-4H-pyrazolo[4,3-c]pyridin-4-one;
1-(2-(4-methoxy-3-(trifluoromethyl)benzyl)pyridin-4-yl)-1,5,6,7-tetrahydro-4H-pyrazolo[4,3-c]pyridin-4-one;
1-(6-(3-fluoro-5-(trifluoromethyl)benzyl)pyrimidin-4-yl)-1,5,6,7-tetrahydro-4H-pyrazolo[4,3-c]pyridin-4-one;
1-(2-(3-methoxy-5-(trifluoromethyl)benzyl)pyridin-4-yl)-1,5,6,7-tetrahydro-4H-pyrazolo[4,3-c]pyridin-4-one;
1-(2-(4-chloro-3,5-difluorobenzyl)pyridin-4-yl)-1,5,6,7-tetrahydro-4H-pyrazolo[4,3-c]pyridin-4-one;
1-(2-(benzo[b]thiophen-5-ylmethyl)pyridin-4-yl)-1,5,6,7-tetrahydro-4H-pyrazolo[4,3-c]pyridin-4-one;
1-(2-(3-methoxy-4-(trifluoromethyl)benzyl)pyridin-4-yl)-1,5,6,7-tetrahydro-4H-pyrazolo[4,3-c]pyridin-4-one;
1-(2-(3-fluoro-5-(pentafluoro-λ⁶-sulfaneyl)benzyl)pyridin-4-yl)-1,5,6,7-tetrahydro-4H-pyrazolo[4,3-c]pyridin-4-one;
1-(2-(3-(difluoromethoxy)-5-fluorobenzyl)pyridin-4-yl)-1,5,6,7-tetrahydro-4H-pyrazolo[4,3-c]pyridin-4-one;
1-(2-(4-(difluoromethoxy)-3-fluorobenzyl)pyridin-4-yl)-1,5,6,7-tetrahydro-4H-pyrazolo[4,3-c]pyridin-4-one;
1-(2-(3,4-difluoro-5-(trifluoromethyl)benzyl)pyridin-4-yl)-1,5,6,7-tetrahydro-4H-pyrazolo[4,3-c]pyridin-4-one;
1-(2-(3-(difluoromethyl)-5-fluorobenzyl)pyridin-4-yl)-1,5,6,7-tetrahydro-4H-pyrazolo[4,3-c]pyridin-4-one;
1-(2-(3-chloro-4,5-difluorobenzyl)pyridin-4-yl)-1,5,6,7-tetrahydro-4H-pyrazolo[4,3-c]pyridin-4-one;
1-(2-(quinolin-3-ylmethyl)pyridin-4-yl)-1,5,6,7-tetrahydro-4H-pyrazolo[4,3-c]pyridin-4-one;
1-(2-(4-chloro-3-fluoro-5-(trifluoromethyl)benzyl)pyridin-4-yl)-1,5,6,7-tetrahydro-4H-pyrazolo[4,3-c]pyridin-4-one;
1-(2-(benzo[b]thiophen-2-ylmethyl)pyridin-4-yl)-1,5,6,7-tetrahydro-4H-pyrazolo[4,3-c]pyridin-4-one;
1-(2-(3,5-difluoro-4-(trifluoromethyl)benzyl)pyridin-4-yl)-1,5,6,7-tetrahydro-4H-pyrazolo[4,3-c]pyridin-4-one;
1-(2-(3-fluoro-5-isopropylbenzyl)pyridin-4-yl)-1,5,6,7-tetrahydro-4H-pyrazolo[4,3-c]pyridin-4-one;
1-(2-((5-fluoro-6-methoxypyridin-3-yl)methyl)pyridin-4-yl)-1,5,6,7-tetrahydro-4H-pyrazolo[4,3-c]pyridin-4-one;
1-(2-(3-chloro-4-fluoro-5-(trifluoromethyl)benzyl)pyridin-4-yl)-1,5,6,7-tetrahydro-4H-pyrazolo[4,3-c]pyridin-4-one;
1-(2-(3-fluoro-5-(1-(trifluoromethyl)cyclopropyl)benzyl)pyridin-4-yl)-1,5,6,7-tetrahydro-4H-pyrazolo[4,3-c]pyridin-4-one;
1-(2-(3-fluoro-5-(trifluoromethyl)benzyl)pyridin-4-yl)-3-methyl-1,5,6,7-tetrahydro-4H-pyrazolo[4,3-c]pyridin-4-one;
1-(2-((6-fluoro-5-methylpyridin-3-yl)methyl)pyridin-4-yl)-3-methyl-1,5,6,7-tetrahydro-4H-pyrazolo[4,3-c]pyridin-4-one;
3-methyl-1-(2-(3,4,5-trifluorobenzyl)pyridin-4-yl)-1,5,6,7-tetrahydro-4H-pyrazolo[4,3-c]pyridin-4-one;
3-methyl-1-(2-(3-(trifluoromethyl)benzyl)pyridin-4-yl)-1,5,6,7-tetrahydro-4H-pyrazolo[4,3-c]pyridin-4-one;
1-(2-(3-chloro-5-fluorobenzyl)pyridin-4-yl)-3-methyl-1,5,6,7-tetrahydro-4H-pyrazolo[4,3-c]pyridin-4-one;
1-(2-(3-chloro-4-fluorobenzyl)pyridin-4-yl)-3-methyl-1,5,6,7-tetrahydro-4H-pyrazolo[4,3-c]pyridin-4-one;
1-(2-(4-(difluoromethoxy)-3-fluorobenzyl)pyridin-4-yl)-3-methyl-1,5,6,7-tetrahydro-4H-pyrazolo[4,3-c]pyridin-4-one;
1-(5-(3-fluoro-5-(trifluoromethyl)benzyl)pyridin-3-yl)-3-methyl-1,5,6,7-tetrahydro-4H-pyrazolo[4,3-c]pyridin-4-one;
1-(4-((6-fluoro-5-methylpyridin-3-yl)methyl)pyridin-2-yl)-3-methyl-1,5,6,7-tetrahydro-4H-pyrazolo[4,3-c]pyridin-4-one;
1-(2-(3-chloro-4,5-difluorobenzyl)pyridin-4-yl)-3-methyl-1,5,6,7-tetrahydro-4H-pyrazolo[4,3-c]pyridin-4-one;
3-fluoro-5-((4-(3-methyl-4-oxo-4,5,6,7-tetrahydro-1H-pyrazolo[4,3-c]pyridin-1-yl)pyridin-2-yl)methyl)benzonitrile;
1-(2-(3,4-difluoro-5-(trifluoromethyl)benzyl)pyridin-4-yl)-3-methyl-1,5,6,7-tetrahydro-4H-pyrazolo[4,3-c]pyridin-4-one;
1-(2-(4-chloro-3-fluoro-5-(trifluoromethyl)benzyl)pyridin-4-yl)-3-methyl-1,5,6,7-tetrahydro-4H-pyrazolo[4,3-c]pyridin-4-one;
1-(2-(3-chloro-4-fluoro-5-(trifluoromethyl)benzyl)pyridin-4-yl)-3-methyl-1,5,6,7-tetrahydro-4H-pyrazolo[4,3-c]pyridin-4-one;
1-(2-(3-(difluoromethoxy)-5-fluorobenzyl)pyridin-4-yl)-3-methyl-1,5,6,7-tetrahydro-4H-pyrazolo[4,3-c]pyridin-4-one;
1-(2-(4-chloro-3,5-difluorobenzyl)pyridin-4-yl)-3-methyl-1,5,6,7-tetrahydro-4H-pyrazolo[4,3-c]pyridin-4-one;
1-(2-(3-(difluoromethyl)-5-fluorobenzyl)pyridin-4-yl)-3-methyl-1,5,6,7-tetrahydro-4H-pyrazolo[4,3-c]pyridin-4-one;
1-(2-((6-methoxypyridin-3-yl)methyl)pyridin-4-yl)-3-methyl-1,5,6,7-tetrahydro-4H-pyrazolo[4,3-c]pyridin-4-one;
1-(2-((5-fluoro-6-methoxypyridin-3-yl)methyl)pyridin-4-yl)-3-methyl-1,5,6,7-tetrahydro-4H-pyrazolo[4,3-c]pyridin-4-one;
1-(2-((6-(difluoromethoxy)pyridin-3-yl)methyl)pyridin-4-yl)-3-methyl-1,5,6,7-tetrahydro-4H-pyrazolo[4,3-c]pyridin-4-one;
1-(4-(3-fluoro-5-(trifluoromethyl)benzyl)pyridin-2-yl)-3-methyl-1,5,6,7-tetrahydro-4H-pyrazolo[4,3-c]pyridin-4-one;
3-fluoro-5-((4-(4-oxo-4,5,6,7-tetrahydro-1H-pyrazolo[4,3-c]pyridin-1-yl)pyridin-2-yl)methyl)benzonitrile;
1-(2-(3-fluoro-5-(trifluoromethyl)benzyl)pyridin-4-yl)-5,6,7,8-tetrahydro-[1,2,3]triazolo[4,5-c]azepin-4(1H)-one;
2-(4-(3-fluoro-5-(trifluoromethyl)benzyl)pyridin-2-yl)-2,5,6,7-tetrahydro-4H-[1,2,3]triazolo[4,5-c]pyridin-4-one;
1-(4-(3-fluoro-5-(trifluoromethyl)benzyl)pyridin-2-yl)-1,5,6,7-tetrahydro-4H-[1,2,3]triazolo[4,5-c]pyridin-4-one;
1-(2-(3-fluoro-5-(trifluoromethyl)benzyl)pyridin-4-yl)-1,5,6,7-tetrahydro-4H-[1,2,3]triazolo[4,5-c]pyridin-4-one;
2-(2-(3-fluoro-5-(trifluoromethyl)benzyl)pyridin-4-yl)-2,5,6,7-tetrahydro-4H-[1,2,3]triazolo[4,5-c]pyridin-4-one;
1-(4-(3-(difluoromethyl)-5-fluorobenzyl)pyridin-2-yl)-1,5,6,7-tetrahydro-4H-pyrazolo[4,3-c]pyridin-4-one;
1-(5-(3-fluoro-5-(trifluoromethyl)phenoxy)pyridin-3-yl)-1,5,6,7-tetrahydro-4H-pyrazolo[4,3-c]pyridin-4-one;
1-(2-(3-fluoro-5-(trifluoromethyl)benzyl)pyridin-4-yl)-5-(2-methoxyethyl)-1,5,6,7-tetrahydro-4H-pyrazolo[4,3-c]pyridin-4-one;
1-(2-(3-fluoro-5-(trifluoromethyl)benzyl)pyridin-4-yl)-5-(2-hydroxyethyl)-1,5,6,7-tetrahydro-4H-pyrazolo[4,3-c]pyridin-4-one;
1-(4-(3-fluoro-5-(trifluoromethyl)benzyl)pyridin-2-yl)-5-(2-methoxyethyl)-1,5,6,7-tetrahydro-4H-pyrazolo[4,3-c]pyridin-4-one;
1-(4-(3-fluoro-5-(trifluoromethyl)benzyl)pyridin-2-yl)-5-(2-hydroxyethyl)-1,5,6,7-tetrahydro-4H-pyrazolo[4,3-c]pyridin-4-one;
1-(2-(3-fluoro-5-(trifluoromethyl)benzyl)-5-methylpyridin-4-yl)-1,5,6,7-tetrahydro-4H-pyrazolo[4,3-c]pyridin-4-one;
1-(3-(3-fluoro-5-(trifluoromethyl)benzyl)phenyl)-1,5,6,7-tetrahydro-4H-pyrazolo[4,3-c]pyridin-4-one;
1-(2-((6-(difluoromethoxy)-5-fluoropyridin-3-yl)methyl)pyridin-4-yl)-1,5,6,7-tetrahydro-4H-pyrazolo[4,3-c]pyridin-4-one;
1-(2-((6-(difluoromethoxy)-5-fluoropyridin-3-yl)methyl)pyridin-4-yl)-3-methyl-1,5,6,7-tetrahydro-4H-pyrazolo[4,3-c]pyridin-4-one;
2-(2-(3-fluoro-5-(trifluoromethyl)benzyl)pyridin-4-yl)-6,7-dihydro-2H-pyrazolo[4,3-f][1,4]oxazepin-4(5H)-one;
1-(4-(3-fluoro-5-(trifluoromethyl)benzyl)pyridin-2-yl)-3-(hydroxymethyl)-1,5,6,7-tetrahydro-4H-pyrazolo[4,3-c]pyridin-4-one;
1-(2-(3-fluoro-5-(trifluoromethyl)benzyl)pyridin-4-yl)-3-(hydroxymethyl)-1,5,6,7-tetrahydro-4H-pyrazolo[4,3-c]pyridin-4-one;
1-(2-(3-fluoro-5-(trifluoromethyl)benzyl)pyridin-4-yl)-4-oxo-4,5,6,7-tetrahydro-1H-pyrazolo[4,3-c]pyridine-3-carbonitrile;
1-(2-(3-fluoro-5-(trifluoromethyl)benzyl)pyridin-4-yl)-3-(trifluoromethyl)-1,5,6,7-tetrahydro-4H-pyrazolo[4,3-c]pyridin-4-one;
1-(2-((3-fluoro-5-(trifluoromethyl)phenyl)(hydroxy)methyl)pyridin-4-yl)-3-methyl-1,5,6,7-tetrahydro-4H-pyrazolo[4,3-c]pyridin-4-one;
1-(4-((3-fluoro-5-(trifluoromethyl)phenyl)(hydroxy)methyl)pyridin-2-yl)-3-methyl-1,5,6,7-tetrahydro-4H-pyrazolo[4,3-c]pyridin-4-one;
or a salt thereof.

21. The compound according to any one of claims 1 to 20 having GPR52 receptor modulator activity.

22. The compound according to any one of claims 1 to 20 for use as a GPR52 receptor agonist.

23. A pharmaceutical composition comprising a compound as defined in any one of claims 1 to 22 and a pharmaceutically acceptable excipient.

24. The compound according to any one of claims 1 to 22 or composition according to claim 23 for use in medicine.

25. The compound according to any one of claims 1 to 22 or composition according to claim 23 for use in the treatment of psychiatric disorders; neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; depressive disorders; hypothalamic disorders; pituitary disorders; prolactin-related disorders; trauma- or stressor-related disorders; disruptive, impulse-control or conduct disorders; sleep-wake disorders; substance-related disorders; addictive disorders; behavioral disorders; hypofrontality; abnormalities in the tuberoinfundibular, mesolimbic, mesocortical, or nigrostriatal pathway; decreased activity in the striatum; cortical dysfunction; neurocognitive dysfunction or conditions or symptoms related thereto.

26. The compound or composition for use according to claim 25, wherein the disorder or symptom is selected from schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, anhedonia, agitation, Alzheimer's disease, Parkinson's disease, Huntington's disease, vascular dementia, Lewy body disease, frontotemporal dementia, Tourette's syndrome, hyperprolactinemia, pituitary adenoma, prolactinoma, craniopharyngioma, Cushing's disease, diabetes insipidus, non-functioning tumours, obesity, posttraumatic stress disorder (PTSD), akathisia and associated movements, athetosis, ataxia, ballismus, hemiballismus, chorea, choreoathetosis, dyskinesia, tardive dyskinesia, neuroleptic-induced dyskinesia, myoclonus, mirror movement disorder, paroxysmal kinesigenic dyskinesia, restless legs syndrome, spasms, stereotypic movement disorder, sterotypy, Tic disorder, tremor, Wilson's disease, schizotypal personality disorder, delusional disorder, brief psychotic disorder, schizophreniform disorder, schizoaffective disorder, substance- or medication-induced psychotic disorder, delusions, hallucinations, disorganized thinking, grossly disorganized or abnormal motor behavior, catatonia, major depressive disorder, bipolar I disorder, bipolar II disorder, cyclothymic disorder, substance- or medication-induced bipolar and related disorders, bipolar and related disorders due to another medical condition, separation anxiety disorder, selective mutism, specific phobia, social anxiety disorder, panic disorder, agoraphobia, generalized anxiety disorder, substance- or medication-induced anxiety disorder, anxiety disorders due to another medical condition, delirium, major neurocognitive disorder, minor neurocognitive disorder, amnesia, dementia, developmental coordination disorder, stereotypic movement disorder, a post-stroke effect, dentatorubral-pallidoluysian atrophy, diminished emotional expression, avolition, alogia and asociality.

27. The compound or composition for use according to claim 25, wherein the disorder or symptom is selected from schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, neurocognitive disorder, delirium, anhedonia, agitation, Alzheimer's disease, Parkinson's disease, Huntington's disease, vascular dementia, Lewy body disease, frontotemporal dementia, Tourette's syndrome, hyperprolactinemia, obesity, and posttraumatic stress disorder (PTSD).
